(19) United States
(12) Patent Application Publication
(10) Pub. No.: US 2024/0110166 A1
(43) Pub. Date: Apr. 4, 2024

(54) EDITING OF CCR5 RECEPTOR GENE TO PROTECT AGAINST HIV INFECTION

(71) Applicant: President and Fellows of Harvard College, Cambridge, MA (US)

(72) Inventors: Juan Pablo Maianti, Cambridge, MA (US); David R. Liu, Cambridge, MA (US)

(73) Assignee: President and Fellows of Harvard College, Cambridge, MA (US)

(21) Appl. No.: 18/484,381

(22) Filed: Oct. 10, 2023

Related U.S. Application Data
(63) Continuation of application No. 16/926,436, filed on Jul. 10, 2020, now Pat. No. 11,820,969.
Application No. 16/926,436 is a division of application No. 15/852,891, filed on Dec. 22, 2017, now Pat. No. 10,745,677.
(60) Provisional application No. 62/438,827, filed on Dec. 23, 2016.

(51) Int. Cl. C12N 9/22; A61P 31/18; C07K 14/715; C12N 9/78; C12N 15/10; C12N 15/113

(52) U.S. Cl. CPC C12N 9/22; A61P 31/18; C07K 14/7158; C12N 9/78; C12N 15/102; C12N 15/1138; C12Y 305/04001; C07K 2319/80; C12N 2310/20; C12N 2740/16062; C12Y 301/21

(57) ABSTRACT

Provided herein are systems, compositions, and methods of introducing protective and/or loss-of-function variants of CCR5 and CCR2. Variants may be introduced using a CRISPR/Cas9-based nucleobase editor or other guide nucleotide sequence-programmable DNA binding protein domain-based fusion protein described herein. Further provided herein are compositions and methods of preventing and treating conditions related to HIV infection and progression as well as to AIDS.

### RELATED APPLICATIONS

This application claims priority under 35 U.S.C. § 119(e) to U.S. provisional patent application. U.S. Ser. No. 62/438,827, filed Dec. 23, 2016, which is incorporated herein by reference.

### GOVERNMENT SUPPORT

This invention was made with government support under grant number GM065865, awarded by the National Institutes of Health (NIH). The government has certain rights in the invention.

### BACKGROUND

C-C chemokine receptor type 5 (also commonly known as CCR5 or CD195) is a protein found on the surface of white blood cells. CCR5 acts as a receptor for chemokines and has demonstrated involvement in several different disease states including, but not limited to, human immunodeficiency virus (HIV) and acquired immune deficiency syndrome (AIDS). Many strains of HIV, the virus that causes AIDS, initially use CCR5 to enter and infect host cells. A mutation known as CCR5-Δ32 in the CCR5 gene has been shown to protect those individuals that carry it against these strains of HIV. Loss-of-function CCR5 mutants have generated significant interest in the biotech and pharmaceutical industries in light of the widespread and devastating effects of HIV/AIDS (“HIV/AIDS Fact sheet Updated July 2016” from the World Health Organization). However, existing methods and technologies for creating CCR5 loss-of-function mutants in vivo have been ineffective due to the large number of cells that need to be modified. Other concerns involve off-target effects, genome instability, or oncogenic modifications that may be caused by genome-editing treatments.

### SUMMARY

Provided herein are systems, compositions, kits, and methods for modifying a polynucleotide (e.g. DNA) encoding a CCR5 protein to produce a loss-of-function CCR5 variant. Also provided are systems, compositions, kits, and methods for modifying a polynucleotide encoding a CCR2 protein to produce loss-of-function CCR2 mutants. The methodology relies on CRISPR/Cas9-based base-editing technology. The precise targeting methods described herein are superior to previously proposed strategies that create random indels in the CCR5 or CCR2 genomic locus using engineered nucleases. The methods also have a more favorable safety profile, due to low probability of off-target effects. Thus, the base editing methods described herein have a low impact on genomic stability, including oncogene activation or tumor suppressor inactivation. The loss-of-function CCR5 and/or CCR2 variants generated have a protective function against HIV infection (including prevention of HIV infection), decrease one or more sympotoms of HIV infection, halt or delay progression of HIV to AIDS, and/or decrease one or more sympotoms of AIDS.

Some aspects of the present disclosure provide a method of editing a polynucleotide encoding a C-C chemokine receptor type five (CCR5) protein, the method comprising contacting the CCR5-encoding polynucleotide with: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in the CCR5-encoding polynucleotide; wherein the contacting results in deamination of the the target C base is by the fusion protein, resulting in a cytosine-guanine pair (C:G) to thymine-adenine pair (T:A) change in the CCR5-encoding polynucleotide. This may occur in any manner, and is not bound by any particular theory.

In one embodiment, the guide nucleotide sequence-programmable DNA binding protein domain is selected from the group consisting of: a nuclease inactive Cas9 (dCas9) domain, a nuclease inactive Cpf1 domain, a nuclease inactive Argonaute domain, and variants and combinations thereof. As a set of non limiting examples, any of the fusion proteins described herein that include a Cas9 domain, can use another guide nucleotide sequence-programmable DNA binding protein, such as CasX, CasY, Cpf1, C2c1, C2c2, C2c3, and Argonaute, in place of the Cas9 domain. Guide nucleotide sequence-programmable DNA binding protein include, without limitation, Cas9 (e.g., dCas9 and nCas9), CasX, CasY. Cpf1, C2c1, C2c2, C2C3, Argonaute, and any of suitable protein described herein.

In another embodiment, the guide nucleotide sequence-programmable DNA-binding protein domain comprises a nuclease inactive Cas9 (dCas9) domain. In some embodiments, the amino acid sequence of the dCas9 domain comprises mutations corresponding to D10A and/or H840A mutation(s) in SEQ ID NO: 1. In another embodiment, the amino acid sequence of the dCas9 domain comprises a mutation corresponding to a D10A mutation in SEQ ID NO: 1, and wherein the dCas9 domain comprises a histidine at the position corresponding to amino acid 840 of SEQ ID NO: 1.

In certain embodiments, the guide nucleotide sequence-programmable DNA-binding protein domain comprises a nuclease inactive Cpf1 (dCpf1) domain. In some embodiments, the dCpf1 domain is from a species of Acidaminococcus or Lachnospiraceae. In an embodiment, the guide nucleotide sequence-programmable DNA-binding protein domain comprises a nuclease inactive Argonaute (dAgo) domain. In a further embodiment, the dAgo domain is from Natronobacterium gregoryi.

As a set of non limiting examples, any of the fusion proteins described herein that include a Cas9 domain can use another guide nucleotide sequence-programmable DNA binding protein, such as CasX, CasY, Cpf1, C2c1, C2c2, C2c3, and Argonaute, in place of the Cas9 domain. These may be nuclease inactive variants of the proteins. Guide nucleotide sequence-programmable DNA binding protein include, without limitation, Cas9 (e.g., dCas9 and nCas9), saCas9 (e.g., saCas9d, saCas9n, saKKH Cas9), CasX, CasY, Cpf1, C2c1, C2c2, C2C3, Argonaute, and any of suitable protein described herein. In some embodiments, the fusion protein described herein comprises a Gam protein, a guide nucleotide sequence-programmable DNA binding protein, and a cytidine deaminase domain.

In some embodiments, the cytosine deaminase domain comprises an apolipoprotein B mRNA-editing complex (APOBEC) family deaminase. In an embodiment, the cytosine deaminase is selected from the group consisting of APOBEC1, APOBEC2, APOBEC3A, APOBEC3B, APOBEC3C, APOBEC3D, APOBEC3F, APOBEC3G deaminase. APOBEC3H deaminase, APOBEC4 deaminase, activation-induced deaminase (AID), and pmCDA1. In an embodiment, the cytosine deaminase comprises an amino acid sequence of any one of SEQ ID NOs: 270-292.

In some embodiments, the fusion protein of (a) further comprises a uracil glycosylase inhibitor (UGI) domain. In certain embodiments, the cytosine deaminase domain is fused to the N-terminus of the guide nucleotide sequence-programmable DNA-binding protein domain. In an embodiment, the UGI domain is fused to the C-terminus of the guide nucleotide sequence-programmable DNA-binding protein domain.

In some embodiments, the cytosine deaminase and the guide nucleotide sequence-programmable DNA-binding protein domain are fused via an optional linker. In another embodiment, the UGI domain is fused to the dCas9 domain via an optional linker.

In certain embodiments, the fusion protein comprises the structure NH₂-[cytosine deaminase domain]-[optional linker sequence]-[guide nucleotide sequence-programmable DNA-binding protein domain]-[optional linker sequence]-[UGI domain]-COOH.

In some embodiments, the linker comprises (GGGS)ₙ (SEQ ID NO: 303), (GGGGS)ₙ (SEQ ID NO: 304), (G)ₙ, (EAAAK)ₙ (SEQ ID NO: 305), (GGS)ₙ, SGSETPGTSESATPES (SEQ ID NO: 306), or (XP)ₙ motif, or a combination of any of these, wherein n is independently an integer between 1 and 30, and wherein X is any amino acid. In an embodiment, the linker comprises the amino acid sequence SGSETPGTSESATPES (SEQ ID NO: 306). In another embodiment, the linker is (GGS)ₙ, and wherein n is 1, 3, or 7.

In certain embodiments, the fusion protein comprises the amino acid sequence of any one of SEQ ID NO: 293-302.

In an embodiment, the polynucleotide encoding the CCR5 protein comprises a coding strand and a complementary strand. In some embodiments, the polynucleotide encoding the CCR5 protein comprises a coding region and a non-coding region. In an embodiment, the C to T change occurs in the coding sequence of the CCR5-encoding polynucleotide. In some embodiments, the C to T change leads to a mutation in the CCR5 protein.

In some embodiments, the mutation in the CCR5 protein is a loss-of-function mutation. In certain embodiments, the mutation is selected from the mutations listed in Tables 1-10. In one embodiment, the guide nucleotide sequence is selected from the guide nucleotide sequences listed in Tables 3-5 and 8-10. In certain embodiments, the loss-of-function mutation introduces a premature stop codon in the CCR5 coding sequence that leads to a truncated or non-functional CCR5 protein. In certain embodiments, the premature stop codon is TAG (Amber), TGA (Opal), or TAA (Ochre).

In some embodiments, the premature stop codon is generated from a CAG to TAG change via the deamination of the first C on the coding strand. In certain embodiments, the premature stop codon is generated from a CGA to TGA change via the deamination of the first C on the coding strand. In an embodiment, the premature stop codon is generated from a CAA to TAA change via the deamination of the first C on the coding strand. In certain embodiments, the premature stop codon is generated from a TGG to TAG change via the deamination of the second C on the complementary strand. In an embodiment, the premature stop codon is generated from a TGG to TGA change via the deamination of the third C on the complementary strand. In an embodiment, the premature stop codon is generated from a TGG to TAA change via the deamination of the second C and third C on the complementary strand. In another embodiment, the premature stop codon is generated from a CGG to TAG or CGA to TAA change via the deamination of C on the coding strand and the deamination of C on the complementary strand.

In some embodiments, the guide nucleotide sequence is selected from the guide nucleotide sequences (SEQ ID NO: 381-657) listed in Table 3, Table 4, Table 5. Table 8, or Table 9. In certain embodiments, tandem premature stop codons are introduced. In one embodiment, the mutation is selected from the group consisting of: Q186X/Q188X, Q277X/Q288X, Q328X/Q329X, Q329X/R334X, or R341X/Q346X. In certain embodiments, the guide nucleotide sequence is selected from the group consisting of: SEQ ID NOs: 381-657. In some embodiments, two guide nucleotides are selected from SEQ ID NOs: 381-657. In some embodiments, three or more guide nucleotides are selected from SEQ ID NOs: 381-657.

In some embodiments, the loss-of-function mutation destabilizes CCR5 protein folding. In certain embodiments, the loss-of-function mutation is selected from the mutations listed in Tables 1-9. In specific embodiments, the guide nucleotide sequence is selected from the guide nucleotide sequences listed in Tables 3-5 and 8-9 (SEQ ID NO: 381-657).

In some embodiments, the C to T change modifies a splicing site in the non-coding region of the CCR5-encoding polynucleotide. In one embodiment, the C to T change modifies at an intron-exon junction. In another embodiment, the C to T change modifies a splicing donor site. In another embodiment, the C to T change modifies a splicing acceptor site. In certain embodiments, the C to T changes occurs at a C base-paired with the G base in a start codon (AUG). In some embodiments, the C to T change prevents CCR5 mRNA maturation or abrogates CCR5 expression.

In some embodiments, the C to T change is selected from the C to T changes listed in Table 2, 8, or 9. In certain embodiments, the guide nucleotide sequence is selected from the guide nucleotide sequences (SEQ ID NOs: 577-657) listed in Tables 8 and 9.

In some embodiments, the C to T change results in a codon change in the CCR5-encoding polynucleotide listed in Table 7. In certain embodiments, a PAM sequence is located 3′ of the C being changed. In certain embodiments, a PAM sequence is located 5′ of the C being changed. In specific embodiments, the PAM sequence is selected from the group consisting of: NGG, NGAN, NGNG, NGAG, NGCG, NNGRRT, NGRRN, NNNRRT, NNNGATT, NNAGAA, NAAAC, NNT, NNNT, and YNT, wherein Y is pyrimidine, R is purine, and N is any nucleobase.

In some embodiments, no PAM sequence is located 3′ of the C being changed. In some embodiments, no PAM sequence is located 5′ of the C being changed. In certain embodiments, no PAM sequence is located 5′ or 3′ of the C being changed. In some embodiments, at least 1, 2, 3, 4, 5, 6, 7, 8, 9, or 10 mutations are introduced into the CCR5-encoding polynucleotide. In certain embodiments, the guide nucleotide sequence is RNA (guide RNA or gRNA). In some embodiments, the guide nucleotide sequence is ssDNA (guide DNA or gDNA).

In some aspects, the disclosure provides a method of editing a polynucleotide encoding a C-C chemokine receptor type 2 (CCR2) protein, the method comprising contacting the CCR2-encoding polynucleotide with: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in the CCR2-encoding polynucleotide, wherein the contacting results in the deamination of the target C base by the fusion protein, resulting in a cytosine-guanine (C:G) to thymine-adenine pair (T:A) change in the CCR2-encoding polynucleotide. In some embodiments, the fusion protein of (i) comprises a Gam protein.

In some embodiments, the C to T change is in the coding sequence of the CCR2-encoding polynucleotide. In some embodiments, the C to T change leads to leads to a mutation in the CCR2 protein.

In some embodiments, the mutation in the CCR2 protein is a loss-of-function mutation. In certain embodiments, the mutation is selected from the mutations listed in Table 1.

In certain embodiments, the method is carried out in vitro. In some embodiments, the method is carried out in a cultured cell. In some embodiments, the method is carried out in vivo. In other embodiments, the method is carried out ex vivo.

In certain embodiments, the method is carried out in a mammal. In some embodiments, the mammal is a rodent. In some embodiments, the mammal is a primate. In some embodiments, the mammal is human.

In some aspects, the disclosure provides a method of editing a polynucleotide encoding a C-C chemokine receptor type five (CCR2) protein, the method comprising contacting the CCR2-encoding polynucleotide with: (i) a fusion protein comprising: (a) a guide nucleotide sequence-programmable DNA binding protein domain; and (b) a cytosine deaminase domain; and (ii) a guide nucleotide sequence targeting the fusion protein of (i) to a target cytosine (C) base in the CCR2-encoding polynucleotide; wherein the target C base is deaminated by the fusion protein, resulting in a cytosine-guanine pair (C:G) to thymine-adenine pair (T:A) change in the CCR2-encoding polynucleotide. In some embodiments, the fusion protein of (i) comprises a Gam protein.

In some embodiments, the guide nucleotide sequence-programmable DNA binding protein domain is selected from the group consisting of: a nuclease inactive Cas9 (dCas9) domain, a nuclease inactive Cpf1 domain, a nuclease inactive Argonaute domain, and variants and combinations thereof. In certain embodiments, the guide nucleotide sequence-programmable DNA-binding protein domain comprises a nuclease inactive Cas9 (dCas9) domain.

In some embodiments, the amino acid sequence of the dCas9 domain comprises mutations corresponding to D10A and/or H840A mutation(s) in SEQ ID NO: 1. In specific embodiments, the amino acid sequence of the dCas9 domain comprises a mutation corresponding to a D10A mutation in SEQ ID NO: 1, and wherein the dCas9 domain comprises a histidine at the position corresponding to amino acid 840 of SEQ ID NO: 1. In some embodiments, the guide nucleotide sequence-programmable DNA-binding protein domain comprises a nuclease inactive Cpf1 (dCpf1) domain. In a specific embodiment, the dCpf1 domain is from a species of Acidaminococcus or Lachnospiraceae. In some embodiments, the guide nucleotide sequence-programmable DNA-binding protein domain comprises a nuclease inactive Argonaute (dAgo) domain. In an embodiment, the dAgo domain is from Natronobacterium gregoryi.

As a set of non limiting examples, any of the fusion proteins described herein that include a Cas9 domain can use another guide nucleotide sequence-programmable DNA binding protein, such as CasX, CasY, Cpf1, C2c1, C2c2, C2c3, and Argonaute, in place of the Cas9 domain. These may be nuclease inactive variants of the proteins. Guide nucleotide sequence-programmable DNA binding protein include, without limitation, Cas9 (e.g., dCas9 and nCas9), saCas9 (e.g., saCas9d, saCas9n, and saKKH Cas9), CasX, CasY, Cpf1, C2c1, C2c2, C2C3. Argonaute, and any of suitable protein described herein. In some embodiments, the fusion protein described herein comprises a Gam protein, a guide nucleotide sequence-programmable DNA binding protein, and a cytidine deaminase domain.

In some embodiments, the cytosine deaminase domain comprises an apolipoprotein B mRNA-editing complex (APOBEC) family deaminase. In specific embodiments, the cytosine deaminase is selected from the group consisting of APOBEC1, APOBEC2, APOBEC3A, APOBEC3B, APOBEC3C. APOBEC3D, APOBEC3F, APOBEC3G deaminase, APOBEC3H deaminase, APOBEC4 deaminase, activation-induced deaminase (AID), and pmCDA1. In an embodiment, the cytosine deaminase comprises an amino acid sequence of any one of SEQ ID NOs: 270-292.

In some embodiments, the fusion protein of (a) further comprises a uracil glycosylase inhibitor (UGI) domain. In certain embodiments, the cytosine deaminase domain is fused to the N-terminus of the guide nucleotide sequence-programmable DNA-binding protein domain. In specific embodiments, the UGI domain is fused to the C-terminus of the guide nucleotide sequence-programmable DNA-binding protein domain. In some embodiments, the cytosine deaminase and the guide nucleotide sequence-programmable DNA-binding protein domain are fused via an optional linker. In an embodiment, the UGI domain is fused to the dCas9 domain via an optional linker.

In certain embodiments, the fusion protein comprises the structure NH₂-[cytosine deaminase domain]-[optional linker sequence]-[guide nucleotide sequence-programmable DNA-binding protein domain]-[optional linker sequence]-[UGI domain]-COOH.

In some embodiments, the linker comprises (GGGS)ₙ (SEQ ID NO: 303), (GGGGS)ₙ (SEQ ID NO: 304), (G)ₙ, (EAAAK)ₙ (SEQ ID NO: 305), (GGS)ₙ, SGSETPGTSESATPES (SEQ ID NO: 306), or (XP)ₙ motif, or a combination of any of these, wherein n is independently an integer between 1 and 30, and wherein X is any amino acid. In an embodiment, linker comprises the amino acid sequence SGSETPGTSESATPES (SEQ ID NO: 306). In some embodiments, the linker is (GGS)ₙ, and wherein n is 1, 3, or 7.

In some aspects, the instant disclosure provides a composition comprising: (i) a fusion protein comprising: (a) a guide nucleotide sequence-programmable DNA binding protein domain; and (b) a cytosine deaminase domain; and (ii) a guide nucleotide sequence targeting the fusion protein of (i) to a polynucleotide encoding a C-C chemokine receptor type five (CCR5) protein. In some embodiments, the fusion protein of (i) comprises a Gam protein.

In some aspects, the instant disclosure provides a composition comprising: (i) a fusion protein comprising: (a) a guide nucleotide sequence-programmable DNA binding protein domain; and (b) a cytosine deaminase domain; and (ii) a guide nucleotide sequence targeting the fusion protein of (i) to a polynucleotide encoding a C-C chemokine receptor type two (CCR2) protein. In some embodiments, the fusion protein of (i) comprises a Gam protein.

In some aspects, the instant disclosure provides a composition comprising: (i) a fusion protein comprising: (a) a guide nucleotide sequence-programmable DNA binding protein domain; and (b) a cytosine deaminase domain; (ii) a guide nucleotide sequence targeting the fusion protein of (i) to a polynucleotide encoding a C-C chemokine receptor type five (CCR5) protein; and (iii) a guide nucleotide sequence targeting the fusion protein of (i) to a polynucleotide encoding a C-C chemokine receptor type 2 (CCR2) protein. In some embodiments, the fusion protein of (i) comprises a Gam protein.

In some embodiments, the guide nucleotide sequence of (ii) is selected from SEQ ID NOs: 381-657.

In certain embodiments, the composition further comprises a pharmaceutically acceptable carrier.

In some embodiments, the instant disclosure provides a method of reducing the binding of gp120 and CCR5 in a subject, the method comprising administering to a subject in need thereof a therapeutically effective amount of a composition of the instant disclosure.

In some embodiments, the instant disclosure provides a method of reducing virus binding to CCR5 in a subject, the method comprising administering to a subject in need thereof a therapeutically effective amount of the composition of the instant disclosure.

In some embodiments, the instant disclosure provides a method of reducing viral infection in a subject, the method comprising administering to a subject in need thereof a therapeutically effective amount of a composition of the instant disclosure.

In some embodiments, the instant disclosure provides a method of reducing functional CCR5 receptors on a cell in a subject, the method comprising administering to a subject in need thereof a therapeutically effective amount of the composition of the instant disclosure.

In some embodiments, the cell is selected from the group consisting of: macrophage, dendritic cell, memory T cell, endothelial cell, epithelial cell, vascular smooth muscle cell, fibroblast, microglia, neuron, and astrocyte.

In some embodiments, the instant disclosure provides a treating a condition, the method comprising administering to a subject in need thereof a therapeutically effective amount of a composition provided by the instant disclosure, wherein the condition is human immunodeficiency virus (HIV) infection, acquired immune deficiency syndrome (AIDS), an immunologic disease, or a combination thereof.

In one embodiment, the condition is human immunodeficiency virus (HIV) infection.

In some embodiments, the instant disclosure provides a method of preventing a condition, the method comprising administering to a subject in need thereof a therapeutically effective amount of a composition provided in the instant disclosure, wherein the condition is human immunodeficiency virus (HIV) infection, acquired immune deficiency syndrome (AIDS), an immunologic disease, or a combination thereof.

In certain embodiments, the condition is human immunodeficiency virus (HIV) infection.

In some embodiments, the instant disclosure provides a kit comprising a composition provided in the instant disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims. The details of certain embodiments of the disclosure are set forth in the Detailed Description of Certain Embodiments, as described below. Other features, objects, and advantages of the presented compositions and methods will be apparent from the Definitions, Examples, Figures. and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1 depicts a CCR5 protein structure which shows HIV-protective variants (C20S, C101X, G106R, C178R, Δ32, R223Q, C269F, and FS299) that can be replicated or imitated using genome/base-editing with APOBEC1-Cas9 tools (Tables 1-10). Arrows indicate disulfide bridges that can be disrupted by mutation of cysteine residues using base-editing reactions (TGT=→TAT or TGC→TAC, Table 3). Grey shading with a double ring around the residue indicates small/hydrophobic residues in a transmembrane domain (TM) that can be targeted for base-editing reactions to engineer CCR5 variants with a destabilizing polar residue that prevents membrane integration of folding (similar to the mutation G106R, Tables 1 and 4) using the guide-RNAs described in Tables 3 and 4. Other structurally important proline and cysteine residues are also shown in grey shading with a double ring around the residue (Table 4). Residues demarcated with grey shading and a single ring not specifically labeled with a mutation (i.e., not G106R or R223Q) are glutamine, tryptophan, and arginine residues, which can be changed into stop codons to prevent the translation of full-length functional protein (Table 5), mimicking the effect of the CCR5-Δ32 and FS299 alleles. The sequence corresponds to SEQ ID NO: 310.

FIGS. 2A to 2C are graphic representations of sequence alignments and structure. FIG. 2A shows a strategy for preventing CCR5 protein production by altering splicing sites: donor site, branch-point, or acceptor sites (Table 2). FIG. 2B shows consensus sequences of the human Lariat-structure branch-point and acceptor sites, suggesting that the guanosine of the acceptor site is an excellent target for Cas9-mediated base-editing of C4T on the complementary strand (Table 2). FIG. 2C shows the genomic sequence of the CCR5 gene showing the junction of intron 2 (lowercase) and exon 2 (capitalized), the cognate start-codon (boldface), potential branch-points (italics), and the cognate donor site (underlined). The sequence corresponds to SEQ ID NO: 311.

FIG. 3 is a graphic representation of protein and open-reading frame sequences of the CCR5 receptor. HIV-protective variants (C20S, C101X, G106R, C178R, Δ32, R223Q, C269F, and FS299) that can be replicated or imitated using genome/base-editing with APOBEC1-Cas9 tools (Tables 1-10) are underlined. Grey shading indicates small/hydrophobic residues in a transmembrane domain (TM) that can be targeted for base-editing reactions to engineer CCR5 variants with a destabilizing polar residue that prevents membrane integration of folding (similar to the mutation G106R, Tables 1 and 4) using the guide-RNAs described in Tables 3 and 4. Other structurally important proline and cysteine residues are also shown in grey shading with a double ring around the residue (Table 4). Residues demarcated with grey shading and a single ring not specifically labeled with a mutation (i.e., not G106R or R223Q) are glutamine, tryptophan, and arginine residues, which can be changed into stop codons to prevent the translation of full-length functional protein (Table 5), mimicking the effect of the CCR5-Δ32 and FS299 alleles. The nucleotide sequence corresponds to SEQ ID NO: 312 and the amino acid sequence corresponds to SEQ ID NO: 313.

FIG. 4 is a graphic representation of a numbering scheme used herein. The numbering scheme is based on the predicted location of the target C within the single stranded stretch of DNA (R-loop) displaced by a programmable guide RNA sequence occurring when a DNA-binding domain (e.g, Cas9, nCas9, dCas9) binds a genomic site. The sequence corresponds to SEQ ID NO: 314.

FIG. 5 is a graphic representation of C to T editing of CCR5 target DNA (SEQ ID NO: 738) in HEK293 cells using KKH-SaBE3 and guide-RNA Q186X-e. The editing was calculated from total reads (MiSeq). Panel A demonstrates that significant editing was observed at position C7 and C13 of SEQ ID NO: 739 (complementary nucleotide sequence is SEQ ID NO: 741), both of which generate premature stop codons in tandem (Q186X and Q188X, see inset graphic of panel A and amino acid sequence of SEQ ID NO: 740). The PAM sequence (SEQ ID NO: 736) is shown as underlined and the last nucleotide of the protospacer (SEQ ID NO: 735) is separated with a line. Raw data used for base-calling and calculating base-editing for KKH-BE3 and Q186X-e treated HEK293 cells is shown in panel B. The indel percentage was 1.97%. Panel C shows raw data collected for untreated control cells.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

As used herein, the term “C-C Chemokine Receptor 2” (also referred to as “C-C Chemokine Receptor type 2,” “CCR2,” “CCR-2,” “cluster of differentiation 192,” and “CD192”) is a chemokine receptor encoded by the CCR2 gene. The CCR2 gene encodes two isoforms of the CCR2 protein, which is expressed on peripheral blood monocytes, activated T cells, B cells, and immature dendritic cells. Known ligands for CCR2 include the monocyte chemotactic proteins (MCPs) MCP-1, -2 and -3, which belong to the family of C-C chemokines.

As used herein, “C-C Chemokine Receptor 5” (also referred to as “C-C Chemokine Receptor type 5,” “CCR5,” “CCR-5,” “cluster of differentiation-195,” and “CD195,” is a member of the beta chemokine receptor family. This protein is expressed by macrophages, dendritic cells, and memory T cells of the immune system; endothelila cells, epithelial cells, vascular smooth muscle cells, and fibroblasts; and microglia, neurons, and astrocytes in the central nervous system. See, e.g., Barmania and Pepper, Applied & Translational Genomics 2 (2013) 3-16, which is incorporated herein by reference.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a fusion protein provided herein, e.g., of a fusion protein comprising a nuclease-inactive Cas9 domain and a nucleic acid-editing domain (e.g., a deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a deaminase, a hybrid protein, a protein dimer, a complex of a protein (or protein dimer) and a polynucleotide, or a polynucleotide, may vary depending on various factors, such as, for example, on the desired biological response, e.g., on the specific allele, genome, or target site to be edited, on the cell or tissue being targeted, and/or on the agent being used.

The term “Gam protein.” as used herein, refers generally to proteins capable of binding to one or more ends of a double strand break of a double stranded nucleic acid (e.g., double stranded DNA). In some embodiments, the Gam protein prevents or inhibits degradation of one or more strands of a nucleic acid at the site of the double strand break. In some embodiments, a Gam protein is a naturally-occurring Gam protein from bacteriophage Mu. or a non-naturally occurring variant thereof.

The term “loss-of-function mutation” or “inactivating mutation” refers to a mutation that results in the gene product having less or no function (being partially or wholly inactivated). When the allele has a complete loss of function (null allele), it is often called an amorphic mutation in the Muller's morphs schema. Phenotypes associated with such mutations are most often recessive. Exceptions are when the organism is haploid, or when the reduced dosage of a normal gene product is not enough for a normal phenotype (this is called haploinsufficiency).

The term “gain-of-function mutation” or “activating mutation” refers to a mutation that changes the gene product such that its effect gets stronger (enhanced activation) or even is superseded by a different and abnormal function. A gain of function mutation may also be referred to as a neomorphic mutation. When the new allele is created, a heterozygote containing the newly created allele as well as the original will express the new allele, genetically defining the mutations as dominant phenotypes.

The terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. Treatment may also be continued after symptoms have resolved, for example, to prevent or delay their recurrence. In one embodiment, the methods and compositions disclosed herein may be used to delay the onset of AIDS in an individual infected with HIV. The terms “prevention,” “prevent.” and “preventing” refer to a clinical intervention aimed to inhibit the onset of a disease or disorder, or one or more symptoms thereof, as described herein. In one embodiment, treatment may be administered in the absence of symptoms, e.g., to prevent or delay onset of a symptom or inhibit onset or progression of a disease. In one embodiment, the methods and compositions disclosed herein may be used to prevent infection of a subject with HIV. In one example, treatment may be administered to a susceptible individual prior to the onset of symptoms (e.g., in light of a history of symptoms and/or in light of genetic or other susceptibility factors) in order to prevent the onset of the disease or symptoms of the disease.

The term “genome” refers to the genetic material of a cell or organism. It typically includes DNA (or RNA in the case of RNA viruses). The genome includes both the genes, the coding regions, the noncoding DNA, and the genomes of the mitochondria and chloroplasts. A genome does not typically include genetic material that is artificially introduced into a cell or organism, e.g., a plasmid that is transformed into a bacteria is not a part of the bacterial genome.

A “programmable DNA-binding protein” refers to DNA binding proteins that can be programmed to target any desired nucleotide sequence within a genome. To program the DNA-binding protein to bind a desired nucleotide sequence, the DNA binding protein may be modified to change its binding specificity. e.g., zinc finger nuclease (ZFN) or transcription activator-like effector proteins (TALE). ZFNs are artificial restriction enzymes generated by fusing a zinc finger DNA-binding domain to a DNA-cleavage domain. Zinc finger domains can be engineered to target specific desired DNA sequences and this enables zinc-fingers to bind unique sequences within complex genomes. Transcription activator-like effector nucleases (TALEN) are engineered restriction enzymes that can be engineered to cut specific sequences of DNA. They are made by fusing a TAL effector DNA-binding domain to a nuclease domain (e.g., Fok1). Transcription activator-like effectors (TALEs) can be engineered to bind practically any desired DNA sequence. Methods for programming ZFNs and TALEs are familiar to one skilled in the art. For example, such methods are described in Maeder, et al., Mol. Cell 31 (2): 294-301, 2008; Carroll et al., Genetics Society of America, 188 (4): 773-782, 2011: Miller el al., Nature Biotechnology 25 (7): 778-785, 2007; Christian et al., Genetics 186 (2): 757-61, 2008; Li et al., Nucleic Acids Res 39 (1): 359-372, 2010; and Moscou et al., Science 326 (5959): 1501, 2009, the entire contents of each of which are incorporated herein by reference.

A “guide nucleotide sequence-programmable DNA-binding protein” refers to a protein, a polypeptide, or a domain that is able to bind DNA, and the binding to its target DNA sequence is mediated by a guide nucleotide sequence. Thus, it is appreciated that the guide nucleotide sequence-programmable DNA-binding protein binds to a guide nucleotide sequence. The “guide nucleotide” may be an RNA or DNA molecule (e.g., a single-stranded DNA or ssDNA molecule) that is complementary to the target sequence and can guide the DNA binding protein to the target sequence. As such, a guide nucleotide sequence-programmable DNA-binding protein may be a RNA-programmable DNA-binding protein (e.g., a Cas9 protein), or an ssDNA-programmable DNA-binding protein (e.g., an Argonaute protein). “Programmable” means the DNA-binding protein may be programmed to bind any DNA sequence that the guide nucleotide targets. Exemplary guide nucleotide sequence-programmable DNA-binding proteins include, but are not limited to, Cas9 (e.g., dCas9 and nCas9), saCas9 (e.g., saCasd, saCasn, and saKKH Cas9), CasX, CasY, Cpf1, C2c1, C2c2, C2c3, Argonaute, and any other suitable protein described herein. In some embodiments, the fusion protein described herein comprises a Gam protein, a guide nucleotide sequence-programmable DNA binding protein, and a cytidine deaminase domain.

In some embodiments, the guide nucleotide sequence exists as a single nucleotide molecule and comprises comprise two domains: (1) a domain that shares homology to a target nucleic acid (e.g., and directs binding of a guide nucleotide sequence-programmable DNA-binding protein to the target); and (2) a domain that binds a guide nucleotide sequence-programmable DNA-binding protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), which is incorporated herein by reference. Other examples of gRNAs (e.g., those including domain (2)) can be found in U.S. Patent Application Publication US20160208288 and U.S. Patent Application Publication US20160200779, each of which is herein incorporated by reference.

Because the guide nucleotide sequence hybridizes to a target DNA sequence, the guide nucleotide sequence-programmable DNA-binding proteins are able to specifically bind, in principle, to any sequence complementary to the guide nucleotide sequence. Methods of using guide nucleotide sequence-programmable DNA-binding protein, such as Cas9, for site-specific cleavage (e.g., to modify a genome) are known in the art (see e.g., Cong, L. et al. Multiplex genome engineering using CRISPR/Cas systems. Science 339, 819-823 (2013): Mali, P. et al. RNA-guided human genome engineering via Cas9. Science 339, 823-826 (2013); Hwang. W. Y. et al. Efficient genome editing in zebrafish using a CRISPR-Cas system. Nature biotechnology 31, 227-229 (2013); Jinek. M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang. W. et al. RNA-guided editing of bacterial genomes using CRISPR-Cas systems. Nature biotechnology 31, 233-239 (2013); the entire contents of each of which are incorporated herein by reference).

As used herein, the term “Cas9” or “Cas9 nuclease” refers to an RNA-guided nuclease comprising a Cas9 protein, fragment, or variant thereof. A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), which is incorporated herein by reference.

Cas9 nuclease sequences and structures are known to those of skill in the art (see. e.g., Ferretti et al., Proc. Natl. Acad. Sci. 98:4658-4663(2001); Deltcheva E. el al., Nature 471:602-607(2011); and Jinek et al., Science 337:816-821(2012), the entire contents of each of which are incorporated herein by reference), Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; which is incorporated herein by reference. In some embodiments, wild type Cas9 corresponds to Cas9 from Streptococcus pyogenes (NCBI Reference Sequence: NC_002737.2. SEQ ID NO: 4 (nucleotide); and Uniprot Reference Sequence: Q99ZW2, SEQ ID NO: 1 (amino acid).

Streptococcus pyogenes Cas9 (Wild Type) Nucleotide Sequence

(SEQ ID NO: 4)
ATGGATAAGAAATACTCAATAGGCTTAGATATCGGCACAAATAGCGTCGG
ATGGGCGGTGATCACTGATGAATATAAGGTTCCGTCTAAAAAGTTCAAGG
TTCTGGGAAATACAGACCGCCACAGTATCAAAAAAAATCTTATAGGGGCT
CTTTTATTTGACAGTGGAGAGACAGCGGAAGCGACTCGTCTCAAACGGAC
AGCTCGTAGAAGGTATACACGTCGGAAGAATCGTATTTGTTATCTACAGG
AGATTTTTTCAAATGAGATGGCGAAAGTAGATGATAGTTTCTTTCATCGA
CTTGAAGAGTCTTTTTTGGTGGAAGAAGACAAGAAGCATGAACGTCATCC
TATTTTTGGAAATATAGTAGATGAAGTTGCTTATCATGAGAAATATCCAA
CTATCTATCATCTGCGAAAAAAATTGGTAGATTCTACTGATAAAGCGGAT
TTGCGCTTAATCTATTTGGCCTTAGCGCATATGATTAAGTTTCGTGGTCA
TTTTTTGATTGAGGGAGATTTAAATCCTGATAATAGTGATGTGGACAAAC
TATTTATCCAGTTGGTACAAACCTACAATCAATTATTTGAAGAAAACCCT
ATTAACGCAAGTGGAGTAGATGCTAAAGCGATTCTTTCTGCACGATTGAG
TAAATCAAGACGATTAGAAAATCTCATTGCTCAGCTCCCCGGTGAGAAGA
AAAATGGCTTATTTGGGAATCTCATTGCTTTGTCATTGGGTTTGACCCCT
AATTTTAAATCAAATTTTGATTTGGCAGAAGATGCTAAATTACAGCTTTC
AAAAGATACTTACGATGATGATTTAGATAATTTATTGGCGCAAATTGGAG
ATCAATATGCTGATTTGTTTTTGGCAGCTAAGAATTTATCAGATGCTATT
TTACTTTCAGATATCCTAAGAGTAAATACTGAAATAACTAAGGCTCCCCT
ATCAGCTTCAATGATTAAACGCTACGATGAACATCATCAAGACTTGACTC
TTTTAAAAGCTTTAGTTCGACAACAACTTCCAGAAAAGTATAAAGAAATC
TTTTTTGATCAATCAAAAAACGGATATGCAGGTTATATTGATGGGGGAGC
TAGCCAAGAAGAATTTTATAAATTTATCAAACCAATTTTAGAAAAAATGG
ATGGTACTGAGGAATTATTGGTGAAACTAAATCGTGAAGATTTGCTGCGC
AAGCAACGGACCTTTGACAACGGCTCTATTCCCCATCAAATTCACTTGGG
TGAGCTGCATGCTATTTTGAGAAGACAAGAAGACTTTTATCCATTTTTAA
AAGACAATCGTGAGAAGATTGAAAAAATCTTGACTTTTCGAATTCCTTAT
TATGTTGGTCCATTGGCGCGTGGCAATAGTCGTTTTGCATGGATGACTCG
GAAGTCTGAAGAAACAATTACCCCATGGAATTTTGAAGAAGTTGTCGATA
AAGGTGCTTCAGCTCAATCATTTATTGAACGCATGACAAACTTTGATAAA
AATCTTCCAAATGAAAAAGTACTACCAAAACATAGTTTGCTTTATGAGTA
TTTTACGGTTTATAACGAATTGACAAAGGTCAAATATGTTACTGAAGGAA
TGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGA
TTATTTCAAAAAAATAGAATGTTTTGATAGTGTTGAAATTTCAGGAGTTG
AAGATAGATTTAATGCTTCATTAGGTACCTACCATGATTTGCTAAAAATT
ATTAAAGATAAAGATTTTTTGGATAATGAAGAAAATGAAGATATCTTAGA
GGATATTGTTTTAACATTGACCTTATTTGAAGATAGGGAGATGATTGAGG
AAAGACTTAAAACATATGCTCACCTCTTTGATGATAAGGTGATGAAACAG
CTTAAACGTCGCCGTTATACTGGTTGGGGACGTTTGTCTCGAAAATTGAT
TAATGGTATTAGGGATAAGCAATCTGGCAAAACAATATTAGATTTTTTGA
AATCAGATGGTTTTGCCAATCGCAATTTTATGCAGCTGATCCATGATGAT
AGTTTGACATTTAAAGAAGACATTCAAAAAGCACAAGTGTCTGGACAAGG
CGATAGTTTACATGAACATATTGCAAATTTAGCTGGTAGCCCTGCTATTA
AAAAAGGTATTTTACAGACTGTAAAAGTTGTTGATGAATTGGTCAAAGTA
ATGGGGCGGCATAAGCCAGAAAATATCGTTATTGAAATGGCACGTGAAAA
TCAGACAACTCAAAAGGGCCAGAAAAATTCGCGAGAGCGTATGAAACGAA
TCGAAGAAGGTATCAAAGAATTAGGAAGTCAGATTCTTAAAGAGCATCCT
GTTGAAAATACTCAATTGCAAAATGAAAAGCTCTATCTCTATTATCTCCA
AAATGGAAGAGACATGTATGTGGACCAAGAATTAGATATTAATCGTTTAA
GTGATTATGATGTCGATCACATTGTTCCACAAAGTTTCCTTAAAGACGAT
TCAATAGACAATAAGGTCTTAACGCGTTCTGATAAAAATCGTGGTAAATC
GGATAACGTTCCAAGTGAAGAAGTAGTCAAAAAGATGAAAAACTATTGGA
GACAACTTCTAAACGCCAAGTTAATCACTCAACGTAAGTTTGATAATTTA
ACGAAAGCTGAACGTGGAGGTTTGAGTGAACTTGATAAAGCTGGTTTTAT
CAAACGCCAATTGGTTGAAACTCGCCAAATCACTAAGCATGTGGCACAAA
TTTTGGATAGTCGCATGAATACTAAATACGATGAAAATGATAAACTTATT
CGAGAGGTTAAAGTGATTACCTTAAAATCTAAATTAGTTTCTGACTTCCG
AAAAGATTTCCAATTCTATAAAGTACGTGAGATTAACAATTACCATCATG
CCCATGATGCGTATCTAAATGCCGTCGTTGGAACTGCTTTGATTAAGAAA
TATCCAAAACTTGAATCGGAGTTTGTCTATGGTGATTATAAAGTTTATGA
TGTTCGTAAAATGATTGCTAAGTCTGAGCAAGAAATAGGCAAAGCAACCG
CAAAATATTTCTTTTACTCTAATATCATGAACTTCTTCAAAACAGAAATT
ACACTTGCAAATGGAGAGATTCGCAAACGCCCTCTAATCGAAACTAATGG
GGAAACTGGAGAAATTGTCTGGGATAAAGGGCGAGATTTTGCCACAGTGC
GCAAAGTATTGTCCATGCCCCAAGTCAATATTGTCAAGAAAACAGAAGTA
CAGACAGGCGGATTCTCCAAGGAGTCAATTTTACCAAAAAGAAATTCGGA
CAAGCTTATTGCTCGTAAAAAAGACTGGGATCCAAAAAAATATGGTGGTT
TTGATAGTCCAACGGTAGCTTATTCAGTCCTAGTGGTTGCTAAGGTGGAA
AAAGGGAAATCGAAGAAGTTAAAATCCGTTAAAGAGTTACTAGGGATCAC
AATTATGGAAAGAAGTTCCTTTGAAAAAAATCCGATTGACTTTTTAGAAG
CTAAAGGATATAAGGAAGTTAAAAAAGACTTAATCATTAAACTACCTAAA
TATAGTCTTTTTGAGTTAGAAAACGGTCGTAAACGGATGCTGGCTAGTGC
CGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAA
GATAACGAACAAAAACAATTGTTTGTGGAGCAGCATAAGCATTATTTAGA
TGAGATTATTGAGCAAATCAGTGAATTTTCTAAGCGTGTTATTTTAGCAG
ATGCCAATTTAGATAAAGTTCTTAGTGCATATAACAAACATAGAGACAAA
CCAATACGTGAACAAGCAGAAAATATTATTCATTTATTTACGTTGACGAA
TCTTGGAGCTCCCGCTGCTTTTAAATATTTTGATACAACAATTGATCGTA
AACGATATACGTCTACAAAAGAAGTTTTAGATGCCACTCTTATCCATCAA
TCCATCACTGGTCTTTATGAAACACGCATTGATTTGAGTCAGCTAGGAGG
TGACTGA

Streptococcus pyogenes Cas9 (Wild Type) Protein Sequence

(SEQ ID NO: 1)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD (single underline: HNH domain;
double underline: RuvC domain)

In some embodiments, wild type Cas9 corresponds to Cas9 from Staphylococcus aureus (NCBI Reference Sequence: WP_001573634.1, SEQ ID NO: 5 (amino acid).

Staphylococcus aureus Cas9 (Wild Type) Protein Sequence

(SEQ ID NO: 5)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

In some embodiments, wild type Cas9 corresponds to Cas9 from Streptococcus pyogenes (NCBI Reference Sequence: NC_017053.1. SEQ ID NO: 679 (nucleotide); SEQ ID NO: 680 (amino acid)).

(SEQ ID NO: 679)
ATGGATAAGAAATACTCAATAGGCTTAGATATCGGCACAAATAGCGTCGG
ATGGGCGGTGATCACTGATGATTATAAGGTTCCGTCTAAAAAGTTCAAGG
TTCTGGGAAATACAGACCGCCACAGTATCAAAAAAAATCTTATAGGGGCT
CTTTTATTTGGCAGTGGAGAGACAGCGGAAGCGACTCGTCTCAAACGGAC
AGCTCGTAGAAGGTATACACGTCGGAAGAATCGTATTTGTTATCTACAGG
AGATTTTTTCAAATGAGATGGCGAAAGTAGATGATAGTTTCTTTCATCGA
CTTGAAGAGTCTTTTTTGGTGGAAGAAGACAAGAAGCATGAACGTCATCC
TATTTTTGGAAATATAGTAGATGAAGTTGCTTATCATGAGAAATATCCAA
CTATCTATCATCTGCGAAAAAAATTGGCAGATTCTACTGATAAAGCGGAT
TTGCGCTTAATCTATTTGGCCTTAGCGCATATGATTAAGTTTCGTGGTCA
TTTTTTGATTGAGGGAGATTTAAATCCTGATAATAGTGATGTGGACAAAC
TATTTATCCAGTTGGTACAAATCTACAATCAATTATTTGAAGAAAACCCT
ATTAACGCAAGTAGAGTAGATGCTAAAGCGATTCTTTCTGCACGATTGAG
TAAATCAAGACGATTAGAAAATCTCATTGCTCAGCTCCCCGGTGAGAAGA
GAAATGGCTTGTTTGGGAATCTCATTGCTTTGTCATTGGGATTGACCCCT
AATTTTAAATCAAATTTTGATTTGGCAGAAGATGCTAAATTACAGCTTTC
AAAAGATACTTACGATGATGATTTAGATAATTTATTGGCGCAAATTGGAG
ATCAATATGCTGATTTGTTTTTGGCAGCTAAGAATTTATCAGATGCTATT
TTACTTTCAGATATCCTAAGAGTAAATAGTGAAATAACTAAGGCTCCCCT
ATCAGCTTCAATGATTAAGCGCTACGATGAACATCATCAAGACTTGACTC
TTTTAAAAGCTTTAGTTCGACAACAACTTCCAGAAAAGTATAAAGAAATC
TTTTTTGATCAATCAAAAAACGGATATGCAGGTTATATTGATGGGGGAGC
TAGCCAAGAAGAATTTTATAAATTTATCAAACCAATTTTAGAAAAAATGG
ATGGTACTGAGGAATTATTGGTGAAACTAAATCGTGAAGATTTGCTGCGC
AAGCAACGGACCTTTGACAACGGCTCTATTCCCCATCAAATTCACTTGGG
TGAGCTGCATGCTATTTTGAGAAGACAAGAAGACTTTTATCCATTTTTAA
AAGACAATCGTGAGAAGATTGAAAAAATCTTGACTTTTCGAATTCCTTAT
TATGTTGGTCCATTGGCGCGTGGCAATAGTCGTTTTGCATGGATGACTCG
GAAGTCTGAAGAAACAATTACCCCATGGAATTTTGAAGAAGTTGTCGATA
AAGGTGCTTCAGCTCAATCATTTATTGAACGCATGACAAACTTTGATAAA
AATCTTCCAAATGAAAAAGTACTACCAAAACATAGTTTGCTTTATGAGTA
TTTTACGGTTTATAACGAATTGACAAAGGTCAAATATGTTACTGAGGGAA
TGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGA
TTATTTCAAAAAAATAGAATGTTTTGATAGTGTTGAAATTTCAGGAGTTG
AAGATAGATTTAATGCTTCATTAGGCGCCTACCATGATTTGCTAAAAATT
ATTAAAGATAAAGATTTTTTGGATAATGAAGAAAATGAAGATATCTTAGA
GGATATTGTTTTAACATTGACCTTATTTGAAGATAGGGGGATGATTGAGG
AAAGACTTAAAACATATGCTCACCTCTTTGATGATAAGGTGATGAAACAG
CTTAAACGTCGCCGTTATACTGGTTGGGGACGTTTGTCTCGAAAATTGAT
TAATGGTATTAGGGATAAGCAATCTGGCAAAACAATATTAGATTTTTTGA
AATCAGATGGTTTTGCCAATCGCAATTTTATGCAGCTGATCCATGATGAT
AGTTTGACATTTAAAGAAGATATTCAAAAAGCACAGGTGTCTGGACAAGG
CCATAGTTTACATGAACAGATTGCTAACTTAGCTGGCAGTCCTGCTATTA
AAAAAGGTATTTTACAGACTGTAAAAATTGTTGATGAACTGGTCAAAGTA
ATGGGGCATAAGCCAGAAAATATCGTTATTGAAATGGCACGTGAAAATCA
GACAACTCAAAAGGGCCAGAAAAATTCGCGAGAGCGTATGAAACGAATCG
AAGAAGGTATCAAAGAATTAGGAAGTCAGATTCTTAAAGAGCATCCTGTT
GAAAATACTCAATTGCAAAATGAAAAGCTCTATCTCTATTATCTACAAAA
TGGAAGAGACATGTATGTGGACCAAGAATTAGATATTAATCGTTTAAGTG
ATTATGATGTCGATCACATTGTTCCACAAAGTTTCATTAAAGACGATTCA
ATAGACAATAAGGTACTAACGCGTTCTGATAAAAATCGTGGTAAATCGGA
TAACGTTCCAAGTGAAGAAGTAGTCAAAAAGATGAAAAACTATTGGAGAC
AACTTCTAAACGCCAAGTTAATCACTCAACGTAAGTTTGATAATTTAACG
AAAGCTGAACGTGGAGGTTTGAGTGAACTTGATAAAGCTGGTTTTATCAA
ACGCCAATTGGTTGAAACTCGCCAAATCACTAAGCATGTGGCACAAATTT
TGGATAGTCGCATGAATACTAAATACGATGAAAATGATAAACTTATTCGA
GAGGTTAAAGTGATTACCTTAAAATCTAAATTAGTTTCTGACTTCCGAAA
AGATTTCCAATTCTATAAAGTACGTGAGATTAACAATTACCATCATGCCC
ATGATGCGTATCTAAATGCCGTCGTTGGAACTGCTTTGATTAAGAAATAT
CCAAAACTTGAATCGGAGTTTGTCTATGGTGATTATAAAGTTTATGATGT
TCGTAAAATGATTGCTAAGTCTGAGCAAGAAATAGGCAAAGCAACCGCAA
AATATTTCTTTTACTCTAATATCATGAACTTCTTCAAAACAGAAATTACA
CTTGCAAATGGAGAGATTCGCAAACGCCCTCTAATCGAAACTAATGGGGA
AACTGGAGAAATTGTCTGGGATAAAGGGCGAGATTTTGCCACAGTGCGCA
AAGTATTGTCCATGCCCCAAGTCAATATTGTCAAGAAAACAGAAGTACAG
ACAGGCGGATTCTCCAAGGAGTCAATTTTACCAAAAAGAAATTCGGACAA
GCTTATTGCTCGTAAAAAAGACTGGGATCCAAAAAAATATGGTGGTTTTG
ATAGTCCAACGGTAGCTTATTCAGTCCTAGTGGTTGCTAAGGTGGAAAAA
GGGAAATCGAAGAAGTTAAAATCCGTTAAAGAGTTACTAGGGATCACAAT
TATGGAAAGAAGTTCCTTTGAAAAAAATCCGATTGACTTTTTAGAAGCTA
AAGGATATAAGGAAGTTAAAAAAGACTTAATCATTAAACTACCTAAATAT
AGTCTTTTTGAGTTAGAAAACGGTCGTAAACGGATGCTGGCTAGTGCCGG
AGAATTACAAAAAGGAAATGAGCTGGCTCTGCCAAGCAAATATGTGAATT
TTTTATATTTAGCTAGTCATTATGAAAAGTTGAAGGGTAGTCCAGAAGAT
AACGAACAAAAACAATTGTTTGTGGAGCAGCATAAGCATTATTTAGATGA
GATTATTGAGCAAATCAGTGAATTTTCTAAGCGTGTTATTTTAGCAGATG
CCAATTTAGATAAAGTTCTTAGTGCATATAACAAACATAGAGACAAACCA
ATACGTGAACAAGCAGAAAATATTATTCATTTATTTACGTTGACGAATCT
TGGAGCTCCCGCTGCTTTTAAATATTTTGATACAACAATTGATCGTAAAC
GATATACGTCTACAAAAGAAGTTTTAGATGCCACTCTTATCCATCAATCC
ATCACTGGTCTTTATGAAACACGCATTGATTTGAGTCAGCTAGGAGGTGA
CTGA
(SEQ ID NO: 680)
MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD (single underline: HNH domain;
double underline: RuvC domain)

In some embodiments, wild type Cas9 corresponds to, or comprises, Streptococcus pyogenes Cas9 (SEQ ID NO: 681 (nucleotide) and/or SEQ ID NO: 682 (amino acid)):

(SEQ ID NO: 681)
ATGGATAAAAAGTATTCTATTGGTTTAGACATCGGCACTAATTCCGTTGG
ATGGGCTGTCATAACCGATGAATACAAAGTACCTTCAAAGAAATTTAAGG
TGTTGGGGAACACAGACCGTCATTCGATTAAAAAGAATCTTATCGGTGCC
CTCCTATTCGATAGTGGCGAAACGGCAGAGGCGACTCGCCTGAAACGAAC
CGCTCGGAGAAGGTATACACGTCGCAAGAACCGAATATGTTACTTACAAG
AAATTTTTAGCAATGAGATGGCCAAAGTTGACGATTCTTTCTTTCACCGT
TTGGAAGAGTCCTTCCTTGTCGAAGAGGACAAGAAACATGAACGGCACCC
CATCTTTGGAAACATAGTAGATGAGGTGGCATATCATGAAAAGTACCCAA
CGATTTATCACCTCAGAAAAAAGCTAGTTGACTCAACTGATAAAGCGGAC
CTGAGGTTAATCTACTTGGCTCTTGCCCATATGATAAAGTTCCGTGGGCA
CTTTCTCATTGAGGGTGATCTAAATCCGGACAACTCGGATGTCGACAAAC
TGTTCATCCAGTTAGTACAAACCTATAATCAGTTGTTTGAAGAGAACCCT
ATAAATGCAAGTGGCGTGGATGCGAAGGCTATTCTTAGCGCCCGCCTCTC
TAAATCCCGACGGCTAGAAAACCTGATCGCACAATTACCCGGAGAGAAGA
AAAATGGGTTGTTCGGTAACCTTATAGCGCTCTCACTAGGCCTGACACCA
AATTTTAAGTCGAACTTCGACTTAGCTGAAGATGCCAAATTGCAGCTTAG
TAAGGACACGTACGATGACGATCTCGACAATCTACTGGCACAAATTGGAG
ATCAGTATGCGGACTTATTTTTGGCTGCCAAAAACCTTAGCGATGCAATC
CTCCTATCTGACATACTGAGAGTTAATACTGAGATTACCAAGGCGCCGTT
ATCCGCTTCAATGATCAAAAGGTACGATGAACATCACCAAGACTTGACAC
TTCTCAAGGCCCTAGTCCGTCAGCAACTGCCTGAGAAATATAAGGAAATA
TTCTTTGATCAGTCGAAAAACGGGTACGCAGGTTATATTGACGGCGGAGC
GAGTCAAGAGGAATTCTACAAGTTTATCAAACCCATATTAGAGAAGATGG
ATGGGACGGAAGAGTTGCTTGTAAAACTCAATCGCGAAGATCTACTGCGA
AAGCAGCGGACTTTCGACAACGGTAGCATTCCACATCAAATCCACTTAGG
CGAATTGCATGCTATACTTAGAAGGCAGGAGGATTTTTATCCGTTCCTCA
AAGACAATCGTGAAAAGATTGAGAAAATCCTAACCTTTCGCATACCTTAC
TATGTGGGACCCCTGGCCCGAGGGAACTCTCGGTTCGCATGGATGACAAG
AAAGTCCGAAGAAACGATTACTCCATGGAATTTTGAGGAAGTTGTCGATA
AAGGTGCGTCAGCTCAATCGTTCATCGAGAGGATGACCAACTTTGACAAG
AATTTACCGAACGAAAAAGTATTGCCTAAGCACAGTTTACTTTACGAGTA
TTTCACAGTGTACAATGAACTCACGAAAGTTAAGTATGTCACTGAGGGCA
TGCGTAAACCCGCCTTTCTAAGCGGAGAACAGAAGAAAGCAATAGTAGAT
CTGTTATTCAAGACCAACCGCAAAGTGACAGTTAAGCAATTGAAAGAGGA
CTACTTTAAGAAAATTGAATGCTTCGATTCTGTCGAGATCTCCGGGGTAG
AAGATCGATTTAATGCGTCACTTGGTACGTATCATGACCTCCTAAAGATA
ATTAAAGATAAGGACTTCCTGGATAACGAAGAGAATGAAGATATCTTAGA
AGATATAGTGTTGACTCTTACCCTCTTTGAAGATCGGGAAATGATTGAGG
AAAGACTAAAAACATACGCTCACCTGTTCGACGATAAGGTTATGAAACAG
TTAAAGAGGCGTCGCTATACGGGCTGGGGACGATTGTCGCGGAAACTTAT
CAACGGGATAAGAGACAAGCAAAGTGGTAAAACTATTCTCGATTTTCTAA
AGAGCGACGGCTTCGCCAATAGGAACTTTATGCAGCTGATCCATGATGAC
TCTTTAACCTTCAAAGAGGATATACAAAAGGCACAGGTTTCCGGACAAGG
GGACTCATTGCACGAACATATTGCGAATCTTGCTGGTTCGCCAGCCATCA
AAAAGGGCATACTCCAGACAGTCAAAGTAGTGGATGAGCTAGTTAAGGTC
ATGGGACGTCACAAACCGGAAAACATTGTAATCGAGATGGCACGCGAAAA
TCAAACGACTCAGAAGGGGCAAAAAAACAGTCGAGAGCGGATGAAGAGAA
TAGAAGAGGGTATTAAAGAACTGGGCAGCCAGATCTTAAAGGAGCATCCT
GTGGAAAATACCCAATTGCAGAACGAGAAACTTTACCTCTATTACCTACA
AAATGGAAGGGACATGTATGTTGATCAGGAACTGGACATAAACCGTTTAT
CTGATTACGACGTCGATCACATTGTACCCCAATCCTTTTTGAAGGACGAT
TCAATCGACAATAAAGTGCTTACACGCTCGGATAAGAACCGAGGGAAAAG
TGACAATGTTCCAAGCGAGGAAGTCGTAAAGAAAATGAAGAACTATTGGC
GGCAGCTCCTAAATGCGAAACTGATAACGCAAAGAAAGTTCGATAACTTA
ACTAAAGCTGAGAGGGGTGGCTTGTCTGAACTTGACAAGGCCGGATTTAT
TAAACGTCAGCTCGTGGAAACCCGCCAAATCACAAAGCATGTTGCACAGA
TACTAGATTCCCGAATGAATACGAAATACGACGAGAACGATAAGCTGATT
CGGGAAGTCAAAGTAATCACTTTAAAGTCAAAATTGGTGTCGGACTTCAG
AAAGGATTTTCAATTCTATAAAGTTAGGGAGATAAATAACTACCACCATG
CGCACGACGCTTATCTTAATGCCGTCGTAGGGACCGCACTCATTAAGAAA
TACCCGAAGCTAGAAAGTGAGTTTGTGTATGGTGATTACAAAGTTTATGA
CGTCCGTAAGATGATCGCGAAAAGCGAACAGGAGATAGGCAAGGCTACAG
CCAAATACTTCTTTTATTCTAACATTATGAATTTCTTTAAGACGGAAATC
ACTCTGGCAAACGGAGAGATACGCAAACGACCTTTAATTGAAACCAATGG
GGAGACAGGTGAAATCGTATGGGATAAGGGCCGGGACTTCGCGACGGTGA
GAAAAGTTTTGTCCATGCCCCAAGTCAACATAGTAAAGAAAACTGAGGTG
CAGACCGGAGGGTTTTCAAAGGAATCGATTCTTCCAAAAAGGAATAGTGA
TAAGCTCATCGCTCGTAAAAAGGACTGGGACCCGAAAAAGTACGGTGGCT
TCGATAGCCCTACAGTTGCCTATTCTGTCCTAGTAGTGGCAAAAGTTGAG
AAGGGAAAATCCAAGAAACTGAAGTCAGTCAAAGAATTATTGGGGATAAC
GATTATGGAGCGCTCGTCTTTTGAAAAGAACCCCATCGACTTCCTTGAGG
CGAAAGGTTACAAGGAAGTAAAAAAGGATCTCATAATTAAACTACCAAAG
TATAGTCTGTTTGAGTTAGAAAATGGCCGAAAACGGATGTTGGCTAGCGC
CGGAGAGCTTCAAAAGGGGAACGAACTCGCACTACCGTCTAAATACGTGA
ATTTCCTGTATTTAGCGTCCCATTACGAGAAGTTGAAAGGTTCACCTGAA
GATAACGAACAGAAGCAACTTTTTGTTGAGCAGCACAAACATTATCTCGA
CGAAATCATAGAGCAAATTTCGGAATTCAGTAAGAGAGTCATCCTAGCTG
ATGCCAATCTGGACAAAGTATTAAGCGCATACAACAAGCACAGGGATAAA
CCCATACGTGAGCAGGCGGAAAATATTATCCATTTGTTTACTCTTACCAA
CCTCGGCGCTCCAGCCGCATTCAAGTATTTTGACACAACGATAGATCGCA
AACGATACACTTCTACCAAGGAGGTGCTAGACGCGACACTGATTCACCAA
TCCATCACGGGATTATATGAAACTCGGATAGATTTGTCACAGCTTGGGGG
TGACGGATCCCCCAAGAAGAAGAGGAAAGTCTCGAGCGACTACAAAGACC
ATGACGGTGATTATAAAGATCATGACATCGATTACAAGGATGACGATGAC
AAGGCTGCAGGA
(SEQ ID NO: 682)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD (single underline: HNH domain;
double underline: RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1. NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria, meningitidis (NCBI Ref: YP_002342100.1) or to a Cas9 from any of the organisms listed in Example 1 (SEQ ID NOs: 1-260, 270-292 or 315-323).

In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, the fragment is at least 100 amino acids in length. In some embodiments, the fragment is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, or at least 1300 amino acids in length.

To be used as in the fusion protein of the present disclosure as the guide nucleotide sequence-programmable DNA binding protein domain, a Cas9 protein typically needs to be nuclease inactive. A nuclease-inactive Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821(2012); Qi et al., (2013) Cell. 28; 152(5):1173-83, which is incorporated herein by reference). For example, the DNA cleavage domain of Cas9 is known to include two subdomains, the HNH nuclease subdomain and the RuvC1 subdomain. The HNK subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)).

S. pyogenes dCas9 (D10A and H840A)

(SEQ ID NO: 2)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQLEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRI (single underline: HNH domain; double
underline: RuvC domain).

The dCas9 of the present disclosure encompasses completely inactive Cas9 or partially inactive Cas9. For example, the dCas9 may have one of the two nuclease domain inactivated, while the other nuclease domain remains active. Such a partially active Cas9 may also be referred to as a Cas9 nickase, due to its ability to cleave one strand of the targeted DNA sequence. The Cas9 nickase suitable for use in accordance with the present disclosure has an active HNH domain and an inactive RuvC domain and is able to cleave only the strand of the target DNA that is bound by the sgRNA (which is the opposite strand of the strand that is being edited via deamination). The Cas9 nickase of the present disclosure may comprise mutations that inactivate the RuvC domain, e.g., a D10A mutation. It is to be understood that any mutation that inactivates the RuvC domain may be included in a Cas9 nickase, e.g., insertion, deletion, or single or multiple amino acid substitution in the RuvC domain. In a Cas9 nickase described herein, while the RuvC domain is inactivated, the HNH domain remains active. Thus, while the Cas9 nickase may comprise mutations other than those that inactivate the RuvC domain (e.g., D10A), those mutations do not affect the activity of the HNH domain. In a non-limiting Cas9 nickase example, the histidine at position 840 remains unchanged. The sequence of exemplary Cas9 nickases suitable for the present disclosure is provided below.

S. pyogenes Cas9 Nickase (D10A)

(SEQ ID NO: 3)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQLEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD (single underline: HNH domain;
double underline: RuvC domain)

S. aureus Cas9 Nickase (D10A)

(SEQ ID NO: 6)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKLAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQLEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKEKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

The targeting range of base editors was further expanded by applying recently engineered Cas9 variants that expand or alter PAM specificities. Joung and coworkers recently reported three SpCas9 mutants that accept NGA (VQR-Cas9), NGAG (EQR-Cas9), or NGCG(VRER-Cas9) PAM sequences (see: Kleinstiver. B. P. et al. Engineered CRISPR-Cas9 nucleases with altered PAM specificities. Nature 523, 481-485; 2015, which is herein incorporated by reference in its entirety). In addition. Joung and coworkers engineered a SaCas9 variant containing three mutations (SaKKH-Cas9) that relax its PAM requirement to NNNRRT (see: Kleinstiver, B. P. et al. Broadening the targeting range of Staphylococcus aureus CRISPR-Cas9 by modifying PAM recognition. Nat. Biotechnol. 33, 1293-1298; 2015, which is herein incorporated by reference in its entirety).

VRER-Cas9 (D1135V/G1218R/R1335E/T1337R) S. pyogenes Cas9

(SEQ ID NO: 7)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQLEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKEYRSTKEVLDATLIHQ
SITGLYETRIDLSQLGGD (single underline: HNH domain;
double underline: RuvC domain)

VRER-nCas9 (D10A/D1135V/G1218R/R1335E/T1337R) S. pyogenes Cas9Nickase

(SEQ ID NO: 8)
MDKKYSIGLAIGTNSVGWAVITDEYKVPSKKEKVLGNTDRHSIKKNLIGA
LLEDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNEDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLEDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRILEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDERKDFQFYKVREINNYHHAHDAYLNAVVGTALIKK
YPKLESEFVYGDYKVYDVRKMIAKSEQFIGKATAKYFFYSNIMNFFKTEI
TLANGEIRKRPLIETNGETGEIVWDKGRDEATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASARELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKEYRSTKEVLDATLIHQ
SITGLYETRIDLSQLGGD (single underline: HNH domain;
double underline: RuvC domain)

VQR-Cas9 (D1135V/R1335Q/T1337R) S. pyogenes Cas9

(SEQ ID NO: 9)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQLEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD (single underline: HNH domain;
double underline: RuvC domain)

VQR-nCas9 (D10A/D1135V/R1335Q/T1337R) S. pyogenes Cas9 Nickase

(SEQ ID NO: 315)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQLEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFVSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD (single underline: HNH domain;
double underline: RuvC domain)

EQR-Cas9 (D1135E/R1335Q/T1337R) S. pyogenes Cas9

(SEQ ID NO: 316)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQLEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFESPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD (single underline: HNH domain;
double underline: RuvC domain)

EQR-nCas9 (D10A/D1135E/R1335Q/T1337R) S. pyogenes Cas9 Nickase

(SEQ ID NO: 317)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQLEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLPEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFESPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKQYRSTKEVLDATLIHQ
SITGLYETRIDLSQLGGD (single underline: HNH domain;
double underline: RuvC domain)

Further variants of Cas9 from S. aureus and S. thermophilius may also be used in the contemplated methods and compositions described herein.

KKH Variant (E782K/N968K/R1015H) S. aureus Cas9

(SEQ ID NO: 318)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRKLINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYKNDLIKINGELYRVIGVNNDLLNRIEVNMIDIT
YREYLENMNDKRPPHIIKTIASKTQSIKKYSTDILGNLYEVKSKKHPQII
KKG

KKH Variant (D10A/E782K/N968K/R1015H) S. aureus Cas9 Nickase

(SEQ ID NO: 319)
MKRNYILGLAIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRKLINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYKNDLIKINGELYRVIGVNNDLLNRIEVNMIDIT
YREYLENMNDKRPPHIIKTIASKTQSIKKYSTDILGNLYEVKSKKHPQII
KKG

Streptococcus thermophilus CRISPR1 Cas9 (St1Cas9)

(SEQ ID NO: 320)
MSDLVLGLDIGIGSVGVGILNKVTGEIIHKNSRIFPAAQAENNLVRRTNR
QGRRLTRRKKHRRVRLNRLFEESGLITDFTKISINLNPYQLRVKGLTDEL
SNEELFIALKNMVKHRGISYLDDASDDGNSSIGDYAQIVKENSKQLETKT
PGQIQLERYQTYGQLRGDFTVEKDGKKHRLINVFPTSAYRSEALRILQTQ
QEFNPQITDEFINRYLEILTGKRKYYHGPGNEKSRTDYGRYRTSGETLDN
IFGILIGKCTFYPDEFRAAKASYTAQEFNLLNDLNNLTVPTETKKLSKEQ
KNQIINYVKNEKAMGPAKLFKYIAKLLSCDVADIKGYRIDKSGKAEIHTF
EAYRKMKTLETLDIEQMDRETLDKLAYVLTLNTEREGIQEALEHEFADGS
FSQKQVDELVQFRKANSSIFGKGWHNFSVKLMMELIPELYETSEEQMTIL
TRLGKQKTTSSSNKTKYIDEKLLTEEIYNPVVAKSVRQAIKIVNAAIKEY
GDFDNIVIEMARETNEDDEKKAIQKIQKANKDEKDAAMLKAANQYNGKAE
LPHSVFHGHKQLATKIRLWHQQGERCLYTGKTISIHDLINNSNQFEVDHI
LPLSITFDDSLANKVLVYATANQEKGQRTPYQALDSMDDAWSFRELKAFV
RESKTLSNKKKEYLLTEEDISKFDVRKKFIERNLVDTRYASRVVLNALQE
HFRAHKIDTKVSVVRGQFTSQLRRHWGIEKTRDTYHHHAVDALIIAASSQ
LNLWKKQKNTLVSYSEDQLLDIETGELISDDEYKESVFKAPYQHFVDTLK
SKEFEDSILFSYQVDSKFNRKISDATIYATRQAKVGKDKADETYVLGKIK
DIYTQDGYDAFMKIYKKDKSKFLMYRHDPQTFEKVIEPILENYPNKQINE
KGKEVPCNPFLKYKEEHGYIRKYSKKGNGPEIKSLKYYDSKLGNHIDITP
KDSNNKVVLQSVSPWRADVYFNKTTGKYEILGLKYADLQFEKGTGTYKIS
QEKYNDIKKKEGVDSDSEFKFTLYKNDLLLVKDTETKEQQLFRFLSRTMP
KQKHYVELKPYDKQKFEGGEALIKVLGNVANSGQCKKGLGKSNISIYKVR
TDVLGNQHIIKNEGDKPKLDF

Streptococcus thermophilus CRISPR1 Cas9 (St1Cas9) Nickase (D9A)

(SEQ ID NO: 321)
MSDLVLGLAIGIGSVGVGILNKVTGEIIHKNSRIFPAAQAENNLVRRTNR
QGRRLTRRKKHRRVRLNRLFEESGLITDFTKISINLNPYQLRVKGLTDEL
SNEELFIALKNMVKHRGISYLDDASDDGNSSIGDYAQIVKENSKQLETKT
PGQIQLERYQTYGQLRGDFTVEKDGKKHRLINVFPTSAYRSEALRILQTQ
QEFNPQITDEFINRYLEILTGKRKYYHGPGNEKSRTDYGRYRTSGETLDN
IFGILIGKCTFYPDEFRAAKASYTAQEFNLLNDLNNLTVPTETKKLSKEQ
KNQIINYVKNEKAMGPAKLFKYIAKLLSCDVADIKGYRIDKSGKAEIHTF
EAYRKMKTLETLDIEQMDRETLDKLAYVLTLNTEREGIQEALEHEFADGS
FSQKQVDELVQFRKANSSIFGKGWHNFSVKLMMELIPELYETSEEQMTIL
TRLGKQKTTSSSNKTKYIDEKLLTEEIYNPVVAKSVRQAIKIVNAAIKEY
GDFDNIVIEMARETNEDDEKKAIQKIQKANKDEKDAAMLKAANQYNGKAE
LPHSVFHGHKQLATKIRLWHQQGERCLYTGKTISIHDLINNSNQFEVDHI
LPLSITFDDSLANKVLVYATANQEKGQRTPYQALDSMDDAWSFRELKAFV
RESKTLSNKKKEYLLTEEDISKFDVRKKFIERNLVDTRYASRVVLNALQE
HFRAHKIDTKVSVVRGQFTSQLRRHWGIEKTRDTYHHHAVDALIIAASSQ
LNLWKKQKNTLVSYSEDQLLDIETGELISDDEYKESVFKAPYQHFVDTLK
SKEFEDSILFSYQVDSKFNRKISDATIYATRQAKVGKDKADETYVLGKIK
DIYTQDGYDAFMKIYKKDKSKFLMYRHDPQTFEKVIEPILENYPNKQINE
KGKEVPCNPFLKYKEEHGYIRKYSKKGNGPEIKSLKYYDSKLGNHIDITP
KDSNNKVVLQSVSPWRADVYFNKTTGKYEILGLKYADLQFEKGTGTYKIS
QEKYNDIKKKEGVDSDSEFKFTLYKNDLLLVKDTETKEQQLFRFLSRTMP
KQKHYVELKPYDKQKFEGGEALIKVLGNVANSGQCKKGLGKSNISIYKVR
TDVLGNQHIIKNEGDKPKLDF

Streptococcus thermophilus CRISPR3Cas9 (St3Cas9)

(SEQ ID NO: 322)
MTKPYSIGLDIGTNSVGWAVITDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKVYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEV
FKDDTKNGYAGYIDGKTNQEDFYVYLKNLLAEFEGADYFLEKIDREDFLR
KQRTFDNGSIPYQIHLQEMRAILDKQAKFYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNFMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSKIDNNALQNDRLYLYYLQNGKDMYTGDDLDIDRLSNYDIDHIIP
QAFLKDNSIDNKVLVSSASNRGKSDDFPSLEVVKKRKTFWYQLLKSKLIS
QRKFDNLTKAERGGLLPEDKAGFIQRQLVETRQITKHVARLLDEKFNNKK
DENNRAVRTVKIITLKSTLVSQFRKDFELYKVREINDFHHAHDAYLNAVI
ASALLKKYPKLEPEFVYGDYPKYNSFRERKSATEKVYFYSNIMNIFKKSI
SLADGRVIERPLIEVNEETGESVWNKESDLATVRRVLSYPQVNVVKKVEE
QNHGLDRGKPKGLFNANLSSKPKPNSNENLVGAKEYLDPKKYGGYAGISN
SFAVLVKGTIEKGAKKKITNVLEFQGISILDRINYRKDKLNFLLEKGYKD
IELIIELPKYSLFELSDGSRRMLASILSTNNKRGEIHKGNQIFLSQKFVK
LLYHAKRISNTINENHRKYVENHKKEFEELFYYILEFNENYVGAKKNGKL
LNSAFQSWQNHSIDELCSSFIGPTGSERKGLFELTSRGSAADFEFLGVKI
PRYRDYTPSSLLKDATLIHQSVTGLYETRIDLAKLGEG

Streptococcus thermophilus CRISPR3Cas9 (St3Cas9) Nickase (D10A)

(SEQ ID NO: 323)
MTKPYSIGLAIGTNSVGWAVITDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKVYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEV
FKDDTKNGYAGYIDGKTNQEDFYVYLKNLLAEFEGADYFLEKIDREDFLR
KQRTFDNGSIPYQIHLQEMRAILDKQAKFYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNFMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSKIDNNALQNDRLYLYYLONGKDMYTGDDLDIDRLSNYDIDHIIP
QAFLKDNSIDNKVLVSSASNRGKSDDFPSLEVVKKRKTFWYQLLKSKLIS
QRKFDNLTKAERGGLLPEDKAGFIQROLVETROITKHVARLLDEKFNNKK
DENNRAVRTVKIITLKSTLVSQFRKDFELYKVREINDFHHAHDAYLNAVI
ASALLKKYPKLEPEFVYGDYPKYNSFRERKSATEKVYFYSNIMNIFKKSI
SLADGRVIERPLIEVNEETGESVWNKESDLATVRRVLSYPQVNVVKKVEE
QNHGLDRGKPKGLFNANLSSKPKPNSNENLVGAKEYLDPKKYGGYAGISN
SFAVLVKGTIEKGAKKKITNVLEFQGISILDRINYRKDKLNFLLEKGYKD
IELIIELPKYSLFELSDGSRRMLASILSTNNKRGEIHKGNQIFLSQKFVK
LLYHAKRISNTINENHRKYVENHKKEFEELFYYILEFNENYVGAKKNGKL
LNSAFQSWQNHSIDELCSSFIGPTGSERKGLFELTSRGSAADFEFLGVKI
PRYRDYTPSSLLKDATLIHQSVTGLYETRIDLAKLGEG

It is appreciated that when the term “dCas9” or “nuclease-inactive Cas9” is used herein, it refers to Cas9 variants that are inactive in both HNH and RuvC domains as well as Cas9 nickases. For example, the dCas9 used in the present disclosure may include the amino acid sequence set forth in SEQ ID NO: 2 or SEQ ID NO: 3. In some embodiments, the dCas9 may comprise other mutations that inactivate RuvC or HNH domain. Additional suitable mutations that inactivate Cas9 will be apparent to those of skill in the art based on this disclosure and knowledge in the field, and are within the scope of this disclosure. Such additional exemplary suitable nuclease-inactive Cas9 domains include, but are not limited to, D839A and/or N863A (See, e.g., Prashant et al., Nature Biotechnology. 2013; 31(9): 833-838, the entire contents of which are incorporated herein by reference), or K603R (See. e.g., Chavez et al., Nature Methods 12, 326-328, 2015, the entire contents of which is incorporated herein by reference). The term Cas9, dCas9, or Cas9 variant also encompasses Cas9, dCas9, or Cas9 variants from any organism. Also appreciated is that dCas9, Cas9 nickase, or other appropriate Cas9 variants from any organisms may be used in accordance with the present disclosure. In one example, the Cas9 variants used herein are the D10A variants of Cas9 from S. pyogenes or S. aureus.

A “deaminase” refers to an enzyme that catalyzes the removal of an amine group from a molecule, or deamination. In some embodiments, the deaminase is a cytidine deaminase, catalyzing the deamination of cytidine or deoxycytidine to uridine or deoxyuridine, respectively. In some embodiments, the deaminase is a cytosine deaminase, catalyzing the hydrolytic deamination of cytosine to uracil (e.g., in RNA) or thymine (e.g., in DNA). In some embodiments, the deaminase is a naturally-occurring deaminase from an organism, such as a human, chimpanzee, gorilla, monkey, cow, dog, rat, or mouse. In some embodiments, the deaminase is a variant of a naturally-occurring deaminase from an organism, and the variant does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

A “cytosine deaminase” refers to an enzyme that catalyzes the chemical reaction “cytosine+H₂O [symbol] uracil+NH₃.” As it may be apparent from the reaction formula, such chemical reactions result in a C to U/T nucleobase change. In the context of a gene, such nucleotide change, or mutation, may in turn lead to an amino acid change in the protein, which may affect the protein's function, e.g., loss-of-function or gain-of-function.

One exemplary suitable class of cytosine deaminases is the apolipoprotein B mRNA-editing complex (APOBEC) family of cytosine deaminases encompassing eleven proteins that serve to initiate mutagenesis in a controlled and beneficial manner. The apolipoprotein B editing complex 3 (APOBEC3) enzyme provides protection to human cells against a certain HIV-1 strain via the deamination of cytosines in reverse-transcribed viral ssDNA. These cytosine deaminases all require a Zn²⁺-coordinating motif (His-X-Glu-X₂₃₋₂₆-Pro-Cys-X₂₋₄-Cys; SEQ ID NO: 324) and bound water molecule for catalytic activity. The glutamic acid residue acts to activate the water molecule to a zinc hydroxide for nucleophilic attack in the deamination reaction. Each family member preferentially deaminates at its own particular “hotspot,” for example, WRC (W is A or T. R is A or G) for hAID, TTC for hAPOBEC3F. A recent crystal structure of the catalytic domain of APOBEC3G revealed a secondary structure comprising a five-stranded β-sheet core flanked by six α-helices, which is believed to be conserved across the entire family. The active center loops have been shown to be responsible for both ssDNA binding and in determining “hotspot” identity. Overexpression of these enzymes has been linked to genomic instability and cancer, thus highlighting the importance of sequence-specific targeting. Another suitable cytosine deaminase is the activation-induced cytidine deaminase (AID), which is responsible for the maturation of antibodies by converting cytosines in ssDNA to uracils in a transcription-dependent, strand-biased fashion.

The term “base editors” or “nucleobase editors.” as used herein, broadly refer to any of the fusion proteins described herein. In some embodiments, the nucleobase editors are capable of precisely deaminating a target base to convert it to a different base, e.g., the base editor may target C bases in a nucleic acid sequence and convert the C to a T. In some embodiments, the base editor comprises a Cas9 (e.g., dCas9 and nCas9), CasX, CasY, Cpf1, C2c1, C2c2, C2c3, or Argonaute protein fused to a cytidine deaminase. For example, in certain embodiments, the base editor may be a cytosine deaminase-dCas9 fusion protein. In some embodiments, the base editor may be a deaminase-dCas9-UGI fusion protein. In some embodiments, the base editor may be a APOBEC1-dCas9-UGI fusion protein. In some embodiments, the base editor may be APOBEC1-Cas9 nickase-UGI fusion protein. In some embodiments, the base editor may be APOBEC1-dCpf1-UGI fusion protein. In some embodiments, the base editor may be APOBEC1-dNgAgo-UGI fusion protein. In some embodiments, the base editor may be APOBEC1-SpCas9 nickase-UGI fusion protein. In some embodiments, the base editor may be APOBEC1-SaCas9 nickase-UGI fusion protein. In some embodiments, the base editor comprises a CasX protein fused to a cytidine deaminase. In some embodiments, the base editor comprises a CasY protein fused to a cytidine deaminase. In some embodiments, the base editor comprises a Cpf1 protein fused to a cytidine deaminase. In some embodiments, the base editor comprises a C2c1 protein fused to a cytidine deaminase. In some embodiments, the base editor comprises a C2c2 protein fused to a cytidine deaminase. In some embodiments, the base editor comprises a C2c3 protein fused to a cytidine deaminase. In some embodiments, the base editor comprises an Argonaute protein fused to a cytidine deaminase. In some embodiments, the fusion protein described herein comprises a Gam protein, a guide nucleotide sequence-programmable DNA binding protein, and a cytidine deaminase domain. In some embodiments, the base editor comprises a Gam protein, fused to a CasX protein, which is fused to a cytidine deaminase. In some embodiments, the base editor comprises a Gam protein, fused to a CasY protein, which is fused to a cytidine deaminase. In some embodiments, the base editor comprises a Gam protein, fused to a Cpf1 protein, which is fused to a cytidine deaminase. In some embodiments, the base editor comprises a Gam protein, fused to a C2c1 protein, which is fused to a cytidine deaminase. In some embodiments, the base editor comprises a Gam protein, fused to a C2c2 protein, which is fused to a cytidine deaminase. In some embodiments, the base editor comprises a Gam protein, fused to a C2c3 protein, which is fused to a cytidine deaminase. In some embodiments, the base editor comprises a Gam protein, fused to an Argonaute protein, which is fused to a cytidine deaminase. Non-limiting exemplary sequences of the nucleobase editors useful in the present disclosure are provided in Example 1. SEQ ID NOs: 1-260, 270-292, or 315-323. Such nucleobase editors and methods of using them for genome editing have been described in the art, e.g., in U.S. Pat. No. 9,068,179, US Patent Application Publications US20150166980, US20150166981, US20150166982, US20150166984, and US20150165054, and U.S. Provisional Applications 62/245,828, 62/279,346, 62/311,763, 62/322,178, 62/357,352, 62/370,700, and 62/398,490 and in Komor et al., Nature, “Programmable editing of a target base in genomic DNA without double-stranded DNA cleavage.” 533, 420-424 (2016), each of which is incorporated herein by reference.

The term “uracil glycosylase inhibitor” or “UGI,” as used herein, refers to a protein that is capable of inhibiting a uracil-DNA glycosylase base-excision repair enzyme.

The term “Cas9 nickase.” as used herein, refers to a Cas9 protein that is capable of cleaving only one strand of a duplexed nucleic acid molecule (e.g., a duplexed DNA molecule). In some embodiments, a Cas9 nickase comprises a D10A mutation and has a histidine at position H840 of a wild type sequence, or a corresponding mutation in any of the Cas9 proteins provided herein. For example, a Cas9 nickase may comprise the amino acid sequence as set forth in SEQ ID NO: 683. Such a Cas9 nickase has an active HNH nuclease domain and is able to cleave the non-targeted strand of DNA, i.e., the strand bound by the gRNA. Further, such a Cas9 nickase has an inactive RuvC nuclease domain and is not able to cleave the targeted strand of the DNA, i.e., the strand where base editing is desired.

Exemplary Cas9 Nickase (Cloning Vector pPlatTET-gRNA2; Accession No. BAV54124).

(SEQ ID NO: 683)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The term “target site” or “target sequence” refers to a sequence within a nucleic acid molecule (e.g., a DNA molecule) that is deaminated by the fusion protein (e.g., a dCas9-deaminase fusion protein or a Gam-nCas9-deaminase fusion protein) provided herein. In some embodiments, the target sequence is a polynucleotide (e.g., a DNA), wherein the polynucleotide comprises a coding strand and a complementary strand. The meaning of a “coding strand” and “complementary strand,” as used herein, is the same as the common meaning of the terms in the art. In some embodiments, the target sequence is a sequence in the genome of a mammal. In some embodiments, the target sequence is a sequence in the genome of a human. In some embodiments, the target sequence is a sequence in the genome of a non-human animal. The term “target codon” refers to the amino acid codon that is edited by the base editor and converted to a different codon via deamination. The term “target base” refers to the nucleotide base that is edited by the base editor and converted to a different base via deamination. In some embodiments, the target codon in the coding strand is edited (e.g., deaminated). In some embodiments, the target codon in the complementary strand is edited (e.g., deaminated).

The term “linker.” as used herein, refers to a chemical group or a molecule linking two molecules or moieties, e.g., two domains of a fusion protein, such as, for example, a nuclease-inactive Cas9 domain and a nucleic acid editing domain (e.g., a deaminase domain). In some embodiments, a linker joins a gRNA binding domain of an RNA-programmable nuclease, including a Cas9 nuclease domain, and a catalytic domain of a nucleic-acid editing domain (e.g., a deaminase domain). In some embodiments, a linker joins a Cas9 domain (e.g., a Cas9 nickase) and a Gam protein. In some embodiments, a linker joins a gRNA binding domain of an RNA-programmable nuclease (e.g., dCas9) and a UGI domain. In some embodiments, a linker joins a a catalytic domain of a nucleic-acid editing domain (e.g., a deaminase domain) and a UGI domain. In some embodiments, a linker joins a catalytic domain of a nucleic-acid editing domain (e.g., a deaminase domain) and a Gam protein. In some embodiments, a linker joins a UGI domain and a Gam protein. Typically, the linker is positioned between, or flanked by, two groups, molecules, domains, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer (e.g. a non-natural polymer, non-peptidic polymer), or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. Linkers may be of any form known in the art. For example, the linker may be a linker from a website such as www[dot]ibi[dot]vu[dot]nl/programs/linkerdbwww/ or from www[dot] ibi[dot]vu[dot]nl/programs/linkerdbwww/src/database.txt. The linkers may also be unstructured, structured, helical, or extended.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press. Cold Spring Harbor, N.Y. (2012)).

The terms “nucleic acid.” “polynucleotide,” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments. “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments. “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine. 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein.” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially well suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), which is incorporated herein by reference.

The term “subject,” as used herein, refers to an individual organism, for example, an individual mammal. In certain embodiments of the aspects described herein, the subject is a mammal. e.g., a primate, e.g., a human. In some embodiments, the subject is a non-human mammal. In some embodiments, the subject is a non-human primate. Non-human primates include, but are not limited to, chimpanzees, cynomolgous monkeys, spider monkeys, and macaques, e.g., Rhesus. In some embodiments, the subject is any rodent, e.g., mice, rats, woodchucks, ferrets, rabbits and hamsters. In other embodiments, the subject is a domestic or game animal which includes, but is not limited to: cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a sheep, a goat, a cattle, a cat, or a dog. In some embodiments, the subject is a research animal. In some embodiments, the subject is genetically engineered, e.g., a genetically engineered non-human subject. The subject may be of either sex and at any stage of development. For example, a subject may be male or female, and can be a fully developed subject (e.g., an adult) or a subject undergoing the developmental process (e.g., a child, infant or fetus). The term “patient” or “subject” includes any subset of the foregoing, e.g., all of the above, but excluding one or more groups or species such as humans, primates or rodents. The terms, “patient” and “subject” are used interchangeably herein.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence. The fusion proteins (e.g., base editors) described herein are made recombinantly. Recombinant technology is familiar to those skilled in the art.

An “intron” refers to any nucleotide sequence within a gene that is removed by RNA splicing during maturation of the final RNA product. The term intron refers to both the DNA sequence within a gene and the corresponding sequence in RNA transcripts. Sequences that are joined together in the final mature RNA after RNA splicing are exons. Introns are found in the genes of most organisms and many viruses, and can be located in a wide range of genes, including those that generate proteins, ribosomal RNA (rRNA), and transfer RNA (tRNA). When proteins are generated from intron-containing genes, RNA splicing takes place as part of the RNA processing pathway that follows transcription and precedes translation.

An “exon” refers to any part of a gene that will become a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

“Splicing” refers to the processing of a newly synthesized messenger RNA transcript (also referred to as a primary mRNA transcript). After splicing, introns are removed and exons are joined together (ligated) for form mature mRNA molecule containing a complete open reading frame that is decoded and translated into a protein. For nuclear-encoded genes, splicing takes place within the nucleus either co-transcriptionally or immediately after transcription. The molecular mechanism of RNA splicing has been extensively described, e.g., in Pagani et al., Nature Reviews Genetics 5, 389-396, 2004: Clancy et al., Nature Education 1 (1): 31, 2011; Cheng et al., Molecular Genetics and Genonmics 286 (5-6): 395-410, 2014; Taggart et al., Nature Structural & Molecular Biology 19 (7): 719-2, 2012, the contents of each of which are incorporated herein by reference. One skilled in the art is familiar with the mechanism of RNA splicing.

“Alternative splicing” refers to a regulated process during gene expression that results in a single gene coding for multiple proteins. In this process, particular exons of a gene may be included within or excluded from the final, processed messenger RNA (mRNA) produced from that gene. Consequently, the proteins translated from alternatively spliced mRNAs will contain differences in their amino acid sequence and, often, in their biological functions. Notably, alternative splicing allows the human genome to direct the synthesis of many more proteins than would be expected from its 20,000 protein-coding genes. Alternative splicing is sometimes also termed differential splicing. Alternative splicing occurs as a normal phenomenon in eukaryotes, where it greatly increases the biodiversity of proteins that can be encoded by the genome; in humans, ˜95% of multi-exonic genes are alternatively spliced. There are numerous modes of alternative splicing observed, of which the most common is exon skipping. In this mode, a particular exon may be included in mRNAs under some conditions or in particular tissues, and omitted from the mRNA in others. Abnormal variations in splicing are also implicated in disease; a large proportion of human genetic disorders result from splicing variants. Abnormal splicing variants are also thought to contribute to the development of cancer, and splicing factor genes are frequently mutated in different types of cancer. The regulation of alternative splicing is also described in the art. e.g., in Douglas et al., Annual Review of Biochemistry 72 (1): 291-336, 2003; Pan et al., Nature Genetics 40 (12): 1413-1415, 2008; Martin et al., Nature Reviews 6 (5): 386-398, 2005; Skotheim et al., The International Journal of Biochemistry & Cell Biology 39 (7-8): 1432-49, 2007, each of which is incorporated herein by reference.

A “coding frame” or “open reading frame” refers to a stretch of codons that encodes a polypeptide. Since DNA is interpreted in groups of three nucleotides (codons), a DNA strand has three distinct reading frames. The double helix of a DNA molecule has two anti-parallel strands so, with the two strands having three reading frames each, there are six possible frame translations. A functional protein may be produced when translation proceeds in the correct coding frame. An insertion or a deletion of one or two bases in the open reading frame causes a shift in the coding frame that is also referred to as a “frameshift mutation.” A frameshift mutation typical results in premature translation termination and/or truncated or non-functional protein.

These and other exemplary substituents are described in more detail in the Detailed Description, Examples, and Claims. The methods and compositions disclosed herein are not intended to be limited in any manner by the above exemplary listing of substituents.

### DETAILED DESCRIPTION OF CERTAIN EMBODIMENTS

Disclosed herein are novel genome/base-editing systems, methods, and compositions for generating engineered and naturally-occurring protective variants of the C-C Chemokine Receptor 5 (CCR5) protein to protect against human immunodeficiency virus (HIV) infection and acquired immune deficiency syndrome (AIDS). C-C Chemokine Receptor 5 (CCR5), also known as cluster of differentiation-195 (CD195), is a member of the beta chemokine receptor family. This protein is expressed by macrophages, dendritic cells, and memory T cells in the immune system; endothelial cells, epithelial cells, vascular smooth muscle cells, and fibroblasts; and microglia, neurons, and astrocytes in the central nervous system. See, e.g., Barmania and Pepper, Applied & Translational Genomics 2 (2013) 3-16, each of which is incorporated herein by reference. Macrophage-tropic (M-tropic) strains of HIV (e.g., M-tropic strains of HIV-1) can bind CCR5 in order to enter host cells.

Certain alleles of CCR5 have been associated with resistance to HIV infection. As one example, CCR5-Δ32 (also known as CCR5-D32, CCR5Δ32, or CCR5 delta 32) is a 32-base-pair deletion that introduces a premature stop codon into the CCR5 receptor locus, resulting in a non-functional receptor. CCR5-Δ32 has a heterozygote allele frequency of 10% and a homozygote frequency of 1% in Europe. Individuals who are homozygous for CCR5-Δ32 do not express functional CCR5 receptors on their cell surfaces and are resistant to HIV-1 infection (see, for example, Liu et al., (August 1996). “Homozygous defect in HIV-1 coreceptor accounts for resistance of some multiply-exposed individuals to HIV-1 infection”, Cell. 86 (3): 367-77). Individuals heterozygous for CCR5-Δ32 have a greater than 50% reduction in functional CCR5 receptors on their cell surfaces which interferes with transport of CCR5 to the cell surface. This level of reduction is due to the dimerization of mutant and wild-type receptors (see, for example. Benkirane et al., (December 1997). “Mechanism of transdominant inhibition of CCR5-mediated HIV-1 infection by ccr5delta32”. The Journal of Biological Chemistry. 272 (49): 30603-6). These heterozygous individuals are resistant to HIV-1 infection and, if infected, exhibit reduced viral loads and a two to three year delay in the development of AIDS (relative to individuals with two wild type CCR5 genes; see, for example, Dean M et al., (September 1996). “Genetic restriction of HIV-1 infection and progression to AIDS by a deletion allele of the CKR5 structural gene. Hemophilia Growth and Development Study, Multicenter AIDS Cohort Study, Multicenter Hemophilia Cohort Study, San Francisco City Cohort, ALIVE Study”. Science. 273 (5283): 1856-62; Liu et al., (August 1996). “Homozygous defect in HIV-1 coreceptor accounts for resistance of some multiply-exposed individuals to HIV-1 infection”. Cell. 86 (3): 367-77; Michael N L et al., (October 1997). “The role of CCR5 and CCR2 polymorphisms in HIV-1 transmission and disease progression”. Nature Medicine. 3 (10): 1160-2). Further, individuals who are homozygous for CCR5-Δ32 also display an improved response to anti-retroviral treatment (see, for example, Laurichesse et al., (May 2007). “Improved virological response to highly active antiretroviral therapy in HIV-1-infected patients carrying the CCR5 Delta32 deletion.” HIV Medicine, 8 (4): 213-9).

The mRNA sequence for human CCR5, which encodes a 352 amino acid protein, can be found under GenBank Accession No. NM_000579.3 (transcript variant A) or GenBank Accession No. NM_001100168.1 (transcript variant B). Mouse and rat CCR5 mRNA sequences have been deposited and can be found under GenBank Accession Nos.: NM_009917.5 and NM_053960.3, respectively. The wild-type CCR5 human, mouse, and rat protein sequences can be found under GenBank Accession Nos.: NP_001093638.1, NP_034047.2, and NP_446412.2, respectively.

Wild Type CCR5 Gene (>Gil154091329|Ref|NM_000579.3| Homo sapiens C-C Motif Chemokine Receptor 5 (Gene/Pseudogene) (CCR5), Transcript Variant a, mRNA. SEQ ID NO: 325)

CTTCAGATAGATTATATCTGGAGTGAAGAATCCTGCCACCTATGTATCTG
GCATAGTATTCTGTGTAGTGGGATGAGCAGAGAACAAAAACAAAATAATC
CAGTGAGAAAAGCCCGTAAATAAACCTTCAGACCAGAGATCTATTCTCTA
GCTTATTTTAAGCTCAACTTAAAAAGAAGAACTGTTCTCTGATTCTTTTC
GCCTTCAATACACTTAATGATTTAACTCCACCCTCCTTCAAAAGAAACAG
CATTTCCTACTTTTATACTGTCTATATGATTGATTTGCACAGCTCATCTG
GCCAGAAGAGCTGAGACATCCGTTCCCCTACAAGAAACTCTCCCCGGGTG
GAACAAGATGGATTATCAAGTGTCAAGTCCAATCTATGACATCAATTATT
ATACATCGGAGCCCTGCCAAAAAATCAATGTGAAGCAAATCGCAGCCCGC
CTCCTGCCTCCGCTCTACTCACTGGTGTTCATCTTTGGTTTTGTGGGCAA
CATGCTGGTCATCCTCATCCTGATAAACTGCAAAAGGCTGAAGAGCATGA
CTGACATCTACCTGCTCAACCTGGCCATCTCTGACCTGTTTTTCCTTCTT
ACTGTCCCCTTCTGGGCTCACTATGCTGCCGCCCAGTGGGACTTTGGAAA
TACAATGTGTCAACTCTTGACAGGGCTCTATTTTATAGGCTTCTTCTCTG
GAATCTTCTTCATCATCCTCCTGACAATCGATAGGTACCTGGCTGTCGTC
CATGCTGTGTTTGCTTTAAAAGCCAGGACGGTCACCTTTGGGGTGGTGAC
AAGTGTGATCACTTGGGTGGTGGCTGTGTTTGCGTCTCTCCCAGGAATCA
TCTTTACCAGATCTCAAAAAGAAGGTCTTCATTACACCTGCAGCTCTCAT
TTTCCATACAGTCAGTATCAATTCTGGAAGAATTTCCAGACATTAAAGAT
AGTCATCTTGGGGCTGGTCCTGCCGCTGCTTGTCATGGTCATCTGCTACT
CGGGAATCCTAAAAACTCTGCTTCGGTGTCGAAATGAGAAGAAGAGGCAC
AGGGCTGTGAGGCTTATCTTCACCATCATGATTGTTTATTTTCTCTTCTG
GGCTCCCTACAACATTGTCCTTCTCCTGAACACCTTCCAGGAATTCTTTG
GCCTGAATAATTGCAGTAGCTCTAACAGGTTGGACCAAGCTATGCAGGTG
ACAGAGACTCTTGGGATGACGCACTGCTGCATCAACCCCATCATCTATGC
CTTTGTCGGGGAGAAGTTCAGAAACTACCTCTTAGTCTTCTTCCAAAAGC
ACATTGCCAAACGCTTCTGCAAATGCTGTTCTATTTTCCAGCAAGAGGCT
CCCGAGCGAGCAAGCTCAGTTTACACCCGATCCACTGGGGAGCAGGAAAT
ATCTGTGGGCTTGTGACACGGACTCAAGTGGGCTGGTGACCCAGTCAGAG
TTGTGCACATGGCTTAGTTTTCATACACAGCCTGGGCTGGGGGTGGGGTG
GGAGAGGTCTTTTTTAAAAGGAAGTTACTGTTATAGAGGGTCTAAGATTC
ATCCATTTATTTGGCATCTGTTTAAAGTAGATTAGATCTTTTAAGCCCAT
CAATTATAGAAAGCCAAATCAAAATATGTTGATGAAAAATAGCAACCTTT
TTATCTCCCCTTCACATGCATCAAGTTATTGACAAACTCTCCCTTCACTC
CGAAAGTTCCTTATGTATATTTAAAAGAAAGCCTCAGAGAATTGCTGATT
CTTGAGTTTAGTGATCTGAACAGAAATACCAAAATTATTTCAGAAATGTA
CAACTTTTTACCTAGTACAAGGCAACATATAGGTTGTAAATGTGTTTAAA
ACAGGTCTTTGTCTTGCTATGGGGAGAAAAGACATGAATATGATTAGTAA
AGAAATGACACTTTTCATGTGTGATTTCCCCTCCAAGGTATGGTTAATAA
GTTTCACTGACTTAGAACCAGGCGAGAGACTTGTGGCCTGGGAGAGCTGG
GGAAGCTTCTTAAATGAGAAGGAATTTGAGTTGGATCATCTATTGCTGGC
AAAGACAGAAGCCTCACTGCAAGCACTGCATGGGCAAGCTTGGCTGTAGA
AGGAGACAGAGCTGGTTGGGAAGACATGGGGAGGAAGGACAAGGCTAGAT
CATGAAGAACCTTGACGGCATTGCTCCGTCTAAGTCATGAGCTGAGCAGG
GAGATCCTGGTTGGTGTTGCAGAAGGTTTACTCTGTGGCCAAAGGAGGGT
CAGGAAGGATGAGCATTTAGGGCAAGGAGACCACCAACAGCCCTCAGGTC
AGGGTGAGGATGGCCTCTGCTAAGCTCAAGGCGTGAGGATGGGAAGGAGG
GAGGTATTCGTAAGGATGGGAAGGAGGGAGGTATTCGTGCAGCATATGAG
GATGCAGAGTCAGCAGAACTGGGGTGGATTTGGGTTGGAAGTGAGGGTCA
GAGAGGAGTCAGAGAGAATCCCTAGTCTTCAAGCAGATTGGAGAAACCCT
TGAAAAGACATCAAGCACAGAAGGAGGAGGAGGAGGTTTAGGTCAAGAAG
AAGATGGATTGGTGTAAAAGGATGGGTCTGGTTTGCAGAGCTTGAACACA
GTCTCACCCAGACTCCAGGCTGTCTTTCACTGAATGCTTCTGACTTCATA
GATTTCCTTCCCATCCCAGCTGAAATACTGAGGGGTCTCCAGGAGGAGAC
TAGATTTATGAATACACGAGGTATGAGGTCTAGGAACATACTTCAGCTCA
CACATGAGATCTAGGTGAGGATTGATTACCTAGTAGTCATTTCATGGGTT
GTTGGGAGGATTCTATGAGGCAACCACAGGCAGCATTTAGCACATACTAC
ACATTCAATAAGCATCAAACTCTTAGTTACTCATTCAGGGATAGCACTGA
GCAAAGCATTGAGCAAAGGGGTCCCATAGAGGTGAGGGAAGCCTGAAAAA
CTAAGATGCTGCCTGCCCAGTGCACACAAGTGTAGGTATCATTTTCTGCA
TTTAACCGTCAATAGGCAAAGGGGGGAAGGGACATATTCATTTGGAAATA
AGCTGCCTTGAGCCTTAAAACCCACAAAAGTACAATTTACCAGCCTCCGT
ATTTCAGACTGAATGGGGGTGGGGGGGGCGCCTTAGGTACTTATTCCAGA
TGCCTTCTCCAGACAAACCAGAAGCAACAGAAAAAATCGTCTCTCCCTCC
CTTTGAAATGAATATACCCCTTAGTGTTTGGGTATATTCATTTCAAAGGG
AGAGAGAGAGGTTTTTTTCTGTTCTGTCTCATATGATTGTGCACATACTT
GAGACTGTTTTGAATTTGGGGGATGGCTAAAACCATCATAGTACAGGTAA
GGTGAGGGAATAGTAAGTGGTGAGAACTACTCAGGGAATGAAGGTGTCAG
AATAATAAGAGGTGCTACTGACTTTCTCAGCCTCTGAATATGAACGGTGA
GCATTGTGGCTGTCAGCAGGAAGCAACGAAGGGAAATGTCTTTCCTTTTG
CTCTTAAGTTGTGGAGAGTGCAACAGTAGCATAGGACCCTACCCTCTGGG
CCAAGTCAAAGACATTCTGACATCTTAGTATTTGCATATTCTTATGTATG
TGAAAGTTACAAATTGCTTGAAAGAAAATATGCATCTAATAAAAAACACC
TTCTAAAATAAAAAAAAAAAAAAAAAAAAAAAAAAA

Wild Type CCR5 Gene, Transcript Variant B (>Gil154091327|ref|NM_001100168.1| Homo sapiens C-C Motif Chemokine Receptor 5 (Gene/Pseudogene) (CCR5), Transcript Variant B, mRNA, SEQ ID NO: 326)

CTTCAGATAGATTATATCTGGAGTGAAGAATCCTGCCACCTATGTATCTG
GCATAGTCTCATCTGGCCAGAAGAGCTGAGACATCCGTTCCCCTACAAGA
AACTCTCCCCGGGTGGAACAAGATGGATTATCAAGTGTCAAGTCCAATCT
ATGACATCAATTATTATACATCGGAGCCCTGCCAAAAAATCAATGTGAAG
CAAATCGCAGCCCGCCTCCTGCCTCCGCTCTACTCACTGGTGTTCATCTT
TGGTTTTGTGGGCAACATGCTGGTCATCCTCATCCTGATAAACTGCAAAA
GGCTGAAGAGCATGACTGACATCTACCTGCTCAACCTGGCCATCTCTGAC
CTGTTTTTCCTTCTTACTGTCCCCTTCTGGGCTCACTATGCTGCCGCCCA
GTGGGACTTTGGAAATACAATGTGTCAACTCTTGACAGGGCTCTATTTTA
TAGGCTTCTTCTCTGGAATCTTCTTCATCATCCTCCTGACAATCGATAGG
TACCTGGCTGTCGTCCATGCTGTGTTTGCTTTAAAAGCCAGGACGGTCAC
CTTTGGGGTGGTGACAAGTGTGATCACTTGGGTGGTGGCTGTGTTTGCGT
CTCTCCCAGGAATCATCTTTACCAGATCTCAAAAAGAAGGTCTTCATTAC
ACCTGCAGCTCTCATTTTCCATACAGTCAGTATCAATTCTGGAAGAATTT
CCAGACATTAAAGATAGTCATCTTGGGGCTGGTCCTGCCGCTGCTTGTCA
TGGTCATCTGCTACTCGGGAATCCTAAAAACTCTGCTTCGGTGTCGAAAT
GAGAAGAAGAGGCACAGGGCTGTGAGGCTTATCTTCACCATCATGATTGT
TTATTTTCTCTTCTGGGCTCCCTACAACATTGTCCTTCTCCTGAACACCT
TCCAGGAATTCTTTGGCCTGAATAATTGCAGTAGCTCTAACAGGTTGGAC
CAAGCTATGCAGGTGACAGAGACTCTTGGGATGACGCACTGCTGCATCAA
CCCCATCATCTATGCCTTTGTCGGGGAGAAGTTCAGAAACTACCTCTTAG
TCTTCTTCCAAAAGCACATTGCCAAACGCTTCTGCAAATGCTGTTCTATT
TTCCAGCAAGAGGCTCCCGAGCGAGCAAGCTCAGTTTACACCCGATCCAC
TGGGGAGCAGGAAATATCTGTGGGCTTGTGACACGGACTCAAGTGGGCTG
GTGACCCAGTCAGAGTTGTGCACATGGCTTAGTTTTCATACACAGCCTGG
GCTGGGGGTGGGGTGGGAGAGGTCTTTTTTAAAAGGAAGTTACTGTTATA
GAGGGTCTAAGATTCATCCATTTATTTGGCATCTGTTTAAAGTAGATTAG
ATCTTTTAAGCCCATCAATTATAGAAAGCCAAATCAAAATATGTTGATGA
AAAATAGCAACCTTTTTATCTCCCCTTCACATGCATCAAGTTATTGACAA
ACTCTCCCTTCACTCCGAAAGTTCCTTATGTATATTTAAAAGAAAGCCTC
AGAGAATTGCTGATTCTTGAGTTTAGTGATCTGAACAGAAATACCAAAAT
TATTTCAGAAATGTACAACTTTTTACCTAGTACAAGGCAACATATAGGTT
GTAAATGTGTTTAAAACAGGTCTTTGTCTTGCTATGGGGAGAAAAGACAT
GAATATGATTAGTAAAGAAATGACACTTTTCATGTGTGATTTCCCCTCCA
AGGTATGGTTAATAAGTTTCACTGACTTAGAACCAGGCGAGAGACTTGTG
GCCTGGGAGAGCTGGGGAAGCTTCTTAAATGAGAAGGAATTTGAGTTGGA
TCATCTATTGCTGGCAAAGACAGAAGCCTCACTGCAAGCACTGCATGGGC
AAGCTTGGCTGTAGAAGGAGACAGAGCTGGTTGGGAAGACATGGGGAGGA
AGGACAAGGCTAGATCATGAAGAACCTTGACGGCATTGCTCCGTCTAAGT
CATGAGCTGAGCAGGGAGATCCTGGTTGGTGTTGCAGAAGGTTTACTCTG
TGGCCAAAGGAGGGTCAGGAAGGATGAGCATTTAGGGCAAGGAGACCACC
AACAGCCCTCAGGTCAGGGTGAGGATGGCCTCTGCTAAGCTCAAGGCGTG
AGGATGGGAAGGAGGGAGGTATTCGTAAGGATGGGAAGGAGGGAGGTATT
CGTGCAGCATATGAGGATGCAGAGTCAGCAGAACTGGGGTGGATTTGGGT
TGGAAGTGAGGGTCAGAGAGGAGTCAGAGAGAATCCCTAGTCTTCAAGCA
GATTGGAGAAACCCTTGAAAAGACATCAAGCACAGAAGGAGGAGGAGGAG
GTTTAGGTCAAGAAGAAGATGGATTGGTGTAAAAGGATGGGTCTGGTTTG
CAGAGCTTGAACACAGTCTCACCCAGACTCCAGGCTGTCTTTCACTGAAT
GCTTCTGACTTCATAGATTTCCTTCCCATCCCAGCTGAAATACTGAGGGG
TCTCCAGGAGGAGACTAGATTTATGAATACACGAGGTATGAGGTCTAGGA
ACATACTTCAGCTCACACATGAGATCTAGGTGAGGATTGATTACCTAGTA
GTCATTTCATGGGTTGTTGGGAGGATTCTATGAGGCAACCACAGGCAGCA
TTTAGCACATACTACACATTCAATAAGCATCAAACTCTTAGTTACTCATT
CAGGGATAGCACTGAGCAAAGCATTGAGCAAAGGGGTCCCATAGAGGTGA
GGGAAGCCTGAAAAACTAAGATGCTGCCTGCCCAGTGCACACAAGTGTAG
GTATCATTTTCTGCATTTAACCGTCAATAGGCAAAGGGGGGAAGGGACAT
ATTCATTTGGAAATAAGCTGCCTTGAGCCTTAAAACCCACAAAAGTACAA
TTTACCAGCCTCCGTATTTCAGACTGAATGGGGGTGGGGGGGGCGCCTTA
GGTACTTATTCCAGATGCCTTCTCCAGACAAACCAGAAGCAACAGAAAAA
ATCGTCTCTCCCTCCCTTTGAAATGAATATACCCCTTAGTGTTTGGGTAT
ATTCATTTCAAAGGGAGAGAGAGAGGTTTTTTTCTGTTCTGTCTCATATG
ATTGTGCACATACTTGAGACTGTTTTGAATTTGGGGGATGGCTAAAACCA
TCATAGTACAGGTAAGGTGAGGGAATAGTAAGTGGTGAGAACTACTCAGG
GAATGAAGGTGTCAGAATAATAAGAGGTGCTACTGACTTTCTCAGCCTCT
GAATATGAACGGTGAGCATTGTGGCTGTCAGCAGGAAGCAACGAAGGGAA
ATGTCTTTCCTTTTGCTCTTAAGTTGTGGAGAGTGCAACAGTAGCATAGG
ACCCTACCCTCTGGGCCAAGTCAAAGACATTCTGACATCTTAGTATTTGC
ATATTCTTATGTATGTGAAAGTTACAAATTGCTTGAAAGAAAATATGCAT
CTAATAAAAAACACCTTCTAAAATAAAAAAAAAAAAAAAAAAAAAAAAAA
A

Human CCR5 Amino Acid Sequence (>Gil154091328|Ref|NP_001093638.1| C-C Chemokine Receptor Type 5 [Homo sapiens], SEQ ID NO: 327)

MDYQVSSPIYDINYYTSEPCQKINVKQIAARLLPPLYSLVFIFGFVGNML
VILILINCKRLKSMTDIYLLNLAISDLFFLLTVPFWAHYAAAQWDFGNTM
CQLLTGLYFIGFFSGIFFIILLTIDRYLAVVHAVFALKARTVTFGVVTSV
ITWVVAVFASLPGIIFTRSQKEGLHYTCSSHFPYSQYQFWKNFQTLKIVI
LGLVLPLLVMVICYSGILKTLLRCRNEKKRHRAVRLIFTIMIVVFLFWAP
YNIVLLLNTFQEFFGLNNCSSSNRLDQAMQVTETLGMTHCCINPIIYAFV
GEKFRNYLLVFFQKHIAKRFCKCCSIFQQEAPERASSVYTRSTGEQEISV
GL

Mouse CCR5 Amino Acid Sequence (>Gi|31542356|Ref|NP_034047.2| C-C Chemokine Receptor Type 5 [Mus musculus], SEQ ID NO: 328)

MDFQGSVPTYSYDIDYGMSAPCQKINVKQIAAQLLPPLYSLVFIFGFVGN
MMVFLILISCKKLKSVTDIYLLNLAISDLLFLLTLPFWAHYAANEWVFGN
IMCKVFTGLYHIGYFGGIFFIILLTIDRYLAIVHAVFALKVRTVNFGVIT
SVVTWAVAVFASLPEIIFTRSQKEGFHYTCSPHFPHTQYHFWKSFQTLKM
VILSLILPLLVMVICYSGILHTLFRCRNEKKRHRAVRLIFAIMIVYFLFW
TPYNIVLLLTTFQEFFGLNNCSSSNRLDQAMQATETLGMTHCCLNPVIYA
FVGEKFRSYLSVFFRKHMVKRFCKRCSIFQQDNPDRASSVYTRSTGEHEV
STGL

Rat CCR5 Amino Acid Sequence (>Gi|51592090|Ref|NP_446412.2| C-C Chemokine Receptor Type 5 [Rattus norvegicus], SEQ ID NO: 329)

MDFQGSIPTYIYDIDYSMSAPCQKFNVKQIAAQLLPPLYSLVFIFGFVGN
MMVFLILISCKKLKSMTDIYLFNLAISDLLFLLTLPFWAHYAANEWVFGN
IMCKLFTGIYHIGYFGGIFFIILLTIDRYLAIVHAVFAIKARTVNFGVIT
SVVTWVVAVFVSLPEIIFMRSQKEGSHYTCSPHFPRIQYRFWKHFQTLKM
VILSLILPLLVMVICYSGILNTLFRCRNEKKRHRAVRLIFAIMIVYFLFW
TPYNIVLLLTTFQEYFGLNNCSSSNRLDQAMQVTETLGMTHCCLNPVIYA
FVGEKFRNYLSVFFRKHIVKRFCKHCSIFQQVNPDRVSSVYTRSTGEQEV
STGL

### Strategies for Generating CCR5 Mutants

Some aspects of the present disclosure provide systems, compositions, and methods of editing polynucleotides encoding the CCR5 protein to introduce mutations into the CCR5 gene. The gene editing methods described herein, rely on nucleobase editors as described in U.S. Pat. No. 9,068,179, US Patent Application Publications US20150166980, US20150166981, US20150166982, US20150166984, and US20150165054, and U.S. Provisional Applications 62/245,828, 62/279,346, 62/311,763, 62/322,178, 62/357,352, 62/370,700, and 62/398,490, and in Komor et al., Nature, Programmable editing of a target base in genomic DNA without double-stranded DNA cleavage, 533, 420-424 (2016), each of which are incorporated herein by reference.

The nucleobase editors are highly efficient at precisely editing a target base in the CCR5 gene, and a DNA double stand break is not necessary for the gene editing, thus reducing genome instability and preventing possible oncogenic modifications that may be caused by other genome editing methods. The nucleobase editors described herein may be programmed to target and modify a single base. In some embodiments, the target base is a cytosine (C) base and may be converted to a thymine (T) base via deamination by the nucleobase editor.

To edit the polynucleotide encoding the CCR5 protein, the polynucleotide is contacted with a nucleobase editors described herein. In some embodiments, the CCR5-encoding polynucleotide is contacted with a nucleobase editor and a guide nucleotide sequence, wherein the guide nucleotide sequence targets the nucleobase editor to the target base (e.g., a C base) in the CCR5-encoding polynucleotide.

In some embodiments, the CCR5-encoding polynucleotide is the CCR5 gene locus in the genomic DNA of a cell. In some embodiments, the cell is a cultured cell. In some embodiments, the cell is in vivo. In some embodiments, the cell is in vitro. In some embodiments, the cell is ex vivo. In some embodiments, the cell is from a mammal. In some embodiments, the mammal is a human. In some embodiments, the mammal is a rodent. In some embodiments, the rodent is a mouse. In some embodiments, the rodent is a rat.

As would be understood be those skilled in the art, the CCR5-encoding polynucleotide may be a DNA molecule comprising a coding strand and a complementary strand, e.g., the CCR5 gene locus in a genome. As such, the CCR5-encoding polynucleotide may also include coding regions (e.g., exons) and non-coding regions (e.g., introns or splicing sites). In some embodiments, the target base (e.g., a C base) is located in a coding region (e.g., an exon) of the CCR5-encoding polynucleotide (e.g., the CCR5 gene locus). As such, the conversion of a base in the coding region may result in an amino acid change in the CCR5 protein sequence, i.e., a mutation. In some embodiments, the mutation is a loss of function mutation. In some embodiments, the CCR5 loss-of-function mutation is identical (or similar) to a naturally occurring CCR5 loss-of-function mutation, e.g., D2V (D2N), C20S (C20Y), C101X (C101Y), G106R, C178R (C178Y), R223Q, C269F (C269Y). In some embodiments, the loss-of-function mutation is engineered (i.e., not naturally occurring), e.g., Q4X, P19S, P19L, Q2IX, P34S, P34L, P35S, P35L, G44R, G44D, G44S, G47R, G47D, G47S, W86X, Q93X, W94X, Q102X, G111R, G111D, G115R, G115D, G115E, G145R, G145E, S149N, G163R, G163E, S149N, P162S, P162L, G163R, G163D, G163E, P183S, P183L, Q186X, Q188X, W190X, G202R, G202E, P206S, P206L, G216S, G216D, W248X, Q261X, Q277X, Q280X, E283R, E283K, C290T, C290Y, C291Y, C291T, P293S, P293L, Q328X, Q329X, P332S, P332L, R334X, A335V, R341X. This engineered mutation may be an engineered truncation.

In some embodiments, the target base is located in a non-coding region of the CCR5 gene, e.g., in an intron or a splicing site. In some embodiments, a target base is located in a splicing site and the editing of such target base causes alternative splicing of the CCR5 mRNA. In some embodiments, the alternative splicing leads to loss-of-function CCR5 mutants. In some embodiments, the alternative splicing leads to the introduction of a premature stop codon in a CCR5 mRNA, resulting in truncated and unstable CCR5 proteins. In some embodiments, CCR5 mutants that are defective in terms of folding are produced.

CCR5 variants that are particularly useful in creating using the present disclosure are variants that may increase resistance to infection by human immunodeficiency virus (HIV), prevent infection by HIV, delay the onset of AIDS, and/or slow the progression of AIDS. In some embodiments, the CCR5 variants are loss-of-function variants produced using the methods of the present disclosure express efficiently in a cell. As described herein, a loss-of function CCR5 variant may have reduced activity or levels (e.g., the CCR5 variant may not be folded correctly, may not be transported to the membrane, may demonstrate reduced binding to a ligand including RANTES, MIP-1β, or MIP-1α, may demonstrate reduced transduction of signals through the G-proteins, or may have a reduced interaction with HIV) compared to a wild type CCR5 protein. For example, the activity or levels of a loss-of-function CCR5 variant may be reduced by at least 20%, at least 30%, at least 40%, at least 50%, at least 60%, at least 70%, at least 80%, at least 90%, at least 99%, or more. In some embodiments, the loss-of-function CCR5 variant has no more than 50%, no more than 40%, no more than 30%, no more than 20%, no more than 10%, no more than 5%, no more than 1%, or less activity (e.g., the CCR5 variant may not be folded correctly, may not be transported to the membrane, may demonstrate reduced binding to a ligand including RANTES, MIP-1β, or MIP-1α, may demonstrate reduced transduction of signals through the G-proteins, or may have a reduced interaction with HIV) compared to a wild type CCR5 protein. In other embodiments, the loss-of-function CCR5 variant inhibits the spread of HIV infection from cell to cell either in vitro or in vivo by more than 90%, more than 80%, more than 70%, more than 60%, more than 50%, more than 40%, more than 30%, more than 20%, or more than 10% compared to a wild type CCR5 protein. Non-limiting, exemplary assays for determining CCR5 activity may be demonstrated by any known methodology, such as the assay for chemokine binding as disclosed by Van Riper et al., J. Exp. Med., 177, 851-856 (1993), which may be readily adapted for measurement of CCR5 binding, which is incorporated herein by reference. Non-limiting, exemplary assays for determining inhibition of the spread of HIV infection between cells may be demonstrated by methods known in the art, such as the HIV quantitation assay disclosed by Nunberg, et al., J. Virology, 65 (9). 4887-4892 (1991).

To change the CCR5 gene, the nucleobase editor interacts with the CCR5 gene (a polynucleotide molecule), wherein the nucleobase editor binds to its target sequence and edits the desired nucleobase. For example, the nucleobase editor may be expressed in a cell where CCR5 gene editing is desired (e.g., macrophages, dendritic cells, and memory T cells of the immune system; endothelial cells, epithelial cells, vascular smooth muscle cells, and fibroblasts; and microglia, neurons, and astrocytes in the central nervous system), to thereby allowing interaction of the CCR5 gene with the nucleobase editor. In some embodiments, the binding of the nucleobase editor to its target sequence in the CCR5 is mediated by a guide nucleotide sequence, e.g., a polynucleotide comprising a nucleotide sequence that is complementary to one of the strands of the target sequence in the CCR5 gene. Thus, by designing the guide nucleotide sequence, the nucleobase editor may be programmed to edit any specific target base in the CCR5 gene. In some embodiments, the guide nucleotide sequence is co-expressed with the nucleobase editor in a cell where editing is desired.

### Codon Change

Using the nucleobase editors described herein, several amino acid codons may be converted to a different codon via deamination of a target base within the codon. For example, in some embodiments, a cytosine (C) base is converted to a thymine (T) base via deamination by a nucleobase editor comprising a cytosine deaminase domain (e.g., APOBEC1 or AID). As it is familiar to one skilled in the art, conversion of a base in an amino acid codon may lead to a change of the encoded amino acid in the protein product. Cytosine deaminases are capable of converting a cytosine (C) base to a deoxyuridine (dU) base via deamination, which is replicated as a thymine (T). Thus, it is envisioned that, for amino acid codons containing a C base, the C base may be converted to T in the CCR5 gene. For example, leucine codon (ETC) may be changed to a TTC (phenylalanine) codon via the deamination of the first C on the coding strand. For amino acid codons that contains a guanine (G) base, a C base is present on the complementary strand; and the G base may be convened to an adenosine (A) via the deamination of the C on the complementary strand. For example, a ATG (Met/M) codon may be converted to a ATA (Ile/I) codon via the deamination of the third C on the complementary strand. In some embodiments, two C to T changes are required to convert a codon to a different codon. Non-limiting examples of possible mutations that may be made in a CCR5-encoding polynucleotide by the nucleobase editors of the present disclosure in order to produce novel CCR5 variants are summarized in Table 7.

In some embodiments, to bind to its target sequence and edit the desired base, the nucleobase editor depends on its guide nucleotide sequence (e.g., a guide RNA). In some embodiments, the guide nucleotide sequence is a gRNA sequence. An gRNA typically comprises a tracrRNA framework allowing for Cas9 binding, and a guide sequence, which confers sequence specificity to fusion proteins disclosed herein. In some embodiments, the guide RNA comprises a structure 5′-[guide sequence]-guuuuagagcuagaaauagcaaguuaaaauaaaggcuaguccguuaucaacuugaaaaaguggcaccgagucggugcuuuuu-3′ (SEQ ID NO: 330), wherein the guide sequence comprises a sequence that is complementary to the target sequence. In some embodiments, the guide RNA comprises a structure 5′-[guide sequence]-guuuuaguacucuggaaacagaaucuacuaaaacaaggcaaaaugccguguuuaucucgucaacuuguuggcgagauuuuuu-3′ (SEQ ID NO: 331), wherein the guide sequence comprises a sequence that is complementary to the target sequence. The guide sequence is typically 20 nucleotides long. For example, the guide sequence may be 15-25 nucleotides long. In certain embodiments, the guide sequence may be 15-20 or 20-25 nucleotides long. In some embodiments, the guide sequence is 15, 16, 17, 18, 19, 20, 21, 22, 23, 24, or 25 nucleotides long. Such suitable guide RNA sequences typically comprise guide sequences that are complementary to a nucleic sequence within 50 nucleotides upstream or downstream of the target nucleotide to be edited. In certain embodiments, the tracerRNA sequence may be guuuuagagcuagaaauagcaaguuaaaauaaaggcuaguccguuaucaacuugaaaaaguggcaccgagucggugcuuuuu (SEQ ID NO: 330) or guuuuaguacucuggaaacagaaucuacuaaaacaaggcaaaaugccguguuuaucucgucaacuuguuggcgagauuuuuu (SEQ ID NO: 331) or may have greater than or equal to 80% homology (e.g., greater than or equal to 80%, greater than or equal to 81%, greater than or equal to 82%, greater than or equal to 83%, greater than or equal to 84%, greater than or equal to 85%, greater than or equal to 86%, greater than or equal to 87%, greater than or equal to 88%, greater than or equal to 89%, greater than or equal to 90%, greater than or equal to 91%, greater than or equal to 92%, greater than or equal to 93%, greater than or equal to 94%, greater than or equal to 95%, greater than or equal to 96%, greater than or equal to 97%, greater than or equal to 98%, or greater than or equal to 99% homology) with one of these sequences.

Guide sequences that may be used to target the nucleobase editor to its target sequence to induce specific mutations are provided in Tables 3-5 and 8-10. The mutations and guide sequences presented herein are for illustration purpose only and are not meant to be limiting.

In some embodiments, cellular CCR5 activity may be reduced by reducing the level of properly folded, active CCR5 protein displayed on the surface of cells. Introducing destabilizing mutations into the wild type CCR5 protein may cause misfolding or deactivation of the protein, lack of maturation or glycosylation, or enhanced recycling by the vesicular system. A CCR5 variant comprising one or more destabilizing mutations described herein may have reduced levels or activity compared to the wild type CCR5 protein (e.g., the CCR5 variant may not be folded correctly, may not be transported to the membrane, may demonstrate reduced binding to a ligand including RANTES, MIP-1β, or MIP-1α, may demonstrate reduced transduction of signals through the G-proteins, or may have a reduced interaction with HIV). For example, the levels or activity of a CCR5 variant comprising one or more destabilizing mutations described herein may be reduced by at least about 20%, at least about 30%, at least about 40%, at least about 50%, at least about 60%, at least about 70%, at least about 80%, at least about 90%, at least about 95%, at least about 99%, or more.

The present disclosure further provides mutations that cause misfolding of CCR5 protein or structural destabilization of the CCR5 protein. Non-limiting, exemplary destabilizing CCR5 mutations that may be made using the methods described herein are shown in Table 1.

In some embodiments, CCR5 variants comprising more than one mutation described herein are contemplated. For example, a CCR5 variant may be produced using the methods described herein that include 1, 2, 3, 4, 5, 6, 7, 8, 9, 10, or more mutations selected from Tables 1-10. To make multiple mutations in the CCR5 gene, a plurality of guide nucleotide sequences may be used, each guide nucleotide sequence targeting one specific base. The nucleobase editor is capable of editing the base dictated by the guide nucleotide sequence. For example, 1, 2, 3, 4, 5, 6, 7, 8, 9, 10, or more guide nucleotide sequences may be used in a gene editing process. In some embodiments, the guide nucleotide sequences are RNAs (e.g., gRNA). In some embodiments, the guide nucleotide sequences are single stranded DNA molecules.

### Premature Stop Codons

Some aspects of the present disclosure provide strategies of editing CCR5 gene to reduce the amount of full-length, functional CCR5 protein being produced. In some embodiments, stop codons may be introduced into the coding sequence of CCR5 gene upstream of the normal stop codon (referred to as a “premature stop codon”). Premature stop codons cause premature translation termination, in turn resulting in truncated and non-functional proteins and induces rapid degradation of the mRNA via the non-sense mediated mRNA decay pathway. See, e.g., Baker et al., Current Opinion in Cell Biology 16 (3): 293-299, 2004; Chang et al., Annual Review of Biochemistry 76: 51-74, 2007; and Behm-Ansmant et al., Genes & Development 20 (4): 391-398, 2006, each of which is incorporated herein by reference.

The nucleobase editors described herein may be used to convert certain amino acid codons to a stop codon (e.g., TAA, TAG, or TGA). For example, nucleobase editors including a cytosine deaminase domain are capable of converting a cytosine (C) base to a thymine (T) base via deamination. Thus, it is envisioned that, for amino acid codons containing a C base, the C base may be converted to T. For example, a CAG (Gln/Q) codon may be changed to a TAG (amber) codon via the deamination of the first C on the coding strand. For sense codons that contain a guanine (G) base, a C base is present on the complementary strand; and the G base may be converted to an adenosine (A) via the deamination of the C on the complementary strand. For example, a TGG (Trp/W) codon may be converted to a TAG (amber) codon via the deamination of the second C on the complementary strand. In some embodiments, two C to T changes are required to convert a codon to a nonsense codon. For example, a CGG (R) codon is converted to a TAG (amber) codon via the deamination of the first C on the coding strand and the deamination of the second C on the complementary strand. Non-limiting examples of the codon changes contemplated herein are provided in Tables 5, 6, and 10.

Accordingly, the present disclosure provides non-limiting examples of amino acid codons that may be converted to premature stop codons in the CCR5 gene. In some embodiments, the introduction of stop codons may be efficacious in generating truncations when the target residue is located in a flexible loop. In some embodiments, two codons adjacent to each other may both be converted to stop codons by the action of the cytidine deaminase, resulting in two stop codons adjacent to each other (also referred to as “tandem stop codons”). “Adjacent” means there are no more than 5 amino acids between the two stop codons. For example, the two stop codons may be immediately adjacent to each other (0 amino acids in between) or have 1, 2, 3, 4, or 5 amino acids in between. The introduction of tandem stop codons may be especially efficacious in generating truncation and non-functional CCR5 variants. As a non-limiting example, the tandem stop codons may be: Q186X/Q188X, Q277X/Q288X, Q328X/Q329X, Q329X/R334X, or R341X/Q346X.

### Target Base in Non-Coding Region of CCR5 Gene Splicing Variants

Some aspects of the present disclosure provide strategies of reducing cellular CCR5 activity via preventing CCR5 mRNA maturation and production. In some embodiments, such strategies involve alterations of splicing sites in the CCR5 gene. Altered splicing site may lead to altered splicing and maturation of the CCR5 mRNA. For example, in some embodiments, an altered splicing site may lead to the skipping of an exon, in turn leading to a truncated protein product or an altered reading frame. In some embodiments, an altered splicing site may lead to translation of an intron sequence and premature translation termination when an inframe stop codon is encountered by the translating ribosome in the intron. In some embodiments, a start codon is edited and protein translation initiates at the next ATG codon, which may not be in the correct coding frame.

The splicing site typically comprises an intron donor site, a Lariat branch point, and an intron acceptor site. The mechanisms of splicing are familiar to those skilled in the art. As illustrated in Table 2, the intron donor site may have a consensus sequence of GGGTRAGT, and the C bases paired with the G bases in the intron donor site consensus sequence may be targeted by a nucleobase editor described herein, thereby altering the intron donor site. The Lariat branch point also has consensus sequences, e.g., TTGTA. The C base paired with the G base in the Lariat branch point consensus sequence may be targeted by a nucleobase editor described herein, leading to the skipping of the following exon. The intron acceptor site has a consensus sequence of YACAGG, wherein Y is a pyrimidine. The C base of the consensus sequence of the intron acceptor site, and the C base paired with the G bases in the consensus sequence of the intron acceptor site may be targeted by a nucleobase editor described herein, thereby altering the intron acceptor site, in turn leading to the skipping of an exon. General strategies of altering intron-exon junctions and the start site to produce a non-functional CCR5 protein, mimicking the HIV protective effect of the CCR5-Δ32 allele are described in Table 2.

In some embodiments, a splicing site in the CCR5-coding sequence (e.g., the CCR5 gene in the genome) is altered by a programmable nuclease. The use of a programmable nuclease (e.g., TALE, ZFN, WT Cas9, or dCas9-FokI fusion protein) in generating indels in a target sequence has been described in the art, e.g., in Maeder, et al., Mol. Cell 31 (2): 294-301, 2008; Carroll et al., Genetics Society of America, 188 (4): 773-782, 2011; Miller et al., Nature Biotechnology 25 (7): 778-785, 2007; Christian et al., Genetics 186 (2): 757-61, 2008; Li et al., Nucleic Acids Res 39 (1): 359-372, 2010; and Moscou et al., Science 326 (5959): 1501, 2009, Guilinger et al., Nature Biotechnology 2014, 32 (6). 577-82. PCT Application Publication WO 2015/089427, US Patent Application Publication US 2016-0153003, and US 2015-0291965, the each of which is incorporated herein by reference.

An “indel” refers to bases inserted or deleted in the DNA of an organism, e.g., the genomic DNA of an organism. An indel may be generated via a non-homologous end joining (NHEJ) pathway following a double-strand DNA break, e.g., by cleavage of a nuclease. During NHEJ, break ends are directly ligated without the need for a homologous template, in contrast to homology directed repair, and is thus prone to generating indels. An indel that occurs in the coding sequence of a gene, will lead to frameshift mutations if the indel is an insertion or a deletion of one or two bases. An indel that occurs in the noncoding sequence of a gene. e.g., the splicing site, may cause skipping of exons or translation of intron sequences, in turn leading to frameshifting mutations and/or premature translation termination. Thus, provided in Tables 1 and 8 are non-limiting examples of splicing sites that may be targeted via programmable nucleases, e.g., WT Cas9 or dCas9-FokI fusion protein, and the guide sequences that may be used for each target site.

### CCR2 Variants

Certain mutations in the C-C chemokine receptor type 2 (CCR2) have also been shown to protect against HIV infection. Thus, some aspects of the present disclosure provide the generation of loss-of-function variants of CCR2 (e.g., A335V and V64I) using the nucleobase editors and strategies described herein. Non-limiting examples of such variants and the guide sequence that may be used to make them are provided in Table 1.

Wild Type CCR2 Gene (>Gi|183979979|Ref|NM_001123041.2| Homo sapiens C-C Motif Chemokine Receptor 2 (CCR2), Transcript Variant A, mRNA, SEQ ID NO: 332)

TTTATTCTCTGGAACATGAAACATTCTGTTGTGCTCATATCATGCAAATT
ATCACTAGTAGGAGAGCAGAGAGTGGAAATGTTCCAGGTATAAAGACCCA
CAAGATAAAGAAGCTCAGAGTCGTTAGAAACAGGAGCAGATGTACAGGGT
TTGCCTGACTCACACTCAAGGTTGCATAAGCAAGATTTCAAAATTAATCC
TATTCTGGAGACCTCAACCCAATGTACAATGTTCCTGACTGGAAAAGAAG
AACTATATTTTTCTGATTTTTTTTTTCAAATCTTTACCATTAGTTGCCCT
GTATCTCCGCCTTCACTTTCTGCAGGAAACTTTATTTCCTACTTCTGCAT
GCCAAGTTTCTACCTCTAGATCTGTTTGGTTCAGTTGCTGAGAAGCCTGA
CATACCAGGACTGCCTGAGACAAGCCACAAGCTGAACAGAGAAAGTGGAT
TGAACAAGGACGCATTTCCCCAGTACATCCACAACATGCTGTCCACATCT
CGTTCTCGGTTTATCAGAAATACCAACGAGAGCGGTGAAGAAGTCACCAC
CTTTTTTGATTATGATTACGGTGCTCCCTGTCATAAATTTGACGTGAAGC
AAATTGGGGCCCAACTCCTGCCTCCGCTCTACTCGCTGGTGTTCATCTTT
GGTTTTGTGGGCAACATGCTGGTCGTCCTCATCTTAATAAACTGCAAAAA
GCTGAAGTGCTTGACTGACATTTACCTGCTCAACCTGGCCATCTCTGATC
TGCTTTTTCTTATTACTCTCCCATTGTGGGCTCACTCTGCTGCAAATGAG
TGGGTCTTTGGGAATGCAATGTGCAAATTATTCACAGGGCTGTATCACAT
CGGTTATTTTGGCGGAATCTTCTTCATCATCCTCCTGACAATCGATAGAT
ACCTGGCTATTGTCCATGCTGTGTTTGCTTTAAAAGCCAGGACGGTCACC
TTTGGGGTGGTGACAAGTGTGATCACCTGGTTGGTGGCTGTGTTTGCTTC
TGTCCCAGGAATCATCTTTACTAAATGCCAGAAAGAAGATTCTGTTTATG
TCTGTGGCCCTTATTTTCCACGAGGATGGAATAATTTCCACACAATAATG
AGGAACATTTTGGGGCTGGTCCTGCCGCTGCTCATCATGGTCATCTGCTA
CTCGGGAATCCTGAAAACCCTGCTTCGGTGTCGAAACGAGAAGAAGAGGC
ATAGGGCAGTGAGAGTCATCTTCACCATCATGATTGTTTACTTTCTCTTC
TGGACTCCCTATAATATTGTCATTCTCCTGAACACCTTCCAGGAATTCTT
CGGCCTGAGTAACTGTGAAAGCACCAGTCAACTGGACCAAGCCACGCAGG
TGACAGAGACTCTTGGGATGACTCACTGCTGCATCAATCCCATCATCTAT
GCCTTCGTTGGGGAGAAGTTCAGAAGCCTTTTTCACATAGCTCTTGGCTG
TAGGATTGCCCCACTCCAAAAACCAGTGTGTGGAGGTCCAGGAGTGAGAC
CAGGAAAGAATGTGAAAGTGACTACACAAGGACTCCTCGATGGTCGTGGA
AAAGGAAAGTCAATTGGCAGAGCCCCTGAAGCCAGTCTTCAGGACAAAGA
AGGAGCCTAGAGACAGAAATGACAGATCTCTGCTTTGGAAATCACACGTC
TGGCTTCACAGATGTGTGATTCACAGTGTGAATCTTGGTGTCTACGTTAC
CAGGCAGGAAGGCTGAGAGGAGAGAGACTCCAGCTGGGTTGGAAAACAGT
ATTTTCCAAACTACCTTCCAGTTCCTCATTTTTGAATACAGGCATAGAGT
TCAGACTTTTTTTAAATAGTAAAAATAAAATTAAAGCTGAAAACTGCAAC
TTGTAAATGTGGTAAAGAGTTAGTTTGAGTTACTATCATGTCAAACGTGA
AAATGCTGTATTAGTCACAGAGATAATTCTAGCTTTGAGCTTAAGAATTT
TGAGCAGGTGGTATGTTTGGGAGACTGCTGAGTCAACCCAATAGTTGTTG
ATTGGCAGGAGTTGGAAGTGTGTGATCTGTGGGCACATTAGCCTATGTGC
ATGCAGCATCTAAGTAATGATGTCGTTTGAATCACAGTATACGCTCCATC
GCTGTCATCTCAGCTGGATCTCCATTCTCTCAGGCTTGCTGCCAAAAGCC
TTTTGTGTTTTGTTTTGTATCATTATGAAGTCATGCGTTTAATCACATTC
GAGTGTTTCAGTGCTTCGCAGATGTCCTTGATGCTCATATTGTTCCCTAT
TTTGCCAGTGGGAACTCCTAAATCAAGTTGGCTTCTAATCAAAGCTTTTA
AACCCTATTGGTAAAGAATGGAAGGTGGAGAAGCTCCCTGAAGTAAGCAA
AGACTTTCCTCTTAGTCGAGCCAAGTTAAGAATGTTCTTATGTTGCCCAG
TGTGTTTCTGATCTGATGCAAGCAAGAAACACTGGGCTTCTAGAACCAGG
CAACTTGGGAACTAGACTCCCAAGCTGGACTATGGCTCTACTTTCAGGCC
ACATGGCTAAAGAAGGTTTCAGAAAGAAGTGGGGACAGAGCAGAACTTTC
ACCTTCATATATTTGTATGATCCTAATGAATGCATAAAATGTTAAGTTGA
TGGTGATGAAATGTAAATACTGTTTTTAACAACTATGATTTGGAAAATAA
ATCAATGCTATAACTATGTTGAAAAAAAAAAAAAAAAAA

Wild Type CCR2 Gene, Transcript Variant B (>Gi|183979981|Ref|NM_001123396.1| Homo sapiens C-C Motif Chemokine Receptor 2 (CCR2), Transcript Variant B, mRNA. SEQ ID NO: 333)

TTTATTCTCTGGAACATGAAACATTCTGTTGTGCTCATATCATGCAAATT
ATCACTAGTAGGAGAGCAGAGAGTGGAAATGTTCCAGGTATAAAGACCCA
CAAGATAAAGAAGCTCAGAGTCGTTAGAAACAGGAGCAGATGTACAGGGT
TTGCCTGACTCACACTCAAGGTTGCATAAGCAAGATTTCAAAATTAATCC
TATTCTGGAGACCTCAACCCAATGTACAATGTTCCTGACTGGAAAAGAAG
AACTATATTTTTCTGATTTTTTTTTTCAAATCTTTACCATTAGTTGCCCT
GTATCTCCGCCTTCACTTTCTGCAGGAAACTTTATTTCCTACTTCTGCAT
GCCAAGTTTCTACCTCTAGATCTGTTTGGTTCAGTTGCTGAGAAGCCTGA
CATACCAGGACTGCCTGAGACAAGCCACAAGCTGAACAGAGAAAGTGGAT
TGAACAAGGACGCATTTCCCCAGTACATCCACAACATGCTGTCCACATCT
CGTTCTCGGTTTATCAGAAATACCAACGAGAGCGGTGAAGAAGTCACCAC
CTTTTTTGATTATGATTACGGTGCTCCCTGTCATAAATTTGACGTGAAGC
AAATTGGGGCCCAACTCCTGCCTCCGCTCTACTCGCTGGTGTTCATCTTT
GGTTTTGTGGGCAACATGCTGGTCGTCCTCATCTTAATAAACTGCAAAAA
GCTGAAGTGCTTGACTGACATTTACCTGCTCAACCTGGCCATCTCTGATC
TGCTTTTTCTTATTACTCTCCCATTGTGGGCTCACTCTGCTGCAAATGAG
TGGGTCTTTGGGAATGCAATGTGCAAATTATTCACAGGGCTGTATCACAT
CGGTTATTTTGGCGGAATCTTCTTCATCATCCTCCTGACAATCGATAGAT
ACCTGGCTATTGTCCATGCTGTGTTTGCTTTAAAAGCCAGGACGGTCACC
TTTGGGGTGGTGACAAGTGTGATCACCTGGTTGGTGGCTGTGTTTGCTTC
TGTCCCAGGAATCATCTTTACTAAATGCCAGAAAGAAGATTCTGTTTATG
TCTGTGGCCCTTATTTTCCACGAGGATGGAATAATTTCCACACAATAATG
AGGAACATTTTGGGGCTGGTCCTGCCGCTGCTCATCATGGTCATCTGCTA
CTCGGGAATCCTGAAAACCCTGCTTCGGTGTCGAAACGAGAAGAAGAGGC
ATAGGGCAGTGAGAGTCATCTTCACCATCATGATTGTTTACTTTCTCTTC
TGGACTCCCTATAATATTGTCATTCTCCTGAACACCTTCCAGGAATTCTT
CGGCCTGAGTAACTGTGAAAGCACCAGTCAACTGGACCAAGCCACGCAGG
TGACAGAGACTCTTGGGATGACTCACTGCTGCATCAATCCCATCATCTAT
GCCTTCGTTGGGGAGAAGTTCAGAAGGTATCTCTCGGTGTTCTTCCGAAA
GCACATCACCAAGCGCTTCTGCAAACAATGTCCAGTTTTCTACAGGGAGA
CAGTGGATGGAGTGACTTCAACAAACACGCCTTCCACTGGGGAGCAGGAA
GTCTCGGCTGGTTTATAAAACGAGGAGCAGTTTGATTGTTGTTTATAAAG
GGAGATAACAATCTGTATATAACAACAAACTTCAAGGGTTTGTTGAACAA
TAGAAACCTGTAAAGCAGGTGCCCAGGAACCTCAGGGCTGTGTGTACTAA
TACAGACTATGTCACCCAATGCATATCCAACATGTGCTCAGGGAATAATC
CAGAAAAACTGTGGGTAGAGACTTTGACTCTCCAGAAAGCTCATCTCAGC
TCCTGAAAAATGCCTCATTACCTTGTGCTAATCCTCTTTTTCTAGTCTTC
ATAATTTCTTCACTCAATCTCTGATTCTGTCAATGTCTTGAAATCAAGGG
CCAGCTGGAGGTGAAGAAGAGAATGTGACAGGCACAGATGAATGGGAGTG
AGGGATAGTGGGGTCAGGGCTGAGAGGAGAAGGAGGGAGACATGAGCATG
GCTGAGCCTGGACAAAGACAAAGGTGAGCAAAGGGCTCACGCATTCAGCC
AGGAGATGATACTGGTCCTTAGCCCCATCTGCCACGTGTATTTAACCTTG
AAGGGTTCACCAGGTCAGGGAGAGTTTGGGAACTGCAATAACCTGGGAGT
TTTGGTGGAGTCCGATGATTCTCTTTTGCATAAGTGCATGACATATTTTT
GCTTTATTACAGTTTATCTATGGCACCCATGCACCTTACATTTGAAATCT
ATGAAATATCATGCTCCATTGTTCAGATGCTTCTTAGGCCACATCCCCCT
GTCTAAAAATTCAGAAAATTTTTGTTTATAAAAGA

Human CCR2 Isoform A, Amino Acid Sequence (>Gi|183979980|Ref|NP_001116513.2| C-C Chemokine Receptor Type 2 Isoform A [Homo sapiens], SEQ ID NO: 334)

MLSTSRSRFIRNTNESGEEVTTFFDYDYGAPCHKFDVKQIGAQLLPPLYS
LVFIFGFVGNMLVVLILINCKKLKCLTDIYLLNLAISDLLFLITLPLWAH
SAANEWVFGNAMCKLFTGLYHIGYFGGIFFIILLTIDRYLAIVHAVFALK
ARTVTFGVVTSVITWLVAVFASVPGIIFTKCQKEDSVYVCGPYFPRGWNN
FHTIMRNILGLVLPLLIMVICYSGILKTLLRCRNEKKRHRAVRVIFTIMI
VYFLFWTPYNIVILLNTFQEFFGLSNCESTSQLDQATQVTETLGMTHCCI
NPIIYAFVGEKFRSLFHIALGCRIAPLQKPVCGGPGVRPGKNVKVTTQGL
LDGRGKGKSIGRAPEASLQDKEGA

Human CCR2 Isoform B, Amino Acid Sequence (>Gi|183979982|Ref|NP_001116868.1| C-C Chemokine Receptor Type 2 Isoform B [Homo sapiens]. SEQ ID NO: 335)

MLSTSRSRFIRNTNESGEEVTTFFDYDYGAPCHKFDVKQIGAQLLPPLYS
LVFIFGFVGNMLVVLILINCKKLKCLTDIYLLNLAISDLLFLITLPLWAH
SAANEWVFGNAMCKLFTGLYHIGYFGGIFFIILLTIDRYLAIVHAVFALK
ARTVTFGVVTSVITWLVAVFASVPGIIFTKCQKEDSVYVCGPYFPRGWNN
FHTIMRNILGLVLPLLIMVICYSGILKTLLRCRNEKKRHRAVRVIFTIMI
VYFLFWTPYNIVILLNTFQEFFGLSNCESTSQLDQATQVTETLGMTHCCI
NPIIYAFVGEKFRRYLSVFFRKHITKRFCKQCPVFYRETVDGVTSTNTPS
TGEQEVSAGL

Mouse CCR2 Amino Acid Sequence (>Gi|6753466|Ref|NP_034045.1| C-C Chemokine Receptor Type 2 [Mus musculus]. SEQ ID NO: 336)

MEDNNMLPQFIHGILSTSHSLFTRSIQELDEGATTPYDYDDGEPCHKTSV
KQIGAWILPPLYSLVFIFGFVGNMLVIIILIGCKKLKSMTDIYLLNLAIS
DLLFLLTLPFWAHYAANEWVFGNIMCKVFTGLYHIGYFGGIFFIILLTID
RYLAIVHAVFALKARTVTFGVITSVVTWVVAVFASLPGIIFTKSKQDDHH
YTCGPYFTQLWKNFQTIMRNILSLILPLLVMVICYSGILHTLFRCRNEKK
RHRAVRLIFAIMIVYFLFWTPYNIVLFLTTFQESLGMSNCVIDKHLDQAM
QVTETLGMTHCCINPVIYAFVGEKFRRYLSIFFRKHIAKRLCKQCPVFYR
ETADRVSSTFTPSTGEQEVSVGL

Rat CCR2 Amino Acid Sequence (>Gi|11177914|Ref|NP_068638.1| C-C Chemokine Receptor Type 2 [Rattus norvegicus], SEQ ID NO: 337)

MEDSNMLPQFIHGILSTSHSLFPRSIQELDEGATTPYDYDDGEPCHKTSV
KQIGAWILPPLYSLVFIFGFVGNMLVIIILISCKKLKSMTDIYLFNLAIS
DLLFLLTLPFWAHYAANEWVFGNIMCKLFTGLYHIGYFGGIFFIILLTID
RYLAIVHAVFALKARTVTFGVITSVVTWVVAVFASLPGIIFTKSEQEDDQ
HTCGPYFPTIWKNFQTIMRNILSLILPLLVMVICYSGILHTLFRCRNEKK
RHRAVRLIFAIMIVYFLFWTPYNIVLFLTTFQEFLGMSNCVVDMHLDQAM
QVTETLGMTHCCVNPIIYAFVGEKFRRYLSIFFRKHIAKNLCKQCPVFYR
ETADRVSSTFTPSTGEQEVSVGL

In some embodiments, simultaneous introduction of loss-of-function mutations into more than one protein factor affecting HIV infection are provided. For example, in some embodiments, a loss-of-function mutation may be simultaneously introduced into CCR5 and CCR2. In some embodiments to simultaneously introduce loss-of-function mutations into more than one protein, multiple guide nucleotide sequences are used. In some embodiments a guide nucleotide matching both gene sequences is used to simultaneously introduce loss-of-function mutations into more than one protein. In some embodiments a guide nucleotide partially matching one or both of the gene sequences is used to simultaneously introduce loss-of-function mutations into more than one protein, wherein one to four mistmatches are allowed between the guide RNA and a target sequence.

Further provided herein are the generation of novel and uncharacterized mutations in any of the protein factors involved in HIV infection. For example, libraries of guide nucleotide sequences may be designed for all possible PAM sequences in the genomic site of these protein factors, and used to generate mutations in these proteins. The function of the protein variants may be evaluated. If a loss-of-function variant is identified, the specific gRNA used for making the mutation may be identified via sequencing of the edited genomic site, e.g., via DNA deep sequencing.

### Nucleobase Editors

The methods of generating loss-of-function CCR5 variants described herein are enabled by the use of the nucleobase editors. As described herein, a nucleobase editor is a fusion protein comprising: (i) a programmable DNA binding protein domain; and (ii) a deaminase domain. It is to be understood that any programmable DNA binding domain may be used in the base editors.

In some embodiments, the programmable DNA binding protein domain comprises the DNA binding domain of a zinc finger nuclease (ZFN) or a transcription activator-like effector domain (TALE). In some embodiments, the programmable DNA binding protein domain may be programmed by a guide nucleotide sequence and is thus referred as a “guide nucleotide sequence-programmable DNA binding-protein domain.” In some embodiments, the guide nucleotide sequence-programmable DNA binding protein is a nuclease inactive Cas9, or dCas9. A dCas9, as used herein, encompasses a Cas9 that is completely inactive in its nuclease activity, or partially inactive in its nuclease activity (e.g., a Cas9 nickase). Thus, in some embodiments, the guide nucleotide sequence-programmable DNA binding protein is a Cas9 nickase. In some embodiments, the guide nucleotide sequence-programmable DNA binding protein is a nuclease inactive Cpf1. In some embodiments, the guide nucleotide sequence-programmable DNA binding protein is a nuclease inactive Argonaute.

In some embodiments, the guide nucleotide sequence-programmable DNA binding protein is a dCas9 domain. In some embodiments, the guide nucleotide sequence-programmable DNA binding protein is a Cas9 nickase. In some embodiments, the dCas9 domain comprises an amino acid sequence of SEQ ID NO: 2 or SEQ ID NO: 3. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 1-260, 270-292, 315-323, 680, or 682), and comprises mutations corresponding to DIOX (X is any amino acid except for D) and/or H840X (X is any amino acid except for H) in SEQ ID NO: 1. In some embodiments, the dCas9 domain comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 1-260, 270-292, 315-323, 680, or 682), and comprises mutations corresponding to D10A and/or H840A in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 1-260, 270-292, 315-323, 680, or 682), and comprises mutations corresponding to D10X (X is any amino acid except for D) in SEQ ID NO: 1 and a histidine at a position correspond to position 840 in SEQ ID NO: 1. In some embodiments, the Cas9 nickase comprises an amino acid sequence that is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% identical to any one of the Cas9 domains provided herein (e.g., SEQ ID NOs: 1-260, 270-292, 315-323, 680, or 682), and comprises mutations corresponding to D10A in SEQ ID NO: 1 and a histidine at a position correspond to position 840 in SEQ ID NO: 1. In some embodiments, variants or homologues of dCas9 or Cas9 nickase (e.g., variants of SEQ ID NO: 2 or SEQ ID NO: 3, respectively) are provided which are at least about 70% identical, at least about 80% identical, at least about 90% identical, at least about 95% identical, at least about 98% identical, at least about 99% identical, at least about 99.5% identical, or at least about 99.9% identical to SEQ ID NO: 2 or SEQ ID NO: 3, respectively, and comprises mutations corresponding to D10A and/or H840A in SEQ ID NO: 1. In some embodiments, variants of Cas9 (e.g., variants of SEQ ID NO: 2) are provided having amino acid sequences which are shorter, or longer than SEQ ID NO: 2, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and/or H840A in SEQ ID NO: 1. In some embodiments, variants of Cas9 nickase (e.g., variants of SEQ ID NO: 3) are provided having amino acid sequences which are shorter, or longer than SEQ ID NO: 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more, provided that the dCas9 variants comprise mutations corresponding to D10A and comprises a histidine at a position corresponding to position 840 in SEQ ID NO: 1.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, D10A/D839A/H840A/N863A mutant domains in SEQ ID NO: 1 (See, e.g., Prashant et al., Nature Biotechnology, 2013; 31(9): 833-838, which is incorporated herein by reference), or K603R (See, e.g., Chavez et al., Nature Methods 12, 326-328, 2015, which is incorporated herein by reference).

In some embodiments, the nucleobase editors described herein comprise a Cas9 domain with decreased electrostatic interactions between the Cas9 domain and a sugar-phosphate backbone of a DNA, as compared to a wild-type Cas9 domain. In some embodiments, a Cas9 domain comprises one or more mutations that decreases the association between the Cas9 domain and a sugar-phosphate backbone of a DNA. In some embodiments, the nucleobase editors described herein comprises a dCas9 (e.g., with D10A and H840A mutations in SEQ ID NO: 1) or a Cas9 nickase (e.g., with D10A mutation in SEQ ID NO: 1), wherein the dCas9 or the Cas9 nickase further comprises one or more of a N497X, a R661X, a Q695X, and/or a Q926X mutation of the amino acid sequence provided in SEQ ID NO: 1, or a corresponding mutation in any of the amino acid sequences provided in SEQ ID NOs: 11-260, wherein X is any amino acid. In some embodiments, the nucleobase editors described herein comprises a dCas9 (e.g., with D10A and H840A mutations in SEQ ID NO: 1) or a Cas9 nickase (e.g., with D10A mutation in SEQ ID NO: 1), wherein the dCas9 or the Cas9 nickase further comprises one or more of a N497A, a R661A, a Q695A, and/or a Q926A mutation of the amino acid sequence provided in SEQ ID NO: 1, or a corresponding mutation in any of the amino acid sequences provided in SEQ ID NOs: 11-260. In some embodiments, the Cas9 domain (e.g., of any of the nucleobase editors provided herein) comprises the amino acid sequence as set forth in SEQ ID NO: 338. In some embodiments, the nucleobase editor comprises the amino acid sequence as set forth in SEQ ID NO: 339.

Cas9 Variant with Decreased Electrostatic Interactions Between the Cas9 and DNA Backbone

DKKYSIGLAIGTNSVGWAVITDEYKVPSKKFKVLGNTDRHSIKKNLIGAL
LFDSGETALATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTAFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKDA
KDKDFLDNEENEDILEDIVLTLTLFEDREMIEERLKTYAHLFDDKVMKQL
KRRRYTGWGALSRKLINGIRDKQSGKTILDFLKSDGFANRNFMALIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRAITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD (SEQ ID NO: 338, mutations
relative to SEQ ID NO: 1 are bolded and underlined)

### High Fidelity Nucleobase Editor (HF-BE3)

(SEQ ID NO: 339)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGSETPGTSESATPESDKKYS
IGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLA
RGNSRFAWMTRKSEETITPWNFEEVVDKGASAQSFIERMTAFDKNLPNEK
VLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRY
TGWGALSRKLINGIRDKQSGKTILDFLKSDGFANRNFMALIHDDSLTFKE
DIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERG
GLSELDKAGFIKRQLVETRAITKHVAQILDSRMNTKYDENDKLIREVKVI
TLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQA
ENIIHLFTLTNLGAPAAFKYFDTTIDRKRYTSTKEVLDATLIHQSITGLY
ETRIDLSQLGGD

The Cas9 protein recognizes a short motif (PAM motif) within the target DNA sequence, which is required for the Cas9-DNA interaction but that is not determined by complementarity to the guide RNA nucleotide sequence. A “PAM motif” or “protospacer adjacent motif,” as used herein, refers to a DNA sequence adjacent to the 5′- or 3′-immediately following the DNA sequence that is complementary to the guide RNA oligonucleotide sequence. Cas9 will not successfully bind to, cleave, or nick the target DNA sequence if it is not followed by an appropriate PAM sequence. Without wishing to be bound by any particular theory, specific amino acid residues in the Cas9 enzyme are responsible for interacting with the bases of the PAM and determine the PAM specificity. Therefore, changes in these residues or nearby residues leads to a different or relaxed PAM specificity. Changing or relaxing the PAM specificity may shift the places where Cas9 can bind on the CCR5 gene sequence, and it may modify the target window available to the fused cytidine deaminase, as it will be apparent to those of skill in the art based on the instant disclosure.

Wild-type Streptococcus pyogenes Cas9 recognizes a canonical PAM sequence (5′-NGG-3′). Other Cas9 nucleases (e.g., Cas9 from Streptococcus thermophiles, Staphylococcus aureus, Neisseria meningitidis, or Treponema denticolaor) and Cas9 variants thereof have been described in the art to have different, or more relaxed PAM requirements. For example, in Kleinstiver et al., Nature 523, 481-485, 2015; Klenstiver et al., Nature 529, 490-495, 2016; Ran et al., Nature, April 9; 520(7546): 186-191, 2015; Kleinstiver et al., Nat Biotechnol. 33(12):1293-1298, 2015; Hou et al., Proc Natl Acad Sci USA, 110(39):15644-9, 2014; Prykhozhij et al., PLoS One, 10(3): e0119372, 2015; Zetsche et al., Cell 163, 759-771, 2015; Gao et al., Nature Biotechnology, doi:10.1038/nbt.3547, 2016; Want et al., Nature 461, 754-761, 2009; Chavez et al., doi: dx.doi dot org/10.1101/058974; Fagerlund et al., Genonme Biol. 2015; 16: 25, 2015; Zetsche et al., Cell, 163, 759-771, 2015: and Swarts et al., Nat Struct Mol Biol. 21(9):743-53, 2014, each of which is incorporated herein by reference.

Thus, the guide nucleotide sequence-programmable DNA-binding protein of the present disclosure may recognize a variety of PAM sequences including, without limitation PAM sequences that are on the 3′ or the 5′ end of the DNA sequence determined by the guide RNA. For example, the sequence may be: NGG, NGAN, NGNG, NGAG, NGCG, NNGRRT, NGRRN, NNNRRT, NNNGATT, NNAGAAW, NAAAC, TTN, TTTN, and YTN, wherein Y is a pyrimidine, R is a purine, and N is any nucleobase.

One example of an RNA-programmable DNA-binding protein that has different PAM specificity is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it may utilize a T-rich protospacer-adjacent motif (e.g., TTN, TTTN, or YTN), which is on the 5′-end of the DNA sequence determined by the guide RNA. Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells.

Also useful in the present compositions and methods are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A. E1006A, or D1255A in Francisella novicida Cpf1 (SEQ ID NO: 340) inactivates Cpf1 nuclease activity. In some embodiments, the dCpf1 of the present disclosure may comprise mutations corresponding to D917A, E1006A, D1255A, D917A/E1006A, D917A/D1255A, E1006A/D1255A, or D917A/E1006A/D1255A in SEQ ID NO: 340. In other embodiments, the Cpf1 nickase of the present disclosure may comprise mutations corresponding to D917A, E1006A, D1255A, D917A/E1006A, D917A/D1255A. E1006A/D1255A, or D917A/E1006A/D1255A in SEQ ID NO: 340. A Cpf1 nickase useful for the embodiments of the instant disclosure may comprise other mutations and/or further mutations known in the field. It is to be understood that any mutations, e.g., substitution mutations, deletions, or insertions that fully or partially inactivates the RuvC domain of Cpf1 may be used in accordance with the present disclosure, and that these mutations of Cpf1 may result in, for example, a dCpf1 or Cpf1 nickase.

Thus, in some embodiments, the guide nucleotide sequence-programmable DNA binding protein is a nuclease inactive Cpf1 (dCpf1). In some embodiments, the dCpf1 comprises an amino acid sequence of any one SEQ ID NOs: 340-347. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of SEQ ID NOs: 340-347, and comprises mutations corresponding to D917A, E1006A, D1255A, D917A/E1006A, D917A/D1255A, E1006A/D1255A, or D917A/E1006A/D1255A in SEQ ID NO: 340. Cpf1 from other bacterial species may also be used in accordance with the present disclosure, as a dCpf1 or Cpf1 nickase.

Wild Type Francisella novicida Cpf1 (SEQ ID NO: 340) (D917. E1006, and D1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

Francisella novicida Cpf1 D917A (SEQ ID NO: 341) (A917, E1006, and D1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

Francisella novicida Cpf1 E1006A (SEQ ID NO: 342) (D917, A1006, and D1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

Francisella novicida Cpf1 D1255A (SEQ ID NO: 343) (D917, E1006, and A1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

Francisella novicida Cpf1 D917A/E1006A (SEQ ID NO: 344) (A917, A1006, and D1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

Francisella novicida Cpf1 D917A/D1255A (SEQ ID NO: 345) (A917, E1006, and A1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

Francisella novicida Cpf1 E1006A/D1255A (SEQ ID NO: 346) (D917, A1006, and A1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

Francisella novicida Cpf1 D917A/E1006A/D1255A (SEQ ID NO: 347) (A917, A1006, and A1255 are Bolded and Underlined)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIARGERHLAYYTLVDGKGNIIKQDTFNIIGNDRMK
TNYHDKLAAIEKDRDSARKDWKKINNIKEMKEGYLSQVVHEIAKLVIEYN
AIVVFADLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDAAANGAYHIGLKGLMLLGRIKNNQEGKKLNLVIKNEEYFEFVQNRNN

In some embodiments, the guide nucleotide sequence-programmable DNA binding protein is a Cpf1 protein from a Acidaminococcus species (AsCpf1). Cpf1 proteins form Acidaminococcus species have been described previously and would be apparent to the skilled artisan. Exemplary Acidaminococcus Cpf1 proteins (AsCpf1) include, without limitation, any of the AsCpf1 proteins provided herin.

### Wild-Type AsCpf1-Residue R912 is Indicated in Bold Underlining and Residues 661-667 are Indicated in Italics and Underlining.

(SEQ ID NO: 684)
TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

### AsCpf1(R912A)—Residue A912 is Indicated in Bold Underlining and Residues 661-667 are Indicated in Italics and Underlining.

(SEQ ID NO: 686)
TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGEANLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In some embodiments, the guide nucleotide sequence-programmable DNA binding protein is a Cpf1 protein from a Lachnospiraceae species (LbCpf1). Cpf1 proteins form Lachnospiraceae species have been described previously have been described previously and would be apparent to the skilled artisan. Exemplary Lachnospiraceae Cpf1 proteins (LbCpf1) include, without limitation, any of the LbCpf1 proteins provided herein.

### Wild-Type LbCpf1—Residues R836 and R1138 is Indicated in Bold Underlining.

(SEQ ID NO: 685)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMRNSITGRTDVDFL
ISPVKNSDGIFYDSRNYEAQENAILPKNADANGAYNIARKVLWAIGQFKK
AEDEKLDKVKIAISNKEWLEYAQTSVKH

### LbCpf1 (R836A)—Residue A836 is Indicated in Bold Underlining.

(SEQ ID NO: 687)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGEANLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMRNSITGRTDVDFL
ISPVKNSDGIFYDSRNYEAQENAILPKNADANGAYNIARKVLWAIGQFKK
AEDEKLDKVKIAISNKEWLEYAQTSVKH

### LbCpf1 (R1138A)—Residue A1138 is Indicated in Bold Underlining.

(SEQ ID NO: 688)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIFRNPKKNNVFDWEEVCLTSAYKELFN
KYGINYQQGDIRALLCEQSDKAFYSSFMALMSLMLQMANSITGRTDVDFL
ISPVKNSDGIFYDSRNYEAQENAILPKNADANGAYNIARKVLWAIGQFKK
AEDEKLDKVKIAISNKEWLEYAQTSVKH

In some embodiments, the Cpf1 protein is a crippled Cpf1 protein. As used herein a “crippled Cpf1” protein is a Cpf1 protein having diminished nuclease activity as compared to a wild-type Cpf1 protein. In some embodiments, the crippled Cpf1 protein preferentially cuts the target strand more efficiently than the non-target strand. For example, the Cpf1 protein preferentially cuts the strand of a duplexed nucleic acid molecule in which a nucleotide to be edited resides. In some embodiments, the crippled Cpf1 protein preferentially cuts the non-target strand more efficiently than the target strand. For example, the Cpf1 protein preferentially cuts the strand of a duplexed nucleic acid molecule in which a nucleotide to be edited does not reside. In some embodiments, the crippled Cpf1 protein preferentially cuts the target strand at least 5% more efficiently than it cuts the non-target strand. In some embodiments, the crippled Cpf1 protein preferentially cuts the target strand at least 5%, at least 10%, at least 15%, at least 20%, at least 25%, at least 30%, at least 35%, at least 40%, at least 50%, at least 60%, at least 70%, at least 80%, at least 90%, or at least 100% more efficiently than it cuts the non-target strand.

In some embodiments, a crippled Cpf1 protein is a non-naturally occurring Cpf1 protein. In some embodiments, the crippled Cpf1 protein comprises one or more mutations relative to a wild-type Cpf1 protein. In some embodiments, the crippled Cpf1 protein comprises 1, 2, 3, 4, 5, 6, 7, 8, 9, 10, 11, 12, 13, 14, 15, 16, 17, 18, 19, or 20 mutations relative to a wild-type Cpf1 protein. In some embodiments, the crippled Cpf1 protein comprises an R836A mutation mutation as set forth in SEQ ID NO: 685, or in a corresponding amino acid in another Cpf1 protein. It should be appreciated that a Cpf1 comprising a homologous residue (e.g., a corresponding amino acid) to R836A of SEQ ID NO: 685 could also be mutated to achieve similar results. In some embodiments, the crippled Cpf1 protein comprises a R1138A mutation as set forth in SEQ ID NO: 685, or in a corresponding amino acid in another Cpf1 protein. In some embodiments, the crippled Cpf1 protein comprises an R912A mutation mutation as set forth in SEQ ID NO: 684, or in a corresponding amino acid in another Cpf1 protein. Without wishing to be bound by any particular theory, residue R838 of SEQ ID NO: 685 (LbCpf1) and residue R912 of SEQ ID NO: 684 (AsCpf1) are examples of corresponding (e.g., homologous) residues. For example, a portion of the alignment between SEQ ID NO: 684 and 685 shows that R912 and R838 are corresponding residues.

| | |
|---|---|
| AsCpf1 | YQAANSPSKFNQRVNAYLKEHPETPIIGIDRGERNLIYITVIDSTGKILEQRSLNTIQ-- |
| LbCpf1 | KCPKN-IFKINTEVRVLLKHDDNPYVIGIDRGERNLLYIVVVDGKGNIVEQYSLNEIINN |
| | * *:* .*.. **.. : :**********:**.*:*..*:*:** *** * |

In some embodiments, any of the Cpf1 proteins provided herein comprises one or more amino acid deletions. In some embodiments, any of the Cpf1 proteins provided herein comprises 1, 2, 3, 4, 5, 6, 7, 8, 9, 10, 11, 12, 13, 14, 15, 16, 17, 18, 19, or 20 amino acid deletions. Without wishing to be bound by any particular theory, there is a helical region in Cpf1, which includes residues 661-667 of AsCpf1 (SEQ ID NO: 684), that may obstruct the function of a deaminase (e.g., APOBEC) that is fused to the Cpf1. This region comprises the amino acid sequence KKTGDQK (SEQ ID NO: 737). Accordingly, aspects of the disclosure provide Cpf1 proteins comprising mutations (e.g., deletions) that disrupt this helical region in Cpf1. In some embodiments, the Cpf1 protein comprises one or more deletions of the following residues in SEQ ID NO: 684, or one or more corresponding deletions in another Cpf1 protein: K661, K662, T663, G664, D665, Q666, and K667, In some embodiments, the Cpf1 protein comprises a T663 and a D665 deletion in SEQ ID NO: 684, or corresponding deletions in another Cpf1 protein. In some embodiments, the Cpf1 protein comprises a K662, T663, D665, and Q666 deletion in SEQ ID NO: 684, or corresponding deletions in another Cpf1 protein. In some embodiments, the Cpf1 protein comprises a K661, K662, T663, D665, Q666 and K667 deletion in SEQ ID NO: 684, or corresponding deletions in another Cpf1 protein.

### AsCpf1 (Deleted T663 and D665)

(SEQ ID NO: 689)
TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKGQKGYREALCKWIDFTRDFLSKYTKTTSIDLSSLRPSS
QYKDLGEYYAELNPLLYHISFQRIAEKEIMDAVETGKLYLFQIYNKDFAK
GHHGKPNLHTLYWTGLFSPENLAKTSIKLNGQAELFYRPKSRMKRMAHRL
GEKMLNKKLKDQKTPIPDTLYQELYDYVNHRLSHDLSDEARALLPNVITK
EVSHEIIKDRRFTSDKFFFHVPITLNYQAANSPSKFNQRVNAYLKEHPET
PIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSK
RTGIAEKAVYQQFEKMLIDKLNCLVLKDYPAEKVGGVLNPYQLTDQFTSF
AKMGTQSGFLFYVPAPYTSKIDPLTGFVDPFVWKTIKNHESRKHFLEGFD
FLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPK
LLENDDSHAIDTMVALIRSVLQMRNSNAATGEDYINSPVRDLNGVCFDSR
FQNPEWPMDADANGAYHIALKGQLLLNHLKESKDLKLQNGISNQDWLAYI
QELRN

### AsCpf1 (Deleted K662, T663, D665, and Q666)

(SEQ ID NO: 690)
TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKGKGYREALCKWIDFTRDFLSKYTKTTSIDLSSLRPSSQY
KDLGEYYAELNPLLYHISFQRIAEKEIMDAVETGKLYLFQIYNKDFAKGH
HGKPNLHTLYWTGLFSPENLAKTSIKLNGQAELFYRPKSRMKRMAHRLGE
KMLNKKLKDQKTPIPDTLYQELYDYVNHRLSHDLSDEARALLPNVITKEV
SHEIIKDRRFTSDKFFFHVPITLNYQAANSPSKFNQRVNAYLKEHPETPI
IGIDRGERNLIYITVIDSTGKILEQRSLNTIQQFDYQKKLDNREKERVAA
RQAWSVVGTIKDLKQGYLSQVIHEIVDLMIHYQAVVVLENLNFGFKSKRT
GIAEKAVYQQFEKMLIDKLNCLVLKDYPAEKVGGVLNPYQLTDQFTSFAK
MGTQSGFLFYVPAPYTSKIDPLTGFVDPFVWKTIKNHESRKHFLEGFDFL
HYDVKTGDFILHFKMNRNLSFQRGLPGFMPAWDIVFEKNETQFDAKGTPF
IAGKRIVPVIENHRFTGRYRDLYPANELIALLEEKGIVFRDGSNILPKLL
ENDDSHAIDTMVALIRSVLQMRNSNAATGEDYINSPVRDLNGVCFDSRFQ
NPEWPMDADANGAYHIALKGQLLLNHLKESKDLKLQNGISNQDWLAYIQE
LRN

### AsCpf1 (Deleted K661, K662, T663, D665, Q666, and K667)

(SEQ ID NO: 691)
TQFEGFTNLYQVSKTLRFELIPQGKTLKHIQEQGFIEEDKARNDHYKELK
PIIDRIYKTYADQCLQLVQLDWENLSAAIDSYRKEKTEETRNALIEEQAT
YRNAIHDYFIGRTDNLTDAINKRHAEIYKGLFKAELFNGKVLKQLGTVTT
TEHENALLRSFDKFTTYFSGFYENRKNVFSAEDISTAIPHRIVQDNFPKF
KENCHIFTRLITAVPSLREHFENVKKAIGIFVSTSIEEVFSFPFYNQLLT
QTQIDLYNQLLGGISREAGTEKIKGLNEVLNLAIQKNDETAHIIASLPHR
FIPLFKQILSDRNTLSFILEEFKSDEEVIQSFCKYKTLLRNENVLETAEA
LFNELNSIDLTHIFISHKKLETISSALCDHWDTLRNALYERRISELTGKI
TKSAKEKVQRSLKHEDINLQEIISAAGKELSEAFKQKTSEILSHAHAALD
QPLPTTMLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAGGYREALCKWIDFTRDFLSKYTKTTSIDLSSLRPSSQYKD
LGEYYAELNPLLYHISFQRIAEKEIMDAVETGKLYLFQIYNKDFAKGHHG
KPNLHTLYWTGLFSPENLAKTSIKLNGQAELFYRPKSRMKRMAHRLGEKM
LNKKLKDQKTPIPDTLYQELYDYVNHRLSHDLSDEARALLPNVITKEVSH
EIIKDRRFTSDKFFFHVPITLNYQAANSPSKFNQRVNAYLKEHPETPIIG
IDRGERNLIYITVIDSTGKILEQRSLNTIQQFDYQKKLDNREKERVAARQ
AWSVVGTIKDLKQGYLSQVIHEIVDLMIHYQAVVVLENLNFGFKSKRTGI
AEKAVYQQFEKMLIDKLNCLVLKDYPAEKVGGVLNPYQLTDQFTSFAKMG
TQSGFLFYVPAPYTSKIDPLTGFVDPFVWKTIKNHESRKHFLEGFDFLHY
DVKTGDFILHFKMNRNLSFQRGLPGFMPAWDIVFEKNETQFDAKGTPFIA
GKRIVPVIENHRFTGRYRDLYPANELIALLEEKGIVFRDGSNILPKLLEN
DDSHAIDTMVALIRSVLQMRNSNAATGEDYINSPVRDLNGVCFDSRFQNP
EWPMDADANGAYHIALKGQLLLNHLKESKDLKLQNGISNQDWLAYIQELR
N

In some embodiments, the guide nucleotide sequence-programmable DNA-binding protein domain of the present disclosure has no requirements for a PAM sequence. One example of such a guide nucleotide sequence-programmable DNA-binding protein may be an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the codons that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference. The sequence of Natronobacterium gregoryi Argonaute is provided in SEQ ID NO: 348.

Wild Type Natronobacterium gregoryi Argonaute (SEQ ID NO: 348)

MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

In some embodiments, the guide nucleotide sequence-programmable DNA-binding protein is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol. Direct. 2009 Aug. 25; 4:29. doi: 10.1186/1745-6150-4-29, which is incorporated herein by reference. In some embodiments, the guide nucleotide sequence-programmable DNA-binding protein is a Marinitoga piezophila Argunaute (MpAgo) protein. The CRISPR-associated Marinitoga piezophila Argonaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. See, e.g., Kaya et al., “A bacterial Argonaute with noncanonical guide RNA specificity”, Proc Natl Acad Sci USA. 2016 Apr. 12; 113(15):4057-62, the entire contents of which are hereby incorporated by reference). It should be appreciated that other Argonaute proteins may be used in any of the fusion proteins (e.g., base editors) described herein, for example, to guide a deaminase (e.g., cytidine deaminase) to a target nucleic acid (e.g., ssRNA).

In some embodiments, the guide nucleotide sequence-programmable DNA-binding protein is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60(3): 385-397, the entire contents of which are herein incorporated by reference. Effectors of two of the systems, C2c1 and C2c3, contain RuvC-like endonuclease domains related to Cpf1. A third system, C2c2 contains an effector with two predicted HEPN RNase domains. Production of mature CRISPR RNA is tracrRN A-independent, unlike production of CRISPR RNA by C2c1, C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See. e.g., East-Seletsky, et al., “Two distinct RNase activities of CRISPR-C2c2 enable guide-RNA processing and RNA detection”, Nature, 2016 Oct. 13; 538(7624):270-273, the entire contents of which are hereby incorporated by reference. In vitro biochemical analysis of C2c2 in Leptotrichia shahii has shown that C2c2 is guided by a single CRISPR RNA and can be programmed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayyeh et al., “C2c2 is a single-component programmable RNA-guided RNA-targeting CRISPR effector.” Science, 2016 Aug. 5; 353(6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See, e.g., Liu et al., “C2c1-sgRNA Complex Structure Reveals RNA-Guided DNA Cleavage Mechanism”, Mol. Cell. 2017 Jan. 19; 65(2):310-322, incorporated herein by reference. The crystal structure has also been reported for Alicyclobacillus acidoterrestris C2c1 bound to target DNAs as ternary complexes. See. e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the guide nucleotide sequence-programmable DNA-binding protein of any of the fusion proteins provided herein is a C2c1, a C2c2, or a C2c3 protein. In some embodiments, the guide nucleotide sequence-programmable DNA-binding protein is a C2c1 protein. In some embodiments, the guide nucleotide sequence-programmable DNA-binding protein is a C2c2 protein. In some embodiments, the guide nucleotide sequence-programmable DNA-binding protein is a C2c3 protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the guide nucleotide sequence-programmable DNA-binding protein is a naturally-occurring C2c1, C2c2, or C2c3 protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 692-694. In some embodiments, the guide nucleotide sequence-programmable DNA-binding protein comprises an amino acid sequence of any one SEQ ID NOs: 692-694. It should be appreciated that C2c1, C2c2, or C2c3 from other bacterial species may also be used in accordance with the present disclosure.

### C2c1 (Uniprot.Org/Uniprot/T0D7A2 #)

sp|T0D7A2|C2C1_ALIAG CRISPR-associated
endonuclease C2c1 OS = Alicyclobacillus
acidoterrestris (strain ATCC 49025/
DSM 3922/CIP 106132/NCIMB 13137/GD3B)
GN = c2c1 PE = 1 SV = 1
(SEQ ID NO: 692)
MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI

### C2c2 (Uniprot.Org/Uniprot/P0DOC6)

>sp|P0DOC6|C2C2_LEPSD CRISPR-associated
endoribonuclease C2c2 OS = Leptotrichia
shahii (strain DSM 19757/CCUG 47503/
CIP 107916/JCM 16776/LB37)
GN = c2c2 PE = 1 SV = 1
(SEQ ID NO: 693)
MGNLFGHKRWYEVRDKKDFKIKRKVKVKRNYDGNKYILNINENNNKEKID
NNKFIRKYINYKKNDNILKEFTRKFHAGNILFKLKGKEGIIRIENNDDFL
ETEEVVLYIEAYGKSEKLKALGITKKKIIDEAIRQGITKDDKKIEIKRQE
NEEEIEIDIRDEYTNKTLNDCSIILRIIENDELETKKSIYEIFKNINMSL
YKIIEKIIENETEKVFENRYYEEHLREKLLKDDKIDVILTNFMEIREKIK
SNLEILGFVKFYLNVGGDKKKSKNKKMLVEKILNINVDLTVEDIADFVIK
ELEFWNITKRIEKVKKVNNEFLEKRRNRTYIKSYVLLDKHEKFKIERENK
KDKIVKFFVENIKNNSIKEKIEKILAEFKIDELIKKLEKELKKGNCDTEI
FGIFKKHYKVNFDSKKFSKKSDEEKELYKIIYRYLKGRIEKILVNEQKVR
LKKMEKIEIEKILNESILSEKILKRVKQYTLEHIMYLGKLRHNDIDMTTV
NTDDFSRLHAKEELDLELITFFASTNMELNKIFSRENINNDENIDFFGGD
REKNYVLDKKILNSKIKIIRDLDFIDNKNNITNNFIRKFTKIGTNERNRI
LHAISKERDLQGTQDDYNKVINIIQNLKISDEEVSKALNLDVVFKDKKNI
ITKINDIKISEENNNDIKYLPSFSKVLPEILNLYRNNPKNEPFDTIETEK
IVLNALIYVNKELYKKLILEDDLEENESKNIFLQELKKTLGNIDEIDENI
IENYYKNAQISASKGNNKAIKKYQKKVIECYIGYLRKNYEELFDFSDFKM
NIQEIKKQIKDINDNKTYERITVKTSDKTIVINDDFEYIISIFALLNSNA
VINKIRNRFFATSVWLNTSEYQNIIDILDEIMQLNTLRNECITENWNLNL
EEFIQKMKEIEKDFDDFKIQTKKEIFNNYYEDIKNNILTEFKDDINGCDV
LEKKLEKIVIFDDETKFEIDKKSNILQDEQRKLSNINKKDLKKKVDQYIK
DKDQEIKSKILCRIIFNSDFLKKYKKEIDNLIEDMESENENKFQEIYYPK
ERKNELYIYKKNLFLNIGNPNFDKIYGLISNDIKMADAKFLFNIDGKNIR
KNKISEIDAILKNLNDKLNGYSKEYKEKYIKKLKENDDFFAKNIQNKNYK
SFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYK
KFEKICYGFGIDLSENSEINKPENESIRNYISHFYIVRNPFADYSIAEQI
DRVSNLLSYSTRYNNSTYASVFEVFKKDVNLDYDELKKKFKLIGNNDILE
RLMKPKKVSVLELESYNSDYIKNLIIELLTKIENTNDTL

C2c3, translated from >CEPX01008730.1 marine metagenome genome assembly TARA_037_MES_0.1-0.22, contig TARA_037_MES_0.1-0.22_scaffold22115_1, whole genome shotgun sequence.

(SEQ ID NO: 694)
MRSNYHGGRNARQWRKQISGLARRTKETVFTYKFPLETDAAEIDFDKAVQ
TYGIAEGVGHGSLIGLVCAFHLSGFRLFSKAGEAMAFRNRSRYPTDAFAE
KLSAIMGIQLPTLSPEGLDLIFQSPPRSRDGIAPVWSENEVRNRLYTNWT
GRGPANKPDEHLLEIAGEIAKQVFPKFGGWDDLASDPDKALAAADKYFQS
QGDFPSIASLPAAIMLSPANSTVDFEGDYIAIDPAAETLLHQAVSRCAAR
LGRERPDLDQNKGPFVSSLQDALVSSQNNGLSWLFGVGFQHWKEKSPKEL
IDEYKVPADQHGAVTQVKSFVDAIPLNPLFDTTHYGEFRASVAGKVRSWV
ANYWKRLLDLKSLLATTEFTLPESISDPKAVSLFSGLLVDPQGLKKVADS
LPARLVSAEEAIDRLMGVGIPTAADIAQVERVADEIGAFIGQVQQFNNQV
KQKLENLQDADDEEFLKGLKIELPSGDKEPPAINTRISGGAPDAAAEISE
LEEKLQRLLDARSEHFQTISEWAEENAVTLDPIAAMVELERLRLAERGAT
GDPEEYALRLLLQRIGRLANRVSPVSAGSIRELLKPVFMEEREFNLFFHN
RLGSLYRSPYSTSRHQPFSIDVGKAKAIDWIAGLDQISSDIEKALSGAGE
ALGDQLRDWINTLAGFAISQRLRGLPDTVPNALAQVRCPDDVRIPPLLAM
LLEEDDIARDVCLKAFNLYVSAINGCLFGALREGFIVRTRFQRIGTDQIH
YVPKDKAWEYPDRLNTAKGPINAAVSSDWIEKDGAVIKPVETVRNLSSTG
FAGAGVSEYLVQAPHDWYTPLDLRDVAHLVTGLPVEKNITKLKRLTNRTA
FRMVGASSFKTHLDSVLLSDKIKLGDFTIIIDQHYRQSVTYGGKVKISYE
PERLQVEAAVPVVDTRDRTVPEPDTLFDHIVAIDLGERSVGFAVFDIKSC
LRTGEVKPIHDNNGNPVVGTVAVPSIRRLMKAVRSHRRRRQPNQKVNQTY
STALQNYRENVIGDVCNRIDTLMERYNAFPVLEFQIKNFQAGAKQLEIVY
GS

In some embodiments, the guide nucleotide sequence-programmable DNA-binding protein of any of the fusion proteins provided herein is a Cas9 from archaea (e.g. nanoarchaea), which constitute a domain and kingdom of single-celled prokaryotic microbes. In some embodiments, the guide nucleotide sequence-programmable DNA-binding protein is CasX or CasY, which have been described in, for example, Burstein et al., “New CRISPR-Cas systems from uncultivated microbes.” Cell Res. 2017 Feb. 21. doi: 10.1038/cr.2017.21, which is incorporated herein by reference. Using genome-resolved metagenomics, a number of CRISPR-Cas systems were identified, including the first reported Cas9 in the archaeal domain of life. This divergent Cas9 protein was found in nanoarchaea as part of an active CRISPR-Cas system. In bacteria, two previously unknown systems were discovered. CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a guide nucleotide sequence-programmable DNA-binding protein and are within the scope of this disclosure.

In some embodiments, the guide nucleotide sequence-programmable DNA-binding protein of any of the fusion proteins provided herein is a CasX or CasY protein. In some embodiments, the guide nucleotide sequence-programmable DNA-binding protein is a CasX protein. In some embodiments, the guide nucleotide sequence-programmable DNA-binding protein is a CasY protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the guide nucleotide sequence-programmable DNA-binding protein is a naturally-occurring CasX or CasY protein. In some embodiments, the guide nucleotide sequence-programmable DNA-binding protein comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 695-697. In some embodiments, the guide nucleotide sequence-programmable DNA-binding protein comprises an amino acid sequence of any one of SEQ ID NOs: 695-697. It should be appreciated that CasX and CasY from other bacterial species may also be used in accordance with the present disclosure.

### CasX (Uniprot.Org/Uniprot/F0NN87; Uniprot.Org/Uniprot/F0NH53)

>tr|F0NN87|F0NN87_SULIH CRISPR-associated
Casx protein OS = Sulfolobus islandicus
(strain HVE10/4) GN = SiH_0402 PE = 4 SV = 1
(SEQ ID NO: 695)
MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYEFGRSPGMVERTRRVKLE
VEPHYLIIAAAGWVLTRLGKAKVSEGDYVGVNVFTPTRGILYSLIQNVNG
IVPGIKPETAFGLWIARKVVSSVTNPNVSVVRIYTISDAVGQNPTTINGG
FSIDLTKLLEKRYLLSERLEAIARNALSISSNMRERYIVLANYIYEYLTG
SKRLEDLLYFANRDLIMNLNSDDGKVRDLKLISAYVNGELIRGEG
>tr|F0NH53|F0NH53_SULIR CRISPR associated
protein, Casx OS = Sulfolobus islandicus
(strain REY15A) GN = SiRe_0771 PE = 4 SV = 1
(SEQ ID NO: 696)
MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYKFGRSPGMVERTRRVKLE
VEPHYLIMAAAGWVLTRLGKAKVSEGDYVGVNVFTPTRGILYSLIQNVNG
IVPGIKPETAFGLWIARKVVSSVTNPNVSVVSIYTISDAVGQNPTTINGG
FSIDLTKLLEKRDLLSERLEAIARNALSISSNMRERYIVLANYIYEYLTG
SKRLEDLLYFANRDLIMNLNSDDGKVRDLKLISAYVNGELIRGEG

### CasY (Ncbi.Nlm.Nih.Gov/Protein/APG80656.1)

>APG80656.1 CRISPR-associated protein CasY
[uncultured Parcubacteria group bacterium]
(SEQ ID NO: 697)
MSKRHPRISGVKGYRLHAQRLEYTGKSGAMRTIKYPLYSSPSGGRTVPRE
IVSAINDDYVGLYGLSNFDDLYNAEKRNEEKVYSVLDFWYDCVQYGAVFS
YTAPGLLKNVAEVRGGSYELTKTLKGSHLYDELQIDKVIKFLNKKEISRA
NGSLDKLKKDIIDCFKAEYRERHKDQCNKLADDIKNAKKDAGASLGERQK
KLFRDFFGISEQSENDKPSFTNPLNLTCCLLPFDTVNNNRNRGEVLFNKL
KEYAQKLDKNEGSLEMWEYIGIGNSGTAFSNFLGEGFLGRLRENKITELK
KAMMDITDAWRGQEQEEELEKRLRILAALTIKLREPKFDNHWGGYRSDIN
GKLSSWLQNYINQTVKIKEDLKGHKKDLKKAKEMINRFGESDTKEEAVVS
SLLESIEKIVPDDSADDEKPDIPAIAIYRRFLSDGRLTLNRFVQREDVQE
ALIKERLEAEKKKKPKKRKKKSDAEDEKETIDFKELFPHLAKPLKLVPNF
YGDSKRELYKKYKNAAIYTDALWKAVEKIYKSAFSSSLKNSFFDTDFDKD
FFIKRLQKIFSVYRRFNTDKWKPIVKNSFAPYCDIVSLAENEVLYKPKQS
RSRKSAAIDKNRVRLPSTENIAKAGIALARELSVAGFDWKDLLKKEEHEE
YIDLIELHKTALALLLAVTETQLDISALDFVENGTVKDFMKTRDGNLVLE
GRFLEMFSQSIVFSELRGLAGLMSRKEFITRSAIQTMNGKQAELLYIPHE
FQSAKITTPKEMSRAFLDLAPAEFATSLEPESLSEKSLLKLKQMRYYPHY
FGYELTRTGQGIDGGVAENALRLEKSPVKKREIKCKQYKTLGRGQNKIVL
YVRSSYYQTQFLEWFLHRPKNVQTDVAVSGSFLIDEKKVKTRWNYDALTV
ALEPVSGSERVFVSQPFTIFPEKSAEEEGQRYLGIDIGEYGIAYTALEIT
GDSAKILDQNFISDPQLKTLREEVKGLKLDQRRGTFAMPSTKIARIRESL
VHSLRNRIHHLALKHKAKIVYELEVSRFEEGKQKIKKVYATLKKADVYSE
IDADKNLQTTVWGKLAVASEISASYTSQFCGACKKLWRAEMQVDETITTQ
ELIGTVRVIKGGTLIDAIKDFMRPPIFDENDTPFPKYRDFCDKHHISKKM
RGNSCLFICPFCRANADADIQASQTIALLRYVKEEKKVEDYFERFRKLKN
IKVLGQMKKI

### Cas9 Domains of Nucleobase Editors

Non-limiting, exemplary Cas9 domains are provided herein. The Cas9 domain may be a nuclease active Cas9 domain, a nucleasae inactive Cas9 domain, or a Cas9 nickase. In some embodiments, the Cas9 domain is a nuclease active domain. For example, the Cas9 domain may be a Cas9 domain that cuts both strands of a duplexed nucleic acid (e.g., both strands of a duplexed DNA molecule). In some embodiments, the Cas9 domain comprises any one of the amino acid sequences as set forth herein. In some embodiments the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth herein. In some embodiments, the Cas9 domain comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth herein. In some embodiments, the Cas9 domain comprises an amino acid sequence that has at least 10, at least 15, at least 20, at least 30, at leat 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, the Cas9 domain is a nuclease-inactive Cas9 domain (dCas9). For example, the dCas9 domain may bind to a duplexed nucleic acid molecule (e.g., via a gRNA molecule) without cleaving either strand of the duplexed nucleic acid molecule. In some embodiments, the nuclease-inactive dCas9 domain comprises a DIOX mutation and a H840X mutation or a corresponding mutation in any of the amino acid sequences provided in any of the Cas9 proteins provided herein, wherein X is any amino acid change. In some embodiments, the nuclease-inactive dCas9 domain comprises a D10A mutation and a H840A mutation or a corresponding mutation in any of the amino acid sequences provided in any of the Cas9 proteins provided herein. As one example, a nuclease-inactive Cas9 domain comprises the amino acid sequence set forth in SEQ ID NO: 698 (Cloning vector pPlatTET-gRNA2. Accession No. BAV54124).

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(SEQ ID NO: 698; see, e.g., Qi et al., Repurposing
CRISPR as an RNA-guided platform for sequence-
specific control of gene expression. Cell. 2013;
152(5): 1173-83, the entire contents of which are
incorporated herein by reference).

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See. e.g., Prashant et al., CAS9 transcriptional activators for target specificity screening and paired nickases for cooperative genome engineering. Nature Biotechnology. 2013; 31(9): 833-838, the entire contents of which are incorporated herein by reference). In some embodiments the dCas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the dCas9 domains provided herein. In some embodiments, the Cas9 domain comprises an amino acid sequences that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences of Cas9 or a Cas9 variant set forth herein. In some embodiments, the Cas9 domain comprises an amino acid sequence that has at least 10, at least 15, at least 20, at least 30, at leat 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences of Cas9 or a Cas9 variant set forth herein.

In some embodiments, the Cas9 domain is a Cas9 nickase. The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. For example, a Cas9 nickase may comprise the amino acid sequence as set forth in SEQ ID NO: 683. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

Cas9 Domains with Reduced PAM Exclusivity

Some aspects of the disclosure provide Cas9 domains that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a four base region (e.g., a “deamination window”), which is approximately 15 bases upstream of the PAM. See Komor. A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence and has relaxed PAM requirements (PAMless Cas9). PAMless Cas9 exhibits an increased activity on a target sequence that does not include a canonical PAM (e.g., NGG) sequence at its 3′-end as compared to Streptococcus pyogenes Cas9 as provided by SEQ ID NO: 1, e.g., increased activity by at least 5-fold, at least 10-fold, at least 50-fold, at least 100-fold, at least 500-fold, at least 1,000-fold, at least 5,000-fold, at least 10,000-fold, at least 50,000-fold, at least 100.000-fold, at least 500,000-fold, or at least 1,000,000-fold, Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference. See also US Provisional Applications, U.S. Ser. No. 62/245,828, filed Oct. 23, 2015; 62/279,346, filed Jan. 15, 2016; 62/311,763, filed Mar. 22, 2016; 62/322,178, filed Apr. 13, 2016; and 62/357,332, filed Jun. 30, 2016, each of which is incorporated herein by reference. In some embodiments, the dCas9 or Cas9 nickase useful in the present disclosure may further comprise mutations that relax the PAM requirements, e.g., mutations that correspond to A262T, K294R, S409I, E480K, E543D, M694I, or E1219V in SEQ ID NO: 1.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises the amino acid sequence SEQ ID NO: 699. In some embodiments, the SaCas9 comprises a N579X mutation of SEQ ID NO: 699, or a corresponding mutation in any of the amino acid sequences provided in any of the Cas9 proteins disclosed herein including, but not limited to, SEQ ID NOs: 1-260, 270-292, 315-323, 680, and 682, wherein X is any amino acid except for N. In some embodiments, the SaCas9 comprises a N579A mutation of SEQ ID NO: 699, or a corresponding mutation in any of the amino acid sequences provided in SEQ ID NOs: 1-260, 272-292, 315-323, 680, and 682. In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence. In some embodiments, the SaCas9 domain comprises one or more of a E781X, a N967X, and a R1014X mutation of SEQ ID NO: 699, or a corresponding mutation in any of the Cas9 amino acid sequences provided herein, including but not limited to in SEQ ID NOs: 1-260, 270-292, 315-323, 680, or 682, wherein X is any amino acid. In some embodiments, the SaCas9 domain comprises one or more of a E781K, a N967K. and a R1014H mutation of SEQ ID NO: 699, or one or more corresponding mutation in any of the Cas9 amino acid sequences provided herein, including but not limited to in SEQ ID NOs: 1-260, 270-292, 315-323, 680, or 682. In some embodiments, the SaCas9 domain comprises a E781K, a N967K, or a R1014H mutation of SEQ ID NO: 699, or one or more corresponding mutation in any of the Cas9 amino acid sequences provided herein, including but not limited to in SEQ ID NOs: 1-260, 270-292, 315-323, 680, or 682.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 699-701. In some embodiments, the Cas9 domain of any of the fusion proteins provided herein comprises the amino acid sequence of any one of SEQ ID NOs: 699-701. In some embodiments, the Cas9 domain of any of the fusion proteins provided herein consists of the amino acid sequence of any one of SEQ ID NOs: 699-701.

### Exemplary SaCas9 Sequence

(SEQ ID NO: 699)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINRFKTSDYVKEAKQLLKVQKAYHQLDQSFIDTY
IDLLETRRTYYEGPGEGSPFGWKDIKEWYEMLMGHCTYFPEELRSVKYAY
NADLYNALNDLNNLVITRDENEKLEYYEKFQIIENVFKQKKKPTLKQIAK
EILVNEEDIKGYRVTSTGKPEFTNLKVYHDIKDITARKEIIENAELLDQI
AKILTIYQSSEDIQEELTNLNSELTQEEIEQISNLKGYTGTHNLSLKAIN
LILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQT
NERIEEIIRTTGKENAKYLIEKIKLHDMQEGKCLYSLEAIPLEDLLNNPF
NYEVDHIIPRSVSFDNSFNNKVLVKQEENSKKGNRTPFQYLSSSDSKISY
ETFKKHILNLAKGKGRISKTKKEYLLEERDINRFSVQKDFINRNLVDTRY
ATRGLMNLLRSYFRVNNLDVKVKSINGGFTSFLRRKWKFKKERNKGYKHH
AEDALIIANADFIFKEWKKLDKAKKVMENQMFEEKQAESMPEIETEQEYK
EIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEK
NPLYKYYEETGNYLTKYSKKDNGPVIKKIKYYGNKLNAHLDITDDYPNSR
NKVVKLSLKPYRFDVYLDNGVYKFVTVKNLDVIKKENYYEVNSKCYEEAK
KLKKISNQAEFIASFYNNDLIKINGELYRVIGVNNDLLNRIEVNMIDITY
REYLENMNDKRPPRIIKTIASKTQSIKKYSTDILGNLYEVKSKKHPQIIK
KG

Residue N579 of SEQ ID NO: 699, which is underlined and in bold, may be mutated (e.g., to a A579) to yield a SaCas9 nickase.

### Exemplary SaCas9d Sequence

(SEQ ID NO: 702)
KRNYILGLAIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINTRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG.

Residue D10 of SEQ ID NO: 702, which is underlined and in bold, may be mutated (e.g., to a A10) to yield a nuclease inactive SaCas9d.

### Exemplary SaCas9n Sequence

(SEQ ID NO: 700)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINTRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEEASKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG.

Residue A579 of SEQ ID NO: 700, which can be mutated from N579 of SEQ ID NO: 699 to yield a SaCas9 nickase, is underlined and in bold.

### Exemplary SaKKH Cas9

(SEQ ID NO: 701)
KRNYILGLDIGITSVGYGIIDYETRDVIDAGVRLFKEANVENNEGRRSKR
GARRLKRRRRHRIQRVKKLLFDYNLLTDHSELSGINPYEARVKGLSQKLS
EEEFSAALLHLAKRRGVHNVNEVEEDTGNELSTKEQISRNSKALEEKYVA
ELQLERLKKDGEVRGSINTRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEEASKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRKLINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYKNDLIKINGELYRVIGVNNDLLNRIEVNMIDIT
YREYLENMNDKRPPHIIKTIASKTQSIKKYSTDILGNLYEVKSKKHPQII
KKG.

Residue A579 of SEQ ID NO: 701, which can be mutated from N579 of SEQ ID NO: 699 to yield a SaCas9 nickase, is underlined and in bold. Residues K781, K967, and H1014 of SEQ ID NO: 701, which can be mutated from E781, N967, and R1014 of SEQ ID NO: 699 to yield a SaKKH Cas9 are underlined and in italics.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises the amino acid sequence SEQ ID NO: 703. In some embodiments, the SpCas9 comprises a D9X mutation of SEQ ID NO: 703, or a corresponding mutation in any of the Cas9 amino acid sequences provided herein, including but not limited to SEQ ID NOs: 1-260, 270-292, 315-323, 680, or 682, wherein X is any amino acid except for D. In some embodiments, the SpCas9 comprises a D9A mutation of SEQ ID NO: 703, or a corresponding mutation in any of the Cas9 amino acid sequences provided herein, including but not limited to SEQ ID NOs: 1-260, 270-292, 315-323, 680, or 682. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having a NGG, a NGA, or a NGCG PAM sequence. In some embodiments, the SpCas9 domain comprises one or more of a D1134X, a R1334X. and a T1336X mutation of SEQ ID NO: 703, or a corresponding mutation in any of the Cas9 amino acid sequences provided herein, including but not limited to SEQ ID NOs: 1-260, 270-292, 315-323, 680, or 682, wherein X is any amino acid. In some embodiments, the SpCas9 domain comprises one or more of a D1134E, R1334Q, and T1336R mutation of SEQ ID NO: 703, or a corresponding mutation in any of the Cas9 amino acid sequences provided herein, including but not limited to SEQ ID NOs: 1-260, 270-292, 315-323, 680, or 682. In some embodiments, the SpCas9 domain comprises a D1134E, a R1334Q, and a T1336R mutation of SEQ ID NO: 703, or a corresponding mutation in any of the Cas9 amino acid sequences provided herein, including but not limited to SEQ ID NOs: 1-260, 270-292, 315-323, 680, or 682. In some embodiments, the SpCas9 domain comprises one or more of a D1134X, a R1334X, and a T1336X mutation of SEQ ID NO: 703, or a corresponding mutation in any of the Cas9 amino acid sequences provided herein, including but not limited to SEQ ID NOs: 1-260, 270-292, 315-323, 680, or 682, wherein X is any amino acid. In some embodiments, the SpCas9 domain comprises one or more of a D1134V, a R1334Q, and a T1336R mutation of SEQ ID NO: 703, or a corresponding mutation in any of the Cas9 amino acid sequences provided herein, including but not limited to SEQ ID NOs: 1-260, 270-292, 315-323, 680, or 682. In some embodiments, the SpCas9 domain comprises a D1134V, a R1334Q, and a T1336R mutation of SEQ ID NO: 703, or a corresponding mutation in any of the Cas9 amino acid sequences provided herein, including but not limited to SEQ ID NOs: 1-260, 270-292, 315-323, 680, or 682. In some embodiments, the SpCas9 domain comprises one or more of a D1134X, a G1217X, a R1334X, and a T1336X mutation of SEQ ID NO: 703, or a corresponding mutation in any of the Cas9 amino acid sequences provided herein, including but not limited to SEQ ID NOs: 1-260, 270-292, 315-323, 680, or 682, wherein X is any amino acid. In some embodiments, the SpCas9 domain comprises one or more of a D1134V, a G1217R, a R1334Q. and a T1336R mutation of SEQ ID NO: 703, or a corresponding mutation in any of the Cas9 amino acid sequences provided herein, including but not limited to, SEQ ID NOs: 1-260, 270-292, 315-323, 680, or 682. In some embodiments, the SpCas9 domain comprises a D1134V, a G1217R, a R1334Q, and a T1336R mutation of SEQ ID NO: 703, or a corresponding mutation in any of the Cas9 amino acid sequences provided herein, including but not limited to SEQ ID NOs: 1-260, 270-292, 315-323, 680, or 682.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 4276-4280. In some embodiments, the Cas9 domain of any of the fusion proteins provided herein comprises the amino acid sequence of any one of SEQ ID NOs: 703-707. In some embodiments, the Cas9 domain of any of the fusion proteins provided herein consists of the amino acid sequence of any one of SEQ ID NOs: 703-707.

### Exemplary SpCas9

(SEQ ID NO: 703)
DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

### Exemplary SpCas9n

(SEQ ID NO: 704)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

### Exemplary SpEQR Cas9

(SEQ ID NO: 705)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFESPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRIDLSQLGGD

Residues E1134, Q1334, and R1336 of SEQ ID NO: 705, which can be mutated from D1134, R1334, and T1336 of SEQ ID NO: 703 to yield a SpEQR Cas9, are underlined and in bold.

### Exemplary SpVQR Cas9

(SEQ ID NO: 706)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKQYRSTKEVLDATLIHQS
ITGLYETRlDLSQLGGD

Residues V1134, Q1334, and R1336 of SEQ ID NO: 706, which can be mutated from D1134, R1334, and T1336 of SEQ ID NO: 703 to yield a SpVQR Cas9, are underlined and in bold.

### Exemplary SpVRER Cas9

(SEQ ID NO: 707)
DKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFVSPTVAYSVLVVAKVEK
GKSKKLKSVKELLGITIMERSSFEKNPIDFLEAKGYKEVKKDLIIKLPKY
SLFELENGRKRMLASARELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKEYRSTKEVLDATLIHQS
ITGLYETRIDLSQLGGD

Residues V1134, R1217, Q1334, and R1336 of SEQ ID NO: 707, which can be mutated from D1134, G1217, R1334, and T1336 of SEQ ID NO: 703 to yield a SpVRER Cas9, are underlined and in bold.

### High Fidelity Base Editors

Some aspects of the disclosure provide Cas9 fusion proteins (e.g., any of the fusion proteins provided herein) comprising a Cas9 domain that has high fidelity. Additional aspects of the disclosure provide Cas9 fusion proteins (e.g., any of the fusion proteins provided herein) comprising a Cas9 domain with decreased electrostatic interactions between the Cas9 domain and a sugar-phosphate backbone of a DNA, as compared to a wild-type Cas9 domain. In some embodiments, a Cas9 domain (e.g., a wild type Cas9 domain) comprises one or more mutations that decreases the association between the Cas9 domain and a sugar-phosphate backbone of a DNA. In some embodiments, any of the Cas9 fusion proteins provided herein comprise one or more of a N497X, a R661X, a Q695X. and/or a Q926X mutation of the amino acid sequence provided in SEQ ID NO: 1, or a corresponding mutation in any of the Cas9 amino acid sequences provided herein, including but not limited to the sequences seen in SEQ ID NOs: 1-260, 270-292, and 315-323, wherein X is any amino acid. In some embodiments, any of the Cas9 fusion proteins provided herein comprise one or more of a N497A, a R661A, a Q695A, and/or a Q926A mutation of the amino acid sequence provided in SEQ ID NO: 1, or a corresponding mutation in any of the Cas9 amino acid sequences provided herein, including but not limited to the sequences seen in SEQ ID NOs: 1-260, 270-292, 315-323, 680, and 682. In some embodiments, the Cas9 domain comprises a D10A mutation of the amino acid sequence provided in SEQ ID NO: 1, or a corresponding mutation in any of the Cas9 amino acid sequences provided herein, including but not limited to the sequences seen in SEQ ID NOs: 1-260, 270-292, 315-323, 680, and 682. In some embodiments, the Cas9 domain (e.g., of any of the fusion proteins provided herein) comprises the amino acid sequence as set forth in SEQ ID NO: 708. In some embodiments, the fusion protein comprises the amino acid sequence as set forth in SEQ ID NO: 709, Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker. I. M., et al. “Rationally engineered Cas9 nucleases with improved specificity.” Science 351, 84-88 (2015); the entire contents of each are incorporated herein by reference.

It should be appreciated that the base editors provided herein, for example, base editor 2 (BE2) or base editor 3 (BE3), may be converted into high fidelity base editors by modifying the Cas9 domain as described herein to generate high fidelity base editors, for example, high fidelity base editor 2 (HF-BE2) or high fidelity base editor 3 (HF-BE3). In some embodiments, base editor 2 (BE2) comprises a deaminase domain, a dCas9 domain, and a UGI domain. In some embodiments, base editor 3 (BE3) comprises a deaminase domain, a nCas9 domain, and a UGI domain.

Cas9 Domain where Mutations Relative to Cas9 of SEQ ID NO: 1 are Shown in Bold and Underlines

(SEQ ID NO: 708)
DKKYSIGLAIGTNSVGWAVITDEYKVPSKKFKVLGNTDRHSIKKNLIGAL
LFDSGETALATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQLEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTAFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGALSRKLINGIRDKQSGKTILDFLKSDGFANRNFMALIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRAITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD

### HF-BE3

(SEQ ID NO: 709)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGSETPGTSESATPESDKKYS
IGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLA
RGNSRFAWMTRKSEETITPWNFEEVVDKGASAQSFIERMTAFDKNLPNEK
VLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRY
TGWGALSRKLINGIRDKQSGKTILDFLKSDGFANRNFMALIHDDSLTFKE
DIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERG
GLSELDKAGFIKRQLVETRAITKHVAQILDSRMNTKYDENDKLIREVKVI
TLKSKLVSDFRKDFQFYKVREINNYHHAHDAYLNAVVGTALIKKYPKLES
EFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQK
QLFVEQHKHYLDEIIEQISEFSKRVILADANLDKVLSAYNKHRDKPIREQ
AENIIHLFTLTNLGAPAAFKYFDTTIDRKRYTSTKEVLDATLIHQSITGL
YETRIDLSQLGGD

### Fusion Proteins Comprising Gam

Some aspects of the disclosure provide fusion proteins comprising a Gam protein. Some aspects of the disclosure provide base editors that further comprise a Gam protein. Base editors are known in the art and have been described previously, for example, in U.S. Patent Application Publication Nos.: U.S. 2015-0166980, published Jun. 18, 2015; U.S. 2015-0166981, published Jun. 18, 2015; U.S. 2015-0166984, published Jun. 18, 2015; U.S. 2015-01669851, published Jun. 18, 2015; U.S. 2016-0304846, published Oct. 20, 2016; U.S. 2017-0121693-A1, published May 4, 2017; and PCT Application publication Nos.: WO 2015089406, published Jun. 18, 2015; and WO2017070632, published Apr. 27, 2017; the entire contents of each of which are hereby incorporated by reference. A skilled artisan would understand, based on the disclosure, how to make and use base editors that further comprise a Gam protein.

In some embodiments, the disclosure provides fusion proteins comprising a guide nucleotide sequence-programmable DNA-binding protein and a Gam protein. In some embodiments, the disclosure provides fusion proteins comprising a cytidine deaminase domain and a Gam protein. In some embodiments, the disclosure provides fusion proteins comprising a UGI domain and a Gam protein. In some embodiments, the disclosure provides fusion proteins comprising a guide nucleotide sequence-programmable DNA-binding protein, a cytidine deaminase domain and a Gam protein. In some embodiments, the disclosure provides fusion proteins comprising a guide nucleotide sequence-programmable DNA-binding protein, a cytidine deaminase domain a Gam protein and a UGI domain.

In some embodiments, the Gam protein is a protein that binds to double strand breaks in DNA and prevents or inhibits degradation of the DNA at the double strand breaks. In some embodiments, the Gam protein is encoded by the bacteriophage Mu, which binds to double stranded breaks in DNA. Without wishing to be bound by any particular theory. Mu transposes itself between bacterial genomes and uses Gam to protect double stranded breaks in the transposition process. Gam can be used to block homologous recombination with sister chromosomes to repair double strand breaks, sometimes leading to cell death. The survival of cells exposed to UV is similar for cells expression Gam and cells where the recB is mutated. This indicates that Gam blocks DNA repair (Cox, 2013). The Gam protein can thus promote Cas9-mediated killing (Cui et al., 2016). GamGFP is used to label double stranded breaks, although this can be difficult in eukaryotic cells as the Gam protein competes with similar eukaryotic protein Ku (Shee et al., 2013).

Gam is related to Ku70 and Ku80, two eukaryotic proteins involved in non-homologous DNA end-joining (Cui et al., 2016). Gam has sequence homology with both subunits of Ku (Ku70 and Ku80), and can have a similar structure to the core DNA-binding region of Ku. Orthologs to Mu Gam are present in the bacterial genomes of Haemophilus influenzae, Salmonella typhi, Neisseria meningitidis, and the enterohemorrhagic O157:H7 strain of E. coli (d'Adda di Fagagna et al., 2003). Gam proteins have been described previously, for example, in COX, Proteins pinpoint double strand breaks, eLife. 2013; 2: e01561.; Cui et al., Consequences of Cas9 cleavage in the chromosome of Escherichia coli. Nucleic Acids Res. 2016 May 19; 44(9):4243-51. doi: 10.1093/nar/gkw223. Epub 2016 Apr. 8.; D'ADDA DI FAGAGNA et al., The Gam protein of bacteriophage Mu is an orthologue of eukaryotic Ku. EMBO Rep. 2003 January; 4(1):47-52.; and SHEE et al., Engineered proteins detect spontaneous DNA breakage in human and bacterial cells. Elife. 2013 Oct. 29; 2:e01222. doi: 10.7554/eLife.01222; the contents of each of which are incorporated herein by reference.

In some embodiments, the Gam protein is a protein that binds double strand breaks in DNA and prevents or inhibits degradation of the DNA at the double strand breaks. In some embodiments, the Gam protein is a naturally occurring Gam protein from any organism (e.g., a bacterium), for example, any of the organisms provided herein. In some embodiments, the Gam protein is a variant of a naturally-occurring Gam protein from an organism. In some embodiments, the Gam protein does not occur in nature. In some embodiments, the Gam protein is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring Gam protein. In some embodiments, the Gam protein is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to any of the Gam proteins provided herein (e.g., SEQ ID NO: 9). Exemplary Gam proteins are provided below. In some embodiments, the Gam protein comprises any of the Gam proteins provided herein (e.g., SEQ ID NO: 710-734). In some embodiments, the Gam protein is a truncated version of any of the Gam proteins provided herein. In some embodiments, the truncated Gam protein is missing 1, 2, 3, 4, 5, 6, 7, 8, 9, 10, 11, 12, 13, 14, 15, 6, 17, 18, 19, or 20 N-terminal amino acid residues relative to a full-length Gam protein. In some embodiments, the truncated Gam protein may be missing 1, 2, 3, 4, 5, 6, 7, 8, 9, 10, 11, 12, 13, 14, 15, 6, 17, 18, 19, or 20 C-terminal amino acid residues relative to a full-length Gam protein. In some embodiments, the Gam protein does not comprise an N-terminal methionine.

In some embodiments, the Gam protein comprises an amino acid sequence that is at least 60%, at least 65%, at least 70%, at least 75%, at least 80%, at least 85%, at least 90%, at least 95, at least 98%, at least 99%, or at least 99.5% identical to any of the Gam proteins provided herein. In some embodiments, the Gam protein comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the Gam Proteins provided herein (e.g., SEQ ID NOs: 710-734). In some embodiments, the Gam protein comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170, identical contiguous amino acid residues as compared to any of the Gam proteins provided herein. In some embodiments, the Gam protein comprises the amino acid sequence of any of the Gam proteins provided herein. In some embodiments, the Gam protein consists of the any of the Gam proteins provided herein (e.g., SEQ ID NO: 710 or 711-734).

### Gam Form Bacteriophage Mu

(SEQ ID NO: 710)
AKPAKRIKSAAAAYVPQNRDAVITDIKRIGDLQREASRLETEMNDAIAEI
TEKFAARIAPIKTDIETLSKGVQGWCEANRDELTNGGKVKTANLVTGDVS
WRVRPPSVSIRGMDAVMETLERLGLQRFIRTKQEINKEAILLEPKAVAGV
AGITVKSGIEDFSIIPFEQEAGI

>WP_001107930.1 MULTISPECIES: host-nuclease inhibitor protein Gam [Enterobacteriaceae]

(SEQ ID NO: 711)
MAKPAKRIKSAAAAYVPQNRDAVITDIKRIGDLQREASRLETEMNDAIAE
ITEKFAARIAPIKTDIETLSKGVQGWCEANRDELTNGGKVKTANLVTGDV
SWRVRPPSVSIRGMDAVMETLERLGLQRFIRTKQEINKEAILLEPKAVAG
VAGITVKSGIEDFSIIPFEQEAGI

>CAA27978.1 unnamed protein product [Escherichia virus Mu]

(SEQ ID NO: 712)
MAKPAKRIKSAAAAYVPQNRDAVITDIKRIGDLQREASRLETEMNDAIAE
ITEKFAARIAPIKTDIETLSKGVQGWCEANRDELTNGGKVKTANLVTGDV
SWRVRPPSVSIRGMDAVMETLERLGLQRFVRTKQEINKEAILLEPKAVAG
VAGITVKSGIEDFSIIPFEQEAGI

>WP_001107932.1 host-nuclease inhibitor protein Gam [Escherichia coli]

(SEQ ID NO: 713)
MAKPAKRIKSAAAAYVPQNRDAVITDIKRIGDLQREASRLETEMNDAIAE
ITEKFAARIAPLKTDIETLSKGVQGWCEANRDELTNGGKVKTANLVTGDV
SWRVRPPSVSIRGMDAVMETLERLGLQRFIRTKQEINKEAILLEPKAVAG
VAGITVKSGIEDFSIIPFEQEAGI

>WP_061335739.1 host-nuclease inhibitor protein Gam [Escherichia coli]

(SEQ ID NO: 714)
MAKPAKRIKSAAAAYVPQNRDAVITDIKRIGDLQREASRLETEMNDAIAE
ITEKFAARIAPIKTDIETLSKGVQGWCEANRDELTNGGKVKTANLITGDV
SWRVRPPSVSIRGMDAVMETLERLGLQRFIRTKQEINKEAILLEPKAVAG
VAGITVKSGIEDFSIIPFEQEAGI

>WP_001107937.1 MULTISPECIES: host-nuclease inhibitor protein Gam [Enterobacteriaceae]>EJL11163.1 bacteriophage Mu Gam like family protein [Shigella sonnei str. Moseley]>CSO81529.1 host-nuclease inhibitor protein [Shigella sonnei]>OCE38605.1 host-nuclease inhibitor protein Gam [Shigella sonnei]>SJK50067.1 host-nuclease inhibitor protein [Shigella sonnei]>SJK19110.1 host-nuclease inhibitor protein [Shigella sonnei]>SIY81859.1 host-nuclease inhibitor protein [Shigella sonnei]>SJJ34359.1 host-nuclease inhibitor protein [Shigella sonnei]>SJK07688.1 host-nuclease inhibitor protein [Shigella sonnei]>SJI95156.1 host-nuclease inhibitor protein [Shigella sonnei]>SIY86865.1 host-nuclease inhibitor protein [Shigella sonnei]>SJJ67303.1 host-nuclease inhibitor protein [Shigella sonnei]>SJJ18596.1 host-nuclease inhibitor protein [Shigella sonnei]>SIX52979.1 host-nuclease inhibitor protein [Shigella sonnei]>SJD05143.1 host-nuclease inhibitor protein [Shigella sonnei]>SJD37118.1 host-nuclease inhibitor protein [Shigella sonnei]>SJE51616.1 host-nuclease inhibitor protein [Shigella sonnei]

(SEQ ID NO: 715)
MAKPAKRIRNAAAAYVPQSRDAVVCDIRRIGDLQREAARLETEMNDAIAE
ITEKYASQIAPLKTSIETLSKGVQGWCEANRDELTNGGKVKTANLVTGDV
SWRQRPPSVSlRGVDAVMETLERLGLQRFIRTKQEINKEAILLEPKAVAG
VAGITVKSGIEDFSIIPFEQEAGI

>WP_089552732.1 host-nuclease inhibitor protein Gam [Escherichia coli]

(SEQ ID NO: 716)
MAKPAKRIKNAAAAYVPQSRDAVVCDIRRIGDLQREAARLETEMNDAIAE
ITEKYASQIAPLKTSIETISKGVQGWCEANRDELTNGGKVKTANLVTGDV
SWRQRPPSVSlRGVDAVMETLERLGLQRFIRTKQEINKEAILLEPKAVAG
VAGITVKSGIEDFSIIPFEQEAGI

>WP_042856719.1 host-nuclease inhibitor protein Gam [Escherichia coli]>CDL02915.1 putative host-nuclease inhibitor protein [Escherichia coli IS35]

(SEQ ID NO: 717)
MAKPAKRIKNAAAAYVPQSRDAVVCDIRRIGDLQREAARLETEMNDAIAD
ITEKYASQIAPLKTSIETLSKGVQGWCEANRDELTNGGKVKTANLVTGDV
SWRQRPPSVSlRGVDAVMETLERLGLQRFIRTKQEINKEAILLEPKAVAG
VAGITVKSGIEDFSIIPFEQEAGI

>WP_001129704.1 host-nuclease inhibitor protein Gam [Escherichia coli]>EDU62392.1 bacteriophage Mu Gam like protein [Escherichia coli 53638]

(SEQ ID NO: 718)
MAKSAKRIRNAAAAYVPQSRDAVVCDIRRIGNLQREAARLETEMNDAIAE
ITEKFAARIAPLKTDIETLSKGVQGWCEANRDELTNGGKVKTANLVTGDV
SWRQRPPSVSlRGVDAVMETLERLGLQRFIRTKQEINREAILLEPKAVAG
VAGITVKSGIEDFSIIPFEQDAGI

>WP_001107936.1 MULTISPECIES: host-nuclease inhibitor protein Gam [Enterobacteriaceae]>EGI94970.1 host-nuclease inhibitor protein gam [Shigella boydii 5216-82]>CSR34065.1 host-nuclease inhibitor protein [Shigella sonnei]>CSQ65903.1 host-nuclease inhibitor protein [Shigella sonnei]>CSQ94361.1 host-nuclease inhibitor protein [Shigella sonnei]>SJK23465.1 host-nuclease inhibitor protein [Shigella sonnei]>SJB59111.1 host-nuclease inhibitor protein [Shigella sonnei]>SJI55768.1 host-nuclease inhibitor protein [Shigella sonnei]>SJI56601.1 host-nuclease inhibitor protein [Shigella sonnei]>SJJ20109.1 host-nuclease inhibitor protein [Shigella sonnei]>SJJ54643.1 host-nuclease inhibitor protein [Shigella sonnei]>SJI29650.1 host-nuclease inhibitor protein [Shigella sonnei]>SIZ53226.1 host-nuclease inhibitor protein [Shigella sonnei]>SJA65714.1 host-nuclease inhibitor protein [Shigella sonnei]>SJJ21793.1 host-nuclease inhibitor protein [Shigella sonnei]>SJD61405.1 host-nuclease inhibitor protein [Shigella sonnei]>SJJ14326.1 host-nuclease inhibitor protein [Shigella sonnei]>SIZ57861.1 host-nuclease inhibitor protein [Shigella sonnei]>SJD58744.1 host-nuclease inhibitor protein [Shigella sonnei]>SJD84738.1 host-nuclease inhibitor protein [Shigella sonnei]>SJJ51125.1 host-nuclease inhibitor protein [Shigella sonnei]>SJD01353.1 host-nuclease inhibitor protein [Shigella sonnei]>SJE63176.1 host-nuclease inhibitor protein [Shigella sonnei]

(SEQ ID NO: 719)
MAKPAKRIRNAAAAYVPQSRDAVVCDIRRIGDLQREAARLETEMNDAIAE
ITEKYASQIAPLKTSIETLSKGVQGWCEANRDELTNGGKVKTANLVTGDV
SWRQRPPSVSIRGVDAVMETLERLGLQRFIRTKQEINKEAILLEPKAVAG
VAGITVKSGIEDFSIIPFEQDAGI

>WP_050939550.1 host-nuclease inhibitor protein Gam [Escherichia coli]>KNF77791.1 host-nuclease inhibitor protein Gam [Escherichia coli]

(SEQ ID NO: 720)
MAKPAKRIKNAAAAYVPQSRDAVVCDIRRIGDLQREAARLETEMNDAIAE
ITEKYASQIAPLKTSIETLSKGVQGWCEANRDELTNGGKVKTANLVTGDV
SWRLRPPSVSIRGVDAVMETLERLGLQRFICTKQEINKEAILLEPKVVAG
VAGITVKSGIEDFSIIPFEQEAGI

>WP_085334715.1 host-nuclease inhibitor protein Gam [Escherichia coli]>OSC16757.1 host-nuclease inhibitor protein Gam [Escherichia coli]

(SEQ ID NO: 721)
MAKPVKRIRNAAAAYVPQSRDAVVCDIRRIGDLQREAARLETEMNDAIAE
ITEKYASQIAPLKTSIETLSKGIQGWCEANRDELTNGGKVKTANLVTGDV
SWRQRPPSVSIRGVDAVMETLERLGLQRFIRTKQEINKEAILLEPKAVAG
VAGITVKSGIEDFSIIPFEQEAGI

>WP_065226797.1 host-nuclease inhibitor protein Gam [Escherichia coli]>ANO88858.1 host-nuclease inhibitor protein Gam [Escherichia coli]>ANO89006.1 host-nuclease inhibitor protein Gam [Escherichia coli]

(SEQ ID NO: 722)
MAKPAKRIRNAAAAYVPQSRDAVVCDIRWIGDLQREAVRLETEMNDAIAE
ITEKYASRIAPLKTRIETLSKGVQGWCEANRDELTNGGKVKTANLVTGDV
SWRQRPPSVSlRGVDAVMETLERLGLQRFIRTKQEINKEAILLEPKAVAG
VAGITVKSGIEDFSIIPFEQEAGI

>WP_032239699.1 host-nuclease inhibitor protein Gam [Escherichia coli]>KDU26235.1 bacteriophage Mu Gam like family protein [Escherichia coli 3-373-03_S4_C2]>KDU49057.1 bacteriophage Mu Gam like family protein [Escherichia coli 3-373-03_S4_C1]>KEL21581.1 bacteriophage Mu Gam like family protein [Escherichia coli 3-373-03_S4_C3]

(SEQ ID NO: 723)
MAKSAKRIRNAAATYVPQSRDAVVCDIRRIGDLQREAARLETEMNDAIAE
ITEKYASQIAPLKTSIETLSKGIQGWCEANRDELTNGGKVKTANLVTGDV
SWRQRPPSVSIRGVDAVMETLERLGLQRFIRTKQEINKEAILLEPKAVAG
VAGITVKSGIEDFSIIPFEQEAGI

>WP_080172138.1 host-nuclease inhibitor protein Gam [Salmonella enterica]

(SEQ ID NO: 724)
MAKSAKRIKSAAATYVPQSRDAVVCDIRRIGDLQREAARLETEMNDAIAE
ITEKYASQIAPLKTSIETLSKGVQGWCEANRDELTNGGKVKSANLVTGDV
QWRQRPPSVSIRGVDAVMETLERLGLQRFIRTKQEINKEAILLEPKAVAG
VAGITVKSGIEDFSIIPFEQEAGI

>WP_077134654.1 host-nuclease inhibitor protein Gam [Shigella sonnei]>SIZ51898.1 host-nuclease inhibitor protein [Shigella sonnei]>SJK07212.1 host-nuclease inhibitor protein [Shigella sonnei]

(SEQ ID NO: 725)
MAKSAKRIRNAAAAYVPQSRDAVVCDIRRIGNLQREAARLETEMNDAIAE
ITEKYASQIAPLKTSIETLSKGVQGWCEANRDELTNGGKVKTANLVTGDV
SWRQRPPSVSlRGVDAVMETLERLGLQRFIRTKQEINKEAILLEPKAVAG
VAGITVKSGIEDFSIIPFEQDAGI

>WP_000261565.1 host-nuclease inhibitor protein Gam [Shigella flexneri]>EGK20651.1 host-nuclease inhibitor protein gam [Shigella flexneri K-272]>EGK34753.1 host-nuclease inhibitor protein gam [Shigella flexneri K-227]

(SEQ ID NO: 726)
MVVSAIASTPHDAVVCDIRRIGDLQREAARLETEMNDAIAEITEKDASQI
APLKTSIETLSKGVQGWCEANRDELTNGGKVKTANLVTGDVSWRQRPPSV
SIRGVDAVMETLERLGLQRFIRTKQEINKEAILLEPKAVAGVAGITVKSG
IEDFSIIPFEQEAGI

>ASG63807.1 host-nuclease inhibitor protein Gam [Kluyvera georgiana]

(SEQ ID NO: 727)
MVSKPKRIKAAAANYVSQSRDAVITDIRKIGDLQREATRLESAMNDEIAV
ITEKYAGLIKPLKADVEMLSKGVQGWCEANRDDLTSNGKVKTANLVTGDI
QWRIRPPSVSVRGPDAVMETLTRLGLSRFIRTKQEINKEAILNEPLAVAG
VAGITVKSGIEDFSIIPFEQTADI

>WP_078000363.1 host-nuclease inhibitor protein Gam [Edwardsiella tarda]

(SEQ ID NO: 728)
MASKPKRIKSAAANYVSQSRDAVIIDIRKIGDLQREATRLESAMNDEIAV
ITEKYAGLIKPLKADVEMLSKGVQGWCEANRDELTCNGKVKTANLVTGDI
QWRIRPPSVSVRGPDSVMETLLRLGLSRFIRTKQEINKEAILNEPLAVAG
VAGITVKTGVEDFSIIPFEQTADI

>WP_047389411.1 host-nuclease inhibitor protein Gam [Citrobacter freundii]>KGY86764.1 host-nuclease inhibitor protein Gam [Citrobacter freundii]>OIZ37450.1 host-nuclease inhibitor protein Gam [Citrobacter freundii]

(SEQ ID NO: 729)
MVSKPKRIKAAAANYVSQSKEAVIADIRKIGDLQREATRLESAMNDEIAV
ITEKYAGLIKPLKTDVEILSKGVQGWCEANRDELTSNGKVKTANLVTGDI
QWRIRPPSVAVRGPDAVMETLLRLGLSRHRTKQEINKEAILNEPLAVAGV
AGITVKSGVEDFSIIPFEQTADI

>WP_058215121.1 host-nuclease inhibitor protein Gam [Salmonella enterica]>KSU39322.1 host-nuclease inhibitor protein Gam [Salmonella enterica subsp. enterica]>OHJ24376.1 host-nuclease inhibitor protein Gam [Salmonella enterica]>ASG15950.1 host-nuclease inhibitor protein Gam [Salmonella enterica subsp. enterica serovar Macclesfield str. S-1643]

(SEQ ID NO: 730)
MASKPKRIKAAAALYVSQSREDVVRDIRMIGDFQREIVRLETEMNDQIAA
VTLKYADKIKPLQEQLKTLSEGVQNWCEANRSDLTNGGKVKTANLVTGDV
QWRVRPPSVTVRGVDSVMETLRRLGLSRFIRIKEEINKEAILNEPGAVAG
VAGITVKSGVEDFSIIPFEQSATN

>WP_016533308.1 phage host-nuclease inhibitor protein Gam [Pasteurella multocida]>EPE65165.1 phage host-nuclease inhibitor protein Gam [Pasteurella multocida P1933]>ESQ71800.1 host-nuclease inhibitor protein Gam [Pasteurella multocida subsp. multocida P1062]>ODS44103.1 host-nuclease inhibitor protein Gam [Pasteurella multocida]>OPC87246.1 host-nuclease inhibitor protein Gam [Pasteurella multocida subsp. multocida]>OPC98402.1 host-nuclease inhibitor protein Gam [Pasteurella multocida subsp. multocida]

(SEQ ID NO: 731)
MAKKATRIKTTAQVYVPQSREDVASDIKTIGDLNREITRLETEMNDKIAE
ITESYKGQFSPIQERIKNLSTGVQFWAEANRDQITNGGKTKTANLITGEV
SWRVRNPSVKITGVDSVLQNLKIHGLTKFIRVKEEINKEAILNEKHEVAG
IAGIKVVSGVEDFVITPFEQEI

>WP_005577487.1 host-nuclease inhibitor protein Gam [Aggregatibacter actinomycetemconitans]>EHK90561.1 phage host-nuclease inhibitor protein Gam [Aggregatibacter actinomycetencomitans RhAA1]>KNE77613.1 host-nuclease inhibitor protein Gam [Aggregatibacter actinomycetemcomitans RhAA1]

(SEQ ID NO: 732)
MAKSATRVKATAQIYVPQTREDAAGDIKTIGDLNREVARLEAEMNDKIAA
ITEDYKDKFAPLQERIKTLSNGVQYWSEANRDQITNGGKTKTANLVTGEV
SWRVRNPSVKVTGVDSVLQNLRIHGLERFIRTKEEINKEAILNEKSAVAG
IAGIKVITGVEDFVITPFEQEAA

>WP_090412521.1 host-nuclease inhibitor protein Gam [Nitrosomonas halophila]>SDX89267.1 Mu-like prophage host-nuclease inhibitor protein Gam [Nitrosomonas halophila]

(SEQ ID NO: 733)
MARNAARLKTKSIAYVPQSRDDAAADIRKIGDLQRQLTRTSTEMNDAIAA
ITQNFQPRMDAIKEQINLLQAGVQGYCEAHRHALTDNGRVKTANLITGEV
QWRQRPPSVSIRGQQVVLETLRRLGLERFIRTKEEVNKEAILNEPDEVRG
VAGLNVITGVEDFVITPFEQEQP

>WP_077926574.1 host-nuclease inhibitor protein Gam [Wohlfahrtiimonas larvae]

(SEQ ID NO: 734)
MAKKRIKAAATVYVPQSKEEVQNDIREIGDISRKNERLETEMNDRIAEIT
NEYAPKFEVNKVRLELLTKGVQSWCEANRDDLTNSGKVKSANLVTGKVEW
RQRPPSISVKGMDAVIEWLQDSKYQRFLRTKVEVNKEAMLNEPEDAKTIP
GITIKSGIEDFAITPFEQEAGV

### Deaminase Domains

In some embodiments, the nucleobase editor useful in the present disclosure comprises: (i) a guide nucleotide sequence-programmable DNA-binding protein domain; and (ii) a deaminase domain. In certain embodiments, the deaminase domain of the fusion protein is a cytosine deaminase. In some embodiments, the deaminase is an APOBEC1 deaminase. In some embodiments, the deaminase is a rat APOBEC1. In some embodiments, the deaminase is a human APOBEC1. In some embodiments, the deaminase is an APOBEC2 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a Lamprey CDA1 (pmCDA1). In some embodiments, the deaminase is a human APOBEC3G or a functional fragment thereof. In some embodiments, the deaminase is an APOBEC3G variant comprising mutations correspond to the D316R/D317R mutations in the human APOBEC3G. Exemplary, non-limiting cytosine deaminase sequences that may be used in accordance with the methods of the present disclosure are provided in Example 1 below.

In some embodiments, the cytosine deaminase is a wild type deaminase or a deaminase as set forth in SEQ ID NOs: 1-260, 270-292, 315-323, 680, or 682. In some embodiments, the cytosine deaminase domains of the fusion proteins provided herein include fragments of deaminases and proteins homologous to either a deaminase or a deaminase fragment. For example, in some embodiments, a deaminase domain may comprise a fragment of the amino acid sequence set forth in any of SEQ ID NOs: 1-260, 270-292, 315-323, 680, or 682. In some embodiments, a deaminase domain comprises an amino acid sequence homologous to the amino acid sequence set forth in any of SEQ ID NOs: 1-260, 270-292, 315-323, 680, or 682, or an amino acid sequence homologous to a fragment of the amino acid sequence set forth in any of SEQ ID NOs: 1-260, 270-292, 315-323, 680, or 682. In some embodiments, proteins comprising a deaminase, a fragment of a deaminase, or a homolog of a deaminase are referred to as “deaminase variants.” A deaminase variant shares homology to a deaminase, or a fragment thereof. For example a deaminase variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to a wild type deaminase or a deaminase as set forth in any of SEQ ID NOs: 1-260, 270-292, or 315-323. In some embodiments, the deaminase variant comprises a fragment of the deaminase, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type deaminase or a deaminase as set forth in any of SEQ ID NOs: 1-260, 270-292, 315-323, 680, or 682. In some embodiments, the cytosine deaminase is at least at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to an APOBEC3G variant as set forth in SEQ ID NO: 291 or SEQ ID NO: 292, and comprises mutations corresponding to the D316F/D317R mutations in SEQ ID NO: 290.

In some embodiments, the cytosine deaminase domain is fused to the N-terminus of the guide nucleotide sequence-programmable DNA-binding protein domain. For example, the fusion protein may have an architecture of NH₂-[cytosine deaminase]-[guide nucleotide sequence-programmable DNA-binding protein domain]-COOH. The “−” used in the general architecture above indicates the presence of an optional linker. The term “linker,” as used herein, refers to a chemical group or a molecule linking two molecules or moieties, e.g., two domains of a fusion protein, such as, for example, a dCas9 domain and a cytosine deaminase domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. Linkers may be of any form known in the art. For example, the linker may be a linker from a website, such as www[dot]ibi[dot]vu[dot]nl/programs/linkerdbwww/ or from www[dot]ibi[dot]vu[dot]nl/programs/linkerdbwww/src/database.txt. The linkers may also be unstructured, structured, helical, or extended.

In some embodiments, the cytosine deaminase domain and the Cas9 domain are fused to each other via a linker. Various linker lengths and flexibilities between the deaminase domain (e.g., APOBEC1) and the Cas9 domain can be employed (e.g., ranging from flexible linkers of the form (GGGS)ₙ (SEQ ID NO: 303). (GGGGS)ₙ (SEQ ID NO: 304), (GGS)ₙ, and (G)ₙ to more rigid linkers of the form (EAAAK)ₙ (SEQ ID NO: 305, SGSETPGTSESATPES (SEQ ID NO: 306) (see. e.g., Guilinger et al., Nat. Biotechnol. 2014; 32(6): 577-82; the entire contents of which is incorporated herein by reference), (XP)ₙ, or a combination of any of these, wherein X is any amino acid, and n is independently an integer between 1 and 30, in order to achieve the optimal length for deaminase activity for the specific application. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS), motif, wherein n is 1, 2, 3, 4, 5, 6, 7, 8, 9, 10, 11, 12, 13, 14 or 15. In some embodiments, the linker comprises a (GGS)ₙ motif, wherein n is 1, 3, or 7. In some embodiments, the linker comprises the amino acid sequence SGSETPGTSESATPES (SEQ ID NO: 306), also referred to as the XTEN linker. In some embodiments, the linker comprises an amino acid sequence chosen from the group including, but not limited to, AGVF (SEQ ID NO: 307), GFLG (SEQ ID NO: 308), FK, AL, ALAL (SEQ ID NO: 349), and ALALA (SEQ ID NO: 309). In some embodiments, suitable linker motifs and configurations include those described in Chen et al., Fusion protein linkers: property, design and functionality. Adv Drug Deliv Rev. 2013; 65(10):1357-69, which is incorporated herein by reference. In some embodiments, the linker may comprise any of the following amino acid sequences: VPFLLEPDNINGKTC (SEQ ID NO: 350), GSAGSAAGSGEF (SEQ ID NO: 351), SIVAQLSRPDPA (SEQ ID NO: 352), MKIIEQLPSA (SEQ ID NO: 353), VRHKLKRVGS (SEQ ID NO: 354), GHGTGSTGSGSS (SEQ ID NO: 355), MSRPDPA (SEQ ID NO: 356), GSAGSAAGSGEF (SEQ ID NO: 357), SGSETPGTSESA (SEQ ID NO: 358), SGSETPGTSESATPEGGSGGS (SEQ ID NO: 359), and GGSM (SEQ ID NO: 360). Additional suitable linker sequences will be apparent to those of skill in the art based on the instant disclosure.

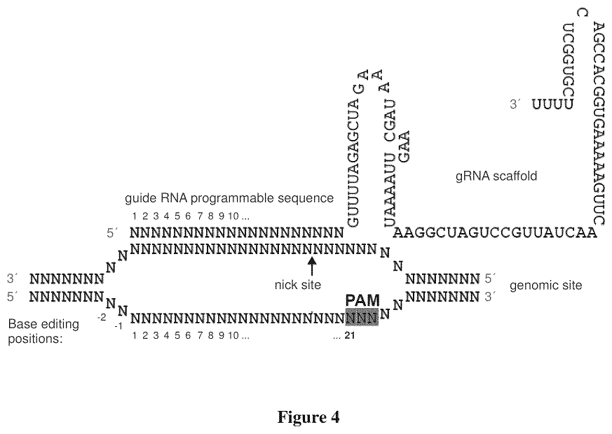

To successfully edit the desired target C base, the linker between Cas9 and APOBEC may be optimized, as described in Komor et al., Nature, 533, 420-424 (2016), which is incorporated herein by reference. The numbering scheme for base editing is based on the predicted location of the target C within the single stranded stretch of DNA (R-loop) displaced by a programmable guide RNA sequence occurring when a DNA-binding domain (e.g, Cas9, nCas9, dCas9) binds a genomic site (see FIG. 4). Conveniently, the sequence immediately surrounding the target C also matches the sequence of the guide RNA, which may be used as a reference as done in the Tables herein. The numbering scheme for base editing is based on a standard 20-mer programmable sequence, and defines position “21” as the first DNA base of the PAM sequence, resulting in position “1” assigned to the first DNA base matching the 5′-end of the 20-mer programmable guide RNA sequence. Therefore, for all Cas9 variants, position “21” is defined as the first base of the PAM sequence (e.g. NGG. NGAN. NGNG, NGAG, NGCG, NNGRRT. NGRRN. NNNRRT. NNNGATT. NNAGAA, NAAAC). When a longer programmable guide RNA sequence is used (e.g. 21-mer) the 5′-end bases are assigned a decreasing negative number starting at “−1”. For other DNA-binding domains that differ in the position of the PAM sequence, or that require no PAM sequence, the programmable guide RNA sequence is used as a reference for numbering. A 3-aa linker gives a 2-5 base editing window (e.g., positions 2, 3, 4, or 5 relative to the PAM sequence in positions 20-23). A 9-aa linker gives a 3-6 base editing window (e.g., positions 3, 4, 5, or 6 relative to the PAM sequence at position 21). A 16-aa linker (e.g., the SGSETPGTSESATPES (SEQ ID NO: 306) linker) gives a 4-7 base editing window (e.g., positions 4, 5, 6, or 7 relative to the PAM sequence at position 21). A 21-aa linker gives a 5-8 base editing window (e.g., positions 5, 6, 7, 8 relative to the PAM sequence at position 21). Each of these windows can be useful for editing different targeted C bases. For example, the targeted C bases may be at different distances from the adjacent PAM sequence, and by varying the linker length, the precise editing of the desired C base is ensured. One skilled in the art, based on the teachings of CRISPR/Cas9 technology, in particular the teachings of U.S. Provisional Applications 62/245,828, 62/279,346, 62/311,763, 62/322,178, 62/357,352, 62/370,700, and 62/398,490, and in Komor et al., Nature, “Programmable editing of a target base in genomic DNA without double-stranded DNA cleavage,” 533, 420-424 (2016), each of which is incorporated herein by reference, will be able to determine the editing window for his/her purpose, and properly design the linker of the cytosine deaminase-dCas9 protein for the precise targeting of the desired C base. To successfully edit the desired target C base, the sequence identity of the homolog of Cas9 attached to APOBEC may be optimized based on the teachings of CRISPR/Cas9 technology. As a non-limiting example, the teachings of any of the following documents may be used: U.S. Provisional Application Nos. 62/245,828, 62/279,346, 62/311,763, 62/322,178, 62/357,352, 62/370,700, and 62/398,490, and Komor et al., Nature, 533, 420-424 (2016), each of which is incorporated herein by reference in its entirety. APOBEC1-XTEN-SaCas9n-UGI gives a 1-12 base editing window (e.g., positions 1, 2, 3, 4, 5, 6, 7, 8, 9, 10, 11, or 12 relative to the NNNRRT PAM sequence in positions 20-26). One skilled in the art, based on the teachings of CRISPR/Cas9 technology, will be able to determine the editing window for his/her purpose, and properly determine the required Cas9 homolog and linker attached to the cytosine deaminase for the precise targeting of the desired C base.

In some embodiments, the fusion protein useful in the present disclosure further comprises a uracil glycosylase inhibitor (UGI) domain. A “uracil glycosylase inhibitor” refers to a protein that inhibits the activity of uracil-DNA glycosylase. The C to T base change induced by deamination results in a U:G heteroduplex, which triggers a cellular DNA-repair response. Uracil DNA glycosylase (UDG) catalyzes removal of U from DNA in cells and initiates base excision repair, with reversion of the U:G pair to a C:G pair as the most common outcome. Thus, such cellular DNA-repair response may be responsible for the decrease in nucleobase editing efficiency in cells. Uracil DNA Glycosylase Inhibitor (UGI) is known in the art to potently blocks human UDG activity. As described in Komor et al., Nature (2016), fusing a UGI domain to the cytidine deaminase-dCas9 fusion protein reduced the activity of UDG and significantly enhanced editing efficiency.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), each of which is incorporated herein by reference. In some embodiments, the UGI comprises the following amino acid sequence:

### Bacillus Phage PBS2 (Bacteriophage PBS2) Uracil-DNA Glycosylase Inhibitor

(SEQ ID NO: 361)
MTNLSDIIEKETGKQLVIQESILMLPEEVEEVIGNKPESDILVHTAYDES
TDENVMLLTSDAPEYKPWALVIQDSNGENKIKML

In some embodiments, the UGI protein comprises a wild type UGI or a UGI as set forth in SEQ ID NO: 361. In some embodiments, the UGI proteins useful in the present disclosure include fragments of UGI and proteins homologous to a UGI or a UGI fragment. For example, in some embodiments, a UGI comprises a fragment of the amino acid sequence set forth in SEQ ID NO: 361. In some embodiments, a UGI comprises an amino acid sequence homologous to the amino acid sequence set forth in SEQ ID NO: 361 or an amino acid sequence homologous to a fragment of the amino acid sequence set forth in SEQ ID NO: 361. In some embodiments, proteins comprising UGI or fragments of UGI or homologs of either UGI or UGI fragments are referred to as “UGI variants.” A UGI variant shares homology with UGI, or a fragment thereof. For example, a UGI variant is at least about 70% identical, at least about 80% identical, at least about 85% identical, at least about 90% identical, at least about 95% identical, at least about 96% identical, at least about 97% identical, at least about 98% identical, at least about 99% identical, at least about 99.5% identical, or at least about 99.9% identical to a wild type UGI or a UGI as set forth in SEQ ID NO: 361. In some embodiments, the UGI variant comprises a fragment of UGI, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type UGI or a UGI as set forth in SEQ ID NO: 361.

It should be appreciated that additional proteins may be uracil glycosylase inhibitors. For example, other proteins that are capable of inhibiting (e.g., sterically blocking) a uracil-DNA glycosylase base-excision repair enzyme are within the scope of this disclosure. In some embodiments, a uracil glycosylase inhibitor is a protein that binds DNA. In some embodiments, a uracil glycosylase inhibitor is a protein that binds single-stranded DNA. For example, a uracil glycosylase inhibitor may be a Erwinia tasmaniensis single-stranded binding protein. In some embodiments, the single-stranded binding protein comprises the amino acid sequence (SEQ ID NO: 362). In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. For example, a uracil glycosylase inhibitor is a UdgX. In some embodiments, the UdgX comprises the amino acid sequence (SEQ ID NO: 363). As another example, a uracil glycosylase inhibitor is a catalytically inactive UDG. In some embodiments, a catalytically inactive UDG comprises the amino acid sequence (SEQ ID NO: 364). It should be appreciated that other uracil glycosylase inhibitors would be apparent to the skilled artisan and are within the scope of this disclosure. In some embodiments, the fusion protein comprises a guide nucleotide sequence-programmable DNA-binding protein, a cytidine deaminase domain, a Gam protein, and a UGI domain. In some embodiments, any of the fusion proteins provided herein that comprise a guide nucleotide sequence-programmable DNA-binding protein (e.g., a Cas9 domain), a cytidine deaminase, and a Gam protein may be further fused to a UGI domain either directly or via a linker. This disclosure also contemplates a fusion protein comprising a Cas9 nickase-nucleic acid editing domain fused to a cytidine deaminase and a Gam protein, which is further fused to a UGI domain.

Erwinia tasmaniensis SSB (Themostable Single-Stranded DNA Binding Protein)

(SEQ ID NO: 362)
MASRGVNKVILVGNLGQDPEVRYMPNGGAVANITLATSESWRDKQTGETK
EKTEWHRVVLFGKLAEVAGEYLRKGSQVYIEGALQTRKWTDQAGVEKYTT
EVVVNVGGTMQMLGGRSQGGGASAGGQNGGSNNGWGQPQQPQGGNQFSGG
AQQQARPQQQPQQNNAPANNEPPIDFDDDIP

UdgX (Binds to Uracil in DNA but does not Excise)

(SEQ ID NO: 363)
MAGAQDFVPHTADLAELAAAAGECRGCGLYRDATQAVFGAGGRSARIMMI
GEQPGDKEDLAGLPFVGPAGRLLDRALEAADIDRDALYVTNAVKHFKFTR
AAGGKRRIHKTPSRTEVVACRPWLIAEMTSVEPDVVVLLGATAAKALLGN
DFRVTQHRGEVLHVDDVPGDPALVATVHPSSLLRGPKEERESAFAGLVDD
LRVAADVRP

UDG (Catalytically Inactive Human UDG, Binds to Uracil in DNA but does not Excise)

(SEQ ID NO: 364)
MIGQKTLYSFFSPSPARKRHAPSPEPAVQGTGVAGVPEESGDAAAIPAKK
APAGQEEPGTPPSSPLSAEQLDRIQRNKAAALLRLAARNVPVGFGESWKK
HLSGEFGKPYFIKLMGFVAEERKHYTVYPPPHQVFTWTQMCDIKDVKVVI
LGQEPYHGPNQAHGLCFSVQRPVPPPPSLENIYKELSTDIEDFVHPGHGD
LSGWAKQGVLLLNAVLTVRAHQANSHKERGWEQFTDAVVSWLNQNSNGLV
FLLWGSYAQKKGSAIDRKRHHVLQTAHPSPLSVYRGFFGCRHFSKTNELL
QKSGKKPIDWKEL

In some embodiments, the UGI domain is fused to the C-terminus of the dCas9 domain in the fusion protein. Thus, the fusion protein would have an architecture of NH₂-[cytosine deaminase]-[guide nucleotide sequence-programmable DNA-binding protein domain]-[UGI]-COOH. In some embodiments, the UGI domain is fused to the N-terminus of the cytosine deaminase domain. As such, the fusion protein would have an architecture of NH₂-[UGI]-[cytosine deaminase]-[guide nucleotide sequence-programmable DNA-binding protein domain]-COOH. In some embodiments, the UGI domain is fused between the guide nucleotide sequence-programmable DNA-binding protein domain and the cytosine deaminase domain. As such, the fusion protein would have an architecture of NH₂-[cytosine deaminase]-[UGI]-[guide nucleotide sequence-programmable DNA-binding protein domain]-COOH. The linker sequences described herein may also be used for the fusion of the UGI domain to the cytosine deaminase-dCas9 fusion proteins.

In some embodiments, the fusion protein comprises the structure:
[cytosine deaminase]-[optional linker sequence]-[guide nucleotide sequence-programmable DNA binding protein]-[optional linker sequence]-[UGI];
[cytosine deaminase]-[optional linker sequence]-[UGI]-[optional linker sequence]-[guide nucleotide sequence-programmable DNA binding protein];
[UGI]-[optional linker sequence]-[cytosine deaminase]-[optional linker sequence]-[guide nucleotide sequence-programmable DNA binding protein];
[UGI]-[optional linker sequence]-[guide nucleotide sequence-programmable DNA binding protein]-[optional linker sequence]-[cytosine deaminase];
[guide nucleotide sequence-programmable DNA binding protein]-[optional linker sequence]-[cytosine deaminase]-[optional linker sequence]-[UGI]; or
[guide nucleotide sequence-programmable DNA binding protein]-[optional linker sequence]-[UGI]-[optional linker sequence]-[cytosine deaminase].

In some embodiments, the fusion protein is of the structure:
[cytosine deaminase]-[optional linker sequence]-[Cas9 nickase]-[optional linker sequence]-[UGI]; [cytosine deaminase]-[optional linker sequence]-[UGI]-[optional linker sequence]-[Cas9 nickase]; [UGI]-[optional linker sequence]-[cytosine deaminase]-[optional linker sequence]-[Cas9 nickase]; [UGI]-[optional linker sequence]-[Cas9 nickase]-[optional linker sequence]-[cytosine deaminase]; [Cas9 nickase]-[optional linker sequence]-[cytosine deaminase]-[optional linker sequence]-[UGI]; or [Cas9 nickase]-[optional linker sequence]-[UGI]-[optional linker sequence]-[cytosine deaminase].

In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the UGI protein. In some embodiments, the NLS is fused to the C-terminus of the UGI protein. In some embodiments, the NLS is fused to the N-terminus of the guide nucleotide sequence-programmable DNA-binding protein domain. In some embodiments, the NLS is fused to the C-terminus of the guide nucleotide sequence-programmable DNA-binding protein domain. In some embodiments, the NLS is fused to the N-terminus of the cytosine deaminase. In some embodiments, the NLS is fused to the C-terminus of the deaminase. In some embodiments, the NLS is fused to the fusion protein via one or more linkers. In some embodiments, the NLS is fused to the fusion protein without a linker. Non-limiting, exemplary NLS sequences may be PKKKRKV (SEQ ID NO: 365) or MDSLLMNRRKFLYQFKNVRWAKGRRETYLC (SEQ ID NO: 366).

Some aspects of the present disclosure provide nucleobase editors described herein associated with a guide nucleotide sequence (e.g., a guide RNA or gRNA), gRNAs can exist as a complex of two or more RNAs. or as a single RNA molecule, gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as a single RNA species comprise two domains: (1) a domain that shares homology to a target nucleic acid (e.g., and directs binding of the Cas9 complex to the target); and (2) a domain that binds the Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” each are hereby incorporated by reference in their entirety. The gRNA comprises a nucleotide sequence that complements a target site, which mediates binding of the nuclease/RNA complex to said target site, providing the sequence specificity of the nuclease:RNA complex. These proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Science 339, 819-823 (2013); Mali, P. et al. Science 339, 823-826 (2013); Hwang, W. Y. et al. Nature Biotechnology 31, 227-229 (2013); Jinek, M. et al. eLife 2, e00471 (2013); Dicarlo, J. E. et al. Nucleic acids research (2013); Jiang, W. et al. Nature Biotechnology 31, 233-239 (2013); the entire contents of each of which are incorporated herein by reference). In particular, examples of guide nucleotide sequences (e.g., sgRNAs) that may be used to target the fusion protein of the present disclosure to its target sequence to deaminate the targeted C bases are described in Komor et al., Nature, 533, 420-424 (2016), which is incorporated herein by reference.

The specific structure of the guide nucleotide sequences (e.g., sgRNAs) depends on its target sequence and the relative distance of a PAM sequence downstream of the target sequence. One skilled in the art will understand, that no unifying structure of guide nucleotide sequence is given, because the target sequences are different for each and every C targeted to be deaminated.

However, the present disclosure provides guidance in how to design the guide nucleotide sequence. e.g., an sgRNA, so that one skilled in the art may use such teachings to design these for a target sequence of interest. A gRNA typically comprises a tracrRNA framework allowing for Cas9 binding, and a guide sequence, which confers sequence specificity to fusion proteins disclosed herein to target the CCR5 gene. In some embodiments, the guide RNA comprises a structure 5′-[guide sequence]-tracrRNA-3′. Non-limiting, exemplary tracrRNA sequences are shown in Table 10. The tracrRNA sequence may vary from the presented sequences.

TABLE 10
TracrRNA othologues and sequences

| | | SEQ ID |
|---|---|---|
| Organism | tracrRNA sequence | NO: |
| C. jejuni | AAGAAAUUUAAAAAGGGACUAAAAUAAAGAGUUUGCG | 367 |
| | GGACUCUGCGGGGUUACAAUCCCCUAAAACCGCUUUU | |
| F. novicida | AUCUAAAAUUAUAAAUGUACCAAAUAAUUAAUGCUCU | 368 |
| | GUAAUCAUUUAAAAGUAUUUUGAACGGACCUCUGUUU | |
| | GACACGUCUGAAUAACUAAAA | |
| S. thermophilus2 | UGUAAGGGACGCCUUACACAGUUACUUAAAUCUUGCA | 369 |
| | GAAGCUACAAAGAUAAGGCUUCAUGCCGAAAUCAACA | |
| | CCCUGUCAUUUUAUGGCAGGGUGUUUUCGUUAUUU | |
| M. mobile | UGUAUUUCGAAAUACAGAUGUACAGUUAAGAAUACAU | 370 |
| | AAGAAUGAUACAUCACUAAAAAAAGGCUUUAUGCCGU | |
| | AACUACUACUUAUUUUCAAAAUAAGUAGUUUUUUUU | |
| L. innocua | AUUGUUAGUAUUCAAAAUAACAUAGCAAGUUAAAAUA | 371 |
| | AGGCUUUGUCCGUUAUCAACUUUUAAUUAAGUAGCGC | |
| | UGUUUCGGCGCUUUUUUU | |
| S. pyogenes | GUUGGAACCAUUCAAAACAGCAUAGCAAGUUAAAAUA | 372 |
| | AGGCUAGUCCGUUAUCAACUUGAAAAAGUGGCACCGA | |
| | GUCGGUGCUUUUUUU | |
| S. mutans | GUUGGAAUCAUUCGAAACAACACAGCAAGUUAAAAUA | 373 |
| | AGGCAGUGAUUUUUAAUCCAGUCCGUACACAACUUGA | |
| | AAAAGUGCGCACCGAUUCGGUGCUUUUUUAUUU | |
| S. thermophilus | UUGUGGUUUGAAACCAUUCGAAACAACACAGCGAGUU | 374 |
| | AAAAUAAGGCUUAGUCCGUACUCAACUUGAAAAGGUG | |
| | GCACCGAUUCGGUGUUUUUUUU | |
| N. meningitidis | ACAUAUUGUCGCACUGCGAAAUGAGAACCGUUGCUAC | 375 |
| | AAUAAGGCCGUCUGAAAAGAUGUGCCGCAACGCUCUG | |
| | CCCCUUAAAGCUUCUGCUUUAAGGGGCA | |
| P. multocida | GCAUAUUGUUGCACUGCGAAAUGAGAGACGUUGCUAC | 376 |
| | AAUAAGGCUUCUGAAAAGAAUGACCGUAACGCUCUGC | |
| | CCCUUGUGAUUCUUAAUUGCAAGGGGCAUCGUUUUU | |
| S. pyogenes | GUUUAAGAGCUAUGCUGGAAAGCCACGGUGAAAAAGU | 377 |
| | UCAACUAUUGCCUGAUCGGAAUAAAUUUGAACGAUAC | |
| | GACAGUCGGUGCUUUUUUU | |
| S. pyogenes | GUUUAAGAGCUAGAAAUAGCAAGUUUAAAUAAGGCUA | 378 |
| | GUCCGUUAUCAACUUGAAAAAGUGGCACCGAGUCGGU | |
| | GCUUUUUU | |
| S. thermophilus | GUUUUUGUACUCUCAAGAUUCAAUAAUCUUGCAGAAG | 379 |
| CRISPR1 | CUACAAAGAUAAGGCUUCAUGCCGAAAUCAACACCCU | |
| | GUCAUUUUAUGGCAGGGUGUUUU | |
| S. thermophilus | GUUUUAGAGCUGUGUUGUUUGUUAAAACAACACAGCG | 380 |
| CRISPR3 | AGUUAAAAUAAGGCUUAGUCCGUACUCAACUUGAAAA | |
| | GGUGGCACCGAUUCGGUGUUUUU | |

The guide sequence of the gRNA comprises a sequence that is complementary to the target sequence. The guide sequence is typically about 20 nucleotides long. For example, the guide sequence may be 15-25 nucleotides long. In some embodiments, the guide sequence is 15, 16, 17, 18, 19, 20, 21, 22, 23, 24, or 25 nucleotides long. In some embodiments, the guide sequence is more than 25 nucleotides long. Such suitable guide RNA sequences typically comprise guide sequences that are complementary to a nucleic sequence within 50 nucleotides upstream or downstream of the target nucleotide to be edited.

In some embodiments, the guide RNA is about 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

### Compositions

Aspects of the present disclosure relate to compositions that may be used for editing CCR5-encoding polynucleotides, CCR2-encoding polynucleotides, or both CCR5-encoding polynucleotides and CCR2-encoding polynucleotides. In some embodiments, the editing is carried out in vitro. In some embodiments, the editing is carried out in a cultured cell. In some embodiments, the editing is carried out in vivo. In some embodiments, the editing is carried out in a mammal. In some embodiments, the mammal is a human. In some embodiments, the mammal may be a rodent. In some embodiments, the editing is carried out ex vivo.

In some embodiments, the composition comprises: (i) a fusion protein comprising: (a) a guide nucleotide sequence-programmable DNA binding protein domain; and (b) a cytosine deaminase domain; and (ii) a guide nucleotide sequence targeting the fusion protein of (i) to a polynucleotide encoding a C-C chemokine receptor type 5 (CCR5) protein.

In some embodiments, the composition comprises: (i) a fusion protein comprising: (a) a guide nucleotide sequence-programmable DNA binding protein domain; and (b) a cytosine deaminase domain; (ii) a guide nucleotide sequence targeting the fusion protein of (i) to a polynucleotide encoding a C-C chemokine receptor type 2 (CCR2) protein.

In some embodiments, the composition comprises: (i) a fusion protein comprising: (a) a guide nucleotide sequence-programmable DNA binding protein domain; and (b) a cytosine deaminase domain; (ii) a guide nucleotide sequence targeting the fusion protein of (i) to a polynucleotide encoding a C-C chemokine receptor type 5 (CCR5) protein; (iii) a guide nucleotide sequence targeting the fusion protein of (i) to a polynucleotide encoding a C-C chemokine receptor type 2 (CCR2) protein.

The guide nucleotide sequence used in the compositions described herein for editing the CCR5-encoding polynucleotide is selected from SEQ ID NOs: 381-657. In some embodiments, the composition comprises a nucleic acid encoding a fusion protein described herein and a guide nucleotide sequence described herein. In some embodiments, the composition described herein further comprises a pharmaceutically acceptable carrier. In some embodiments, the nucleobase editor (i.e., the fusion protein) and the gRNA are provided in two different compositions.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, having physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose, and cellulose acetate; (4) powdered tragacanth; (5) malt; (6) gelatin; (7) lubricating agents, such as magnesium stearate, sodium lauryl sulfate, and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms such as “excipient,” “carrier,” “pharmaceutically acceptable carrier.” or the like are used interchangeably herein.

In some embodiments, the nucleobase editors and the guide nucleotides in a composition of the present disclosure are administered by injection, by means of a catheter, by means of a suppository, or by means of an implant, the implant being of a porous, non-porous, or gelatinous material, including a membrane, such as a sialastic membrane, or a fiber. In some embodiments, the nucleobase editors and the guide nucleotides in a composition of the present disclosure are administered by injection into the bloodstream.

In other embodiments, the nucleobase editors and the guide nucleotides are delivered in a controlled release system. In one embodiment, a pump may be used (see, e.g., Langer, 1990. Science 249:1527-1533; Sefton. 1989, CRC Crit. Ref. Biomed. Eng. 14:201; Buchwald et al., 1980, Surgery 88:507; Saudek et al., 1989, N. Engl. J. Med. 321:574, the entire contents of each of which are incorporated herein by reference). In another embodiment, polymeric materials can be used (See, e.g., Medical Applications of Controlled Release (Langer and Wise eds., CRC Press. Boca Raton, Fla., 1974); Controlled Drug Bioavailability. Drug Product Design and Performance (Smolen and Ball eds., Wiley, New York, 1984); Ranger and Peppas, 1983, Macromol. Sci. Rev. Macromol. Chem. 23:61; See also: Levy et al., 1985. Science 228:190; During et al., 1989, Ann. Neurol. 25:351; Howard et al., 1989, J. Neurosurg. 71:105, each of which is incorporated herein by reference). Other controlled release systems are discussed, for example, in Langer, supra.

In typical embodiments, the pharmaceutical composition is formulated in accordance with routine procedures as a pharmaceutical composition adapted for intravenous or subcutaneous administration to a subject, e.g., a human. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in a unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A pharmaceutical composition for systemic administration may be a liquid. e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47, the entire contents of which is incorporated herein by reference). Positively charged lipids such as N-[1-(2,3-dioleoyloxi)propyl]-N,N,N-trimethyl-amoniummethylsulfate, or “DOTAP;” are particularly preferred for such particles and vesicles. The preparation of such lipid particles is well known. See. e.g., U.S. Pat. Nos. 4,880,635; 4,906,477; 4,911,928; 4,917,951; 4,920,016; and 4,921,757, each of which is incorporated herein by reference.

The pharmaceutical compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

In some embodiments, the nucleobase editors or the guide nucleotides described herein may be conjugated to a therapeutic moiety, e.g., an anti-inflammatory agent. Techniques for conjugating such therapeutic moieties to polypeptides, including e.g., Fc domains, are well known; see. e.g., Amon et al., “Monoclonal Antibodies For Immunotargeting Of Drugs In Cancer Therapy”, in Monoclonal Antibodies And Cancer Therapy. Reisfeld et al. (eds.), 1985, pp. 243-56, Alan R. Liss. Inc.); Hellstrom et al., “Antibodies For Drug Delivery”, in Controlled Drug Delivery (2nd Ed.), Robinson et al. (eds.), 1987. pp. 623-53, Marcel Dekker. Inc.); Thorpe. “Antibody Carriers Of Cytotoxic Agents In Cancer Therapy: A Review”, in Monoclonal Antibodies '84: Biological And Clinical Applications, Pinchera et al. (eds.), 1985. pp. 475-506); “Analysis, Results, And Future Prospective Of The Therapeutic Use Of Radiolabeled Antibody In Cancer Therapy”, in Monoclonal Antibodies For Cancer Detection And Therapy. Baldwin et al. (eds.), 1985, pp. 303-16. Academic Press; and Thorpe et al. (1982) “The Preparation And Cytotoxic Properties Of Antibody-Toxin Conjugates,” Immunol. Rev., 62:119-158; each of which is incorporated herein by reference.

Further, the compositions of the present disclosure may be assembled into kits. In some embodiments, the kit comprises nucleic acid vectors for the expression of the nucleobase editors described herein. In some embodiments, the kit further comprises appropriate guide nucleotide sequences (e.g., gRNAs) or nucleic acid vectors for the expression of such guide nucleotide sequences, to target the nucleobase editors to the desired target sequence.

The kit described herein may include one or more containers housing components for performing the methods described herein and optionally instructions of uses. Any of the kit described herein may further comprise components needed for performing the assay methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (for example, water or certain organic solvents), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape. DVD, etc.). Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric such as gauze, for applying or removing a disinfecting agent, disposable gloves, a support for the agents prior to administration, etc.

### Therapeutics

The compositions and kits described herein may be administered to a subject in need thereof, in a therapeutically effective amount, to prevent or treat conditions related to HIV infection and/or AIDS. The compositions and kits are effective in preventing or treating HIV infection in the subject or reducing the potential for HIV infection in the subject (including prevention of HIV infection in a subject).

“A therapeutically effective amount” as used herein refers to the amount of each therapeutic agent of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong the half-life of the polypeptide and to prevent the polypeptide being attacked by the host's immune system.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide (e.g., RNA or DNA) may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen (including the polypeptide or the polynucleotide used) can vary over time. In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide or the polynucleotide (such as the half-life of the polypeptide or the polynucleotide, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the antagonist, and the discretion of the attending physician. Typically the clinician will administer a polypeptide or a polynucleotide until a dosage is reached that achieves the desired result.

Administration of one or more polypeptides or polynucleotides can be continuous or intermittent, depending, for example, upon the recipient's physiological condition, whether the purpose of the administration is therapeutic or prophylactic, and other factors known to skilled practitioners. The administration of a polypeptide or a polynucleotide may be essentially continuous over a preselected period of time or may be in a series of spaced dose, e.g., either before, during, or after developing a disease. As used herein, the term “treating” refers to the application or administration of a polypeptide or a polynucleotide or composition including the polypeptide or the polynucleotide to a subject in need thereof. As used herein, “treating” a disease includes preventing disease onset. e.g., preventing HIV infection and/or preventing the onset of AIDS.

“A subject in need thereof” refers to an individual who has a disease, a symptom of the disease, or a predisposition or susceptibility toward the disease, with the purpose to prevent, cure, heal, alleviate, relieve, alter, remedy, ameliorate, improve, or affect the disease, one or more symptoms of the disease, or predisposition toward the disease. In some embodiments, the subject is at risk of becoming infected with HIV. In some embodiments, the subject is infected with HIV. In some embodiments, the subject has AIDS. In some embodiments, the subject is a mammal. In some embodiments, the subject is a non-human primate. In some embodiments, the subject is human. Alleviating a disease includes delaying the development or progression of the disease (i.e., AIDS), or reducing disease severity. Alleviating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally, or via an implanted reservoir.

The term “parenteral,” as used herein, includes subcutaneous, intracutaneous, intravenous, intramuscular, intraarticular, intraarterial, intrasynovial, intrasternal, intrathecal, intralesional, and intracranial injection or infusion techniques. In addition, the compositions described herein can be administered to the subject via injectable depot routes of administration such as using 1-, 3-, or 6-month depot injectable or biodegradable materials and methods.

### Host Cells and Organisms

Other aspects of the present disclosure provide host cells and organisms for the production and/or isolation of the nucleobase editors, e.g., for in vitro editing. Host cells are genetically engineered to express the nucleobase editors and components of the translation system described herein. In some embodiments, host cells comprise vectors encoding the nucleobase editors and components of the translation system (e.g., transformed, transduced, or transfected), which can be, for example, a cloning vector or an expression vector. The vector can be, for example, in the form of a plasmid, a bacterium, a virus, a naked polynucleotide, or a conjugated polynucleotide. The vectors are introduced into cells and/or microorganisms by standard methods including electroporation, infection by viral vectors, high velocity ballistic penetration by small particles with the nucleic acid either within the matrix of small beads or particles, or on the surface (Klein et al., Nature 327, 70-73 (1987), which is incorporated herein by reference). In some embodiments, the host cell is a prokaryotic cell. In some embodiments, the host cell is a eukaryotic cell. In some embodiments, the host cell is a bacterial cell. In some embodiments, the host cell is a yeast cell. In some embodiments, the host cell is a mammalian cell. In some embodiments, the host cell is a human cell. In some embodiments, the host cell is a cultured cell. In some embodiments, the host cell is within a tissue or an organism.

The engineered host cells can be cultured in conventional nutrient media modified as appropriate for such activities as, for example, screening steps, activating promoters or selecting transformants. These cells can optionally be cultured into transgenic organisms.

Several well-known methods of introducing target nucleic acids into bacterial cells are available, any of which can be used in the present disclosure. These include: fusion of the recipient cells with bacterial protoplasts containing the DNA, electroporation, projectile bombardment, and infection with viral vectors (discussed further, below), etc. Bacterial cells can be used to amplify the number of plasmids containing DNA constructs of the present disclosure. The bacteria are grown to log phase and the plasmids within the bacteria can be isolated by a variety of methods known in the art (see, for instance, Sambrook). In addition, a plethora of kits are commercially available for the purification of plasmids from bacteria, (see, e.g., EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See. Giliman & Smith, Gene 8:81 (1979); Roberts, et al., Nature, 328:731 (1987); and Schneider, B., et al., Protein Expr. Purifi 6435:10 (1995)), the entire contents of each of which are incorporated herein by reference.

Bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books. NY, the entire contents of which is incorporated herein by reference.

Other useful references, e.g., for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, NY; Gamborg and Phillips (eds) (1995) Plant Cell. Tissue and Organ Culture; Fundamental Methods Springer Lab Manual. Springer-Verlag (Berlin Heidelberg New York) and Atlas and Parks (eds) The Handbook of Microbiological Media (1993) CRC Press, Boca Raton. FL, the entire contents of each of which are incorporated herein by reference. In addition, essentially any nucleic acid (and virtually any labeled nucleic acid, whether standard or non-standard) can be custom or standard ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda. CA), and many others.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the compositions and methods described herein may be more fully understood, the following examples are set forth. The synthetic examples described in this application are offered to illustrate the compounds and methods provided herein and are not to be construed in any way as limiting their scope.

### Example 1: Guide Nucleotide Sequence-Programmable DNA-Binding Protein Domains, Deaminases, and Base Editors

Non-limiting examples of suitable guide nucleotide sequence-programmable DNA-binding protein domains are provided. The disclosure provides Cas9 variants, for example. Cas9 proteins from one or more organisms, which may comprise one or more mutations (e.g., to generate dCas9 or Cas9 nickase). In some embodiments, one or more of the amino acid residues, identified below by an asterisk, of a Cas9 protein may be mutated. In some embodiments, the D10 and/or H840 residues of the amino acid sequence provided in SEQ ID NO: 1, or a corresponding mutation in any of the amino acid sequences provided in SEQ ID NOs: 11-260, are mutated. In some embodiments, the D10 residue of the amino acid sequence provided in SEQ ID NO: 1, or a corresponding mutation in any of the amino acid sequences provided in SEQ ID NOs: 11-260, is mutated to any amino acid residue, except for D. In some embodiments, the D10 residue of the amino acid sequence provided in SEQ ID NO: 1, or a corresponding mutation in any of the amino acid sequences provided in SEQ ID NOs: 11-260, is mutated to an A. In some embodiments, the H840 residue of the amino acid sequence provided in SEQ ID NO: 1, or a corresponding residue in any of the amino acid sequences provided in SEQ ID NOs: 11-260, is an H. In some embodiments, the H840 residue of the amino acid sequence provided in SEQ ID NO: 1, or a corresponding mutation in any of the amino acid sequences provided in SEQ ID NOs: 11-260, is mutated to any amino acid residue, except for H. In some embodiments, the H840 residue of the amino acid sequence provided in SEQ ID NO: 1, or a corresponding mutation in any of the amino acid sequences provided in SEQ ID NOs: 11-260, is mutated to an A. In some embodiments, the D10 residue of the amino acid sequence provided in SEQ ID NO: 1, or a corresponding residue in any of the amino acid sequences provided in SEQ ID NOs: 11-260, is a D.

A number of Cas9 sequences from various species were aligned to determine whether corresponding homologous amino acid residues of D10 and H840 of SEQ ID NO: 1 or SEQ ID NO: 11 can be identified in other Cas9 proteins, allowing the generation of Cas9 variants with corresponding mutations of the homologous amino acid residues. The alignment was carried out using the NCBI Constraint-based Multiple Alignment Tool (COBALT (accessible at st-va.ncbi.nlm.nih.gov/tools/cobalt), with the following parameters. Alignment parameters: Gap penalties −11, −1; End-Gap penalties −5, −1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four Cas9 sequences is provided below. The Cas9 sequences in the alignment are: Sequence 1 (S1): SEQ ID NO: 11|WP_010922251|gi 499224711| type II CRISPR RNA-guided endonuclease Cas9 [Streptococcus pyogenes]; Sequence 2 (S2): SEQ ID NO: 12|WP_039695303|gi 746743737| type II CRISPR RNA-guided endonuclease Cas9 [Streptococcus gallolyticus]; Sequence 3 (S3): SEQ ID NO: 13|WP_045635197|gi 782887988|type II CRISPR RNA-guided endonuclease Cas9 [Streptococcus mitis]; Sequence 4 (S4): SEQ ID NO: 14|5AXW_A|gi 924443546| Staphylococcus aureus Cas9. The HNH domain (bold and underlined) and the RuvC domain (boxed) are identified for each of the four sequences. Amino acid residues 10 and 840 in S1 and the homologous amino acids in the aligned sequences are identified with an asterisk following the respective amino acid residue.

| | | | |
|---|---|---|---|
| S1 | 1 | --MDKK-YSIGLD*IGTNSVGWAVITDEYKVPSKKFKVLGNTDRHSIKKNLI--GALLFDSG--ETAEATRLKRTARRRYT | 73 |
| S2 | 1 | --MTKKNYSIGLD*IGTNSVGWAVITDDYKVPAKKMKVLGNTDKKYIKKNLL--GALLFDSG--ETAEATRLKRTARRRYT | 74 |
| S3 | 1 | --M-KKGYSIGLD*IGTNSVGFAVITDDYKVPSKKMKVLGNTDKRFIKKNLI--GALLFDEG--TTAEARRLKRTARRRYT | 73 |
| S4 | 1 | GSHMKRNYILGLD*IGITSVGYGII--DYET-----------------RDVIDAGVRLFKEANVENNEGRRSKRGARRLKR | 61 |
| S1 | 74 | RRKNRICYLQEIFSNEMAKVDDSFFHRLEESFLVEEDKKHERHPIFGNIVDEVAYHEKYPTIYHLRKKLVDSTDKADLRL | 153 |
| S2 | 75 | RRKNRLRYLQEIFANEIAKVDESFFQRLDESFLTDDDKTFDSHPIFGNKAEEDAYHQKFPTIYHLRKHLADSSEKADLRL | 154 |
| S3 | 74 | RRKNRLRYLQEIFSEEMSKVDSSFFHRLDDSFLIPEDKRESKYPIFATLTEEKEYHKQFPTIYHLRKQLADSKEKTDLRL | 153 |
| S4 | 62 | RRRHRIQRVKKLL--------------FDYNLLTD--------------------HSELSGINPYEARVKGLSQKLSEEE | 107 |
| S1 | 154 | IYLALAHMIKFRGHFLIEGDLNPDNSDVDKLFIQLVQTYNQLFEENPINASGVDAKAILSARLSKSRRLENLIAQLPGEK | 233 |
| S2 | 155 | VYLALAHMIKFRGHFLIEGELNAENTDVQKIFADFVGVYNRTFDDSHLSEITVDVASILTEKISKSRRLENLIKYYPTEK | 234 |
| S3 | 154 | IYLALAHMIKYRGHFLYEEAFDIKNNDIQKIFNEFISIYDNTFEGSSLSGQNAQVEAIFTDKISKSAKRERVLKLFPDEK | 233 |
| S4 | 108 | FSAALLHLAKRRG----------------------VHNVNEVEEDT---------------------------------- | 131 |
| S1 | 234 | KNGLFGNLIALSLGLTPNFKSNFDLAEDAKLQLSKDTYDDDLDNLLAQIGDQYADLFLAAKNLSDAILLSDILRVNTEIT | 313 |
| S2 | 235 | KNTLFGNLIALALGLQPNFKTNFKLSEDAKLQFSKDTYEEDLEELLGKIGDDYADLFTSAKNLYDAILLSGILTVDDNST | 314 |
| S3 | 234 | STGLFSEFLKLIVGNQADFKKHFDLEDKAPLQFSKDTYDEDLENLLGQIGDDFTDLFVSAKKLYDAILLSGILTVTDPST | 313 |
| S4 | 132 | -----GNELS------------------TKEQISRN-------------------------------------------- | 144 |
| S1 | 314 | KAPLSASMIKRYDEHHQDLTLLKALVRQQLPEKYKEIFFDQSKNGYAGYIDGGASQEEFYKFIKPILEKM--DGTEELLV | 391 |
| S2 | 315 | KAPLSASMIKRYVEHHEDLEKLKEFIKANKSELYHDIFKDKNKNGYAGYIENGVKQDEFYKYLKNILSKIKIDGSDYFLD | 394 |
| S3 | 314 | KAPLSASMIERYENHQNDLAALKQFIKNNLPEKYDEVFSDQSKDGYAGYIDGKTTQETFYKYIKNLLSKF--EGTDYFLD | 391 |
| S4 | 145 | ----SKALEEKYVAELQ-------------------------------------------------LERLKKDG------ | 165 |
| S1 | 392 | KLNREDLLRKQRTFDNGSIPHQIHLGELHAILRRQEDFYPFLKDNREKIEKILTFRIPYYVGPLARGNSRFAWMTRKSEE | 471 |
| S2 | 395 | KIEREDFLRKQRTFDNGSIPHQIHLQEMHAILRRQGDYYPFLKEKQDRIEKILTFRIPYYVGPLVRKDSRFAWAEYRSDE | 474 |
| S3 | 392 | KIEREDFLRKQRTFDNGSIPHQIHLQEMNAILRRQGEYYPFLKDNKEKIEKILTFRIPYYVGPLARGNRDFAWLTRNSDE | 471 |
| S4 | 166 | --EVRGSINRFKTSD--------YVKEAKQLLKVQKAYHQLDQSFIDTYIDLLETRRTYYEGP--GEGSPFGW------K | 227 |
| S1 | 472 | TITPWNFEEVVDKGASAQSFIERMTNFDKNLPNEKVLPKHSLLYEYFTVYNELTKVKYVTEGMRKPAFLSGEQKKAIVDL | 551 |
| S2 | 475 | KITPWNFDKVIDKEKSAEKFITRMTLNDLYLPEEKVLPKHSHVYETYAVYNELTKIKYVNEQGKE-SFFDSNMKQEIFDH | 553 |
| S3 | 472 | AIRPWNFEEIVDKASSAEDFINKMTNYDLYLPEEKVLPKHSLLYETFAVYNELTKVKFIAEGLRDYQFLDSGQKKQIVNQ | 551 |
| S4 | 228 | DIKEW---------------YEMLMGHCTYFPEELRSVKYAYNADLYNALNDLNNLVITRDENEK---LEYYEKFQIIEN | 289 |
| S1 | 552 | LFKTNRKVTVKQLKEDYFKKIECFDSVEISGVEDR---FNASLGTYHDLLKIIKDKDFLDNEENEDILEDIVLTLTLFED | 628 |
| S2 | 554 | VFKENRKVTKEKLLNYLNKEFPEYRIKDLIGLDKENKSFNASLGTYHDLKKIL-DKAFLDDKVNEEVIEDIIKTLTLFED | 632 |
| S3 | 552 | LFKENRKVTEKDIIHYLHN-VDGYDGIELKGIEKQ---FNASLSTYHDLLKIIKDKEFMDDAKNEAILENIVHTLTIFED | 627 |
| S4 | 290 | VFKQKKKPTLKQIAKEILVNEEDIKGYRVTSTGKPEF---TNLKVYHDIKDITARKEII---ENAELLDQIAKILTIYQS | 363 |
| S1 | 629 | REMIEERLKTYAHLFDDKVMKQLKR-RRYTGWGRLSRKLINGIRDKQSGKTILDFLKSDGFANRNFMQLIHDDSLTFKED | 707 |
| S2 | 633 | KDMIHERLQKYSDIFTANQLKKLER-RHYTGWGRLSYKLINGIRNKENNKTILDYLIDDGSANRNFMQLINDDTLPFKQI | 711 |
| S3 | 628 | REMIKQRLAQYDSLFDEKVIKALTR-RHYTGWGKLSAKLINGICDKQTGNTILDYLIDDGKINRNFMQLINDDGLSFKEI | 706 |
| S4 | 364 | SEDIQEELTNLNSELTQEEIEQISNLKGYTGTHNLSLKAINLILDE------LWHTNDNQIAIFNRLKLVP--------- | 428 |
| S1 | 708 | 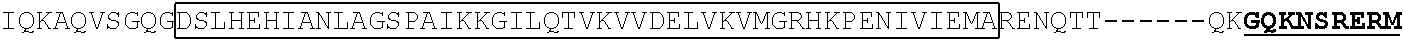 | 781 |
| S2 | 712 | 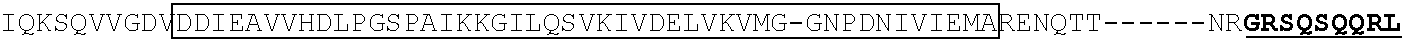 | 784 |
| S3 | 707 | 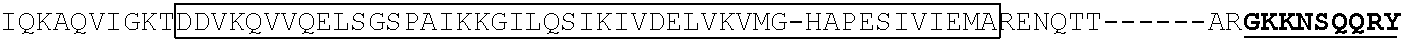 | 779 |
| S4 | 429 | 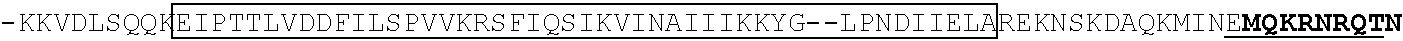 | 505 |
| S1 | 782 | KRIEEGIKELGSQIL-------KEHPVENTQLQNEKLYLYYLQNGRDMYVDQELDINRLSD----YDVDH*IVPQSFLKDD | 850 |
| S2 | 785 | KKLQNSLKELGSNILNEEKPSYIEDKVENSHLQNDQLFLYYIQNGKDMYTGDELDIDHLSD----YDIDH*IIPQAFIKDD | 860 |
| S3 | 780 | KRIEDSLKILASGL---DSNILKENPTDNNQLQNDRLFLYYLQNGKDMYTGEALDINQLSS----YDIDH*IIPQAFIKDD | 852 |
| S4 | 506 | ERIEEIIRTTGK---------------ENAKYLIEKIKLHDMQEGKCLYSLEAIPLEDLLNNPFNYEVDH*IIPRSVSFDN | 570 |
| S1 | 851 | 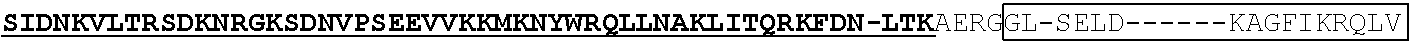 | 922 |
| S2 | 861 | 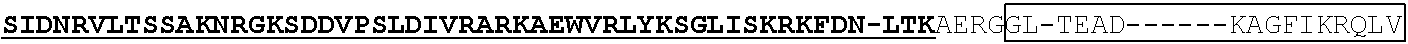 | 932 |
| S3 | 853 | 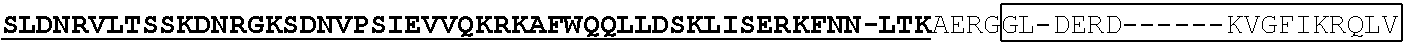 | 924 |
| S4 | 571 | 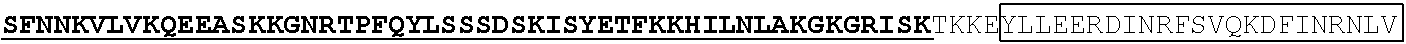 | 650 |
| S1 | 923 | 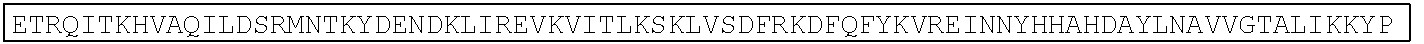 | 1002 |
| S2 | 933 | 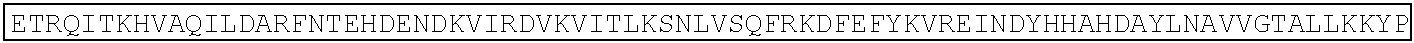 | 1012 |
| S3 | 925 | 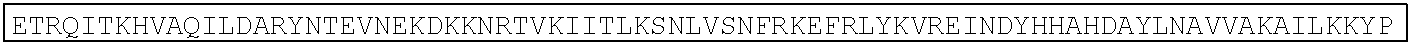 | 1004 |
| S4 | 651 | 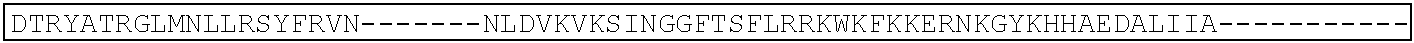 | 712 |
| S1 | 1003 | 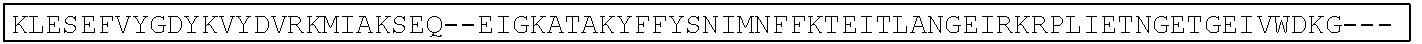 | 1077 |
| S2 | 1013 | 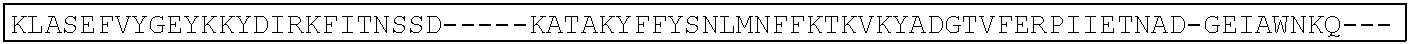 | 1083 |
| S3 | 1005 | 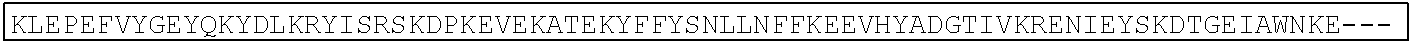 | 1081 |
| S4 | 713 | 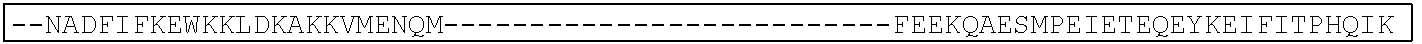 | 764 |
| S1 | 1078 | 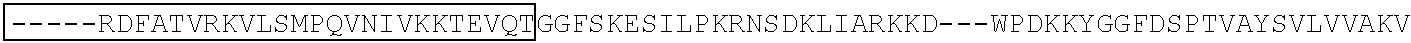 | 1149 |
| S2 | 1084 | 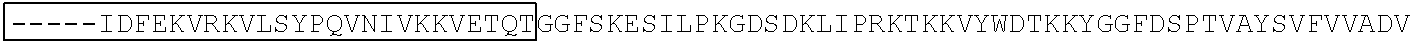 | 1158 |
| S3 | 1082 | 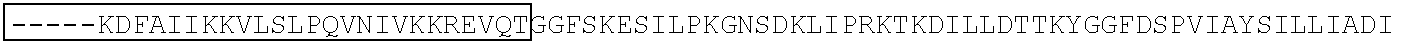 | 1156 |
| S4 | 765 | 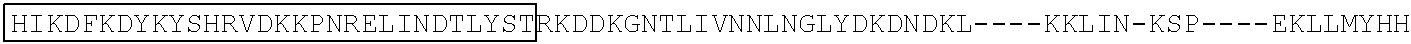 | 835 |
| S1 | 1150 | EKGKSKKLKSVKELLGITIMERSSFEKNPI-DFLEAKG-----YKEVKKDLIIKLPKYSLFELENGRKRMLASAGELQKG | 1223 |
| S2 | 1159 | EKGKAKKLKTVKELVGISIMERSFFEENPV-EFLENKG-----YHNIREDKLIKLPKYSLFEFEGGRRRLLASASELQKG | 1232 |
| S3 | 1157 | EKGKAKKLKTVKTLVGITIMEKAAFEENPI-TFLENKG-----YHNVRKENILCLPKYSLFELENGRRRLLASAKELQKG | 1230 |
| S4 | 836 | DPQTYQKLK--------LIMEQYGDEKNPLYKYYEETGNYLTKYSKKDNGPVIKKIKYYGNKLNAHLDITDDYPNSRNKV | 907 |
| S1 | 1224 | NELALPSKYVNFLYLASHYEKLKGSPEDNEQKQLFVEQHKHYLDEIIEQISEFSKRVILADANLDKVLSAYNKH------ | 1297 |
| S2 | 1233 | NEMVLPGYLVELLYHAHRADNF-----NSTEYLNYVSEHKKEFEKVLSCVEDFANLYVDVEKNLSKIRAVADSM------ | 1301 |
| S3 | 1231 | NEIVLPVYLTTLLYHSKNVHKL-----DEPGHLEYIQKHRNEFKDLLNLVSEFSQKYVLADANLEKIKSLYADN------ | 1299 |
| S4 | 908 | VKLSLKPYRFD-VYLDNGVYKFV-----TVKNLDVIK--KENYYEVNSKAYEEAKKLKKISNQAEFIASFYNNDLIKING | 979 |
| S1 | 1298 | RDKPIREQAENIIHLFTLTNLGAPAAFKYFDTTIDRKRYTSTKEVLDATLIHQSIT--------GLYETRI----DLSQL | 1365 |
| S2 | 1302 | DNFSIEEISNSFINLLTLTALGAPADFNFLGEKIPRKRYTSTKECLNATLIHQSIT--------GLYETRI----DLSKL | 1369 |
| S3 | 1300 | EQADIEILANSFINLLTFTALGAPAAFKFFGKDIDRKRYTTVSEILNATLIHQSIT--------GLYETWI----DLSKL | 1367 |
| S4 | 980 | ELYRVIGVNNDLLNRIEVNMIDITYR-EYLENMNDKRPPRIIKTIASKT---QSIKKYSTDILGNLYEVKSKKHPQIIKK | 1055 |
| S1 | 1366 | GGD | 1368 |
| S2 | 1370 | GEE | 1372 |
| S3 | 1368 | GED | 1370 |
| 54 | 1056 | G-- | 1056 |

The alignment demonstrates that amino acid sequences and amino acid residues that are homologous to a reference Cas9 amino acid sequence or amino acid residue can be identified across Cas9 sequence variants, including, but not limited to Cas9 sequences from different species, by identifying the amino acid sequence or residue that aligns with the reference sequence or the reference residue using alignment programs and algorithms known in the art. This disclosure provides Cas9 variants in which one or more of the amino acid residues identified by an asterisk in SEQ ID NOs: 11-14 (e.g., S1, S2, S3, and S4, respectively) are mutated as described herein. The residues D10 and H840 in Cas9 of SEQ ID NO: 1 that correspond to the residues identified in SEQ ID NOs: 11-14 by an asterisk are referred to herein as “homologous” or “corresponding” residues. Such homologous residues can be identified by sequence alignment, e.g., as described above, and by identifying the sequence or residue that aligns with the reference sequence or residue. Similarly, mutations in Cas9 sequences that correspond to mutations identified in SEQ ID NO: 1 herein, e.g., mutations of residues 10, and 840 in SEQ ID NO: 1, are referred to herein as “homologous” or “corresponding” mutations. For example, the mutations corresponding to the D10A mutation in SEQ ID NO: 1 or S1 (SEQ ID NO: 11) for the four aligned sequences above are D11A for S2, D10A for S3, and D13A for S4; the corresponding mutations for H840A in SEQ ID NO: 1 or S1 (SEQ ID NO: 11) are H850A for S2, H842A for S3, and H560A for S4.

A total of 250 Cas9 sequences (SEQ ID NOs: 11-260) from different species are provided. Amino acid residues homologous to residues 10, and 840 of SEQ ID NO: 1 may be identified in the same manner as outlined above. All of these Cas9 sequences may be used in accordance with the present disclosure.

| | | |
|---|---|---|
| WP_010922251.1 | type II CRISPR RNA-guided endonuclease Cas9 [Streptococcus pyogenes] | SEQ ID NO: 11 |
| WP_039695303.1 | type II CRISPR RNA-guided endonuclease Cas9 [Streptococcus gallolyticus] | SEQ ID NO: 12 |
| WP_045635197.1 | type II CRISPR RNA-guided endonuclease Cas9 [Streptococcus mitis] | SEQ ID NO: 13 |
| 5AXW_A | Cas9, Chain A, Crystal Structure [Staphylococcus Aureus] | SEQ ID NO: 14 |
| WP_009880683.1 | type II CRISPR RNA-guided endonuclease Cas9 [Streptococcus pyogenes] | SEQ ID NO: 15 |
| WP_010922251.1 | type II CRISPR RNA-guided endonuclease Cas9 [Streptococcus pyogenes] | SEQ ID NO: 16 |
| WP_011054416.1 | type II CRISPR RNA-guided endonuclease Cas9 [Streptococcus pyogenes ] | SEQ ID NO: 17 |
| WP_011284745.1 | type II CRISPR RNA-guided endonuclease Cas9 [ Streptococcus pyogenes] | SEQ ID NO: 18 |
| WP_011285506.1 | type II CRISPR RNA-guided endonuclease Cas9 [ Streptococcus pyogenes] | SEQ ID NO: 19 |
| WP_011527619.1 | type II CRISPR RNA-guided endonuclease Cas9 [Streptococcus pyogenes] | SEQ ID NO: 20 |
| WP_012560673.1 | type II CRISPR RNA-guided endonuclease Cas9 [Streptococcus pyogenes] | SEQ ID NO: 21 |
| WP_014407541.1 | type II CRISPR RNA-guided endonuclease Cas9 [Streptococcus pyogenes] | SEQ ID NO: 22 |
| WP_020905136.1 | type II CRISPR RNA-guided endonuclease Cas9 [Streptococcus pyogenes] | SEQ ID NO: 23 |
| WP_023080005.1 | type II CRISPR RNA-guided endonuclease Cas9 [Streptococcus pyogenes] | SEQ ID NO: 24 |
| WP_023610282.1 | type II CRISPR RNA-guided endonuclease Cas9 [Streptococcus pyogenes] | SEQ ID NO: 25 |
| WP_030125963.1 | type II CRISPR RNA-guided endonuclease Cas9 [Streptococcus pyogenes] | SEQ ID NO: 26 |
| WP_030126706.1 | type II CRISPR RNA-guided endonuclease Cas9 [Streptococcus pyogenes] | SEQ ID NO: 27 |
| WP_031488318.1 | type II CRISPR RNA-guided endonuclease Cas9 [Streptococcus pyogenes] | SEQ ID NO: 28 |
| WP_032460140.1 | type II CRISPR RNA-guided endonuclease Cas9 [Streptococcus pyogenes] | SEQ ID NO: 29 |
| WP_032461047.1 | type II CRISPR RNA-guided endonuclease Cas9 [Streptococcus pyogenes] | SEQ ID NO: 30 |
| WP_032462016.1 | type II CRISPR RNA-guided endonuclease Cas9 [Streptococcus pyogenes] | SEQ ID NO: 31 |
| WP_032462936.1 | type II CRISPR RNA-guided endonuclease Cas9 [Streptococcus pyogenes] | SEQ ID NO: 32 |
| WP_032464890.1 | type II CRISPR RNA-guided endonuclease Cas9 [Streptococcus pyogenes] | SEQ ID NO: 33 |
| WP_033888930.1 | type II CRISPR RNA-guided endonuclease Cas9 [Streptococcus pyogenes] | SEQ ID NO: 34 |
| WP_038431314.1 | type II CRISPR RNA-guided endonuclease Cas9 [Streptococcus pyogenes] | SEQ ID NO: 35 |
| WP_038432938.1 | type II CRISPR RNA-guided endonuclease Cas9 [Streptococcus pyogenes] | SEQ ID NO: 36 |
| WP_038434062.1 | type II CRISPR RNA-guided endonuclease Cas9 [Streptococcus pyogenes] | SEQ ID NO: 37 |
| BAQ51233.1 | CRISPR-associated protein, Csn1 family [Streptococcus pyogenes] | SEQ ID NO: 38 |
| KGE60162.1 | hypothetical protein MGAS2111_0903 [Streptococcus pyogenes MGAS2111] | SEQ ID NO: 39 |
| KGE60856.1 | CRISPR-associated endonuclease protein [Streptococcus pyogenes SS1447] | SEQ ID NO: 40 |
| WP_002989955.1 | MULTISPECIES: type II CRISPR RNA-guided endonuclease Cas9 [Streptococcus] | SEQ ID NO: 41 |
| WP_003030002.1 | MULTISPECIES: type II CRISPR RNA-guided endonuclease Cas9 [Streptococcus] | SEQ ID NO: 42 |
| WP_003065552.1 | MULTISPECIES: type II CRISPR RNA-guided endonuclease Cas9 [Streptococcus] | SEQ ID NO: 43 |
| WP_001040076.1 | type II CRISPR RNA-guided endonuclease Cas9 [Streptococcus agalactiae] | SEQ ID NO: 44 |
| WP_001040078.1 | type II CRISPR RNA-guided endonuclease Cas9 [Streptococcus agalactiae] | SEQ ID NO: 45 |
| WP_001040080.1 | type II CRISPR RNA-guided endonuclease Cas9 [Streptococcus agalactiae] | SEQ ID NO: 46 |
| WP_001040081.1 | type II CRISPR RNA-guided endonuclease Cas9 [Streptococcus agalactiae] | SEQ ID NO: 47 |
| WP_001040083.1 | type II CRISPR RNA-guided endonuclease Cas9 [Streptococcus agalactiae] | SEQ ID NO: 48 |
| WP_001040085.1 | type II CRISPR RNA-guided endonuclease Cas9 [Streptococcus agalactiae] | SEQ ID NO: 49 |
| WP_001040087.1 | type II CRISPR RNA-guided endonuclease Cas9 [Streptococcus agalactiae] | SEQ ID NO: 50 |
| WP_001040088.1 | type II CRISPR RNA-guided endonuclease Cas9 [Streptococcus agalactiae] | SEQ ID NO: 51 |
| WP_001040089.1 | type II CRISPR RNA-guided endonuclease Cas9 [Streptococcus agalactiae] | SEQ ID NO: 52 |
| WP_001040090.1 | type II CRISPR RNA-guided endonuclease Cas9 [Streptococcus agalactiae] | SEQ ID NO: 53 |
| WP_001040091.1 | type II CRISPR RNA-guided endonuclease Cas9 [Streptococcus agalactiae] | SEQ ID NO: 54 |
| WP_001040092.1 | type II CRISPR RNA-guided endonuclease Cas9 [Streptococcus agalactiae] | SEQ ID NO: 55 |
| WP_001040094.1 | type II CRISPR RNA-guided endonuclease Cas9 [Streptococcus agalactiae] | SEQ ID NO: 56 |
| WP_001040095.1 | type II CRISPR RNA-guided endonuclease Cas9 [Streptococcus agalactiae] | SEQ ID NO: 57 |
| WP_001040096.1 | type II CRISPR RNA-guided endonuclease Cas9 [Streptococcus agalactiae] | SEQ ID NO: 58 |
| WP_001040097.1 | type II CRISPR RNA-guided endonuclease Cas9 [Streptococcus agalactiae] | SEQ ID NO: 59 |
| WP_001040098.1 | type II CRISPR RNA-guided endonuclease Cas9 [Streptococcus agalactiae] | SEQ ID NO: 60 |
| WP_001040099.1 | type II CRISPR RNA-guided endonuclease Cas9 [Streptococcus agalactiae] | SEQ ID NO: 61 |
| WP_001040100.1 | type II CRISPR RNA-guided endonuclease Cas9 [Streptococcus agalactiae] | SEQ ID NO: 62 |
| WP_001040104.1 | type II CRISPR RNA-guided endonuclease Cas9 [Streptococcus agalactiae] | SEQ ID NO: 63 |
| WP_001040105.1 | type II CRISPR RNA-guided endonuclease Cas9 [Streptococcus agalactiae] | SEQ ID NO: 64 |
| WP_001040106.1 | type II CRISPR RNA-guided endonuclease Cas9 [Streptococcus agalactiae] | SEQ ID NO: 65 |
| WP_001040107.1 | type II CRISPR RNA-guided endonuclease Cas9 [Streptococcus agalactiae] | SEQ ID NO: 66 |
| WP_001040108.1 | type II CRISPR RNA-guided endonuclease Cas9 [Streptococcus agalactiae] | SEQ ID NO: 67 |
| WP_001040109.1 | type II CRISPR RNA-guided endonuclease Cas9 [Streptococcus agalactiae] | SEQ ID NO: 68 |
| WP_001040110.1 | type II CRISPR RNA-guided endonuclease Cas9 [Streptococcus agalactiae] | SEQ ID NO: 69 |
| WP_015058523.1 | type II CRISPR RNA-guided endonuclease Cas9 [Streptococcus agalactiae] | SEQ ID NO: 70 |
| WP_017643650.1 | type II CRISPR RNA-guided endonuclease Cas9 [Streptococcus agalactiae] | SEQ ID NO: 71 |
| WP_017647151.1 | type II CRISPR RNA-guided endonuclease Cas9 [Streptococcus agalactiae] | SEQ ID NO: 72 |
| WP_017648376.1 | type II CRISPR RNA-guided endonuclease Cas9 [Streptococcus agalactiae] | SEQ ID NO: 73 |
| WP_017649527.1 | type II CRISPR RNA-guided endonuclease Cas9 [Streptococcus agalactiae] | SEQ ID NO: 74 |
| WP_017771611.1 | type II CRISPR RNA-guided endonuclease Cas9 [Streptococcus agalactiae] | SEQ ID NO: 75 |
| WP_017771984.1 | type II CRISPR RNA-guided endonuclease Cas9 [Streptococcus agalactiae] | SEQ ID NO: 76 |
| CFQ25032.1 | CRISPR-associated protein [Streptococcus agalactiae] | SEQ ID NO: 77 |
| CFV16040.1 | CRISPR-associated protein [Streptococcus agalactiae] | SEQ ID NO: 78 |
| KLJ37842.1 | CRISPR-associated protein Csn1 [Streptococcus agalactiae] | SEQ ID NO: 79 |
| KLJ72361.1 | CRISPR-associated protein Csn1 [Streptococcus agalactiae] | SEQ ID NO: 80 |
| KLL20707.1 | CRISPR-associated protein Csn1 [Streptococcus agalactiae] | SEQ ID NO: 81 |
| KLL42645.1 | CRISPR-associated protein Csn1 [Streptococcus agalactiae] | SEQ ID NO: 82 |
| WP_047207273.1 | type II CRISPR RNA-guided endonuclease Cas9 [Streptococcus agalactiae] | SEQ ID NO: 83 |
| WP_047209694.1 | type II CRISPR RNA-guided endonuclease Cas9 [Streptococcus agalactiae] | SEQ ID NO: 84 |
| WP_050198062.1 | type II CRISPR RNA-guided endonuclease Cas9 [Streptococcus agalactiae] | SEQ ID NO: 85 |
| WP_050201642.1 | type II CRISPR RNA-guided endonuclease Cas9 [Streptococcus agalactiae] | SEQ ID NO: 86 |
| WP_050204027.1 | type II CRISPR RNA-guided endonuclease Cas9 [Streptococcus agalactiae] | SEQ ID NO: 87 |
| WP_050881965.1 | type II CRISPR RNA-guided endonuclease Cas9 [Streptococcus agalactiae] | SEQ ID NO: 88 |
| WP_050886065.1 | type II CRISPR RNA-guided endonuclease Cas9 [Streptococcus agalactiae] | SEQ ID NO: 89 |
| AHN30376.1 | CRSPR-associated protein Csn1 [Streptococcus agalactiae 138P] | SEQ ID NO: 90 |
| EAO78426.1 | reticulocyte binding protein [Streptococcus agalactiae H36B] | SEQ ID NO: 91 |
| CCW42055.1 | CRISPR-associated protein, SAG0894 family [Streptococcus agalactiae ILRI112] | SEQ ID NO: 92 |
| WP_003041502.1 | type II CRISPR RNA-guided endonuclease Cas9 [Streptococcus anginosus] | SEQ ID NO: 93 |
| WP_037593752.1 | type II CRISPR RNA-guided endonuclease Cas9 [Streptococcus anginosus] | SEQ ID NO: 94 |
| WP_049516684.1 | CRISPR-associated protein Csn1 [Streptococcus anginosus] | SEQ ID NO: 95 |
| GAD46167.1 | hypothetical protein ANG6_0662 [Streptococcus anginosus T5] | SEQ ID NO: 96 |
| WP_018363470.1 | type II CRISPR RNA-guided endonuclease Cas9 [Streptococcus caballi] | SEQ ID NO: 97 |
| WP_003043819.1 | type II CRISPR RNA-guided endonuclease Cas9 [Streptococcus canis] | SEQ ID NO: 98 |
| WP_006269658.1 | type II CRISPR RNA-guided endonuclease Cas9 [Streptococcus constellatus] | SEQ ID NO: 99 |
| WP_048800889.1 | type II CRISPR RNA-guided endonuclease Cas9 [Streptococcus constellatus] | SEQ ID NO: 100 |
| WP_012767106.1 | type II CRISPR RNA-guided endonuclease Cas9 [Streptococcus dysgalactiae] | SEQ ID NO: 101 |
| WP_014612333.1 | type II CRISPR RNA-guided endonuclease Cas9 [Streptococcus dysgalactiae] | SEQ ID NO: 102 |
| WP_015017095.1 | type II CRISPR RNA-guided endonuclease Cas9 [Streptococcus dysgalactiae] | SEQ ID NO: 103 |
| WP_015057649.1 | type II CRISPR RNA-guided endonuclease Cas9 [Streptococcus dysgalactiae] | SEQ ID NO: 104 |
| WP_048327215.1 | type II CRISPR RNA-guided endonuclease Cas9 [Streptococcus dysgalactiae] | SEQ ID NO: 105 |
| WP_049519324.1 | CRISPR-associated protein Csn1 [Streptococcus dysgalactiae] | SEQ ID NO: 106 |
| WP_012515931.1 | type II CRISPR RNA-guided endonuclease Cas9 [Streptococcus equi] | SEQ ID NO: 107 |
| WP_021320964.1 | type II CRISPR RNA-guided endonuclease Cas9 [Streptococcus equi] | SEQ ID NO: 108 |
| WP_037581760.1 | type II CRISPR RNA-guided endonuclease Cas9 [Streptococcus equi] | SEQ ID NO: 109 |
| WP_004232481.1 | type II CRISPR RNA-guided endonuclease Cas9 [Streptococcus equinus] | SEQ ID NO: 110 |
| WP_009854540.1 | type II CRISPR RNA-guided endonuclease Cas9 [Streptococcus gallolyticus] | SEQ ID NO: 111 |
| WP_012962174.1 | type II CRISPR RNA-guided endonuclease Cas9 [Streptococcus gallolyticus] | SEQ ID NO: 112 |
| WP_039695303.1 | type II CRISPR RNA-guided endonuclease Cas9 [Streptococcus gallolyticus] | SEQ ID NO: 113 |
| WP_014334983.1 | type II CRISPR RNA-guided endonuclease Cas9 [Streptococcus infantarius] | SEQ ID NO: 114 |
| WP_003099269.1 | type II CRISPR RNA-guided endonuclease Cas9 [Streptococcus iniae] | SEQ ID NO: 115 |
| AHY15608.1 | CRISPR-associated protein Csn1 [Streptococcus iniae] | SEQ ID NO: 116 |
| AHY17476.1 | CRISPR-associated protein Csn1 [Streptococcus iniae] | SEQ ID NO: 117 |
| ESR09100.1 | hypothetical protein IUSA1_08595 [Streptococcus iniae IUSA1] | SEQ ID NO: 118 |
| AGM98575.1 | CRISPR-associated protein Cas9/Csn1, subtype II/NMEMI [Streptococcus iniae SF1] | SEQ ID NO: 119 |
| ALF27331.1 | CRISPR-associated protein Csn1 [Streptococcus intermedius] | SEQ ID NO: 120 |
| WP_018372492.1 | type II CRISPR RNA-guided endonuclease Cas9 [Streptococcus massiliensis] | SEQ ID NO: 121 |
| WP_045618028.1 | type II CRISPR RNA-guided endonuclease Cas9 [Streptococcus mitis] | SEQ ID NO: 122 |
| WP_045635197.1 | type II CRISPR RNA-guided endonuclease Cas9 [Streptococcus mitis] | SEQ ID NO: 123 |
| WP_002263549.1 | type II CRISPR RNA-guided endonuclease Cas9 [Streptococcus mutans] | SEQ ID NO: 124 |
| WP_002263887.1 | type II CRISPR RNA-guided endonuclease Cas9 [Streptococcus mutans] | SEQ ID NO: 125 |
| WP_002264920.1 | type II CRISPR RNA-guided endonuclease Cas9 [Streptococcus mutans] | SEQ ID NO: 126 |
| WP_002269043.1 | type II CRISPR RNA-guided endonuclease Cas9 [Streptococcus mutans] | SEQ ID NO: 127 |
| WP_002269448.1 | type II CRISPR RNA-guided endonuclease Cas9 [Streptococcus mutans] | SEQ ID NO: 128 |
| WP_002271977.1 | type II CRISPR RNA-guided endonuclease Cas9 [Streptococcus mutans] | SEQ ID NO: 129 |
| WP_002272766.1 | type II CRISPR RNA-guided endonuclease Cas9 [Streptococcus mutans] | SEQ ID NO: 130 |
| WP_002273241.1 | type II CRISPR RNA-guided endonuclease Cas9 [Streptococcus mutans] | SEQ ID NO: 131 |
| WP_002275430.1 | type II CRISPR RNA-guided endonuclease Cas9 [Streptococcus mutans] | SEQ ID NO: 132 |
| WP_002276448.1 | type II CRISPR RNA-guided endonuclease Cas9 [Streptococcus mutans] | SEQ ID NO: 133 |
| WP_002277050.1 | type II CRISPR RNA-guided endonuclease Cas9 [Streptococcus mutans] | SEQ ID NO: 134 |
| WP_002277364.1 | type II CRISPR RNA-guided endonuclease Cas9 [Streptococcus mutans] | SEQ ID NO: 135 |
| WP_002279025.1 | type II CRISPR RNA-guided endonuclease Cas9 [Streptococcus mutans] | SEQ ID NO: 136 |
| WP_002279859.1 | type II CRISPR RNA-guided endonuclease Cas9 [Streptococcus mutans] | SEQ ID NO: 137 |
| WP_002280230.1 | type II CRISPR RNA-guided endonuclease Cas9 [Streptococcus mutans] | SEQ ID NO: 138 |
| WP_002281696.1 | type II CRISPR RNA-guided endonuclease Cas9 [Streptococcus mutans] | SEQ ID NO: 139 |
| WP_002282247.1 | type II CRISPR RNA-guided endonuclease Cas9 [Streptococcus mutans] | SEQ ID NO: 140 |
| WP_002282906.1 | type II CRISPR RNA-guided endonuclease Cas9 [Streptococcus mutans] | SEQ ID NO: 141 |
| WP_002283846.1 | type II CRISPR RNA-guided endonuclease Cas9 [Streptococcus mutans] | SEQ ID NO: 142 |
| WP_002287255.1 | type II CRISPR RNA-guided endonuclease Cas9 [Streptococcus mutans] | SEQ ID NO: 143 |
| WP_002288990.1 | type II CRISPR RNA-guided endonuclease Cas9 [Streptococcus mutans] | SEQ ID NO: 144 |
| WP_002289641.1 | type II CRISPR RNA-guided endonuclease Cas9 [Streptococcus mutans] | SEQ ID NO: 145 |
| WP_002290427.1 | type II CRISPR RNA-guided endonuclease Cas9 [Streptococcus mutans] | SEQ ID NO: 146 |
| WP_002295753.1 | type II CRISPR RNA-guided endonuclease Cas9 [Streptococcus mutans] | SEQ ID NO: 147 |
| WP_002296423.1 | type II CRISPR RNA-guided endonuclease Cas9 [Streptococcus mutans] | SEQ ID NO: 148 |
| WP_002304487.1 | type II CRISPR RNA-guided endonuclease Cas9 [Streptococcus mutans] | SEQ ID NO: 149 |
| WP_002305844.1 | type II CRISPR RNA-guided endonuclease Cas9 [Streptococcus mutans] | SEQ ID NO: 150 |
| WP_002307203.1 | type II CRISPR RNA-guided endonuclease Cas9 [Streptococcus mutans] | SEQ ID NO: 151 |
| WP_002310390.1 | type II CRISPR RNA-guided endonuclease Cas9 [Streptococcus mutans] | SEQ ID NO: 152 |
| WP_002352408.1 | type II CRISPR RNA-guided endonuclease Cas9 [Streptococcus mutans] | SEQ ID NO: 153 |
| WP_012997688.1 | type II CRISPR RNA-guided endonuclease Cas9 [Streptococcus mutans] | SEQ ID NO: 154 |
| WP_014677909.1 | type II CRISPR RNA-guided endonuclease Cas9 [Streptococcus mutans] | SEQ ID NO: 155 |
| WP_019312892.1 | type II CRISPR RNA-guided endonuclease Cas9 [Streptococcus mutans] | SEQ ID NO: 156 |
| WP_019313659.1 | type II CRISPR RNA-guided endonuclease Cas9 [Streptococcus mutans] | SEQ ID NO: 157 |
| WP_019314093.1 | type II CRISPR RNA-guided endonuclease Cas9 [Streptococcus mutans] | SEQ ID NO: 158 |
| WP_019315370.1 | type II CRISPR RNA-guided endonuclease Cas9 [Streptococcus mutans] | SEQ ID NO: 159 |
| WP_019803776.1 | type II CRISPR RNA-guided endonuclease Cas9 [Streptococcus mutans] | SEQ ID NO: 160 |
| WP_019805234.1 | type II CRISPR RNA-guided endonuclease Cas9 [Streptococcus mutans] | SEQ ID NO: 161 |
| WP_024783594.1 | type II CRISPR RNA-guided endonuclease Cas9 [Streptococcus mutans] | SEQ ID NO: 162 |
| WP_024784288.1 | type II CRISPR RNA-guided endonuclease Cas9 [Streptococcus mutans] | SEQ ID NO: 163 |
| WP_024784666.1 | type II CRISPR RNA-guided endonuclease Cas9 [Streptococcus mutans] | SEQ ID NO: 164 |
| WP_024784894.1 | type II CRISPR RNA-guided endonuclease Cas9 [Streptococcus mutans] | SEQ ID NO: 165 |
| WP_024786433.1 | type II CRISPR RNA-guided endonuclease Cas9 [Streptococcus mutans] | SEQ ID NO: 166 |
| WP_049473442.1 | CRISPR-associated protein Csn1 [Streptococcus mutans] | SEQ ID NO: 167 |
| WP_049474547.1 | CRISPR-associated protein Csn1 [Streptococcus mutans] | SEQ ID NO: 168 |
| EMC03581.1 | hypothetical protein SMU69_09359 [Streptococcus mutans NLML4] | SEQ ID NO: 169 |
| WP_000428612.1 | type II CRISPR RNA-guided endonuclease Cas9 [Streptococcus oralis] | SEQ ID NO: 170 |
| WP_000428613.1 | type II CRISPR RNA-guided endonuclease Cas9 [Streptococcus oralis] | SEQ ID NO: 171 |
| WP_049523028.1 | CRISPR-associated protein Csn1 [Streptococcus parasanguinis] | SEQ ID NO: 172 |
| WP_003107102.1 | type II CRISPR RNA-guided endonuclease Cas9 [Streptococcus parauberis] | SEQ ID NO: 173 |
| WP_054279288.1 | type II CRISPR RNA-guided endonuclease Cas9 [Streptococcus phocae] | SEQ ID NO: 174 |
| WP_049531101.1 | CRISPR-associated protein Csn1 [Streptococcus pseudopneumoniae] | SEQ ID NO: 175 |
| WP_049538452.1 | CRISPR-associated protein Csn1 [Streptococcus pseudopneumoniae] | SEQ ID NO: 176 |
| WP_049549711.1 | CRISPR-associated protein Csn1 [Streptococcus pseudopneumoniae] | SEQ ID NO: 177 |
| WP_007896501.1 | type II CRISPR RNA-guided endonuclease Cas9 [Streptococcus pseudoporcinus] | SEQ ID NO: 178 |
| EFR44625.1 | CRISPR-associated protein, Csn1 family [Streptococcus pseudoporcinus SPIN 20026] | SEQ ID NO: 179 |
| WP_002897477.1 | type II CRISPR RNA-guided endonuclease Cas9 [Streptococcus sanguinis] | SEQ ID NO: 180 |
| WP_002906454.1 | type II CRISPR RNA-guided endonuclease Cas9 [Streptococcus sanguinis] | SEQ ID NO: 181 |
| WP_009729476.1 | type II CRISPR RNA-guided endonuclease Cas9 [Streptococcus sp. F0441] | SEQ ID NO: 182 |
| CQR24647.1 | CRISPR-associated protein [Streptococcus sp. FF10] | SEQ ID NO: 183 |
| WP_000066813.1 | type II CRISPR RNA-guided endonuclease Cas9 [Streptococcus sp. M334] | SEQ ID NO: 184 |
| WP_009754323.1 | type II CRISPR RNA-guided endonuclease Cas9 [Streptococcus sp. taxon 056] | SEQ ID NO: 185 |
| WP_044674937.1 | type II CRISPR RNA-guided endonuclease Cas9 [Streptococcus suis] | SEQ ID NO: 186 |
| WP_044676715.1 | type II CRISPR RNA-guided endonuclease Cas9 [Streptococcus suis] | SEQ ID NO: 187 |
| WP_044680361.1 | type II CRISPR RNA-guided endonuclease Cas9 [Streptococcus suis] | SEQ ID NO: 188 |
| WP_044681799.1 | type II CRISPR RNA-guided endonuclease Cas9 [Streptococcus suis] | SEQ ID NO: 189 |
| WP_049533112.1 | CRISPR-associated protein Csn1 [Streptococcus suis] | SEQ ID NO: 190 |
| WP_029090905.1 | type II CRISPR RNA-guided endonuclease Cas9 [Brochothrix thermosphacta] | SEQ ID NO: 191 |
| WP_006506696.1 | type II CRISPR RNA-guided endonuclease Cas9 [Catenibacterium mitsuokai] | SEQ ID NO: 192 |
| AIT42264.1 | Cas9hc:NLS: HA [Cloning vector pYB196] | SEQ ID NO: 193 |
| WP_034440723.1 | type II CRISPR endonuclease Cas9 [Clostridiales bacterium S5-A11] | SEQ ID NO: 194 |
| AKQ21048.1 | Cas9 [CRISPR-mediated gene targeting vector p (bhsp68-Cas9)] | SEQ ID NO: 195 |
| WP_004636532.1 | type II CRISPR RNA-guided endonuclease Cas9 [Dolosigranulum pigrum] | SEQ ID NO: 196 |
| WP_002364836.1 | MULTISPECIES: type II CRISPR RNA-guided endonuclease Cas9 [Enterococcus] | SEQ ID NO: 197 |
| WP_016631044.1 | MULTISPECIES: type II CRISPR RNA-guided endonuclease Cas9 [Enterococcus] | SEQ ID NO: 198 |
| EMS75795.1 | hypothetical protein H318_06676 [Enterococcus durans IPLA 655] | SEQ ID NO: 199 |
| WP_002373311.1 | type II CRISPR RNA-guided endonuclease Cas9 [Enterococcus faecalis] | SEQ ID NO: 200 |
| WP_002378009.1 | type II CRISPR RNA-guided endonuclease Cas9 [Enterococcus faecalis] | SEQ ID NO: 201 |
| WP_002407324.1 | type II CRISPR RNA-guided endonuclease Cas9 [Enterococcus faecalis] | SEQ ID NO: 202 |
| WP_002413717.1 | type II CRISPR RNA-guided endonuclease Cas9 [Enterococcus faecalis] | SEQ ID NO: 203 |
| WP_010775580.1 | type II CRISPR RNA-guided endonuclease Cas9 [Enterococcus faecalis] | SEQ ID NO: 204 |
| WP_010818269.1 | type II CRISPR RNA-guided endonuclease Cas9 [Enterococcus faecalis] | SEQ ID NO: 205 |
| WP_010824395.1 | type II CRISPR RNA-guided endonuclease Cas9 [Enterococcus faecalis] | SEQ ID NO: 206 |
| WP_016622645.1 | type II CRISPR RNA-guided endonuclease Cas9 [Enterococcus faecalis] | SEQ ID NO: 207 |
| WP_033624816.1 | type II CRISPR RNA-guided endonuclease Cas9 [Enterococcus faecalis] | SEQ ID NO: 208 |
| WP_033625576.1 | type II CRISPR RNA-guided endonuclease Cas9 [Enterococcus faecalis] | SEQ ID NO: 209 |
| WP_033789179.1 | type II CRISPR RNA-guided endonuclease Cas9 [Enterococcus faecalis] | SEQ ID NO: 210 |
| WP_002310644.1 | type II CRISPR RNA-guided endonuclease Cas9 [Enterococcus faecium] | SEQ ID NO: 211 |
| WP_002312694.1 | type II CRISPR RNA-guided endonuclease Cas9 [Enterococcus faecium] | SEQ ID NO: 212 |
| WP_002314015.1 | type II CRISPR RNA-guided endonuclease Cas9 [Enterococcus faecium] | SEQ ID NO: 213 |
| WP_002320716.1 | type II CRISPR RNA-guided endonuclease Cas9 [Enterococcus faecium] | SEQ ID NO: 214 |
| WP_002330729.1 | type II CRISPR RNA-guided endonuclease Cas9 [Enterococcus faecium] | SEQ ID NO: 215 |
| WP_002335161.1 | type II CRISPR RNA-guided endonuclease Cas9 [Enterococcus faecium] | SEQ ID NO: 216 |
| WP_002345439.1 | type II CRISPR RNA-guided endonuclease Cas9 [Enterococcus faecium] | SEQ ID NO: 217 |
| WP_034867970.1 | type II CRISPR RNA-guided endonuclease Cas9 [Enterococcus faecium] | SEQ ID NO: 218 |
| WP_047937432.1 | type II CRISPR RNA-guided endonuclease Cas9 [Enterococcus faecium] | SEQ ID NO: 219 |
| WP_010720994.1 | type II CRISPR RNA-guided endonuclease Cas9 [Enterococcus hirae] | SEQ ID NO: 220 |
| WP_010737004.1 | type II CRISPR RNA-guided endonuclease Cas9 [Enterococcus hirae] | SEQ ID NO: 221 |
| WP_034700478.1 | type II CRISPR RNA-guided endonuclease Cas9 [Enterococcus hirae] | SEQ ID NO: 222 |
| WP_007209003.1 | type II CRISPR RNA-guided endonuclease Cas9 [Enterococcus italicus] | SEQ ID NO: 223 |
| WP_023519017.1 | type II CRISPR RNA-guided endonuclease Cas9 [Enterococcus mundtii] | SEQ ID NO: 224 |
| WP_010770040.1 | type II CRISPR RNA-guided endonuclease Cas9 [Enterococcus phoeniculicola] | SEQ ID NO: 225 |
| WP_048604708.1 | type II CRISPR RNA-guided endonuclease Cas9 [Enterococcus sp. AM1] | SEQ ID NO: 226 |
| WP_010750235.1 | type II CRISPR RNA-guided endonuclease Cas9 [Enterococcus villorum] | SEQ ID NO: 227 |
| AII16583.1 | Cas9 endonuclease [Expression vector pCas9] | SEQ ID NO: 228 |
| WP_029073316.1 | type II CRISPR RNA-guided endonuclease Cas9 [Kandleria vitulina] | SEQ ID NO: 229 |
| WP_031589969.1 | type II CRISPR RNA-guided endonuclease Cas9 [Kandleria vitulina] | SEQ ID NO: 230 |
| KDA45870.1 | CRISPR-associated protein Cas9/Csn1, subtype II/NMEMI [Lactobacillus animalis] | SEQ ID NO: 231 |
| WP_039099354.1 | type II CRISPR RNA-guided endonuclease Cas9 [Lactobacillus curvatus] | SEQ ID NO: 232 |
| AKP02966.1 | hypothetical protein ABB45_04605 [Lactobacillus farciminis] | SEQ ID NO: 233 |
| WP_010991369.1 | type II CRISPR RNA-guided endonuclease Cas9 [Listeria innocua] | SEQ ID NO: 234 |
| WP_033838504.1 | type II CRISPR RNA-guided endonuclease Cas9 [Listeria innocua] | SEQ ID NO: 235 |
| EHN60060.1 | CRISPR-associated protein, Csn1 family [Listeria innocua ATCC 33091] | SEQ ID NO: 236 |
| EFR89594.1 | crispr-associated protein, Csn1 family [Listeria innocua FSL S4-378] | SEQ ID NO: 237 |
| WP_038409211.1 | type II CRISPR RNA-guided endonuclease Cas9 [Listeria ivanovii] | SEQ ID NO: 238 |
| EFR95520.1 | crispr-associated protein Csn1 [Listeria ivanovii FSL F6-596] | SEQ ID NO: 239 |
| WP_003723650.1 | type II CRISPR RNA-guided endonuclease Cas9 [Listeria monocytogenes] | SEQ ID NO: 240 |
| WP_003727705.1 | type II CRISPR RNA-guided endonuclease Cas9 [Listeria monocytogenes] | SEQ ID NO: 241 |
| WP_003730785.1 | type II CRISPR RNA-guided endonuclease Cas9 [Listeria monocytogenes] | SEQ ID NO: 242 |
| WP_003733029.1 | type II CRISPR RNA-guided endonuclease Cas9 [Listeria monocytogenes] | SEQ ID NO: 243 |
| WP_003739838.1 | type II CRISPR RNA-guided endonuclease Cas9 [Listeria monocytogenes] | SEQ ID NO: 244 |
| WP_014601172.1 | type II CRISPR RNA-guided endonuclease Cas9 [Listeria monocytogenes] | SEQ ID NO: 245 |
| WP_023548323.1 | type II CRISPR RNA-guided endonuclease Cas9 [Listeria monocytogenes] | SEQ ID NO: 246 |
| WP_031665337.1 | type II CRISPR RNA-guided endonuclease Cas9 [Listeria monocytogenes] | SEQ ID NO: 247 |
| WP_031669209.1 | type II CRISPR RNA-guided endonuclease Cas9 [Listeria monocytogenes] | SEQ ID NO: 248 |
| WP_033920898.1 | type II CRISPR RNA-guided endonuclease Cas9 [Listeria monocytogenes] | SEQ ID NO: 249 |
| AKI42028.1 | CRISPR-associated protein [Listeria monocytogenes] | SEQ ID NO: 250 |
| AKI50529.1 | CRISPR-associated protein [Listeria monocytogenes] | SEQ ID NO: 251 |
| EFR83390.1 | crispr-associated protein Csn1 [Listeria monocytogenes FSL F2-208] | SEQ ID NO: 252 |
| WP_046323366.1 | type II CRISPR RNA-guided endonuclease Cas9 [Listeria seeligeri] | SEQ ID NO: 253 |
| AKE81011.1 | Cas9 [Plant multiplex genome editing vector pYLCRISPR/Cas9Pubi-H] | SEQ ID NO: 254 |
| CUO82355.1 | Uncharacterized protein conserved in bacteria [Roseburia hominis] | SEQ ID NO: 255 |
| WP_033162887.1 | type II CRISPR RNA-guided endonuclease Cas9 [Sharpea azabuensis] | SEQ ID NO: 256 |
| AGZ01981.1 | Cas9 endonuclease [synthetic construct] | SEQ ID NO: 257 |
| AKA60242.1 | nuclease deficient Cas9 [synthetic construct] | SEQ ID NO: 258 |
| AKS40380.1 | Cas9 [Synthetic plasmid pFC330] | SEQ ID NO: 259 |
| 4UN5_B | Cas9, Chain B, Crystal Structure | SEQ ID NO: 260 |

Non-Limiting Examples of Suitable Deaminase Domains are Provided.

Human AID
(SEQ ID NO: 270)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
(underline: nuclear localization signal;
double underline: nuclear export signal)
Mouse AID
(SEQ ID NO: 271)
MDSLLMKQKKFLYHFKNVRWAKGRHETYLCYVVKRRDSATSCSLDFGHLR
NKSGCHVELLFLRYISDWDLDPGRCYRVTWFTSWSPCYDCARHVAEFLRW
NPNLSLRIFTARLYFCEDRKAEPEGLRRLHRAGVQIGIMTFKDYFYCWNT
FVENRERTFKAWEGLHENSVRLTRQLRRILLPLYEVDDLRDAFRMLGF
(underline: nuclear localization signal;
double underline: nuclear export signal)
Dog AID
(SEQ ID NO: 272)
MDSLLMKQRKFLYHFKNVRWAKGRHETYLCYVVKRRDSATSFSLDFGHLR
NKSGCHVELLFLRYISDWDLDPGRCYRVTWFTSWSPCYDCARHVADFLRG
YPNLSLRIFAARLYFCEDRKAEPEGLRRLHRAGVQIAIMTFKDYFYCWNT
FVENREKTFKAWEGLHENSVRLSRQLRRILLPLYEVDDLRDAFRTLGL
(underline: nuclear localization signal;
double underline: nuclear export signal)
Bovine AID
(SEQ ID NO: 273)
MDSLLKKQRQFLYQFKNVRWAKGRHETYLCYVVKRRDSPTSFSLDFGHLR
NKAGCHVELLFLRYISDWDLDPGRCYRVTWFTSWSPCYDCARHVADFLRG
YPNLSLRIFTARLYFCDKERKAEPEGLRRLHRAGVQIAIMTFKDYFYCWN
TFVENHERTFKAWEGLHENSVRLSRQLRRILLPLYEVDDLRDAFRTLGL
(underline: nuclear localization signal;
double underline: nuclear export signal)
Mouse APOBEC-3
(SEQ ID NO: 274)
MGPFCLGCSHRKCYSPIRNLISQETFKFHFKNLGYAKGRKDTFLCYEVTR
KDCDSPVSLHHGVFKNKDNIHAEICFLYWFHDKVLKVLSPREEFKITWYM
SWSPCFECAEQIVRFLATHHNLSLDIFSSRLYNVQDPETQQNLCRLVQEG
AQVAAMDLYEFKKCWKKFVDNGGRRFRPWKRLLTNFRYQDSKLQEILRPC
YIPVPSSSSSTLSNICLTKGLPETRFCVEGRRMDPLSEEEFYSQFYNQRV
KHLCYYHRMKPYLCYQLEQFNGQAPLKGCLLSEKGKQHAEILFLDKIRSM
ELSQVTITCYLTWSPCPNCAWQLAAFKRDRPDLILHIYTSRLYFHWKRPF
QKGLCSLWQSGILVDVMDLPQFTDCWTNFVNPKRPFWPWKGLEIISRRTQ
RRLRRIKESWGLQDLVNDFGNLQLGPPMS
(italic: nucleic acid editing domain)
Rat APOBEC-3
(SEQ ID NO: 275)
MGPFCLGCSHRKCYSPIRNLISQETFKFHFKNLRYAIDRKDTFLCYEVTR
KDCDSPVSLHHGVFKNKDNIHAEICFLYWFHDKVLKVLSPREEFKITWYM
SWSPCFECAEQVLRFLATHHNLSLDIFSSRLYNIRDPENQQNLCRLVQEG
AQVAAMDLYEFKKCWKKFVDNGGRRFRPWKKLLTNFRYQDSKLQEILRPC
YIPVPSSSSSTLSNICLTKGLPETRFCVERRRVHLLSEEEFYSQFYNQRV
KHLCYYHGVKPYLCYQLEQFNGQAPLKGCLLSEKGKQHAEILFLDKIRSM
ELSQVIITCYLTWSPCPNCAWQLAAFKRDRPDLILHIYTSRLYFHWKRPF
QKGLCSLWQSGILVDVMDLPQFTDCWTNFVNPKRPFWPWKGLEIISRRTQ
RRLHRIKESWGLQDLVNDFGNLQLGPPMS
(italic: nucleic acid editing domain)
Rhesus macaque APOBEC-3G
(SEQ ID NO: 276)
MVEPMDPRTFVSNFNNRPILSGLNTVWLCCEVKTKDPSGPPLDAKIFQGK
VYSKAKYHPEMRFLRWFHKWRQLHHDQEYKVTWYVSWSPCTRCANSVATF
LAKDPKVTLTIFVARLYYFWKPDYQQALRILCQKRGGPHATMKIMNYNEF
QDCWNKFVDGRGKPFKPRNNLPKHYTLLQATLGELLRHLMDPGTFTSNFN
NKPWVSGQHETYLCYKVERLHNDTWVPLNQHRGFLRNQAPNIHGFPKGRH
AELCFLDLIPFWKLDGQQYRWCFTSWSPCFSCAQEMAKFISNNEHVSLCI
FAARIYDDQGRYQEGLRALHRDGAKIAMMNYSEFEYCWDTFVDRQGRPFQ
PWDGLDEHSQALSGRLRAI
(italic: nucleic acid editing domain;
underline: cytoplasmic localization signal)
Chimpanzee APOBEC-3G
(SEQ ID NO: 277)
MKPHFRNPVERMYQDTFSDNFYNRPILSHRNTVWLCYEVKTKGPSRPPLD
AKIFRGQVYSKLKYHPEMRFFHWFSKWRKLHRDQEYEVTWYISWSPCTKC
TRDVATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTSNFNNELWVRGRHETYLCYEVERLHNDTWVLLNQRRGFLCNQAPHKH
GFLEGRHAELCFLDVIPFWKLDLHQDYRWCFTSWSPCFSCAQEMAKFISN
NKHVSLCIFAARIYDDQGRCQEGLRTLAKAGAKISIMTYSEFKHCWDTFV
DHQGCPFQPWDGLEEHSQALSGRLRAILQNQGN
(italic: nucleic acid editing domain;
underline: cytoplasmic localization signal)
Green monkey APOBEC-3G
(SEQ ID NO: 278)
MNPQIRNMVEQMEPDIFVYYFNNRPILSGRNTVWLCYEVKTKDPSGPPLD
ANIFQGKLYPEAKDHPEMKFLHWFRKWRQLHRDQEYEVTWYVSWSPCTRC
ANSVATFLAEDPKVTLTIFVARLYYFWKPDYQQALRILCQERGGPHATMK
IMNYNEFQHCWNEFVDGQGKPFKPRKNLPKHYTLLHATLGELLRHVMDPG
TFTSNFNNKPWVSGQRETYLCYKVERSHNDTWVLLNQHRGFLRNQAPDRH
GFPKGRHAELCFLDLIPFWKLDDQQYRVTCFTSWSPCFSCAQKMAKFISN
NKHVSLCIFAARIYDDQGRCQEGLRTLHRDGAKIAVMNYSEFEYCWDTFV
DRQGRPFQPWDGLDEHSQALSGRLRAI
(italic: nucleic acid editing domain;
underline: cytoplasmic localization signal)
Human APOBEC-3G
(SEQ ID NO: 279)
MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKC
TRDMATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTFNFNNEPWVRGRHETYLCYEVERMHNDTWVLLNQRRGFLCNQAPHKH
GFLEGRHAELCFLDVIPFWKLDLDQDYRVTCFTSWSPCFSCAQEMAKFIS
KNKHVSLCIFTARIYDDQGRCQEGLRTLAEAGAKISIMTYSEFKHCWDTF
VDHQGCPFQPWDGLDEHSQDLSGRLRAILQNQEN
(italic: nucleic acid editing domain;
underline: cytoplasmic localization signal)
Human APOBEC-3F
(SEQ ID NO: 280)
MKPHFRNTVERMYRDTFSYNFYNRPILSRRNTVWLCYEVKTKGPSRPRLD
AKIFRGQVYSQPEHHAEMCFLSWFCGNQLPAYKCFQITWFVSWTPCPDCV
AKLAEFLAEHPNVTLTISAARLYYYWERDYRRALCRLSQAGARVKIMDDE
EFAYCWENFVYSEGQPFMPWYKFDDNYAFLHRTLKEILRNPMEAMYPHIF
YFHFKNLRKAYGRNESWLCFTMEVVKHHSPVSWKRGVFRNQVDPETHCHA
ERCFLSWFCDDILSPNTNYEVTWYTSWSPCPECAGEVAEFLARHSNVNLT
IFTARLYYFWDTDYQEGLRSLSQEGASVEIMGYKDFKYCWENFVYNDDEP
FKPWKGLKYNFLFLDSKLQEILE
(italic: nucleic acid editing domain)
Human APOBEC-3B
(SEQ ID NO: 281)
MNPQIRNPMERMYRDTFYDNFENEPILYGRSYTWLCYEVKIKRGRSNLLW
DTGVFRGQVYFKPQYHAEMCFLSWFCGNQLPAYKCFQITWFVSWTPCPDC
VAKLAEFLSEHPNVTLTISAARLYYYWERDYRRALCRLSQAGARVTIMDY
EEFAYCWENFVYNEGQQFMPWYKFDENYAFLHRTLKEILRYLMDPDTFTF
NFNNDPLVLRRRQTYLCYEVERLDNGTWVLMDQHMGFLCNEAKNLLCGFY
GRHAELRFLDLVPSLQLDPAQIYRVTWFISWSPCFSWGCAGEVRAFLQEN
THVRLRIFAARIYDYDPLYKEALQMLRDAGAQVSIMTYDEPEYCWDTFVY
RQGCPFQPWDGLEEHSQALSGRLRAILQNQGN
(italic: nucleic acid editing domain)
Human APOBEC-3C:
(SEQ ID NO: 282)
MNPQIRNPMKAMYPGTFYFQFKNLWEANDRNETWLCFTVEGIKRRSVVSW
KTGVFRNQVDSETHCHAERCFLSWFCDDILSPNTKYQVTWYTSWSPCPDC
AGEVAEFLARHSNVNLTIFTARLYYFQYPCYQEGLRSLSQEGVAVEIMDY
EDFKYCWENFVYNDNEPFKPWKGLKTNFRLLKRRLRESLQ
(italic: nucleic acid editing domain)
Human APOBEC-3A:
(SEQ ID NO: 283)
MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSP
CFSWGCAGEVRAFLQENTHVRLRIFAARIYDYDPLYKEALQMLRDAGAQV
SIMTYDEFKHCWDTFVDHQGCPFQPWDGLDEHSQALSGRLRAILQNQGN
(italic: nucleic acid editing domain)
Human APOBEC-3H:
(SEQ ID NO: 284)
MALLTAETFRLQFNNKRRLRRPYYPRKALLCYQLTPQNGSTPTRGYFENK
KKCHAEICHNEIKSMGLDETQCYQVTCYLTWSPCSSCAWELVDFIKAHDH
LNLGIFASRLYYHWCKPQQKGLRLLCGSQVPVEVMGFPKFADCWENFVDH
EKPLSFNPYKMLEELDKNSRAIKRRLERIKIPGVRAQGRYMDILCDAEV
(italic: nucleic acid editing domain)
Human APOBEC-3D
(SEQ ID NO: 285)
MNPQIRNPMERMYRDTFYDNFENEPILYGRSYTWLCYEVKIKRGRSNLLW
DTGVFRGPVLPKRQSNHRQEVYFRFENHAEMCFLSWFCGNRLPANRRFQI
TWFVSWNPCLPCVVKVTKFLAEHPNVTLTISAARLYYYRDRDWRWVLLRL
HKAGARVKIMDYEDFAYCWENFVCNEGQPFMPWYKFDDNYASLHRTLKEI
LRNPMEAMYPHIFYFHFKNLLKACGRNESWLCFTMEVTKHHSAVFRKRGV
FRNQVDPETHCHAERCFLSWFCDDILSPNTNYEVTWYTSWSPCPECAGEV
AEFLARHSNVNLTIFTARLCYFWDTDYQEGLCSLSQEGASVKIMGYKDFV
SCWKNFVYSDDEPFKPWKGLQTNFRLLKRRLREILQ
(italic: nucleic acid editing domain)
Human APOBEC-1
(SEQ ID NO: 286)
MTSEKGPSTGDPTLRRRIEPWEFDVFYDPRELRKEACLLYEIKWGMSRKI
WRSSGKNTTNHVEVNFIKKFTSERDFHPSMSCSITWFLSWSPCWECSQAI
REFLSRHPGVTLVIYVARLFWHMDQQNRQGLRDLVNSGVTIQIMRASEYY
HCWRNFVNYPPGDEAHWPQYPPLWMMLYALELHCIILSLPPCLKISRRWQ
NHLTFFRLHLQNCHYQTIPPHILLATGLIHPSVAWR
Mouse APOBEC-1
(SEQ ID NO: 287)
MSSETGPVAVDPTLRRRIEPHEFEVFFDPRELRKETCLLYEINWGGRHSV
WRHTSQNTSNHVEVNFLEKFTTERYFRPNTRCSITWFLSWSPCGECSRAI
TEFLSRHPYVTLFIYIARLYHHTDQRNRQGLRDLISSGVTIQIMTEQEYC
YCWRNFVNYPPSNEAYWPRYPHLWVKLYVLELYCIILGLPPCLKILRRKQ
PQLTFFTITLQTCHYQRIPPHLLWATGLK
Rat APOBEC-1
(SEQ ID NO: 288)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK
Petromyzon marinus CDA1 (pmCDA1)
(SEQ ID NO: 289)
MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADC
AEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKIL
HTTKSPAV
Human APOBEC3G D316R_D317R
(SEQ ID NO: 290)
MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKC
TRDMATFLAEDPKVTLTIFVARLYYFWDPDYQEALRSLCQKRDGPRATMK
IMNYDEFQHCWSKFVYSQRELFEPWNNLPKYYILLHIMLGEILRHSMDPP
TFTFNFNNEPWVRGRHETYLCYEVERMHNDTWVLLNQRRGFLCNQAPHKH
GFLEGRHAELCFLDVIPFWKLDLDQDYRVTCFTSWSPCFSCAQEMAKFIS
KNKHVSLCIFTARIYRRQGRCQEGLRTLAEAGAKISIMTYSEFKHCWDTF
VDHQGCPFQPWDGLDEHSQDLSGRLRAILQNQEN
Human APOBEC3G chain A
(SEQ ID NO: 291)
MDPPTFTFNFNNEPWVRGRHETYLCYEVERMHNDTWVLLNQRRGFLCNQA
PHKHGFLEGRHAELCFLDVIPFWKLDLDQDYRVTCFTSWSPCFSCAQEMA
KFISKNKHVSLCIFTARIYDDQGRCQEGLRTLAEAGAKISIMTYSEFKHC
WDTFVDHQGCPFQPWDGLDEHSQDLSGRLRAILQ
Human APOBEC3G chain A D120R_D121R
(SEQ ID NO: 292)
MDPPTFTFNFNNEPWVRGRHETYLCYEVERMHNDTWVLLNQRRGFLCNQA
PHKHGFLEGRHAELCFLDVIPFWKLDLDQDYRVTCFTSWSPCFSCAQEMA
KFISKNKHVSLCIFTARIYRRQGRCQEGLRTLAEAGAKISIMTYSEFKHC
WDTFVDHQGCPFQPWDGLDEHSQDLSGRLRAILQ

Non-Limiting Examples of Fusion Proteins/Nucleobase Editors are Provided.

His₆-rAPOBEC1-XTEN-dCas9 for Escherichia coli expression
(SEQ ID NO: 293)
MGSSHHHHHHMSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHHADPRNRQG
LRDLISSGVTIQIMTEQESGYCWRNFVNYSPSNEAHWPRYPHLWVRLYVLELYCIILGLPPCLNILRRKQ
PQLTFFTIALQSCHYQRLPPHILWATGLKSGSETPGTSESATPESDKKYSIGLAIGTNSVGWAVITDEYK
VPSKKFKVLGNTDRHSIKKNLIGALLFDSGETALATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLENLIAQLPGEKKNI
GLFGNLIALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQLEFYKF
IKPILEKMDGTEELLVKLNREDLLRKQRTFDNGSIPHQIHLGELHAILRRQEDFYPFLKDNREKIEKILT
FRIPYYVGPLARGNSRFAWMTRKSEETITPWNFEEVVDKGASAQSFIERMTNFDKNLPNEKVLPKHSLLY
EYFTVYNELTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVMKQLKRRR
YTGWGRLSRKLINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVLTRSDKNRG
KSDNVPSEEVVKKMKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGGFDSPTVAYSVLV
VAKVEKGKSKKLKSVKELLGITIMERSSFEKNPIDFLEAKGYKEVKKDLIIKLPKYSLFELENGRKRMLA
ESAGELQKGNELALPSKYVNFLYLASHYEKLKGSPEDNQKQLFVEQHKHYLDEIIEQISEFSKRVILADA
NLDKVLSAYNKHRDKPIREQAENIIHLFTLTNLGAPAAFKYFDTTIDRKRYTSTKEVLDATLIHQSITGL
YETRIDLSQLGGDSGGSPKKKRKV
rAPOBEC1-XTEN-dCas9-NLS for Mammalian expression
(SEQ ID NO: 294)
MSSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPCLNILRRKQPQLTFFTIAL
QSCHYQRLPPHILWATGLKSGSETPGTSESATPESDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQLEFYKFIKPILEKMDGT
EELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSDNVPSEEVV
KKMKNYWRQLLNAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGRDFATVRKV
LSMPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLYETRIDLSQLG
GDSGGSPKKKRKV
hAPOBEC1-XTEN-dCas9-NLS for Mammalian expression
(SEQ ID NO: 295)
MTSEKGPSTGDPTLRRRIEPWEFDVFYDPRELRKEACLLYEIKWGMSRKIWRSSGKNTTNHVEVNFIKKF
TSERDFHPSMSCSITWFLSWSPCWECSQAIREFLSRHPGVTLVIYVARLFWHMDQQNRQGLRDLVNSGVT
IQIMRASEYYHCWRNFVNYPPGDEAHWPQYPPLWMMLYALELHCIILSLPPCLKISRRWQNHLTFFRLHL
QNCHYQTIPPHILLATGLIHPSVAWRSGSETPGTSESATPESDKKYSIGLAIGTNSVGWAVITDEYKVPS
KKFKVLGNTDRHSIKKNLIGALLFDSGETALATRLKRTARRRYTRRKNRICYLQEIFSNEMAKVDDSFFH
RLEESFLVEEDKKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQLEFYKFIKPI
LEKMDGTEELLVKLNREDLLRKQRTFDNGSIPHQIHLGELHAILRRQEDFYPFLKDNREKIEKILTFRIP
YYVGPLARGNSRFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVMKQLKRRRYTGW
GRLSRKLINGIRDKQSGKTILDFLKSDGFANRNFMQLIHDDSLTFKEDIQKAQVSGQGDSLHEHIANLAG
SPAIKKGILQTVKVVDELVKVMGRHKPENIVIEMARENQTTQKGQKNSRERMKRIEEGIKELGSQILKEH
PVENTQLQNEKLYLYYLQNGRDMYVDQELDINRLSDYDVDAIVPQSFLKDDSIDNKVLTRSDKNRGKSDN
VPSEEVVKKMKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKYPKLESEFV
YGDYKVYDVRKMIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITGLYETR
IDLSQLGGDSGGSPKKKRKV
rAPOBEC1-XTEN-dCas9-UGI-NLS
(SEQ ID NO: 296)
MSSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPCLNILRRKQPQLTFFTIAL
QSCHYQRLPPHILWATGLKSGSETPGTSESATPESDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQLEFYKFIKPILEKMDGT
EELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSDNVPSEEVV
KKMKNYWRQLLNAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGRDFATVRKV
LSMPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLYETRIDLSQLG
GDSGGSTNLSDIIEKETGKQLVIQESILMLPEEVEEVIGNKPESDILVHTAYDESTDENVMLLTSDAPEY
KPWALVIQDSNGENKIKMLSGGSPKKKRKV
rAPOBEC1-XTEN-Cas9 nickase-UGI-NLS
(BE3, SEQ ID NO: 297)
MSSETGPVAVDPTLRRRIEPHEFEVFFDPRELRKETCLLYEINWGGRHSIWRHTSQNTNKHVEVNFIEKF
TTERYFCPNTRCSITWFLSWSPCGECSRAITEFLSRYPHVTLFIYIARLYHHADPRNRQGLRDLISSGVT
IQIMTEQESGYCWRNFVNYSPSNEAHWPRYPHLWVRLYVLELYCIILGLPPCLNILRRKQPQLTFFTIAL
QSCHYQRLPPHILWATGLKSGSETPGTSESATPESDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGGASQLEFYKFIKPILEKMDGT
EELLVKLNREDLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIECFDSVEISGVEDRFNASLGTYHD
LLKIIKDKDFLDNEENEDILEDIVLTITLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSDNVPSEEVV
KKMKNYWRQLLNAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGRDFATVRKV
LSMPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLYETRIDLSQLG
GDSGGSTNLSDIIEKETGKQLVIQESILMLPEEVEEVIGNKPESDILVHTAYDESTDENVMLLTSDAPEY
KPWALVIQDSNGENKIKMLSGGSPKKKRKV
pmCDA1-XTEN-dCas9-UGI (bacteria)
(SEQ ID NO: 298)
MTDAEYVRIHEKLDIYTFKKQFFNNKKSVSHRCYVLFELKRRGERRACFWGYAVNKPQSGTERGIHAEIF
SIRKVEEYLRDNPGQFTINWYSSWSPCADCAEKILEWYNQELRGNGHTLKIWACKLYYEKNARNQIGLWN
LRDNGVGLNVMVSEHYQCCRKIFIQSSHNQLNENRWLEKTLKRAEKRRSELSIMIQVKILHTTKSPAVSG
SETPGTSESATPESDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLYYLQNGRDMYVDQE
LDINRLSDYDVDAIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGSMTNLSDIIEKETGKQ
LVIQESILMLPEEVEEVIGNKPESDILVHTAYDESTDENVMLLTSDAPEYKPWALVIQDSNGENKIKML
pmCDA1-XTEN-nCas9-UGI-NLS (mammalian construct) (SEQ ID NO: 299):
MTDAEYVRIHEKLDIYTFKKQFFNNKKSVSHRCYVLFELKRRGERRACFWGYAVNKPQSGTERGIHAEIF
SIRKVEEYLRDNPGQFTINWYSSWSPCADCAEKILEWYNQELRGNGHTLKIWACKLYYEKNARNQIGLWN
LRDNGVGLNVMVSEHYQCCRKIFIQSSHNQLNENRWLEKTLKRAEKRRSELSIMIQVKILHTTKSPAVSG
SETPGTSESATPESDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHERHPIFGNIVDEVA
YHEKYPTIYHLRKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMRKPAFLSGEQKKA
IVDLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKGILQTVKVVDELVKVMGRHKPE
NIVIEMARENQTTQKGQKNSRERMKRIEEGIKELGSQILKEHPVENTQLQNEKLYLYYLQNGRDMYVDQE
LDINRLSDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGSTNLSDIIEKETGKQL
VIQESILMLPEEVEEVIGNKPESDILVHTAYDESTDENVMLLTSDAPEYKPWALVIQDSNGENKIKMLSG
GSPKKKRKV
huAPOBEC3G-XTEN-dCas9-UGI (bacteria) (SEQ ID NO: 300)
MDPPTFTFNFNNEPWVRGRHETYLCYEVERMHNDTWVLLNQRRGFLCNQAPHKHGFLEGRHAELCFLDVI
PFWKLDLDQDYRVTCFTSWSPCFSCAQEMAKFISKNKHVSLCIFTARIYDDQGRCQEGLRTLAEAGAKIS
IMTYSEFKHCWDTFVDHQGCPFQPWDGLDEHSQDLSGRLRAILQSGSETPGTSESATPESDKKYSIGLAI
GTNSVGWAVITDEYKVPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAILRRQEDFYP
FLKDNREKIEKILTFRIPYYVGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNRKVTVKQLKEDYFKK
IECFDSVETSGVEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSFLKDDS
IDNKVLTRSDKNRGKSDNVPSEEVVKKMKNYWRQLLNAKLITQRKFDNLTKAERGGLSELDKAGFIKRQL
VETRQITKHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKY
SLFELENGRKRMLASAGELQKGNELALPSKYVNFLYLASHYEKLKGSPEDNEQKQLFVEQHKHYLDEIIE
QISEFSKRVILADANLDKVLSAYNKHRDKPIREQAENIIHLFTLTNLGAPAAFKYFDTTIDRKRYTSTKE
VLDATLIHQSITGLYETRIDLSQLGGDSGGSMTNLSDIIEKETGKQLVIQESILMLPEEVEEVIGNKPES
DILVHTAYDESTDENVMLLTSDAPEYKPWALVIQDSNGENKIKML
huAPOBEC3G-XTEN-nCas9-UGI-NLS (mammalian construct) (SEQ ID NO: 301)
MDPPTFTFNFNNEPWVRGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGAKI
SIMTYSEFKHCWDTFVDHQGCPFQPWDGLDEHSQDLSGRLRAILQSGSETPGTSESATPESDKKYSIGLA
IGTNSVGWAVITDEYKVPSKKFKVLGNTDRHSIKKNLIGALLFDSGETAEATRLKRTARRRYTRRKNRIC
YLQEIFSNEMAKVDDSFFHRLEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKVTVKQLKEDYFK
KIECFDSVETSGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDTTIDRKRYTSTK
EVLDATLIHQSITGLYETRIDLSQLGGDSGGSTNLSDIIEKETGKQLVIQESILMLPEEVEEVIGNKPES
DILVHTAYDESTDENVMLLTSDAPEYKPWALVIQDSNGENKIKMLSGGSPKKKRKV
huAPOBEC3G (D316R_D317R)-XTEN-nCas9-UGI-NLS (mammalian construct)
(SEQ ID NO: 302)
MDPPTFTFNFNNEPWVRGRHETYLCYEVERMHNDTWVLLNQRRGFLCNQAPHKHGFLEGRHAELCFLDVI
PFWKLDLDQDYRVTCFTSWSPCFSCAQEMAKFISKNKHVSLCIFTARIYRRQGRCQEGLRTLAEAGAKIS
IMTYSEFKHCWDTFVDHQGCPFQPWDGLDEHSQDLSGRLRAILQSGSETPGTSESATPESDKKYSIGLAI
GTNSVGWAVITDEYKVPSKKFKVLGNTDRHSIKKNLIGALLFDSGETALATRLKRTARRRYTRRKNRICY
LQEIFSNEMAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILSARLSKSRR
LENLIAQLPGEKKNIGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQSKNGYA
GYIDGGASQLEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDTTIDRKRYTSTK
EVLDATLIHQSITGLYETRIDLSQLGGDSGGSTNLSDIIEKETGKQLVIQESILMLPEEVEEVIGNKPES
DILVHTAYDESTDENVMLLTSDAPEYKPWALVIQDSNGENKIKMLSGGSPKKKRKV

### Example 2: Genome/Base-Editing Methods for Modifying the CCR5 Receptor Gene to Protect Against HIV Infection

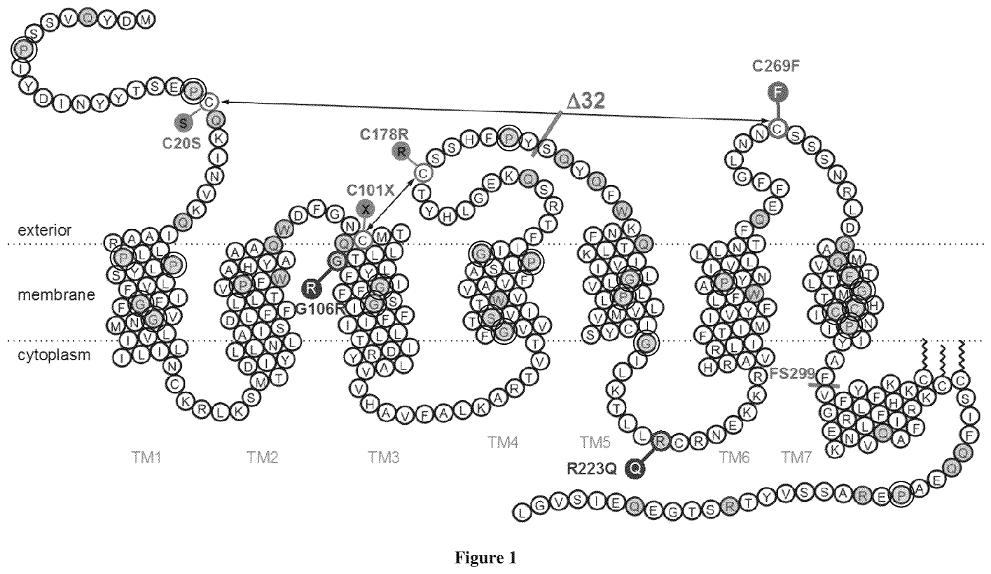

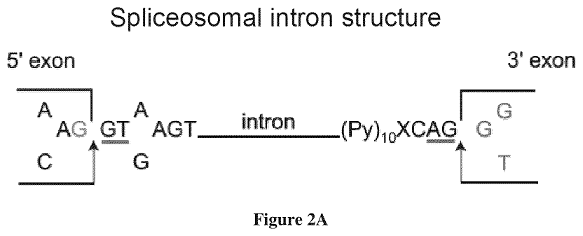

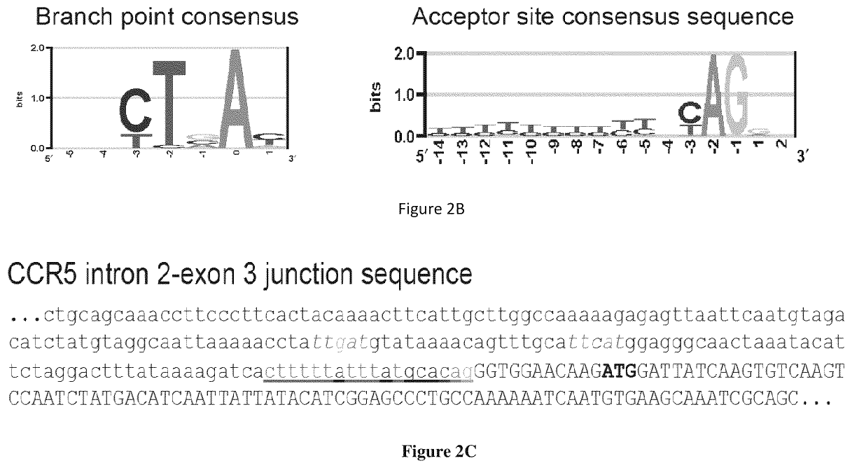

Disclosed herein are new ways for introducing novel engineered variants, as well as naturally-occurring allelic variants, of the co-receptor C-C Chemokine Receptor 5 (CCR5) that prevent or hinder cellular entry of the Human Immunodeficiency Virus (HIV). These methods include CRISPR-Cas9-based tools programmed by guide RNAs requiring either: (i) “base-editors” that catalyze chemical reactions on nucleobases (e.g., cytidine deaminase-Cas9 fusion, e.g. BE3¹); (ii) an engineered nuclease with DNA cutting activity (e.g., WT Cas9,² Cas9 nickases³ or Fok1-nuclease-dCas9 fusions⁴). The variants selected (FIG. 1, Tables 1-5) include residues that directly alter the affinity for the HIV coat protein and/or destabilize the CCR5 protein folding, which mimics the potentially curative effects of the CCR5Δ32 variant.⁵ Using a similar strategy, the intron-exon splicing junction adjacent to the open-reading frame of CCR5 can be altered to prevent the maturation and/or destabilize the mRNA transcript (FIGS. 2A to 2C, Table 2).

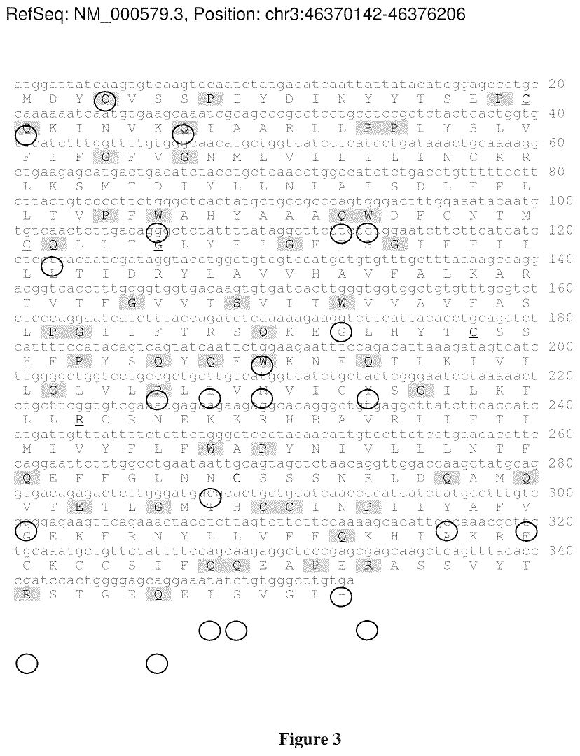

Subsequently, other natural protective variants may be identified in human populations that can be replicated in the same manner (FIG. 3. Tables 6 and 7). Moreover, new protective variants of CCR5 (Tables 8-10), and also CCR2,⁶ could be identified by treating cells in vitro with guide-RNA libraries designed for all possible PAMs in these gene, coupled with FACS sorting using reporters/labeling methods and DNA-deep sequencing, to find the guide-RNAs that programmed base-editing reactions that lower CCR5 protein expression, prevent gp120 binding, and/or hinder HIV entry into the cell. For example, engineered alterations to destabilize CCR5 may follow a simple design of switching hydrophobic to polar residues on the transmembrane helices (FIG. 1). The precisely-targeted methods for CCR5 modifications proposed herein are complementary to previous methods that create random indels in the CCR5 genomic site using engineered nucleases such as CRISPR/Cas9. TALEN, or zinc-finger nucleases in hematopoietic cells ex vivo.⁷ Moreover, “base-editors” such as BE3 may have a more favorable safety profile, due to the relatively low impact that off-target cytosine deamination has on genomic stability,⁸ including oncogene activation or tumor suppressor inactivation⁹.

### Example 3: Exemplary C to T Editing Demonstrating Modification of the CCR5 Receptor Gene to Generate Q186X and Q188X Stop Codons

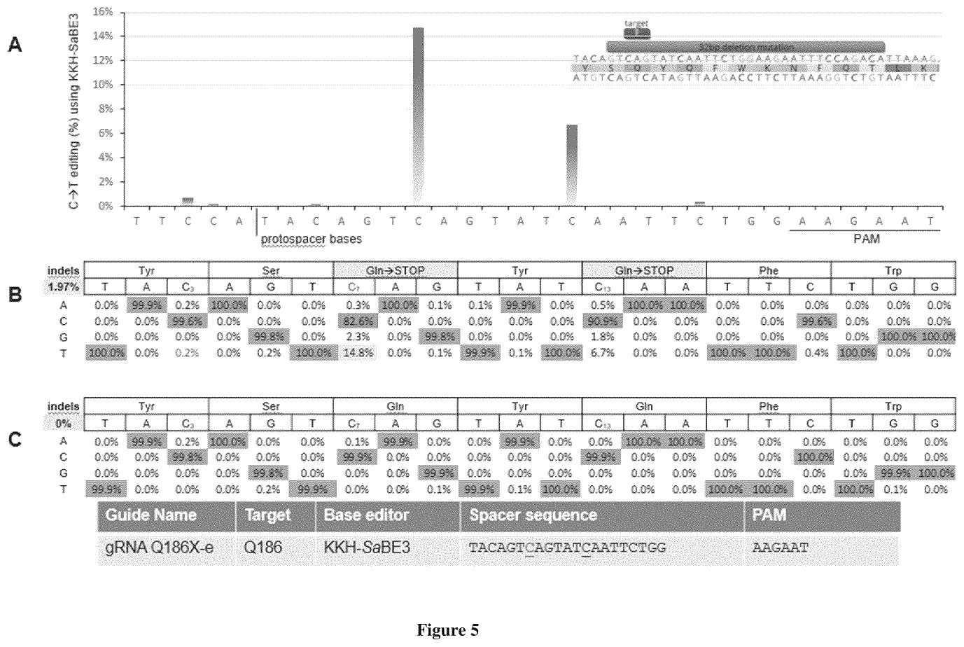

C to T editing of CCR5 was performed in HEK293 cells using KKH-SaBE3 and guide-RNA Q186X-e [spacer sequence TACAGTCAGTATCAATTCTGG (SEQ ID NO: 735); PAM sequence: AAGAAT (SEQ ID NO: 736)]. The results from these experiments are shown in FIG. 5, panels A-C. The editing was calculated from total reads (MiSeq). FIG. 5, panel A demonstrates that significant editing was observed at position C7 and C13, both of which generate premature stop codons in tandem (Q186X and Q188X, see inset graphic of FIG. 5, panel A). The PAM sequence is shown as underlined and the last nucleotide of the protospacer is separated with a line. Raw data used for base-calling and calculating base-editing for KKH-BE3 and Q186X-e treated HEK293 cells is shown in FIG. 5, panel B. The indel percentage was 1.97%. FIG. 5, panel C shows raw data collected for untreated control cells.

TABLE 1
Introduction of HIV-protective naturally-occurring allelic variants of
CCR5 and CCR2 using genome/base-editing with APOBEC1-Cas9 tools (e.g. BE3¹).

| Known | Target | Genome-editing | Edited | Match/ | |
|---|---|---|---|---|---|
| variant | codon | reaction(s) | codon | mimic | Predicted outcome (ref) |
| CCR5 | GAT | 1^{st} base C→T on | AAT | Asparagine | Charge neutralized, unfolding, |
| (D2V) | | complementary strand | | (mimic) | and destabilization^{5c} |
| CCR5 | TGC | 2^{nd} base C→T on | TAC | Tyrosine | Lack of major disulfide bridge, |
| (C20S) | | complementary strand | | (mimic) | unfolding^{5c} |
| CCR5 | TGT | 2^{nd} base C→T on | TAT | Tyrosine | Lack of minor disulfide bridge, |
| (C101X) | | complementary strand | | (mimic) | unfolding, destabilization^{5c} |
| CCR5 | GGG | 1^{st} base C→T on | AGG | Arginine | Transmembrane helix |
| (G106R) | | complementary strand | | (match) | disruption, destabilization^{5a, 5c} |
| CCR5 | TGC | 2^{nd} base C→T on | TAC | Tyrosine | Lack of minor disulfide bridge, |
| (C178R) | | complementary strand | | (mimic) | destabilization^{5c} |
| CCR5 | CGG | 2^{nd} base C→T on | CAG | Glutamine | Charge neutralized, |
| (R223Q) | | complementary strand | | (match) | destabilization^{5c} |
| CCR5 | TGC | 2^{nd} base C→T on | TAC | Tyrosine | Lack of major disulfide bridge, |
| (C269F) | | complementary strand | | (mimic) | unfolding, destabilization^{5a, 5c} |
| CCR2 | GCA | 2^{nd} base C→T on coding | GTA | Valine | Hydrophobic patch, unfolding, |
| (A335V) | | strand | | (match) | destabilization^{5c} |
| CCR2 | GTC | 2^{nd} base C→T on | TAA | Isoleucine | Affects CCR5 stability⁶ |
| (V64I) | | complementary strand | | (match) | |

TABLE 2
Examples of genome-editing reactions to alter intron-exon junctions and
the START site and produce non-functional CCR5 protein, mimicking the HIV protective effect
of the CCR5-Δ32 allele.

| Target | Consensus | | Genome-editing | Edited | |
|---|---|---|---|---|---|
| site | sequence | Method | reaction(s) | sequence | Outcome |
| Intron | G-G-G-T-R-A- | Base- | 2^{nd} or 3^{rd} base C→T | G-A-G-T-R- | Intron sequence is |
| donor | G-T | editing | on complementary | A-G-T | translated as exon, next |
| | | | strand | (example) | TAG, TGA, or TAA |
| | | | | | sequence is used as |
| | | | | | STOP codon |
| Lariat | T-T-G-T-A | Base- | 3^{th} base C→T on | T-T-A-T-A | The following exon is |
| branch | | editing | coding strand | | skipped from the mature |
| point | | | | | mRNA, which may affect |
| | | | | | the coding frame |
| Intron | Y(rich)-A-C-A- | Base- | 2^{nd} to last base C→T | Y(rich)-A-C- | The exon is skipped from |
| acceptor | G-G | editing | on complementary | A-A-G | the mature mRNA, which |
| | | | strand | | may affect the coding |
| | | | | | frame |
| Start | ATG | Base- | 3^{rd} base C→T on | ATA | The next ATG is used as |
| codon | (Methionine) | editing | complementary | (Isoleucine) | start, which may affect |
| | | | strand | | the coding frame |
| Intron | G-G-G-T-R-A- | Cas9 | random insertions | indels | Intron sequence is |
| donor | G-T | Nickase | and deletions due to | | translated as exon, next |
| | | Fok-1 | NHEJ | | TAG, TGA, or TAA |
| | | | | | sequence is used as |
| | | | | | STOP codon |
| Lariat | T-T-N-T-A | Cas9 | random insertions | indels | The following exon is |
| branch | | Nickase | and deletions due to | | skipped from the mature |
| point | | Fok-1 | NHEJ | | mRNA, which may affect |
| | | | | | the coding frame |
| Intron | Y(rich)-A-C-A- | Cas9 | random insertions | indels | The exon is skipped from |
| acceptor | G-G | Nickase | and deletions due to | | the mature mRNA, which |
| | | Fok-1 | NHEJ | | may affect the coding |
| | | | | | frame |

TABLE 3
Guide-RNAs designed for introducing
naturally-occurring HIV-protective variants
of genome/base editing of CCR5 using
base-editor BE3 or WT Cas9.

| | | | | SEQ | | | |
|---|---|---|---|---|---|---|---|
| Target | Target | Edited | Guide-RNA | ID | | | GE/BE |
| variant | codon | codon | sequence | NO: | (PAM) | Size (C#) | method |
| CCR5 | GAT | AAT | UAAUCCAUCU | 381 | (TGTG) | 20 (C5) | VRER- |
| (D2N) | | | UGUUCCACCC | | | | SpBE3 |
| | | | CCAUCUUGUUC | 382 | {ATAAAT) | 21 (C−1) | KKH- |
| | | | CACCCUGUGC | | | | SaBE3 |
| CCR5 | TGC | TAC | CAGGGCUCCG | 383 | (ATTGAT) | 20 (C1) | KKH- |
| (C20Y) | | | AUGUAUAAUA | | | | SaBE3 |
| | | | UUGGCAGGGC | 384 | (AATAAT) | 20 (C5) | KKH- |
| | | | UCCGAUGUAU | | | | SaBE3 |
| CCR5 | TGT | TAT | GUUGACACAU | 385 | (AAG) | 20 (C6) | SpBE3 |
| (C101Y) | | | UGUAUUUCCA | | | | |
| | | | GAGUUGACAC | 386 | (CAAAGT) | 20 (C8) | KKH- |
| | | | AUUGUAUUUC | | | | SaBE3 |
| CCR5 | GGG | AGG | AUAAAAUAGA | 387 | (GAG) | 20 (C13) | VQR- |
| (G106R) | | | GCCCUGUCAA | | | | SpBE3 |
| CCR5 | TGC | TAC | UGAGAGCUGC | 388 | (AAG) | 20 (C10) | SpBE3 |
| (C178Y) | | | AGGUGUAAUG | | | | |
| | | | GAGAGCUGCA | 389 | (AGA) | 20 (C9) | VQR- |
| | | | GGUGUAAUGA | | | | SpBE3 |
| CCR5 | CGG | CAG | CGACACCGAA | 390 | (TAG) | 20 (C7) | SpBE3 |
| (R223Q) | | | GCAGAGUUUU | | | | |
| | | | GACACCGAAG | 391 | (AGG) | 20 (C6) | SpBE3 |
| | | | CAGAGUUUUU | | | | |
| | | | ACACCGAAGC | 392 | (GGA) | 20 (C5) | VQR- |
| | | | AGAGUUUUUA | | | | SpBE3 |
| | | | CGACACCGAA | 393 | (TAGGAT) | 20 (C7) | SaBE3 |
| | | | GCAGAGUUUU | | | | |
| | | | CGAAGCAGAGU | 394 | (CCGAGT) | 22 (C-2) | SaBE3 |
| | | | UUUUAGGAUUC | | | | |
| CCR5 | TGC | TAC | UACUGCAAUU | 395 | (AAG) | 20 (C6) | SpBE3 |
| (C269Y) | | | AUUCAGGCCA | | | | |
| | | | ACUGCAAUUA | 396 | (AGA) | 20 (C5) | VQR- |
| | | | UUCAGGCCAA | | | | SpBE3 |
| | | | UACUGCAAUU | 397 | (AAGAAT) | 20 (C6) | SaBE3 |
| | | | AUUCAGGCCA | | | | |
| splicing | CAGG | CAAG | CCACCCUGUG | 398 | (AAG) | 20 (C6/5) | SpBE3 |
| acceptor | | | CAUAAAUAAA | | | | |
| site | | | CCCUGUGCAU | 399 | (TGA) | 20 (C3/2) | VQR- |
| | | | AAAUAAAAAG | | | | SpBE3 |
| | | | CACCCUGUGC | 400 | (AGTG) | 20 (C5/4) | VRER- |
| | | | AUAAAUAAAA | | | | SpBE3 |
| | | | CACCCUGUGC | 401 | (AGTGAT) | 20 (C5/4) | KKH- |
| | | | AUAAAUAAAA | | | | SaBE3 |
| | | | UUCCACCCUG | 402 | (AAAAGT) | 20 (C7/8) | KKH- |
| | | | UGCAUAAAUA | | | | SaBE3VQR- |
| | | | UUUAUGC | | | | |
| | | | ACAGGGU | 403 | (AGAT) | 20 (C9) | SpBE3 |
| | | | GGAACA | | | | |
| | | | UGCACAGGGU | 404 | (GGAT) | 20 (C5) | VQR- |
| | | | GGAACAAGAU | | | | SpBE3 |
| | | | AUUUAUGCAC | 405 | (AAG) | 20 (C10) | SpBE3 |
| | | | AGGGUGGAAC | | | | |
| | | | AUGCACAGGG | 406 | (TGG) | 20 (C6) | SpBE3 |
| | | | UGGAACAAGA | | | | |
| splicing | RTNA | indels | GAGGGCAACU | 407 | (TAG) | 20 | WT |
| branch | | | AAAUACAUUC | | | | SpCas9 |
| point | | | AGGGCAACUA | 408 | (AGG) | 20 | WT |
| | | | AAUACAUUCU | | | | SpCas9 |
| | | | AAACUGUUUU | 409 | (AGG) | 20 | WT |
| | | | AUACAUCAAU | | | | SpCas9 |
| | | | CAAACUGUUU | 410 | (TAG) | 20 | WT |
| | | | UAUACAUCAA | | | | SpCas9 |

Base editors: SpBE3-APOBEC1-SpCas9n-UGI; VQR-SpBE3-APOBEC1-VQR-SpCas9n-UGI; EQR-SpBE3 = APOBEC1-EQR-SpCas9n-UGI; VRER-SpBE3 = APOBEC1-VRER-Sp0as9n-UGI; SaBE3 = APOBEC1-SaCas9n-UGI; KKH-SaBE3 = APOBEC1-KKH-SaCas9n-UGI.

TABLE 4
Guide-RNAs designed for engineering new HIV-protective variants
of genome/base editing of CCR5 using base-editor BE3.

| | | | | SEQ | | | |
|---|---|---|---|---|---|---|---|
| Target | Target | Edited | | ID | | | GE/BE |
| variant(s) | codon | codon | Guide-RNA | NO: | (PAM) | Size (C#) | method |
| P19S/L | ccc | TCC | CCCUGCCAAAAAAUCAAUGUG | 411 | (AAG) | 21 (C1/−1) | SpBE3 |
| | | or | CCUGCCAAAAAAUCAAUGUG | 412 | (AAG) | 20 (C1) | SpBE3 |
| | | CTC | GAGCCCUGCCAAAAAAUCAA | 413 | (TGTG) | 20 (C5/6) | VQR-SpBE3 |
| | | | GCCCUGCCAAAAAAUCAAUG | 414 | (TGAA) | 20 (C2/3) | VQR-SpBE3 |
| | | | UAUACAUCGGAGCCCUGCCA | 415 | (AAAAAT) | 20 (C13) | KKH-SaBE3 |
| | | | CAUCGGAGCCCUGCCAAAAA | 416 | (ATCAAT) | 20 (C9/10) | KKH-SaBE3 |
| P34S/L | CCT | TCT | CCUGCCUCCGCUCUACUCAC | 417 | (TGG) | 20 (C5/6) | SpBE3 |
| | | or | CUGCCUCCGCUCUACUCACU | 418 | (GGTG) | 20 (C4/5) | VQR-SpBE3 |
| | | CTT | CUCCUGCCUCCGCUCUACUC | 419 | (ACTGGT) | 20 (C7/8) | KKH-SaBE3 |
| P35S/L | CCG | TCG | same as above for P34S/L | | | | |
| | | or | | | | | |
| | | CTG | | | | | |
| G44S/D | GGT | AGT | AACCAAAGAUGAACACCAGU | 420 | (GAG) | 20 (C3/4) | SpBE3 |
| | | or | CAAAACCAAAGAUGAACACC | 421 | (AGTG) | 20 (C5/6) | VQR-SpBE3 |
| | | GAT | ACAAAACCAAAGAUGAACAC | 422 | (CAG) | 20 (C7/8) | SpBE3 |
| | | | AAAACCAAAGAUGAACACCA | 423 | (GTGAGT) | 20 (C5/6) | SaBE3 |
| | | | CCACAAAACCAAAGAUGAAC | 424 | (ACCAGT) | 20 (C9/10) | KKH-SaBE3 |
| G47S/D | GGC | AGC | GCAUGUUGCCCACAAAACCA | 425 | (AAG) | 20 (C9/10) | SpBE3 |
| | | or | GUUGCCCACAAAACCAAAGA | 426 | (TGAA) | 20 (C5/6) | VQR-SpBE3 |
| | | GAC | CAUGUUGCCCACAAAACCAA | 427 | (AGAT) | 20 (C8/9) | VQR-SpBE3 |
| | | | AGCAUGUUGCCCACAAAACC | 428 | (AAAGAT) | 20 (10/11) | KKH-SaBE3 |
| G115S/D | GGC | AGC | GAGAAGAAGCCUAUAAAAUA | 429 | (GAG) | 20 (C10) | SpBE3 |
| | | or | CAGAGAAGAAGCCUAUAAAA | 430 | (TAG) | 20 (C12) | SpBE3 |
| | | GAC | | | | | |
| G115R/E | GGA | AGA | CCAGAGAAGAAGCCUAUAAA | 431 | (TAG) | 21(C1/−1) | SpBE3 |
| | | or | AUGAAGA | | | | |
| | | GAA | AGAUUCCAGAGAAG | 432 | (AAG) | 20 (C12) | SpBE3 |
| | | | GAUUCCAGAGAAGAAGCCUA | 433 | (TAAAAT) | 20 (C4/5) | KKH-SaBE3 |
| G145R/E | GGG | AGG | CACCCCAAAGGUGACCGUCC | 434 | (TGG) | 20 (C5/6) | SpBE3 |
| | | or | | | | | |
| | | GAG | | | | | |
| S149N | AGT | AAT | CACACUUGUCACCACCCCAA | 435 | (AGG) | 20 (C5) | SpBE3 |
| | | | UCACACUUGUCACCACCCCA | 436 | (AAG) | 20 (C6) | SpBE3 |
| | | | ACUUGUCACCACCCCAAAGG | 437 | (TGAC) | 20 (C2) | VQR-SpBE3 |
| | | | ACACUUGUCACCACCCCAAA | 438 | (GGTG) | 20 (C4) | VQR-SpBE3 |
| | | | AUCACACUUGUCACCACCCC | 439 | (AAAGGT) | 20 (C7) | KKH-SaBE3 |
| P162S/L | CCA | TCA | CUCCCAGGAAUCAUCUUUAC | 440 | (CAG) | 20 (C4/5) | SpBE3 |
| | | or | UCCCAGGAAUCAUCUUUACC | 441 | (AGAT) | 20 (C3/4) | VQR-SpBE3 |
| | | CTA | UCUCCCAGGAAUCAUCUUUA | 442 | (CCAGAT) | 20 (C5/6) | KKH-SaBE3 |
| G163R/E | GGA | AGA | UCCUGGGAGAGACGCAAACA | 443 | (CAG) | 20 (C3/2) | SpBE3 |
| | | or | GUAAAGAUGAUUCCUGGGAG | 444 | (AGAC) | 20 (C13) | VQR-SpBE3 |
| | | GAA | | | | | |
| P183S/L | CCA | TCA | CCAUACAGUCAGUAUCAAUUC | 445 | (TGG) | 21 (C1/−1) | SpBE3 |
| | | or | CAUACAGUCAGUAUCAAUUC | 446 | (TGG) | 20 (C1) | SpBE3 |
| | | CTA | GCUCUCAUUUUCCAUACAGU | 447 | (CAG) | 20 (C12) | SpBE3 |
| | | | UCAUUUUCCAUACAGUCAGU | 448 | (ATCAAT) | 20 (C8) | KKH-SaBE3 |
| G202R/E | GGG | AGG | UCAUUUUCCAUACAGUCAGU | 449 | (ATCAAT) | 20 (C6/C7) | KKH-SaBE3 |
| | | or | | | | | |
| | | GAG | | | | | |
| P206S/L | CCG | TCG | GGUCCUGCCGCUGCUUGUCA | 450 | (TGG) | 20 (C8/9) | SpBE3 |
| | | or | CUGGUCCUGCCGCUGCUUGU | 451 | (CATGGT) | 20 (10/11) | KKH-SaBE3 |
| | | CTG | | | | | |
| G216R/E | GGA | AGA | UCCCGAGUAGCAGAUGACCA | 452 | (TGAC) | 20 (C2/3) | VQR-SpBE3 |
| | | or | UAGGAUUCCCGAGUAGCAGA | 453 | (TGAC) | 20 (C8/9) | VQR-SpBE3 |
| | | GAA | UUUUAGGAUUCCCGAGUAGC | 454 | (AGAT) | 20 (11/12) | VQR-SpBE3 |
| E283K | GAG | AAG | UCUCUGUCACCUGCAUAGCU | 455 | (TGG) | 20 (C4) | SpBE3 |
| | | | AAGAGUCUCUGUCACCUGCA | 456 | (TAG) | 20 (C9) | SpBE3 |
| | | | AGUCUCUGUCACCUGCAUAG | 457 | (CTTGGT) | 20 (C6) | KKH-SaBE3 |
| G286R/E | GGG | AGG | CCAAGAGUCUCUGUCACCUGCA | 458 | (TAG) | 22 (−1/−2) | SpBE3 |
| | | or | | | | | |
| | | GAG | | | | | |
| C290Y | TGC | TAC | GCAGCAGUGCGUCAUCCCAA | 459 | (GAG) | 20 (C5) | SpBE3 |
| | | | UGCAGCAGUGCGUCAUCCCA | 460 | (AGAG) | 20 (C6) | VQR-SpBE3 |
| | | | AUGCAGCAGUGCGUCAUCCC | 461 | (AAG) | 20 (C1) | SpBE3 |
| | | | AUGCAGCAGUGCGUCAUCCC | 462 | (AAGAGT) | 20 (C1) | SaBE3 |
| C291Y | TGC | TAC | GCAGCAGUGCGUCAUCCCAA | 463 | (GAG) | 20 (C2) | VQR-SpBE3 |
| | | | AUGCAGCAGUGCGUCAUCCC | 464 | (AAG) | 20 (C4) | SpBE3 |
| | | | AUGCAGCAGUGCGUCAUCCC | 465 | (AAGAGT) | 20 (C4) | SaBE3 |
| P293S/L | CCC | TCC | CCCAUCAUCUAUGCCUUUGU | 466 | (CGG) | 20 (C1/2) | SpBE3 |
| | | or | CCAUCAUCUAUGCCUUUGU | 467 | (CGG) | 19 (C2) | SpBE3 |
| | | CTC | | | | | |
| P332S/L | CCC | TCC | GGCUCCCGAGCGAGCAAGCU | 468 | (CAG) | 20 (C4/5) | SpBE3 |
| | | or | CAAGAGGCUCCCGAGCGAGC | 469 | (AAG) | 20 (10/11) | SpBE3 |
| | | CTC | GAGGCUCCCGAGCGAGCAAG | 470 | (CTCAGT) | 20 (C7/8) | KKH-SaBE3 |

Base editors: SpBE3-APOBEC1-SpCas9n-UGI; VQR-SpBE3-APOBEC1-VQR-SpCas9n-UGI; EQR-SpBE3-APOBEC1-EQR-SpCas9n-UGI; VRER-SpBE3-APOBEC1-VRER-SpCas9n-UGI; SaBE3 = APOBEC1-SaCas9n-UGI; KKH-SaBE3 = APOBECl-KKH-SaCas9n-UGI.

TABLE 5
Guide-RNAs designed for engineering new HIV-protective variants
of genome/base editing of CCR5 using BE3.

| Target | Target | Stop | | SEQ ID | | | GE/BE |
|---|---|---|---|---|---|---|---|
| variant | codon | codon | Designed guide-RNAs | NO: | (PAM) | Size (C#) | method |
| Q4X | CAA | TAA | CAAGUGUCAAGUCCAAUCUA | 471 | (UGAC) | 20 (C1) | VQR-SpBE3 |
| | | (Ochre) | AAGAUGGAUUAUCAAGUGUC | 472 | (AAG) | 20 (C13) | SpBE3 |
| Q21X | CAA | TAA | CCUGCCAAAAAAUCAAUGUG | 473 | (AAG) | 20 (C6) | SpBE3 |
| | | (Ochre) | UGCCAAAAAAUCAAUGUGAA | 474 | (GCAAAT) | 20 (C4) | SaBE3 |
| W86X | TGG | TAG | CCCAGAAGGGGACAGUAAGA | 475 | (AGG) | 20 (C3/2) | SpBE3 |
| | | (Amber) | GCCCAGAAGGGGACAGUAAG | 476 | (AAG) | 20 (C4/3) | SpBE3 |
| | | or | GAGCCCAGAAGGGGACAGUA | 477 | (AGAA) | 20 (C5/4) | VQR-SpBE3 |
| | | TGA | UGAGCCCAGAAGGGGACAGU | 478 | (AAG) | 20 (C6/7) | SpBE3 |
| | | (Opal) | AGCAUAGUGAGCCCAGAAGGG | 479 | (GACAGT) | 21 (C13) | KKH-SaBE3 |
| Q93X | CAG | TAG | GCUGCCGCCCAGUGGGACUU | 480 | (TGG) | 20 (C9) | SpBE3 |
| | | (Amber) | CUGCCGCCCAGUGGGACUUU | 481 | (GGAA) | 20 (C9) | VQR-SpBE3 |
| | | | CUGCCGCCCAGUGGGACUUU | 482 | (GGAAAT) | 20 (C9) | KKH-SaBE3 |
| | | | GCCCAGUGGGACUUUGGAAA | 483 | (TACAAT) | 20 (C4) | KKH-SaBE3 |
| W94X | TGG | TAG | AGUCCCACUGGGCGGCAGCA | 484 | (TAG) | 20 (C5/6) | SpBE3 |
| | | (Amber) | UCCAAAGUCCCACUGGGCGG | 485 | (CAG) | 20 (C10) | SpBE3 |
| | | or | CCCACUGGGCGGCAGCAUAG | 486 | (TGAG) | 20 (C2/1) | VQR-SpBE3 |
| | | TGA | GUCCCACUGGGCGGCAGCAU | 487 | (AGTG) | 20 (C4/3) | VQR-SpBE3 |
| | | (Opal) | CAAAGUCCCACUGGGCGGCAG | 488 | (CATAGT) | 21 (C8/9) | KKH-SaBE3 |
| Q102X | CAA | TAA | CAAUGUGUCAACUCUUGACA | 489 | (GGG) | 20 (C9) | SpBE3 |
| | | (Ochre) | ACAAUGUGUCAACUCUUGAC | 490 | (AGG) | 20 (C10) | SpBE3 |
| Q170X | CAA | TAA | UUUACCAGAUCUCAAAAAGA | 491 | (AGG) | 20 (C13) | SpBE3 |
| | | (Ochre) | | | | | |
| Q186X | CAG | TAG | ACAGUCAGUAUCAAUUCUGG | 492 | (AAG) | 20 (C6) | SpBE3 |
| | | (Amber) | CAUACAGUCAGUAUCAAUUC | 493 | (TGG) | 20 (C9) | SpBE3 |
| | | | AUACAGUCAGUAUCAAUUCU | 494 | (GGAA) | 20 (C8) | VQR-SpBE3 |
| | | | CAGUCAGUAUCAAUUCUGGA | 495 | (AGAA) | 20 (C5) | VQR-SpBE3 |
| | | | ACAGUCAGUAUCAAUUCUGG | 496 | (AAGAAT) | 20 (C6) | SaBE3 |
| Q188X | CAA | TAA | AUCAAUUCUGGAAGAAUUUC | 497 | (CAG) | 20 (C3) | SpBE3 |
| | | (Ochre) | ACAGUCAGUAUCAAUUCUGG | 498 | (AAG) | 20 (C12) | SpBE3 |
| | | | CAGUCAGUAUCAAUUCUGGA | 499 | (AGAA) | 20 (C11) | VQR-SpBE3 |
| | | | UCAAUUCUGGAAGAAUUUCC | 500 | (AGAC) | 20 (C2) | VQR-SpBE3 |
| | | | ACAGUCAGUAUCAAUUCUGG | 501 | (AAGAAT) | 20 (C12) | SaBE3 |
| W190X | TGG | TAG | CAGAAUUGAUACUGACUGUA | 502 | (TGG) | 20 (C1) | SpBE3 |
| | | (Amber) | AAUUCUUCCAGAAUUGAUAC | 503 | (TGA) | 20 (C8/9) | SpBE3 |
| | | or | | | | | |
| | | TGA | | | | | |
| | | (Opal) | | | | | |
| Q194X | CAG | TAG | GAAUUUCCAGACAUUAAAGA | 504 | (TAG) | 20 (C8) | SpBE3 |
| | | (Amber) | GGAAGAAUUUCCAGACAUUA | 505 | (AAG) | 20 (C12) | SpBE3 |
| | | | GAAGAAUUUCCAGACAUUAA | 506 | (AGAT) | 20 (C11) | VQR-SpBE3 |
| | | | UGGAAGAAUUUCCAGACAUU | 507 | (AAAGAT) | 20 (C13) | KKH-SaBE3 |
| | | | AAGAAUUUCCAGACAUUAAA | 508 | (GATAGT) | 20 (C10) | KKH-SaBE3 |
| W248X | TGG | TAG | CCAGAAGAGAAAAUAAACAAU | 509 | (CATGAT) | 21 (C1/−1) | KKH-SaBE3 |
| | | (Amber) | GGAGCCCAGAAGAGAAAAUA | 510 | (AACAAT) | 20 (C7/6) | KKH-SaBE3 |
| | | or | | | | | |
| | | TGA | | | | | |
| | | (Opal) | | | | | |
| Q261X | CAG | TAG | AACACCUUCCAGGAAUUCUU | 511 | (TGG) | 20 (C10) | 20 (C10) |
| | | (Amber) | CUUCCAGGAAUUCUUUGGCC | 512 | (TGAA) | 20 (C5) | 20 (C5) |
| | | | CCUUCCAGGAAUUCUUUGGC | 513 | (CTGAAT) | 20 (C6) | 20 (C6) |
| | | | UCCAGGAAUUCUUUGGCCUG | 514 | (AATAAT) | 20 (C3) | 20 (C3) |
| Q277X | CAA | TAA | GGACCAAGCUAUGCAGGUGA | 515 | (CAG) | 20 (C5) | SpBE3 |
| | | (Ochre) | ACCAAGCUAUGCAGGUGACA | 516 | (GAG) | 20 (C3) | SpBE3 |
| | | | ACAGGUUGGACCAAGCUAUG | 517 | (CAG) | 20 (C12) | SpBE3 |
| | | | CAGGUUGGACCAAGCUAUGC | 518 | (AGG) | 20 (C11) | SpBE3 |
| | | | AGGUUGGACCAAGCUAUGCA | 519 | (GGTG) | 20 (C10) | VQR-SpBE3 |
| | | | GUUGGACCAAGCUAUGCAGG | 520 | (TGAC) | 20 (C8) | VQR-SpBE3 |
| | | | GACCAAGCUAUGCAGGUGAC | 521 | (AGAG) | 20 (C4) | VQR-SpBE3 |
| | | | AACAGGUUGGACCAAGCUAU | 522 | (GCAGGT) | 20 (C13) | KKH-SaBE3 |
| Q280X | CAG | TAG | AUGCAGGUGACAGAGACUCU | 523 | (UGG) | 20 (C4) | SpBE3 |
| | | (Amber) | UGCAGGUGACAGAGACUCUU | 524 | (GGG) | 20 (C3) | SpBE3 |
| | | | GACCAAGCUAUGCAGGUGAC | 525 | (AGAG) | 20 (C13) | VQR-SpBE3 |
| | | | ACCAAGCUAUGCAGGUGACA | 526 | (GAG) | 20 (C12) | SpBE3 |
| | | | CCAAGCUAUGCAGGUGACAG | 527 | (AGAC) | 20 (C11) | VQR-SpBE3 |
| | | | GCAGGUGACAGAGACUCUUG | 528 | (GGAU) | 20 (C2) | VQR-SpBE3 |
| | | | AUGCAGGUGACAGAGACUCU | 529 | (UGGGAU) | 20 (C4) | SaBE3 |
| Q328X | CAG | TAG | UUUUCCAGCAAGAGGCUCCC | 530 | (GAG) | 20 (C6) | VQR-SpBE3 |
| | | (Amber) | AUUUUCCAGCAAGAGGCUCC | 531 | (CGAG) | 20 (C7) | EQR-SpBE3 |
| | | | UUUCCAGCAAGAGGCUCCCG | 532 | (AGCG) | 20 (C5) | VRER-SpBE3 |
| | | | UCCAGCAAGAGGCUCCCGAG | 533 | (CGAG) | 20 (C3) | EQR-SpBE3 |
| | | | CCAGCAAGAGGCUCCCGAGC | 534 | (GAG) | 20 (C2) | EQR-SpBE3 |
| Q329X | CAA | TAA | same as above for Q328X | | | | |
| | | (Ochre) | | | | | |
| R334X | CGA | TGA | GGCUCCCGAGCGAGCAAGCU | 535 | (CAG) | 20 (C13) | SpBE3 |
| | | (Opal) | GAGGCUCCCGAGCGAGCAAG | 536 | (CUCAGU) | 20 (C13) | KKH-SaBE3 |
| | | | GCGAGCAAGCUCAGUUUACA | 537 | (CCCGAU) | 20 (C2) | KKH-SaBE3 |
| R341X | CGA | TGA | GUUUACACCCGAUCCACUGG | 538 | (GGAG) | 20 (C10) | VQR-SpBE3 |
| | | (Opal) | ACACCCGAUCCACUGGGGAG | 539 | (CAG) | 20 (C6) | SpBE3 |
| | | | CACCCGAUCCACUGGGGAGC | 540 | (AGG) | 20 (C5) | SpBE3 |
| | | | ACCCGAUCCACUGGGGAGCA | 541 | (GGAA) | 20 (C4) | VQR-SpBE3 |
| | | | ACCCGAUCCACUGGGGAGCA | 542 | (GGAAAU) | 20 (C4) | KKH-SaBE3 |
| Q346X | CAA | TAA | GGGGAGCAGGAAAUAUCUGU | 543 | (GGG) | 20 (C7) | SpBE3 |
| | | (Ochre) | UGGGGAGCAGGAAAUAUCUG | 544 | (UGG) | 20 (C8) | SpBE3 |
| | | | ACUGGGGAGCAGGAAAUAUC | 545 | (UGUG) | 20 (C10) | VQR-SpBE3 |
| | | | GCAGGAAAUAUCUGUGGGCU | 546 | (UGUG) | 20 (C2) | VQR-SpBE3 |

Base editors: SpBE3-APOBEC1-SpCas9n-UGI; VQR-SpBE3-APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI.

TABLE 6
Examples of genome-editing reactions to
introduce STOP codons todestabilize or prevent
the translation of full-length functional CCR5
protein (FIG.3), mimicking the HIV
protective effect of the CCR5-Δ32 allele.

| | Amino acid | | Genome- | Edited | Stop |
|---|---|---|---|---|---|
| Target | (abbre- | | editing | out- | codon |
| codon | viation) | Method | reaction(s) | come | name |
| CAG | Glutamine | Base- | 1^{st} base | TAG | Amber |
| | (Gln/Q) | editing | C→T coding | | |
| | | | strand | | |
| TGG | Tryptophan | Base- | 2^{nd} base | TAG | Amber |
| | (Trp/W) | editing | C→T on | | |
| | | | comple- | | |
| | | | mentary | | |
| | | | strand | | |
| CGA | Arginine | Base- | 1^{st} base | TGA | Opal |
| | (Arg/R) | editing | C→T | | |
| | | | coding | | |
| | | | strand | | |
| CAA | Glutamine | Base- | 1^{st} base | TAA | Ochre |
| | (Gln/Q) | editing | C→T | | |
| | | | coding | | |
| | | | strand | | |
| TGG | Tryptophan | Base- | 3^{rd} base | UGA | Opal |
| | (Trp/W) | editing | C→T on | | |
| | | | comple- | | |
| | | | mentary | | |
| | | | strand | | |
| CGG | Arginine | Base- | 1^{st} base | TAG | Amber |
| | (Arg/R) | editing | C→T on | | |
| | | | coding | | |
| | | | strand | | |
| | | | and 2^{nd} | | |
| | | | base C→T | | |
| | | | on comple- | | |
| | | | mentary | | |
| | | | strand | | |
| CGA | Arginine | Base- | 1^{st} base | TAA | Ochre |
| | (Arg/R) | editing | C→T on | | |
| | | | coding | | |
| | | | strand | | |
| | | | and 2^{nd} | | |
| | | | base C→T | | |
| | | | on comple- | | |
| | | | mentary | | |
| | | | strand | | |

TABLE 7
Examples of base-editing reactions to alter amino acid
codons in order to produce novel CCR5 variants (FIG. 3).

| Target | Amino acid | Base-editing | Edited | Edited amino acid |
|---|---|---|---|---|
| codon | (abbreviations) | reaction(s) | codon | (abbreviations) |
| CTT | Leucine (Leu/L) | 1^{st} base C→T on | TTT | Phenylalanine (Phe, F) |
| | | coding strand | | |
| CTC | Leucine (Leu/L) | 1^{st} base C→T on | TTC | Phenylalanine (Phe, F) |
| | | coding strand | | |
| ATG | Methionine (Met/M) | 3^{rd} base C→T on | ATA | Isoleucine (Ile, I) |
| | | complementary strand | | |
| GTT | Valine (Val/V) | 1^{st} base C→T on | ATT | Isoleucine (Ile, I) |
| | | complementary strand | | |
| GTC | Valine (Val/V) | 1^{st} base C→T on | ATC | Isoleucine (Ile, I) |
| | | complementary strand | | |
| GTA | Valine (Val/V) | 1^{st} base C→T on | ATA | Isoleucine (Ile, I) |
| | | complementary strand | | |
| GTG | Valine (Val/V) | 1^{st} base C→T on | ATG | Methionine (Met/M) |
| | | complementary strand | | |
| TCT | Serine (Ser/S) | 2^{nd} base C→T on | TTT | Phenylalanine (Phe, F) |
| | | coding strand | | |
| TCC | Serine (Ser/S) | 2^{nd} base C→T on | TTC | Phenylalanine (Phe, F) |
| | | coding strand | | |
| TCA | Serine (Ser/S) | 2^{nd} base C→T on | TTA | Leucine (Leu/L) |
| | | coding strand | | |
| TCG | Serine (Ser/S) | 2^{nd} base C→T on | TTG | Leucine (Leu/L) |
| | | coding strand | | |
| AGT | Serine (Ser/S) | 2^{nd} base C→T on | AAT | Asparagine (Asp/N) |
| | | complementary strand | | |
| AGC | Serine (Ser/S) | 2^{nd} base C→T on | AAC | Asparagine (Asp/N) |
| | | complementary strand | | |
| CCT | Proline (Pro/P) | 1^{st} base C→T on | TCT | Serine (Ser/S) |
| | | coding strand | | |
| CCC | Proline (Pro/P) | 1^{st} base C→T on | TCC | Serine (Ser/S) |
| | | coding strand | | |
| CCA | Proline (Pro/P) | 1^{st} base C→T on | TCA | Serine (Ser/S) |
| | | coding strand | | |
| CCG | Proline (Pro/P) | 1^{st} base C→T on | TCG | Serine (Ser/S) |
| | | coding strand | | |
| CCT | Proline (Pro/P) | 2^{nd} base C→T on | CTT | Leucine (Leu/L) |
| | | coding strand | | |
| CCC | Proline (Pro/P) | 2^{nd} base C→T on | CTC | Leucine (Leu/L) |
| | | coding strand | | |
| CCA | Proline (Pro/P) | 2^{nd} base C→T on | CTA | Leucine (Leu/L) |
| | | coding strand | | |
| CCG | Proline (Pro/P) | 2^{nd} base C→T on | CTG | Leucine (Leu/L) |
| | | coding strand | | |
| ACT | Threonine (Thr/T) | 2^{nd} base C→T on | ATT | Isoleucine (11e/1) |
| | | coding strand | | |
| ACC | Threonine (Thr/T) | 2^{nd} base C→T on | ATC | Isoleucine (11e/1) |
| | | coding strand | | |
| ACA | Threonine (Thr/T) | 2^{nd} base C→T on | ATA | Isoleucine (11e/1) |
| | | coding strand | | |
| ACG | Threonine (Thr/T) | 2^{nd} base C→T on | ATG | Methionine (Met/M) |
| | | coding strand | | |
| GCT | Alanine (Ala/A) | 2^{nd} base C→T on | GTT | Valine (Val/V) |
| | | coding strand | | |
| GCC | Alanine (Ala/A) | 2^{nd} base C→T on | GTC | Valine (Val/V) |
| | | coding strand | | |
| GCA | Alanine (Ala/A) | 2^{nd} base C→T on | GTA | Valine (Val/V) |
| | | coding strand | | |
| GCG | Alanine (Ala/A) | 2^{nd} base C→T on | GTG | Valine (Val/V) |
| | | coding strand | | |
| GCT | Alanine (Ala/A) | 1^{st} base C→T on | ACT | Threonine (Thr/T) |
| | | complementary strand | | |
| GCC | Alanine (Ala/A) | 1^{st} base C→T on | ACC | Threonine (Thr/T) |
| | | complementary strand | | |
| GCA | Alanine (Ala/A) | 1^{st} base C→T on | ACA | Threonine (Thr/T) |
| | | complementary strand | | |
| GCG | Alanine (Ala/A) | 1^{st} base C→T on | ACG | Threonine (Thr/T) |
| | | complementary strand | | |
| CAT | Histidine (His/H) | 1^{st} base C→T on | TAT | Tyrosine (Tyr/Y) |
| | | complementary strand | | |
| CAC | Histidine (His/H) | 1^{st} base C→T on | TAC | Tyrosine (Tyr/Y) |
| | | complementary strand | | |
| GAT | Aspartate (Asp/D) | 1^{st} base C→T on | AAT | Asparagine (Asp/N) |
| | | complementary strand | | |
| GAC | Aspartate (Asp/D) | 1^{st} base C→T on | AAC | Asparagine (Asp/N) |
| | | complementary strand | | |
| GAA | Glutamate (Glu/E) | 1^{st} base C→T on | AAA | Lysine (Lys/K) |
| | | complementary strand | | |
| GAG | Glutamate (Glu/E) | 1^{st} base C→T on | AAG | Lysine (Lys/K) |
| | | complementary strand | | |
| TGT | Cysteine (Cys/C) | 2^{nd} base C→T on | TAT | Tyrosine (Tyr/Y) |
| | | complementary strand | | |
| TGC | Cysteine (Cys/C) | 2^{nd} base C→T on | TAC | Tyrosine (Tyr/Y) |
| | | complementary strand | | |
| CGT | Arginine (Arg/R) | 1^{st} base C→T on | TGT | Cysteine (Cys/C) |
| | | coding strand | | |
| CGC | Arginine (Arg/R) | 1^{st} base C→T on | TGC | Cysteine (Cys/C) |
| | | coding strand | | |
| CGC | Arginine (Arg/R) | 1^{st} base C→T on | TGC | Cysteine (Cys/C) |
| | | coding strand | | |
| AGA | Arginine (Arg/R) | 2^{nd} base C→T on | AAA | Lysine (Lys/K) |
| | | complementary strand | | |
| AGG | Arginine (Arg/R) | 2^{nd} base C→T on | AAG | Lysine (Lys/K) |
| | | complementary strand | | |
| GGT | Glycine (Gly/G) | 2^{nd} base C→T on | GAT | Aspartate (Asp/D) |
| | | complementary strand | | |
| GGC | Glycine (Gly/G) | 2^{nd} base C→T on | GAC | Aspartate (Asp/D) |
| | | complementary strand | | |
| GGA | Glycine (Gly/G) | 2^{nd} base C→T on | GAA | Glutamate (Glu/E) |
| | | complementary strand | | |
| GGG | Glycine (Gly/G) | 2^{nd} base C→T on | GAG | Glutamate (Glu/E) |
| | | complementary strand | | |
| GGT | Glycine (Gly/G) | 1^{st} base C→T on | AGT | Serine (Ser/S) |
| | | complementary strand | | |
| GGC | Glycine (Gly/G) | 1^{st} base C→T on | AGC | Serine (Ser/S) |
| | | complementary strand | | |
| GGA | Glycine (Gly/G) | 1^{st} base C→T on | AGA | Arginine (Arg/R) |
| | | complementary strand | | |
| GGG | Glycine (Gly/G) | 1^{st} base C→T on | AGG | Arginine (Arg/R) |
| | | complementary strand | | |

TABLE 8
Examples of specific guide RNA sequences used for making variants.
The sequences, from top to bottom, correspond to SEQ ID NOs: 547-636.

| CCR5 | | guide RNA | | C | | | | | | | M.- | Hous | Prox/ | Off- |
|---|---|---|---|---|---|---|---|---|---|---|---|---|---|---|
| variant | Cas9-BE^{a} | sequence | PAM | target | Eff.^{b} | Hsu^{c} | Fusi | Chari | Doench | Wang | M. | den | GC | targets^{d} |
| P332S/L | KKH-SaBE3 | GAGGCUCCCG | (CTCAGT) | C7/C8 | 4.9 | 97 | — | 85 | 38 | 80 | 92 | 4 | − | 0-0-0- |
| | | AGCGAGCAAG | | | | | | | | | | | | 1-8 |
| R334X | KKH-SaBE3 | GAGGCUCCCG | (CTCAGT) | C13 | 4.9 | 97 | — | 85 | 38 | 80 | 92 | 4 | − | 0-0-0- |
| | | AGCGAGCAAG | | | | | | | | | | | | 1-8 |
| W94X | SpBE3 | UCCAAAGUCC | (CAG) | C10/C11 | 7.8 | 82 | 51 | 91 | 69 | 85 | 57 | 7 | +GG | 0-0-0- |
| | | CACUGGGCGG | | | | | | | | | | | | 12-109 |
| C290Y, | SpBE3 | GCAGCAGUGC | (GAG) | C4/C−1 | 7.2 | 46 | 64 | 88 | 87 | 84 | 60 | 7 | − | 0-1-0- |
| C291Y | | GUCAUCCCAA | | | | | | | | | | | | 8-88 |
| P19S/L | VQR-SpBE3 | GAGCCCUGCC | (TGTG) | C5/C6 | 6.2 | 100 | — | 85 | 49 | 83 | 41 | 6 | − | 0-0-0- |
| | | AAAAAAUCAA | | | | | | | | | | | | 0-2 |
| W94X | KKH-SaBE3 | CAAAGUCCCAC | (CATAGT) | C8/C9 | 5.0 | 98 | — | 65 | 19 | 76 | 76 | 7 | + | 0-0-0- |
| | | UGGGCGGCAG | | | | | | | | | | | | 1-19 |
| Q328X, | VRER-SpBE3 | UUUCCAGCAA | (AGCG) | C5/C8 | 5.5 | 95 | — | 96 | 38 | 73 | 53 | 5 | + | 0-0-0- |
| Q329X | | GAGGCUCCCG | | | | | | | | | | | | 2-6 |
| Q188X | SaBE3 | ACAGUCAGUAU | (AAGAAT) | C12 | 4.5 | 92 | — | 84 | 39 | 87 | 36 | 4 | −GG | 0-0-0- |
| | | CAAUUCUGG | | | | | | | | | | | | 2-31 |
| G115R/E | KKH-SaBE3 | GAUUCCAGAG | (TAAAAT) | C4/C5 | 5.4 | 87 | — | 95 | 44 | 78 | 45 | 5 | − | 0-0-0- |
| | | AAGAAGCCUA | | | | | | | | | | | | 4-46 |
| P19S/L | KKH-SaBE3 | UAUACAUCGG | (AAAAAT) | C13 | 4.8 | 97 | — | 36 | 30 | 78 | 48 | 4 | + | 0-0-0- |
| | | AGCCCUGCCA | | | | | | | | | | | | 1-9 |
| A335V | VQR-SpBE3 | GAGCAAGCUC | (CGAT) | C4 | 7.8 | 82 | — | 10 | 53 | 70 | 39 | 7 | − | 0-0-0- |
| | | AGUUUACACC | | | | | | | | | | | | 8-88 |
| R341X | VQR-SpBE3 | GUUUACACCC | (GGAG) | C10 | 6.8 | 91 | — | 87 | 30 | 83 | 37 | 6 | +GG | 0-0-0- |
| | | GAUCCACUGG | | | | | | | | | | | | 2-31 |
| Q277X | VQR-SpBE3 | AGGUUGGACC | (GGTG) | C10 | 7.6 | 99 | — | 67 | 27 | 71 | 35 | 7 | − | 0-0-0- |
| | | AAGCUAUGCA | | | | | | | | | | | | 1-5 |
| E283K | KKH-SaBE3 | AGUCUCUGUC | (CTTGGT) | C6 | 9.0 | 91 | — | 81 | 39 | 62 | 40 | 9 | − | 0-0-0- |
| | | ACCUGCAUAG | | | | | | | | | | | | 6-42 |
| G44D/S | SaBE3 | AAAACCAAAG | (GTGAGT) | C5/C6 | 4.6 | 44 | — | 94 | 54 | 86 | 45 | 4 | − | 1-0-0- |
| | | AUGAACACCA | | | | | | | | | | | | 13-190 |
| G163R/E | VQR-SpBE3 | GUAAAGAUGA | (AGAC) | C13 | 4.9 | 41 | — | 70 | 51 | 84 | 50 | 4 | + | 0-1-2- |
| | | UUCCUGGGAG | | | | | | | | | | | | 37-211 |
| Q186X | SpBE3 | ACAGUCAGUA | (AAG) | C6 | 4.5 | 62 | 66 | 84 | 37 | 87 | 36 | 4 | −GG | 0-0-2- |
| | | UCAAUUCUGG | | | | | | | | | | | | 25-95 |
| W248X | KKH-SaBE3 | GGAGCCCAGA | (AACAAT) | C7/6 | 5.2 | 82 | — | 86 | 12 | 77 | 48 | 5 | − | 0-0-1- |
| | | AGAGAAAAUA | | | | | | | | | | | | 3-95 |
| G47S/D | VQR-SpBE3 | CAUGUUGCCC | (AGAT) | C8/C9 | 7.1 | 39 | — | 65 | 41 | 81 | 58 | 7 | − | 1-0-0- |
| | | ACAAAACCAA | | | | | | | | | | | | 17-207 |
| Q277X | SpBE3 | ACAGGUUGGA | (CAG) | C12 | 5.5 | 81 | 68 | 95 | 21 | 47 | 69 | 5 | − | 0-0-0- |
| | | CCAAGCUAUG | | | | | | | | | | | | 11-78 |
| Q277X | KKM-SaBE3 | AACAGGUUGG | (GCAGGT) | C13 | 5.6 | 95 | — | 18 | 17 | 46 | 54 | 5 | − | 0-0-0- |
| | | ACCAAGCUAU | | | | | | | | | | | | 3-15 |
| P183S/L | KKH-SaBE3 | UCAUUUUCCA | (ATCAAT) | C8 | 3.7 | 89 | — | 42 | 43 | 52 | 28 | 3 | − | 0-0-0- |
| | | UACAGUCACU | | | | | | | | | | | | 9-53 |
| G202R/E | KKH-SaBE3 | UCAUUUUCCA | (ATCAAT) | C6/C7 | 3.7 | 89 | — | 42 | 43 | 52 | 28 | 3 | − | 0-0-0- |
| | | UACAGUCAGU | | | | | | | | | | | | 9-53 |
| R334X | KKH-SaBE3 | GCGAGCAAGC | (CCCGAT) | C2 | 7.2 | 95 | — | 60 | 29 | 41 | 46 | 7 | − | 0-0-0- |
| | | UCAGUUUACA | | | | | | | | | | | | 1-14 |
| S149N | KKH-SaBE3 | AUCACACUUG | (AAAGGT) | C7 | 4.7 | 90 | — | 53 | 1 | 60 | 58 | 4 | + | 0-0-0- |
| | | UCACCACCCC | | | | | | | | | | | | 4-36 |
| C20Y | KKH-SaBE3 | UUGGCAGGGC | (AATAAT) | C5 | 7.5 | 99 | — | 8 | 2 | 36 | 71 | 7 | − | 0-0-0- |
| | | UCCGAUGUAU | | | | | | | | | | | | 1-6 |
| Q4X | VQR-SpBE3 | CAAGUGUCAA | (TGAC) | C1 | 3.5 | 81 | — | 23 | 5 | 72 | 48 | 3 | − | 0-0-1- |
| | | GUCCAAUCUA | | | | | | | | | | | | 9-139 |
| C17RY | SpBE3 | UGAGAGCUGC | (AAG) | C10/C11 | 10.1 | 70 | 58 | 85 | 31 | 66 | 38 | 10 | − | 0-0-0- |
| | | AGGUGUAAUG | | | | | | | | | | | | 26-226 |
| P332S/L | SpBE3 | CAAGAGGCUC | (AAG) | C10/C11 | 6.8 | 87 | 47 | 60 | 3 | 77 | 35 | 6 | + | 0-0-0- |
| | | CCGAGCGAGC | | | | | | | | | | | | 4-100 |
| Q93X | KKH-SaBE3 | GCCCAGUGGG | (TACAAT) | C4 | 6.0 | 92 | — | 28 | 11 | 59 | 39 | 6 | − | 0-0-0- |
| | | ACUUUGGAAA | | | | | | | | | | | | 6-38 |
| C20Y | KKH-SaBE3 | CACCCCUCCC | (ATTCAT) | Cl | 6.9 | 96 | — | 9 | 8 | 41 | 55 | 6 | − | 0-0-0- |
| | | AUCUAUAAUA | | | | | | | | | | | | 1-15 |
| D2N | VRER-SpBE3 | UAAUCCAUCU | (TGTG) | C5 | 5.6 | 99 | — | 35 | 12 | 57 | 30 | 5 | + | 0-0-0- |
| | | UGUUCCACCC | | | | | | | | | | | | 0-3 |
| P332S/L | SpBE3 | GGCUCCCGAG | (CAG) | C5/C6 | 4.2 | 88 | 43 | 68 | 9 | 47 | 50 | 4 | − | 0-0-0- |
| | | CGAGCAAGCU | | | | | | | | | | | | 4-61 |
| R334X | SpBE3 | GGCUCCCGAG | (CAG) | C13 | 4.2 | 88 | 43 | 68 | 9 | 47 | 50 | 4 | − | 0-0-0- |
| | | CGAGCAAGCU | | | | | | | | | | | | 4-61 |
| G216S/D | VQR-SpBE3 | UAGGAUUCCC | (TGAC) | C8/C9 | 7.7 | 45 | — | 48 | 27 | 76 | 46 | 7 | − | 0-1-0- |
| | | GAGUAGCAGA | | | | | | | | | | | | 5-99 |
| W86X | VQR-SpBE3 | GAGCCCAGAA | (AGAA) | C5/C6 | 5.2 | 54 | — | 93 | 8 | 63 | 68 | 4 | − | 0-0-2- |
| | | GGGGACAGUA | | | | | | | | | | | | 29-348 |
| C290Y, | SaBE3 | AUGCAGCAGU | (AAGAGT) | C4/C7 | 8.2 | 65 | — | 65 | 8 | 57 | 62 | 8 | + | 0-1-0- |
| C291Y | | GCGUCAUCCC | | | | | | | | | | | | 2-26 |
| S149N | VQR-SpBE3 | ACUUGUCACC | (TGAC) | C2 | 7.4 | 40 | — | 95 | 23 | 78 | 51 | 7 | −GG | 1-0-0- |
| | | ACCCCAAAGG | | | | | | | | | | | | 10-148 |
| C269Y | VQR-SpBE3 | ACUGCAAUUA | (AGA) | C5 | 5.3 | 58 | — | 70 | 7 | 61 | 65 | 5 | + | 0-0-2- |
| | | UUCAGGCCAA | | | | | | | | | | | | 26-277 |
| D2N | KKG-SaBE3 | CCAUCUUGUU | (ATAAAT) | C-l | 4.2 | 94 | — | 19 | 7 | 57 | 30 | 6 | + | 0-0-0- |
| | | CCACCCUGUGC | | | | | | | | | | | | 3-29 |
| C178Y | VQR-SpBE3 | GAGAGCUGCA | (AGA) | C9 | 3.2 | 70 | — | 53 | 6 | 76 | 36 | 3 | − | 0-0-0- |
| | | GGUGUAAUGA | | | | | | | | | | | | 22-251 |
| S149N | SpBE3 | CACACUUGUC | (AGG) | C5 | 6.2 | 39 | 64 | 87 | 21 | 65 | 63 | 6 | + | 1-0-0- |
| | | ACCACCCCAA | | | | | | | | | | | | 22-147 |
| P19S/L | KKH-SaBE3 | CAUCGGAGCC | (ATCAAT) | C9/10 | 7.4 | 93 | — | 66 | 15 | 38 | 40 | 7 | − | 0-0-0- |
| | | CUCCCAAAAA | | | | | | | | | | | | 0-19 |
| Q261X | KKH-SaBE3 | UCCAGGAAUU | (AATAAT) | C3 | 6.5 | 88 | — | 79 | 8 | 41 | 49 | 6 | + | 0-0-0- |
| | | CUUUGGCCUG | | | | | | | | | | | | 3-59 |
| C290Y, | SpBE3 | AUGCAGCAGU | (AAG) | C4/C7 | 8.2 | 59 | 50 | 65 | 7 | 57 | 62 | 8 | + | 0-1-1- |
| C291Y | | GCGUCAUCCC | | | | | | | | | | | | 5-83 |
| Q93X | KKH-SaBE3 | CUGCCGCCCA | (GGAAAT) | C9 | 6.2 | 96 | 40 | 16 | 3 | 18 | 67 | 6 | − | 0-0-0- |
| | | GUGGGACUUU | | | | | | | | | | | | 0-16 |
| C269Y | SaBEJ | UACUGCAAUU | (AAGAAT) | C6 | 4.4 | 93 | — | 15 | 11 | 57 | 22 | 4 | + | 0-0-0- |
| | | AUUCAGGCCA | | | | | | | | | | | | 4-38 |
| P206S/L | KKH-SaBE3 | CUGGUCCUGC | (CATGGT) | C10/C11 | 9.7 | 88 | — | 22 | 6 | 29 | 60 | 9 | − | 0-0-1- |
| | | CGCUGCUUGU | | | | | | | | | | | | 3-48 |
| G47S/I) | VQR-SpBE3 | GUUGCCCACA | (TGAA) | C5/C6 | 5.1 | 37 | — | 94 | 24 | 88 | 32 | 5 | − | 1-1-1- |
| | | AAACCAAAGA | | | | | | | | | | | | 15-198 |
| G47S/D | KKH-SaBE3 | AGCAUGUUGC | (AAAGAT) | C10/C11 | 6.2 | 90 | — | 27 | 13 | 56 | 21 | 6 | − | 0-0-0- |
| | | CCACAAAACC | | | | | | | | | | | | 6-36 |
| Q93X | SpBE3 | GCUGCCGCCC | (TGG) | C10 | 7.4 | 70 | 42 | 32 | 3 | 56 | 51 | 7 | − | 0-0-2- |
| | | AGUGGGACUU | | | | | | | | | | | | 13-126 |
| R341X | KKH-SaBE3 | ACCCGAUCCA | (GGAAAT) | C4 | 4.4 | 94 | 55 | 28 | 7 | 26 | 51 | 4 | + | 0-0-0- |
| | | CUGGGGAGCA | | | | | | | | | | | | 1-18 |
| P34S/L, | SpBE3 | CCUGCCUCCG | (TGG) | C5-C9 | 6.3 | 55 | 47 | 25 | 17 | 47 | 56 | 6 | − | 0-1-0- |
| P35S/L | | CUCUACUCAC | | | | | | | | | | | | 26-175 |
| G216S/ | VQR-SpBE3 | UCCCGAGUAG | (TGAC) | C2/C3 | 3.9 | 46 | — | 68 | 31 | 53 | 44 | 3 | − | 1-0-0- |
| I) | | CAGAUGACCA | | | | | | | | | | | | 4-60 |
| Splice | VQR-SpBE3 | UGCACAGGCU | (GGAT) | C5 | 5.5 | 65 | — | 47 | 8 | 29 | 71 | 5 | − | 0-0-1- |
| site | | GGAACAACAU | | | | | | | | | | | | 15-253 |
| Splice | SpBE3 | AUGCACAGGG | (TGG) | C6 | 4.9 | 42 | 55 | 71 | 11 | 65 | 55 | 4 | − | 0-0-1- |
| site | | UGGAACAAGA | | | | | | | | | | | | 43-421 |
| Splice | KKH-SaBE3 | UUCCACCCUG | (AAAAGT) | C7/8 | 3.1 | 93 | — | 31 | 11 | 24 | 43 | 3 | − | 0-0-0- |
| site | | UGCAUAAAUA | | | | | | | | | | | | 43-421 |
| Splice | VQR-SpBE3 | CCCUGUGCAU | (TGA) | C3/2 | 6.3 | 36 | — | 72 | 44 | 67 | 21 | 6 | − | 0-1-3- |
| site | | AAAUAAAAAG | | | | | | | | | | | | 40-394 |
| Q277X | SpBE3 | CAGGUUGGAC | (AGG) | C11 | 6.1 | 79 | 47 | 44 | 4 | 50 | 33 | 6 | − | 0-0-2- |
| | | CAAGCUAUGC | | | | | | | | | | | | 9-77 |
| Q93X | VQR-SpBE3 | CUGCCGCCCA | (GGAA) | C9 | 6.2 | 78 | — | 16 | 3 | 18 | 67 | 6 | − | 0-0-1- |
| | | GUGGGACUUU | | | | | | | | | | | | 4-88 |
| R223Q | SaBE3 | CGAAGCAGAGU | (CCGAGT) | C-2 | 6.5 | 86 | — | 4 | 2 | 19 | 57 | 4 | − | 0-0-1- |
| | | UUUUAGGAUUC | | | | | | | | | | | | 9-74 |
| G44D/S | KKH-SaBE3 | CCACAAAACC | (ACCAGT) | C9/C10 | 6.1 | 85 | — | 39 | 9 | 54 | 16 | 6 | − | 0-0-1- |
| | | AAAGAUGAAC | | | | | | | | | | | | 6-77 |
| P206S/L | SpBE3 | GGUCCUGCCG | (TGG) | C8/C9 | 4.9 | 65 | 46 | 14 | 4 | 62 | 33 | 4 | − | 0-0-3- |
| | | CUGCUUGUCA | | | | | | | | | | | | 18-149 |
| P34S/L, | KKH-SaBE3 | CUCCUGCCUC | (ACTGGT) | C3-C8 | 7.2 | 93 | — | 12 | 3 | 29 | 37 | 7 | − | 0-0-0- |
| P35S/L | | CGCUCUACUC | | | | | | | | | | | | 6-47 |
| W94X | VQR-SpBE3 | CCCACUGCCC | (TGAG) | C2/1 | 7.3 | 85 | — | 94 | 1 | 42 | 31 | 7 | − | 0-0-0- |
| | | GGCAGCAUAG | | | | | | | | | | | | 7-98 |
| Splice | SpBE3 | AUUUAUGCAC | (AAG) | C10 | 7.7 | 61 | 39 | 11 | 13 | 41 | 44 | 7 | − | 0-0-3- |
| site | | AGGGUGGAAC | | | | | | | | | | | | 11-172 |
| Splice | VQR-SpBE3 | UUUAUGCACA | (AGAT) | C9 | 6.9 | 58 | — | 82 | 9 | 45 | 46 | 6 | − | 0-0-2- |
| site | | GGGUGGAACA | | | | | | | | | | | | 18-283 |
| C290Y, | VQR-SpBE3 | UGCAGCAGUGC | (AGAG) | C6/C3 | 7.2 | 59 | — | 51 | 7 | 40 | 51 | 3 | − | 0-1-0- |
| C291Y | | GUCAUCCCA | | | | | | | | | | | | 7-73 |
| W190X | SpBE3 | CAGAAUUGAU | (TGG) | C1 | 3.7 | 73 | 42 | 41 | 3 | 32 | 48 | 3 | − | 0-0-1- |
| | | ACUGACUGUA | | | | | | | | | | | | 14-140 |
| Q102X | SpBE3 | ACAAUGUGUC | (AGG) | C10 | 8.3 | 77 | 50 | 55 | 9 | 48 | 21 | 8 | − | 0-0-1- |
| | | AACUCUUGAC | | | | | | | | | | | | 7-96 |
| Q21X | SaBE3 | UGCCAAAAAA | (GCAAAT) | C4 | 3.7 | 75 | — | 48 | 22 | 24 | 30 | 3 | − | 0-0-0- |
| | | UCAAUGUGAA | | | | | | | | | | | | 18-172 |
| Splice | WT SpCas9 | GAGGGCAACU | (TAG) | n/a | 6.5 | 69 | 40 | 51 | 2 | 69 | 10 | 6 | − | 0-0-0- |
| site | | AAAUACAUUC | | | | | | | | | | | | 15-134 |
| Q280X | SaBE3 | AUCCACCUCA | (TCCCAT) | C4 | 6.4 | 47 | 49 | 48 | 3 | 54 | 43 | 6 | − | 0-1-0- |
| | | CAGAGACUCU | | | | | | | | | | | | 6-55 |
| R34IX | SpBE3 | CACCCGAUCC | (AGG) | C5 | 6.9 | 68 | 45 | 32 | 1 | 42 | 34 | 6 | + | 0-0-1- |
| | | ACUGGGGAGC | | | | | | | | | | | | 17-100 |
| R223Q | VQR-SpBE3 | ACACCGAAGC | (GGA) | C5 | 7.1 | 63 | — | 47 | 21 | 30 | 30 | 7 | − | 0-0-3- |
| | | AGAGUUUUUA | | | | | | | | | | | | 10-160 |
| P162S/L | KKH-SaBE3 | UCUCCCAGCA | (CCAGAT) | C5/C6 | 6 | 91 | — | 56 | 1 | 23 | 24 | 6 | − | 0-0-0- |
| | | AUCAUCUUUA | | | | | | | | | | | | 1-50 |
| Q261X | VQR-SpBE3 | CUUCCAGGAA | (TGAA) | C5 | 6.3 | 56 | — | 10 | 7 | 49 | 27 | 6 | + | 0-1-2- |
| | | UUCUUUGGCC | | | | | | | | | | | | 20-207 |
| Splice | WT SpCas9 | AGGGCAACUA | (AGG) | n/a | 5.3 | 40 | 40 | 14 | 2 | 56 | 40 | 5 | − | 0-0-6- |
| site | | AAUACAUUCU | | | | | | | | | | | | 39-217 |
| W190X | SpBE3 | AAUUCUUCCA | (TGA) | C8/9 | 5.6 | 61 | — | 14 | 12 | 49 | 11 | 5 | − | 0-0-0- |
| | | GAAUUGAUAC | | | | | | | | | | | | 34-335 |
| P162S/L | VQR-SpBE3 | UCCCAGGAAU | (AGAT) | C3/C4 | 3.3 | 74 | — | 35 | 10 | 21 | 25 | 3 | − | 0-0-2- |
| | | CAUCUUUACC | | | | | | | | | | | | 16-168 |
| Q328X, | EQR-SpBE3 | AUUUUCCAGC | (CGAG) | C7/C10 | 9.8 | 54 | — | 56 | 5 | 39 | 32 | 9 | + | 0-0-3- |
| Q329X | | AAGAGGCUCC | | | | | | | | | | | | 18-484 |
| Splice | WT SpCas9 | AAACUGUUUU | (AGG) | n/a | 4.4 | 49 | 36 | 5 | 6 | 48 | 25 | 4 | − | 0-0-7- |
| site | | AUACAUCAAU | | | | | | | | | | | | 33-312 |
| R223Q | SaBE3 | CGACACCGAA | (TAGGAT) | C7 | 4.7 | 77 | — | 21 | 3 | 14 | 33 | 4 | − | 0-1-0- |
| | | GCAGAGUUUU | | | | | | | | | | | | 0-11 |
| Q261X | SaBE3 | CCUUCCAGGA | (CTGAAT) | C6 | 6.1 | 61 | — | 16 | 10 | 38 | 14 | 6 | − | 0-1-2- |
| | | AUUCUUUGGC | | | | | | | | | | | | 2-54 |
| G145R/E | SpBE3 | CACCCCAAAG | (TGG) | C5/C6 | 5.4 | 48 | 51 | 32 | 0 | 29 | 44 | 5 | + | 1-0-0- |
| | | GUGACCGUCC | | | | | | | | | | | | 3-71 |
| R223Q | SpBE3 | CGACACCGAA | (TAG) | C7 | 4.7 | 68 | 14 | 21 | 2 | 14 | 33 | 4 | − | 0-1-0- |
| | | GCAGAGUUUU | | | | | | | | | | | | 4-43 |
| P293S/L | SpBE3 | CCCAUCAUCU | (CGG) | C1/C2 | 6.3 | 58 | 44 | 2 | 5 | 18 | 35 | 6 | − | 0-1-2- |
| | | AUGCCUUUGU | | | | | | | | | | | | 23-127 |
| R223Q | SpBE3 | GACACCGAAG | (AGG) | C6 | 5.3 | 70 | 22 | 76 | 3 | 17 | 25 | 5 | − | 0-0-4- |
| | | CAGAGUUUUU | | | | | | | | | | | | 10-92 |
| Q261X | SpBE3 | AACACCUUCC | (TGG) | C10 | 7 | 34 | 31 | 15 | 13 | 41 | 21 | 7 | − | 1-0-3- |
| | | AGGAAUUCUU | | | | | | | | | | | | 29-202 |
| PI83S/L | SpBE3 | CAUACAGUCA | (TGG) | C1/−1 | 7.1 | 41 | 27 | 15 | 5 | 36 | 25 | 7 | − | 0-1-2- |
| | | GUAUCAAUUC | | | | | | | | | | | | 17-133 |

a)Base editors: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI.
b)Efficiency score, based on Housden et al (Science Signaling, 2015, 8(393):rs9), which is herein incorporated by reference in its entirety.
c)Specificity scores based on Hsu et al (Nature biotechnology, 2013, 31(9):827-832), Fusi et al (bioRxiv 021568; doi: http://dx.doi.org/10.1101/021568), Chari et al (Nature Methods, 2015, 12(9):823-6), Doench et al (Nature Biotechnology, 2014, 32(12): 1262-7), Wang et al (Science, 2014, 343(6166):80-4), Moreno-Mateos et al (Nature Methods, 2015, 12(10):982-8), Housden et al (Science Signaling, 2015, 8(393):rs9), and the “Prox/GC” column show's “+” if the proximal 6 bp to the PAM has a GC count >= 4, and GG if the guide ends with GG, based on Farboud et al (Genetics, 2015, 199(4):959-71). Each of the foregoing references is hereby incorporated by reference in its entirety.
d)Number of predicted off-target binding sites in the human genome allowing up to 0, 1,2, 3 or 4 mismatches, respectively shown in the format 0-1-2-3-4. Algorithm used: Haeussler et al. Genome Biol. 2016; 17: 148, which is herein incorporated by reference in its entirety.

TABLE 9
Examples of specific guide RNA sequences used for making variants.
The guide RNA sequences, from top to bottom, correspond to
SEQ ID NOs: 637-657 and the CCR2 sequences, from top to bottom,
correspond to SEQ ID NOs: 658-678.

| | | | | | CCR2 seq. | | | | | | Off- |
|---|---|---|---|---|---|---|---|---|---|---|---|
| CCR5 | | guide RNA | | C | (gRNA | | | | | M. | targets^{c} |
| variant | Cas9-BE | sequence | PAM | target | mismatches) | (m) | Eff.^{a} | Hsu^{b} | Doench | -M. | (corrected) |
| C290Y/ | SpBE3 | GCAGCAGUGC | (GAG) | C5 | GCAGCAGTGA | 1 | 7.2 | 46 | 87 | 60 | 0-0-0- |
| C291Y | | GUCAUCCCAA | | | GTCATCCCAAGAG | | | | | | 8-88 |
| G44D/S | SaBE3 | AAAACCAAAG | (GTGAGT) | C5/C6 | AAAACCAAAGATGA | 0 | 4.6 | 44 | 54 | 45 | 0-0-0- |
| | | AUGAACACCA | | | ACACCAGCGAGT | | | | | | 13-190 |
| G163R/ | VQR-SpBE3 | GUAAAGAUGA | (AGAC) | C13 | GTAAAGATGATTC | 1 | 4.9 | 41 | 51 | 50 | 0-0-2- |
| E | | UUCCUGGGAG | | | CTGGGACAGAC | | | | | | 37-211 |
| G47S/D | VQR-SpBE3 | CAUGUUGCCC | (AGAT) | C8/C9 | CATGTTGCCCACA | 0 | 7.1 | 39 | 41 | 58 | 0-0-0- |
| | | ACAAAACCAA | | | AAACCAAAGAT | | | | | | 17-207 |
| G216S/ | VQR-SpBE3 | UAGGAUUCCC | (TGAC) | C8/C9 | CAGGATTCCCGAG | 1 | 7.7 | 45 | 27 | 46 | 0-0-0- |
| D | | GAGUAGCAGA | | | TAGCAGATGAC | | | | | | 5-99 |
| C290Y/ | SaBE3 | AUGCAGCAGU | (AAGAGT) | C4/C7 | ATGCAGCAGTGA | 1 | 8.2 | 65 | 8 | 62 | 0-0-0- |
| C291Y | | GCGUCAUCCC | | | GTCATCCCAAGAGT | | | | | | 2-26 |
| S149N | VQR-SpBE3 | ACUUGUCACC | (TGAC) | C2 | ACTTGTCACCACC | 0 | 7.4 | 40 | 23 | 51 | 0-0-0- |
| | | ACCCCAAAGG | | | CCAAAGGTGAC | | | | | | 10-148 |
| S149N | SpBE3 | CACACUUGUC | (AGG) | C5 | CACACTTGTCAC | 0 | 6.2 | 39 | 21 | 63 | 0-0-0- |
| | | ACCACCCCAA | | | CACCCCAAAGG | | | | | | 22-147 |
| C290Y/ | SpBE3 | AUGCAGCAGU | (AAG) | C4/C7 | ATGCAGCAGTGA | 1 | 8.2 | 59 | 7 | 62 | 0-0-1- |
| C291Y | | GCGUCAUCCC | | | GTCATCCCAAG | | | | | | 5-83 |
| G47S/D | VQR-SpBE3 | GUUGCCCACA | (TGAA) | C5/C6 | GTTGCCCACAAA | 0 | 5.1 | 37 | 24 | 32 | 0-1-1- |
| | | AAACCAAAGA | | | ACCAAAGATGAA | | | | | | 15-198 |
| P34S/L/ | SpBE3 | CCUGCCUCCG | (TGG) | C5-C9 | CCTGCCTCCGCT | 1 | 6.3 | 55 | 17 | 56 | 0-0-0- |
| P35S/L | | CUCUACUCAC | | | CTACTCGCTGG | | | | | | 26-175 |
| G216S/ | VQR-SpBE3 | UCCCGAGUAG | (TGAC) | C2/C3 | TCCCGAGTAGCA | 0 | 3.9 | 46 | 31 | 44 | 0-0-0- |
| D | | CAGAUGACCA | | | GATGACCATGAC | | | | | | 4-60 |
| C290Y/ | VQR-SpBE3 | UGCAGCAGUG | (AGAG) | C6 | TGCAGCAGTGA | 1 | 7.2 | 59 | 7 | 51 | 0-0-0- |
| C291Y | | CGUCAUCCCA | | | GTCATCCCAAGAG | | | | | | 7-73 |
| Q280X | SaBE3 | AUGCAGGUGA | (TGGGAT) | C4 | ACGCAGGTGACAG | 1 | 6.4 | 47 | 3 | 43 | 0-0-0- |
| | | CAGAGACUCU | | | AGACTCTTGGGAT | | | | | | 6-55 |
| Q261X | VQR-SpBE3 | CUUCCAGGAA | (TGAA) | C5 | CTTCCAGGAATTC | 1 | 6.3 | 56 | 7 | 27 | 0-0-2- |
| | | UUCUUUGGCC | | | TTCGGCCTGAA | | | | | | 20-207 |
| R223Q | SaBE3 | CGACACCGAA | (TAGGAT) | C7 | CGACACCGAAGCA | 1 | 4.7 | 77 | 3 | 33 | 0-0-0- |
| | | GCAGAGUUUU | | | GGGTTTTCAGGAT | | | | | | 0-11 |
| Q261X | SaBE3 | CCUUCCAGGA | (CTGAAT) | C6 | CCTTCCAGGAATT | 1 | 6.1 | 61 | 10 | 14 | 0-0-2- |
| | | AUUCUUUGGC | | | CTTCGGCCTGAGT | | | | | | 2-54 |
| G145R/ | SpBE3 | CACCCCAAAG | (TGG) | C5/C6 | CACCCCAAAGGTG | 0 | 5.4 | 48 | 0 | 44 | 0-0-0- |
| E | | GUGACCGUCC | | | ACCGTCCTGG | | | | | | 3-71 |
| R223Q | SpBE3 | CGACACCGAA | (TAG) | C7 | CGACACCGAAGC | 1 | 4.7 | 68 | 2 | 33 | 0-0-0- |
| | | GCAGAGUUUU | | | AGGGTTTTCAG | | | | | | 4-43 |
| P293S/ | SpBE3 | CCCAUCAUCU | (CGG) | C1/C2 | CCCATCATCTATG | 1 | 6.3 | 58 | 5 | 35 | 0-0-2 |
| L | | AUGCCUUUGU | | | CCTTCGTTGG | | | | | | 23-127 |
| Q261X | SpBE3 | AACACCUUCC | (TGG) | C10 | AACACCTTCCAGG | 0 | 7.0 | 34 | 13 | 21 | 0-0-3 |
| | | AGGAAUUCUU | | | AATTCTTCGG | | | | | | 29-202 |

a)Base editors: SpBE3 = APOBEC1-SpCas9n-UGI; VQR-SpBE3 = APOBEC1-VQR-SpCas9n-UGI; EQR-SpBE3 = APOBEC1-EQR-SpCas9n-UGI; VRER-SpBE3 = APOBEC1-VRER-SpCas9n-UGI; SaBE3 = APOBEC1-SaCas9n-UGI; KKH-SaBE3 = APOBEC1-KKH-SaCas9n-UGI.
b)Efficiency score, based on Housden et al (Science Signaling, 2015, 8(393):rs9), which is herein incorporated by reference in its entirety.
c)Specificity scores based on Hsu et al (Nature biotechnology, 2013, 31(9):827-832), Doench et al (Nature Biotechnology, 2014. 32(12): 1262-7), Moreno-Mateos et al (Nature Methods, 2015, 12(10):982-8), each of which is herein incorporated by reference in its entirety.
d)Number of predicted off-target binding sites in the human genome allowing up to 0, 1, 2, 3 or 4 mismatches, respectively shown in the format 0-1-2-3-4. These numbers were corrected to the CCR2 gene as an off-target, therefore, the specificity scores are expected to be higher. Algorithm used: Haeussler et al. Genome Biol. 2016; 17: 148, which is herein incorporated by reference in its entirety.

### REFERENCES

1. Komor, A. C.; Kim, Y. B.; Packer, M. S.; Zuris, J. A.; Liu, D. R., Programmable editing of a target base in genomic DNA without double-stranded DNA cleavage. Nature 2016, advance online publication.
2. (a) Cong. L.; Ran, F. A.; Cox, D.; Lin, S.; Barretto, R.; Habib. N.; Hsu. P. D.; Wu, X.; Jiang. W.; Marraffini, L. A.; Zhang, F., Multiplex genome engineering using CRISPR/Cas systems. Science 2013, 339 (6121), 819-23; (b) Jinek, M.; Chylinski, K.; Fonfara, I.; Hauer. M.; Doudna, J. A.; Charpentier. E., A programmable dual-RNA-guided DNA endonuclease in adaptive bacterial immunity. Science 2012, 337 (6096). 816-21; (c) Mali. P.; Yang. L.; Esvelt. K. M.; Aach, J.; Guell, M.; DiCarlo, J. E.; Norville, J. E.; Church, G. M., RNA-guided human genome engineering via Cas9. Science 2013, 339 (6121). 823-6.
3. Ran, F. A.; Hsu, P. D.; Lin, C. Y.; Gootenberg, J. S.; Konermann, S.; Trevino, A. E.; Scott. D. A.; Inoue, A.; Matoba, S.; Zhang, Y.; Zhang, F., Double nicking by RNA-guided CRISPR Cas9 for enhanced genome editing specificity. Cell 2013, 154 (6), 1380-9.
4. (a) Guilinger, J. P.; Thompson, D. B.; Liu, D. R., Fusion of catalytically inactive Cas9 to FokI nuclease improves the specificity of genome modification. Nature biotechnology 2014, 32 (6). 577-82; (b) Tsai. S. Q.; Wyvekens, N.; Khayter, C.; Foden, J. A.; Thapar, V.; Reyon. D.; Goodwin, M. J.; Aryee, M. J.; Joung, J. K., Dimeric CRISPR RNA-guided FokI nucleases for highly specific genome editing. Nature biotechnology 2014, 32 (6), 569-76.
5. (a) Capoulade-Metay, C.; Ma, L.; Truong, L. X.; Dudoit, Y.; Versmisse, P.; Nguyen, N. V.; Nguyen, M.; Scott-Algara, D.; Barre-Sinoussi, F.; Debre, P.; Bismuth, G.; Pancino. G.; Theodorou, I., New CCR5 variants associated with reduced HIV coreceptor function in southeast Asia. AIDS 2004, 18 (17), 2243-52; (b) Carrington. M.; Kissner, T.; Gerrard, B.; Ivanov, S.; O'Brien. S. J.; Dean, M., Novel alleles of the chemokine-receptor gene CCR5. American journal of human genetics 1997, 61 (6), 1261-7; (c) Barmania. F.; Pepper, M. S., C-C chemokine receptor type five (CCR5): An emerging target for the control of HIV infection. Applied & Translational Genomics 2013, 2, 3-16; (d) Cox, D. B.; Platt, R. J.; Zhang, F., Therapeutic genome editing: prospects and challenges. Nature medicine 2015, 21 (2), 121-31; (e) Dean, M.; Carrington, M.; Winkler, C.; Huttley, G. A.; Smith, M. W.; Allikmets, R.; Goedert, J. J.; Buchbinder, S. P.; Vittinghoff, E.; Gomperts. E.; Donfield. S.; Vlahov, D.; Kaslow, R.; Saah, A.; Rinaldo, C.; Detels, R.; O'Brien, S. J., Genetic restriction of HIV-1 infection and progression to AIDS by a deletion allele of the CKR5 structural gene. Hemophilia Growth and Development Study, Multicenter AIDS Cohort Study. Multicenter Hemophilia Cohort Study, San Francisco City Cohort. ALIVE Study. Science 19%, 273 (5283), 1856-62.
6. (a) Lee. B.; Doranz. B. J.; Rana. S.; Yi, Y.; Mellado. M.; Frade, J. M.; Martinez, A. C.; O'Brien, S. J.; Dean, M.; Coltman. R. G.; Doms, R. W., Influence of the CCR2-V64I polymorphism on human immunodeficiency virus type 1 coreceptor activity and on chemokine receptor function of CCR2b, CCR3, CCR5, and CXCR4. Journal of virology 1998, 72 (9). 7450-8; (b) Apostolakis, S.; Baritaki, S.; Krambovitis, E.; Spandidos, D. A., Distribution of HIV/AIDS protective SDF1, CCR5 and CCR2 gene variants within Cretan population. Journal of clinical virology: the official publication of the Pan American Society for Clinical Virology 2005, 34 (4), 310-4; (c) Nakayama. E. E.; Tanaka, Y.; Nagai, Y.; Iwamoto, A.; Shioda, T., A CCR2-V64I polymorphism affects stability of CCR2A isoform. AIDS 2004, 18 (5), 729-38.
7. (a) Cradick, T. J.; Fine, E. J.; Antico, C. J.; Bao, G., CRISPR/Cas9 systems targeting β-globin and CCR5 genes have substantial off-target activity. Nucleic acids research 2013; (b) Holt. N.; Wang. J.; Kim. K.; Friedman, G.; Wang, X.; Taupin, V.; Crooks. G. M.; Kohn, D. B.; Gregory, P. D.; Holmes. M. C.; Cannon, P. M., Human hematopoietic stem/progenitor cells modified by zinc-finger nucleases targeted to CCR5 control HIV-1 in vivo. Nature biotechnology 2010, 28 (8), 839-47.
8. Koonin, E. V.; Novozhilov, A. S., Origin and evolution of the genetic code: the universal enigma. IUBMB life 2009, 61 (2), 99-111.
9. (a) Thomas, M. A.; Weston. B.; Joseph, M.; Wu, W.; Nekrutenko, A.; Tonellato. P. J., Evolutionary dynamics of oncogenes and tumor suppressor genes: higher intensities of purifying selection than other genes. Molecular biology and evolution 2003, 20 (6), 964-8; (b) Iengar, P., An analysis of substitution, deletion and insertion mutations in cancer genes. Nucleic acids research 2012, 40 (14), 6401-13.

All publications, patents, patent applications, publication, and database entries (e.g., sequence database entries) mentioned herein, e.g., in the Background. Summary. Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the instant compositions and methods encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present compositions and methods may be explicitly excluded from any one or more of the claims. Where ranges are given, any value within the range may explicitly be excluded from any one or more of the claims. Any embodiment, element, feature, application, or aspect of the compositions and/or methods of the disclosure can be excluded from any one or more claims. For purposes of brevity, all of the embodiments in which one or more elements, features, purposes, or aspects is excluded are not set forth explicitly herein.

What is claimed is:

1. A method of editing a polynucleotide encoding a C-C chemokine receptor type five (CCR5) protein, the method comprising contacting the CCR5-encoding polynucleotide with:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target cytosine (C) base in the CCR5-encoding polynucleotide;
wherein the contacting results in the deamination of the target C base by the fusion protein, resulting in a cytosine-guanine (C:G) to thymine-adenine pair (T:A) change in the CCR5-encoding polynucleotide.

2. The method of claim 1, wherein the guide nucleotide sequence-programmable DNA binding protein is a nickase.

3. The method of claim 2, wherein the nickase is a Cas9 nickase.

4. The method of claim 3, wherein the Cas9 nickase comprises a mutation corresponding to a D10A mutation or an H840A mutation in SEQ ID NO: 1.

5. The method of claim 4, wherein the Cas9 nickase comprises a mutation corresponding to the D10A mutation in SEQ ID NO: 1.

6. The method of claim 1, wherein the guide nucleotide sequence-programmable DNA binding protein domain is selected from the group consisting of: a nuclease inactive Cas9 (dCas9) domain, a nuclease inactive Cpf1 domain, a nuclease inactive Argonaute domain, and variants and combinations thereof.

7. The method of claim 6, wherein the guide nucleotide sequence-programmable DNA-binding protein domain comprises a nuclease inactive Cas9 (dCas9) domain.

8. The method of claim 7, wherein the amino acid sequence of the dCas9 domain comprises mutations corresponding to D10A and/or H840A mutation(s) in SEQ ID NO: 1.

9. The method of claim 8, wherein the amino acid sequence of the dCas9 domain comprises a mutation corresponding to a D10A mutation in SEQ ID NO: 1, and wherein the dCas9 domain comprises a histidine at the position corresponding to amino acid 840 of SEQ ID NO: 1.

10. The method of claim 6, wherein the guide nucleotide sequence-programmable DNA-binding protein domain comprises a nuclease inactive Cpf1 (dCpf1) domain.

11. The method of claim 10, wherein the dCpf1 domain is from a species of Acidaminococcus or Lachnospiraceae.

12. The method of claim 6, wherein the guide nucleotide sequence-programmable DNA-binding protein domain comprises a nuclease inactive Argonaute (dAgo) domain.

13. The method of claim 12, wherein the dAgo domain is from Natronobacterium gregoryi.

14. The method of any one of claims 1-13, wherein the cytosine deaminase domain comprises an apolipoprotein B mRNA-editing complex (APOBEC) family deaminase.

15. The method of any one of claims 1-14, wherein the cytosine deaminase is selected from the group consisting of APOBEC1, APOBEC2, APOBEC3A, APOBEC3B, APOBEC3C, APOBEC3D, APOBEC3F, APOBEC3G deaminase, APOBEC3H deaminase, APOBEC4 deaminase, activation-induced deaminase (AID), and pmCDA1.

16. The method of any one of claims 1-15, wherein the cytosine deaminase comprises an amino acid sequence of any one of SEQ ID NOs: 1-260, 270-292, or 315-323.

17. The method of any one of claims 1-16, wherein the fusion protein of (a) further comprises a uracil glycosylase inhibitor (UGI) domain.

18. The method of any one of claims 1-17, wherein the cytosine deaminase domain is fused to the N-terminus of the guide nucleotide sequence-programmable DNA-binding protein domain.

19. The method of claim 17 or 18, wherein the UGI domain is fused to the C-terminus of the guide nucleotide sequence-programmable DNA-binding protein domain.

20. The method of any one of claims 1-19, wherein the cytosine deaminase and the guide nucleotide sequence-programmable DNA-binding protein domain are fused via an optional linker.

21. The method of any one of claims 17-20, wherein the UGI domain is fused to the dCas9 domain via an optional linker.

22. The method of claim 21, wherein the fusion protein comprises the structure NH₂-[cytosine deaminase domain]-[optional linker sequence]-[guide nucleotide sequence-programmable DNA-binding protein domain]-[optional linker sequence]-[UGI domain]-COOH.

23. The method of claim 21, wherein the fusion protein comprises the structure NH₂-[UGI domain]-[optional linker sequence]-[cytosine deaminase domain]-[optional linker sequence]-[guide nucleotide sequence-programmable DNA-binding protein domain]-COOH.

24. The method of any one of claims 20-23, wherein the linker comprises (GGGS)ₙ (SEQ ID NO: 303), (GGGGS)ₙ (SEQ ID NO: 304), (G)ₙ, (EAAAK)ₙ (SEQ ID NO: 305), (GGS)ₙ, SGSETPGTSESATPES (SEQ ID NO: 306), or an (XP)ₙ motif, or a combination of any of these, wherein n is independently an integer between 1 and 30, and wherein X is any amino acid.

25. The method of any one of claims 20-23, wherein the linker is unstructured, structured, helical, or extended.

26. The method of claim 24, wherein the linker comprises the amino acid sequence SGSETPGTSESATPES (SEQ ID NO: 306).

27. The method of claim 24, wherein the linker is (GGS)ₙ, and wherein n is 1, 3, or 7.

28. The method of any one of claims 1-27, wherein the fusion protein comprises the amino acid sequence of any one of SEQ ID NO: 293-302.

29. The method of any one of claims 1-28, wherein the fusion protein of (i) further comprises a Gam protein.

30. The method of claim 29, wherein the Gam protein comprises the amino acid sequence of any one of SEQ ID NOs: 710-734.

31. The method of any one of claims 1-30, wherein the polynucleotide encoding the CCR5 protein comprises a coding strand and a complementary strand.

32. The method of any one of claims 1-31, wherein the polynucleotide encoding the CCR5 protein comprises a coding region and a non-coding region.

33. The method of any one of claims 1-32, wherein the C to T change occurs in the coding sequence of the CCR5-encoding polynucleotide.

34. The method of claim 33, wherein the C to T change leads to a mutation in the CCR5 protein.

35. The method of claim 34, wherein the mutation in the CCR5 protein is a loss-of-function mutation.

36. The method of claim 34 or 35, wherein the mutation is selected from the mutations listed in Tables 1-9.

37. The method of any one of claims 1-36, wherein the guide nucleotide sequence is selected from the guide nucleotide sequences listed in Tables 3-5 and 8-9.

38. The method of any one of claims 35-37, wherein the loss-of-function mutation introduces a premature stop codon in the CCR5 coding sequence that leads to a truncated or non-functional CCR5 protein.

39. The method of claim 38, wherein the premature stop codon is TAG (Amber), TGA (Opal), or TAA (Ochre).

40. The method of claim 38 or 39, wherein the premature stop codon is generated from a CAG to TAG change via the deamination of the first C on the coding strand.

41. The method of claim 38 or 39, wherein the premature stop codon is generated from a CGA to TGA change via the deamination of the first C on the coding strand.

42. The method of claim 38 or 39, wherein the premature stop codon is generated from a CAA to TAA change via the deamination of the first C on the coding strand.

43. The method of claim 38 or 39, wherein the premature stop codon is generated from a TGG to TAG change via the deamination of the second C on the complementary strand.

44. The method of claim 38 or 39, wherein the premature stop codon is generated from a TGG to TGA change via the deamination of the third C on the complementary strand.

45. The method of claim 38 or 39, wherein the premature stop codon is generated from a TGG to TAA change via the deamination of the second C and third C on the complementary strand.

46. The method of claim 38 or 39, wherein the premature stop codon is generated from a CGG to TAG or CGA to TAA change via the deamination of C on the coding strand and the deamination of C on the complementary strand.

47. The method of any one of claims 39-46, wherein the guide nucleotide sequence is selected from the guide nucleotide sequences (SEQ ID NO: 471-657) listed in Table 5, Table 8, or Table 9.

48. The method of claim 39, wherein tandem premature stop codons are introduced.

49. The method of claim 48, wherein the mutation is selected from the group consisting of: Q186/Q188, Q277/Q288, Q328/Q329, Q329/R334, or R341/Q346.

50. The method of claim 49, wherein the guide nucleotide sequence is selected from the group consisting of: SEQ ID NOs: 381-657.

51. The method of any one of claims 35-50, wherein the loss-of-function mutation destabilizes CCR5 protein folding.

52. The method of claim 51, wherein the loss-of-function mutation is selected from the mutations listed in Tables 1, 4, 8, or 9.

53. The method of claim 52, wherein the guide nucleotide sequence is selected from the guide nucleotide sequences listed in Tables 3, 4, 8 or 9 (SEQ ID NO: 381-410, 411-470).

54. The method of any one of claims 1-30, wherein the C to T change modifies a splicing site in the non-coding region of the CCR5-encoding polynucleotide.

55. The method of claim 54, wherein the C to T change modifies an intron-exon junction.

56. The method of claim 54, wherein the C to T change modifies a splicing donor site.

57. The method of claim 54, wherein the C to T change modifies a splicing acceptor site.

58. The method of claim 54, wherein the C to T changes occurs at a C base-paired with the G base in a start codon (AUG).

59. The method of any one of claims 54-58, wherein the C to T change prevents CCR5 mRNA maturation or abrogates CCR5 expression.

60. The method of claim 54-59, wherein the C to T change is selected from the C to T changes listed in Table 2, 8 or 9.

61. The method of any one of claims 54-60, wherein the guide nucleotide sequence is selected from the guide nucleotide sequences (SEQ ID NOs: 577-657) listed in Tables 8 and 9.

62. The method of any one of claims 1-30, wherein the C to T change results in a codon change in the CCR5-encoding polynucleotide listed in Table 8 or 9.

63. The method of any one of claims 1-62, wherein a PAM sequence is located 3′ of the C being changed.

64. The method of claim 63, wherein the PAM sequence is selected from the group consisting of: NGG, NGAN, NGNG, NGAG, NGCG, NNGRRT, NGRRN, NNNRRT, NNNGATT, NNAGAA, NAAAC, NNT, NNNT, and YNT, wherein Y is pyrimidine, R is purine, and N is any nucleobase.

65. The method of any one of claims 1-62, wherein no PAM sequence is located 3′ of the C being changed.

66. The method of any of claim 1-65, wherein at least 1, 2, 3, 4, 5, 6, 7, 8, 9, or 10 mutations are introduced into the CCR5-encoding polynucleotide.

67. The method of any one of claims 1-66, wherein the guide nucleotide sequence is RNA (gRNA).

68. The method of any one of claims 1-66, wherein the guide nucleotide sequence is ssDNA (gDNA).

69. A method of editing a polynucleotide encoding a C-C chemokine receptor type 2 (CCR2) protein, the method comprising contacting the CCR2-encoding polynucleotide with:
(i) a fusion protein comprising: (a) a guide nucleotide sequence-programmable DNA binding protein domain; and (b) a cytosine deaminase domain, and
(ii) a guide nucleotide sequence targeting the fusion protein of (i) to a target cytosine (C) base in the CCR2-encoding polynucleotide,
wherein the contacting results in the deamination of the target C base by the fusion protein, resulting in a cytosine-guanine (C:G) to thymine-adenine pair (T:A) change in the CCR2-encoding polynucleotide.

70. The method of claim 69, wherein the C to T change is in the coding sequence of the CCR2-encoding polynucleotide.

71. The method of claim 69 or 70, wherein the C to T change leads to leads to a mutation in the CCR2 protein.

72. The method of claim 71, wherein the mutation in the CCR2 protein is a loss-of-function mutation.

73. The method of claim 71 or 72, wherein the mutation is selected from the mutations listed in Table 1.

74. The method of claims 1-73, wherein the method is carried out in vitro.

75. The method of claim 74, wherein the method is carried out in a cultured cell.

76. The method of any one of claims 1-73, wherein the method is carried out in vivo.

77. The method of any one of claims 1-73, wherein the method is carried out ex vivo.

78. The method of claim 76, wherein the method is carried out in a mammal.

79. The method of claim 76 or 78, wherein the mammal is a rodent.

80. The method of claim 76 or 78, wherein the mammal is human.

81. A method of editing a polynucleotide encoding a C-C chemokine receptor type five (CCR2) protein, the method comprising contacting the CCR2-encoding polynucleotide with:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a target cytosine (C) base in the CCR2-encoding polynucleotide;
wherein the contacting results in the deamination of the target C base by the fusion protein, resulting in a cytosine-guanine (C:G) to thymine-adenine pair (T:A) change in the CCR2-encoding polynucleotide.

82. The method of claim 81, wherein the guide nucleotide sequence-programmable DNA binding protein domain is selected from the group consisting of: a nuclease inactive Cas9 (dCas9) domain, a nuclease inactive Cpf1 domain, a nuclease inactive Argonaute domain, and variants and combinations thereof.

83. The method of claim 81 or 82, wherein the guide nucleotide sequence-programmable DNA-binding protein domain comprises a nuclease inactive Cas9 (dCas9) domain.

84. The method of claim 83, wherein the amino acid sequence of the dCas9 domain comprises mutations corresponding to D10A and/or H840A mutation(s) in SEQ ID NO: 1.

85. The method of claim 84, wherein the amino acid sequence of the dCas9 domain comprises a mutation corresponding to a D10A mutation in SEQ ID NO: 1, and wherein the dCas9 domain comprises a histidine at the position corresponding to amino acid 840 of SEQ ID NO: 1.

86. The method of claim 81 or 82, wherein the guide nucleotide sequence-programmable DNA-binding protein domain comprises a nuclease inactive Cpf1 (dCpf1) domain.

87. The method of claim 86, wherein the dCpf1 domain is from a species of Acidaminococcus or Lachnospiraceae.

88. The method of claim 81 or 82, wherein the guide nucleotide sequence-programmable DNA-binding protein domain comprises a nuclease inactive Argonaute (dAgo) domain.

89. The method of claim 88, wherein the dAgo domain is from Natronobacterium gregoryi.

90. The method of any one of claims 81-89, wherein the cytosine deaminase domain comprises an apolipoprotein B mRNA-editing complex (APOBEC) family deaminase.

91. The method of any one of claims 81-90, wherein the cytosine deaminase is selected from the group consisting of APOBEC1, APOBEC2, APOBEC3A, APOBEC3B, APOBEC3C, APOBEC3D, APOBEC3F, APOBEC3G deaminase, APOBEC3H deaminase, APOBEC4 deaminase, activation-induced deaminase (AID), and pmCDA1.

92. The method of any one of claims 81-91, wherein the cytosine deaminase comprises an amino acid sequence of any one of SEQ ID NOs: 1-260, 270-292, or 315-323.

93. The method of any one of claims 81-92, wherein the fusion protein of (a) further comprises a uracil glycosylase inhibitor (UGI) domain.

94. The method of any one of claims 81-93, wherein the cytosine deaminase domain is fused to the N-terminus of the guide nucleotide sequence-programmable DNA-binding protein domain.

95. The method of claim 93 or 94, wherein the UGI domain is fused to the C-terminus of the guide nucleotide sequence-programmable DNA-binding protein domain.

96. The method of any one of claims 81-95, wherein the cytosine deaminase and the guide nucleotide sequence-programmable DNA-binding protein domain are fused via an optional linker.

97. The method of any one of claims 93-96, wherein the UGI domain is fused to the dCas9 domain via an optional linker.

98. The method of claim 97, wherein the fusion protein comprises the structure NH₂-[cytosine deaminase domain]-[optional linker sequence]-[guide nucleotide sequence-programmable DNA-binding protein domain]-[optional linker sequence]-[UGI domain]-COOH.

99. The method of claim 97, wherein the fusion protein comprises the structure NH₂-[UGI domain]-[optional linker sequence]-[cytosine deaminase domain]-[optional linker sequence]-[guide nucleotide sequence-programmable DNA-binding protein domain]-COOH.

100. The method of any one of claims 98-99, wherein the linker comprises (GGGS)ₙ(SEQ ID NO: 303), (GGGGS)ₙ (SEQ ID NO: 304), (G)ₙ, (EAAAK)ₙ (SEQ ID NO: 305), (GGS)ₙ, SGSETPGTSESATPES (SEQ ID NO: 306), or an (XP)ₙ motif, or a combination of any of these, wherein n is independently an integer between 1 and 30, and wherein X is any amino acid.

101. The method of any one of claims 98-99, wherein the linker is unstructured, structured, helical, or extended.

102. The method of claim 100, wherein the linker comprises the amino acid sequence SGSETPGTSESATPES (SEQ ID NO: 306).

103. The method of claim 100, wherein the linker is (GGS)ₙ, and wherein n is 1, 3, or 7.

104. A composition comprising:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a polynucleotide encoding a C-C chemokine receptor type five (CCR5) protein.

105. A composition comprising:
(i) a fusion protein comprising: (a) a guide nucleotide sequence-programmable DNA binding protein domain; and (b) a cytosine deaminase domain; and
(ii) a guide nucleotide sequence targeting the fusion protein of (i) to a polynucleotide encoding a C-C chemokine receptor type two (CCR2) protein.

106. A composition comprising:
(i) a fusion protein comprising: (a) a guide nucleotide sequence-programmable DNA binding protein domain; and (b) a cytosine deaminase domain;
(ii) a guide nucleotide sequence targeting the fusion protein of (i) to a polynucleotide encoding a C-C chemokine receptor type five (CCR5) protein; and
(iii) a guide nucleotide sequence targeting the fusion protein of (i) to a polynucleotide encoding a C-C chemokine receptor type 2 (CCR2) protein.

107. The method of claim 1, wherein the guide nucleotide sequence-programmable DNA binding protein is a nickase.

108. The method of claim 107, wherein the nickase is a Cas9 nickase.

109. The method of claim 108, wherein the Cas9 nickase comprises a mutation corresponding to a D10A mutation or an H840A mutation in SEQ ID NO: 1.

110. The method of claim 109, wherein the Cas9 nickase comprises a mutation corresponding to the D10A mutation in SEQ ID NO: 1.

111. The composition of any one of claims 104-110, wherein the guide nucleotide sequence of (ii) is selected from SEQ ID NOs: 381-657.

112. The composition of any one of claims 104-111 further comprising a pharmaceutically acceptable carrier.

113. A method of reducing the binding of gp120 and CCR5 in a subject, the method comprising administering to a subject in need thereof a therapeutically effective amount of the composition of any one of claims 104-112.

114. A method of reducing virus binding to CCR5 in a subject, the method comprising administering to a subject in need thereof a therapeutically effective amount of the composition of any one of claims 104-112.

115. A method of reducing viral infection in a subject, the method comprising administering to a subject in need thereof a therapeutically effective amount of the composition of any one of claims 104-112.

116. A method of reducing functional CCR5 receptors on a cell in a subject, the method comprising administering to a subject in need thereof a therapeutically effective amount of the composition of any one of claims 104-112.

117. The method of claim C2, wherein the cell is selected from the group consisting of: macrophage, dendritic cell, memory T cell, endothelial cell, epithelial cell, vascular smooth muscle cell, fibroblast, microglia, neuron, and astrocyte.

118. A method of treating a condition, the method comprising administering to a subject in need thereof a therapeutically effective amount of the composition of any one of claims 104-112, wherein the condition is human immunodeficiency virus (HIV) infection, re-infection, or activation from latency, acquired immune deficiency syndrome (AIDS), an immunologic disease, or a combination thereof.

119. The method of claim 118, wherein the condition is human immunodeficiency virus (HIV) infection.

120. The method of claim 18, wherein the condition is latent human immunodeficiency virus (HIV).

121. The method of claim 118, wherein the condition is a virus that targets CCR5 on white blood cells.

122. A method of preventing a condition, the method comprising administering to a subject in need thereof a therapeutically effective amount of the composition of any one of claims 104-112, wherein the condition is human immunodeficiency virus (HIV) infection, re-infection, or activation from latency, acquired immune deficiency syndrome (AIDS), an immunologic disease, or a combination thereof.

123. The method of claim 122, wherein the condition is human immunodeficiency virus (HIV) infection.

124. The method of claim 122, wherein the condition is latent human immunodeficiency virus (HIV).

125. The method of claim 122, wherein the condition is a virus that targets CCR5 on white blood cells.

126. A kit comprising the composition of any one of claims 104-112.
